(19) United States
(12) Patent Application Publication
(10) Pub. No.: US 2026/0124420 A1
(43) Pub. Date: May 7, 2026

(54) MASK ASSEMBLIES FOR RESPIRATORY THERAPY

(71) Applicant: Fisher & Paykel Healthcare Limited, Auckland (NZ)

(72) Inventors: Mark Richard TOMLINSON, Auckland (NZ); Adam Alexander TEBBUTT, Auckland (NZ); Paul Mathew FREESTONE, Auckland (NZ); Jeroen HAMMER, Auckland (NZ); Tony William SPEAR, Auckland (NZ); Matthew Roger STEPHENSON, Auckland (NZ); Katie O'BRIEN, Auckland (NZ); Xueyan BIAN, Auckland (NZ); Mitesh PATEL, Auckland (NZ); Christopher Jaye Norman AMADIO, Auckland (NZ); Craig Robert PRENTICE, Auckland (NZ); Simon MITTERMEIER, Auckland (NZ); Andrew SHARP, Auckland (NZ)

(21) Appl. No.: 19/130,629

(22) PCT Filed: Nov. 17, 2023
(86) PCT No.: PCT/IB2023/061626

§ 371 Date: May 16, 2025

Related U.S. Application Data
(60) Provisional application No. 63/384,332, filed on Nov. 18, 2022.
(60) Provisional application No. 63/476,743, filed on Dec. 22, 2022.
(60) Provisional application No. 63/500,515, filed on May 5, 2023.

(51) Int. Cl. A61M 16/06; A61M 16/08; A61M 39/00; A61M 39/10

(52) U.S. Cl. CPC A61M 16/0622; A61M 16/0683; A61M 16/0816; A61M 39/10; A61M 2039/0009; A61M 2202/0007; A61M 2202/02

(57) ABSTRACT

A mask assembly is provided comprising a cushion with a proximal face contacting portion, a distal inlet aperture, and a side wall between the proximal face contacting portion and the distal inlet aperture, a frame; and an inlet connector configured to connect to a source of pressurised gas. The frame comprises upper and lower headgear connectors both of which are below the cushion uppermost portion. The frame comprises a central connection portion removably connected to the cushion and the inlet connector. The central connection portion is relatively inflexible, whilst the remainder of the frame may be relatively flexible. The inlet connector comprises an inlet port for connection to a gas delivery conduit, and comprises a downwardly directed central axis.

This application includes subject matter from our earlier international patent application PCT/NZ 2022/050086 filed 1 Jul. 2022, our earlier U.S. patent application U.S. 63/384332 filed 18 Nov. 2022, our earlier U.S. patent application U.S. 63/476743 filed 22 Dec. 2022, and our earlier U.S. patent application U.S. 63/500515 filed 5 May 2023, the entire contents of each of which are herein incorporated by reference in their entirety.

### BACKGROUND

### Field

The present disclosure relates to mask assemblies for respiratory therapy. In particular, the present disclosure relates to full-face mask assemblies that cover the mouth and nose of the user.

### Description of Related Art

Obstructive sleep apnea is commonly treated with the application of positive airway pressure (PAP) therapy. PAP therapy involves delivering a flow of gas to a patient at a therapeutic pressure above atmospheric pressure that will reduce the frequency and/or duration of apneas, hypopneas, and/or flow limitations. The therapy is often implemented by using a positive airway pressure device to deliver a pressurized stream of air through a conduit to a patient through a mask assembly positioned on the face of the patient.

Typical mask assemblies are configured to provide sealed communication with a user's airway by sealing around parts of the user's nose and/or mouth. These mask assemblies are commonly used to provide therapies such as, but not limited to, non-invasive ventilation (NIV) and continuous positive airway pressure (CPAP). CPAP therapy is commonly used to treat obstructive sleep apnea (OSA) and involves providing a constant supply of pressurized air to a user's airway.

Various problems exist in respect of such full-face mask assemblies. One such problem can be that when the user is side sleeping, one or more parts of the mask assembly can contact the bed or pillow, move and deform, and allow inspiratory gas to leak from the mask assembly, reducing the effectiveness of the therapy, and also causing noise and draughts. Further such movement and deformation can be uncomfortable for the patient. Another problem is the noise made by the inspiratory gas flowing into the mask assembly during therapy. Such noise can be annoying to the patient and others around the patient. A general problem in providing a mask assembly is to improve patient adherence in wearing the mask assembly when required, and for as long as required.

A need exists to provide mask assembly with improved comfort and/or sealing performance; and/or to improve on any one or more of the problems mentioned above; and/or to provide the public with a useful choice.

### SUMMARY

The mask assemblies described herein have innovative aspects, no single one of which is indispensable or solely responsible for their desirable attributes. Without limiting the scope of the claims, some of the advantageous features will now be summarized.

According to an aspect of this disclosure there is provided a mask assembly comprising
a cushion with a proximal face contacting portion, a distal inlet aperture, and a side wall between the proximal face contacting portion and the distal inlet aperture, and
a frame comprising an inlet opening configured to connect to a source of pressurised gas,
the distal inlet aperture of the cushion is removably connected to the inlet opening of the frame, and
the frame further comprising a pair of upper and a pair of lower headgear connectors;
the cushion comprising an uppermost portion;
both pairs of upper and lower headgear connectors being below the cushion uppermost portion.

The frame may comprise a central connection portion, configured to be removably connected to the cushion. The inlet opening may be formed in the central connection portion. The frame material at the central connection portion may circumscribe the inlet opening.

The cushion may comprise a lower portion adjacent the inlet aperture, and an upper portion defining the top of the cushion.

The frame may comprise an elongate frame member extending across the upper portion of the cushion.

The elongate member may extend down the side wall.

The lower portion of the frame may be wider than the upper portion, the upper and lower portions being spaced apart.

The lower portion of the frame may comprise lateral arms which extend laterally outwardly from the inlet opening.

The lateral arms may be relatively narrow as compared to the lower portion, the lower portion being relatively wide.

The inlet opening may be provided in the relatively wide, lower portion.

The lower portion of the frame may extend symmetrically about a central axis of the inlet opening of the frame.

The lower portion of the frame may be positioned centrally, when the mask assembly is viewed from the front.

The frame inlet opening may comprise a central axis, the central axis extending generally downwardly from the mask assembly.

The inlet aperture of the cushion may taper from the bottom of the inlet aperture to the top of the inlet aperture.

The inlet aperture of the cushion may comprise a substantially planar base and inwardly inclined sides.

The mask assembly may comprise an inlet connector configured to be connected to the frame at the central connection portion, the inlet connector being configured to be connected to the source of pressurised gas.

The inlet connector, when connected to the central connection portion, may be received in the inlet opening, The cushion may be mounted on the inlet connector and/or the frame so as to receive gas from the inlet connector. The inlet connector may provide the connection between the cushion and the frame.

The inlet connector may form a gas flow path, for the pressurised gas, between the source of pressurised gas and the cushion.

The inlet aperture of the cushion may be configured to be fluidly coupled to a breathing gas delivery conduit via the inlet connector.

The inlet connector may be configured to be positioned between the cushion and the frame.

The inlet connector may comprise an inlet boss configured to receive breathing gas from the gas delivery conduit.

The inlet boss may comprise an inlet port.

The inlet connector may comprise an outlet boss in fluid communication with both the inlet boss and the cushion.

The outlet boss may comprise an outlet port.

The inlet boss may comprise a conduit connector configured to connect with the breathing gas delivery conduit.

The outlet boss may be larger than the inlet boss.

The outlet boss may taper from the bottom of the outlet opening to the top of the outlet opening.

The outlet boss may comprise a substantially planar base and inwardly inclined sides.

The inlet connector may taper from the bottom to the top of the inlet connector.

The inlet connector may comprise a substantially planar base and inwardly inclined sides.

The inlet connector may be integrally formed with the cushion, at the cushion inlet aperture.

The inlet connector may be permanently mounted on the cushion, at the cushion inlet aperture.

The inlet connector may be removably mounted on the cushion, at the cushion inlet aperture.

The inlet connector may be integrally formed with the frame, at the frame inlet opening.

The inlet connector may be permanently mounted on the frame, at the frame inlet opening.

The inlet connector may be removably mounted on the frame, at the frame inlet opening.

The inlet opening may be provided in a planar region of the lower portion the frame, the plane of the planar region being less than 30° from horizontal, or less than 20° from horizontal, or less than 10° from horizontal, or less than 5° from horizontal, or substantially horizontal.

The planar region may comprise a generally planar base of the frame.

The mask assembly may comprise one or more vent holes and/or one or more anti-asphyxia valve openings.

The one or more vent holes and/or one or more anti-asphyxia valve openings may be adjacent the inlet opening of the frame.

The one or more vent holes and/or one or more anti-asphyxia valve openings, may be above the inlet opening of the frame.

The lower portion of the frame may comprise a recess, adjacent the inlet opening, the one or more vent holes and/or one or more anti-asphyxia valve openings being exposed through the recess.

The cushion may comprise a transparent region extending around at least part of the inlet aperture of the cushion, the frame being configured such that the transparent region is exposed.

The face contacting portion may comprise a sealing surface comprising an inner margin, the inner margin comprising a thin flap.

The cushion may be removably mounted on the frame.

The cushion may be permanently mounted on the frame.

The cushion may comprise a region configured to buckle in a single direction to define a roll of material that extends over the mask frame.

The cushion may comprise a distal non-face contacting surface or side, the uppermost portion of the cushion comprising a rolling bridge portion and being pivotable relative to a lower portion of the cushion, the uppermost portion comprising a region of reduced stiffness that is positioned between a first boundary, the first boundary being defined by a stiffness greater than that in the region of reduced stiffness, the second boundary being defined by a stiffness greater than that in the region of reduced stiffness, wherein when the first boundary is moved toward the second boundary, the region of reduced stiffness buckles to define a roll of material that increases in size away from the face-contacting portion as the first boundary continues to move toward the second boundary to facilitate movement of the rolling bridge portion of the cushion relative to the lower portion of the cushion; wherein the mask frame is configured to overlay an exterior portion of the second boundary such that the roll of material extends over/overlaps with the mask frame.

The cushion may comprise an interior surface positioned to be adjacent the nose of the patient in use, the interior surface comprising an internally recessed portion, adjacent the patient's nose. This can assist in providing greater clearance between the interior surface of the cushion and the patient's nose. The recessed region may be located centrally directly below a rolling bridge portion of the cushion.

The frame may be configured to overlay an exterior portion of the side wall;
wherein a lateral dimension is defined between the lower or the upper pair of headgear connectors;
wherein the frame is configured to be laterally compliant so as to resiliently deform such that the lateral dimension can change when subject to lateral forces.

The frame may be configured to overlay an exterior portion of the side wall;
wherein the distal inlet aperture of the cushion forms a connection with the frame by way of being connected to the inlet opening of the frame;
the remainder of the cushion not being connected to the frame.

The frame may be configured to be:
laterally compliant so as to resiliently deform such that the lateral dimension between the headgear connectors can change when subject to lateral forces; and
longitudinally non-compliant in a longitudinal direction between the proximal face contacting portion and the distal non-face contacting portion, so as to resist deformation of the mask frame when subject to longitudinal forces.

The distal inlet aperture of the cushion may be removably connected to the inlet opening of the frame, and the frame is configured to overlap an exterior portion of the side wall.

The frame may comprise:
a) an upper frame portion;
b) opposed side frame portions;
c) a lower frame portion comprising the inlet opening configured to connect to a source of pressurised gas; wherein
a frame aperture is defined between the upper, side and lower frame portions.

The frame aperture may be configured such that the cushion is exposed through the frame aperture.

The mask assembly may be fluidly coupled to a breathing gas delivery conduit via an inlet connector, the inlet connector being configured to be positioned between the cushion and the frame, wherein the inlet connector is exposed through the frame aperture.

The upper, side and lower frame portions may be configured to overlap with and support the cushion, except at the inlet opening and the frame aperture.

The upper frame portion may comprise an elongate frame element extending laterally across an upper portion of the cushion. The elongate frame element preferably extends below an uppermost portion of the cushion.

The upper frame portion may comprise a rear surface which rests against the upper portion of the cushion, when the cushion is mounted on the frame. The upper portion of the cushion may comprise an elongate recess extending laterally across the cushion, the upper frame portion being received in the elongate recess. The upper frame portion may be received in the elongate recess with an interference fit. The elongate recess may be configured to resiliently deform to receive the upper frame portion.

The upper frame portion may comprise a plurality of elongate frame elements. In one example the upper frame portion comprises two elongate frame elements. The elongate frame elements may be spaced apart so as to define a space between them.

The cushion may be exposed through the space. The elongate frame elements may be configured such the space between the elongate frame elements can be adjusted. One, both or each elongate frame element may be movably mounted on the frame, so as to be movable towards or away from another elongate frame element. One, both or each elongate frame element may be pivotally mounted on the frame. One both or each elongate frame element may be resiliently deformable, or comprise one or more resiliently deformable regions, such that an elongate frame element may be resiliently deformed towards or away from another elongate frame element. One, both or each elongate frame element may be adjustable between two or more predetermined positions.

The frame may be configured to deform the cushion when the cushion is mounted on the frame. For example, the frame may laterally squeeze the cushion. In other words, the lateral width of the cushion when mounted on the frame, may be less than the lateral width of the cushion when not mounted on the frame.

The opposed side portions of the frame may define a lateral distance between them which is less than the corresponding lateral distance between side walls of the cushion. In particular, the lateral distance between cushion engaging surfaces of the opposed side portions may be less than the distance between the side walls of the cushion. For example, the lateral distance between the opposed side portions may be 90% of the lateral distance between side walls of the cushion.

The distal inlet aperture of the cushion may form a connection with the frame by way of being connected to the inlet opening of the frame, the remainder of the cushion not being connected to the frame.

The cushion may be cantilevered on the frame by way of the distal inlet aperture of the cushion being connected to the inlet opening of the frame.

The inlet opening may be provided at a lower part of the frame; wherein
the distal inlet aperture of the cushion is removably connected to the inlet opening of the frame; and
the side wall comprises a proximal portion adjacent the face contacting portion and a distal portion distal from the face contacting portion, the frame being configured to overlap the proximal portion of the cushion side wall and to expose the distal portion of the cushion side wall.

The distal inlet aperture of the cushion may be removably connected to the inlet opening of the frame; and
the inlet opening is provided at a lower part of the frame; and
the frame comprises an elongate frame member extending up the side wall and across an upper portion of the cushion.

The mask assembly may comprise lateral arms on either side of the inlet opening that extend towards the face contacting portion of the cushion; and an elongate frame member that extends over an upper portion of the cushion and connects the lateral arms.

Each headgear connector may comprise an elongate post on which a respective headgear strap is configured to be mounted; wherein the elongate posts of the lower pair of headgear connectors are inclined relative to the elongate posts of the upper pair of elongate posts.

The cushion may comprise a rolling bridge portion which is pivotable about a pivot axis relative to the remainder of the cushion towards or away from the user; wherein each headgear connector is positioned such that headgear straps overlap the pivot axis when connected to the headgear connectors.

The frame may comprise opposed lateral arms extending laterally outwardly from the frame, each lateral arm comprising at least one of the headgear connectors onto which a respective headgear strap can be connected; wherein the lateral arms each extend laterally outwardly from the frame in a direction substantially aligned with a primary force vector from the headgear straps when connected to the headgear connectors.

The frame may comprise at least one guide surface configured to guide headgear straps, during connection of the headgear straps to the headgear connectors, toward a respective headgear connector.

The mask assembly may comprise one or more headgear clips configured to connect the lower and /r upper headgear straps to the lower and/or upper headgear connectors.

The headgear clip may comprise a clip body. The clip body may be elongate. The clip body may comprise a slot to receive a headgear strap. The clip body may comprise a hook to receive a post of a headgear connector.

The headgear clip may comprise an extension configured to extend away from the body and support the side wall of the cushion. This can assist in reducing or preventing cushion blow out.

Each headgear clip may comprise a pair of upper and lower extensions. The extensions may be shaped to match the shape of the side wall of the cushion, and each comprise a support surface against which the cushion side wall can rest. The lower extension may comprise a distal end which terminates below the lateral frame arm.

The upper extension may comprise a distal end which terminates adjacent upper headgear connector.

The cushion may comprise an exterior indent configured to be positioned adjacent a headgear clip when the cushion is mounted on the frame and a headgear strap is mounted on an upper or lower headgear connector, the indent providing clearance between the cushion and the headgear clip.

The indent may be provided on a region of the side wall of the cushion adjacent a headgear connector of the frame. The indent may be adjacent a lower headgear connector. The indent may be adjacent an upper headgear connector. A plurality of indents may be provided. Each indent may be elongate and may extend rearwardly. The indent may be configured such that the headgear clip can move relative to the lower headgear connector, without contacting the cushion.

The cushion may be provided with one or more support ribs, internally of the cushion, to resist deformation of the cushion.

The cushion may comprise a pair of laterally spaced apart, generally laterally extending support ribs, one on each side of the central vertical axis of the cushion.

The laterally extending support ribs may be located in an upper portion of the cushion adjacent a rolling bridge portion of the cushion.

The cushion may comprise a pair of laterally spaced apart, generally vertically extending support ribs. Each rib may be at or adjacent a pivot axis of a rolling bridge portion of the cushion. These vertically extending support ribs can limit by how much the nose portion of the cushion balloons under pressure, reducing the likelihood of eye leaks for example.

Each rib may be integrally formed with the cushion, from the same cushion material.

The lower part of the frame may comprise a pair of finger grips, the inlet opening being intermediate and adjacent the finger grips.

The inlet opening may be provided at a lower part of the frame; wherein the inlet opening comprises a central axis, the central axis extending generally downwardly.

The central axis of the frame inlet opening may extend through the cushion inlet aperture and below the lower part of the frame.

The inlet opening may be provided at a lower part of the frame, the inlet opening comprising a central axis; wherein the assembly further comprises an anti-asphyxia valve comprising a valve member and a valve outlet opening, the valve being movable between a closed position in which the valve member closes the valve outlet opening, and an open position in which the valve member opens the valve outlet opening, the valve outlet opening comprising a valve outlet central axis; wherein
the inlet opening central axis is substantially perpendicular to the valve outlet central axis.

The lower part of the frame may comprise a vent hole and a diffuser configured to cover the vent hole.

The mask assembly may comprise an anti-asphyxia valve provided between the cushion inlet aperture and the frame inlet opening, and comprising a valve member and a valve outlet opening, the valve member being movable between a closed position in which the valve member closes the valve outlet opening, and an open position in which the valve member opens the valve outlet opening, the valve outlet opening comprising a valve outlet opening central axis; wherein the valve member is pivotally mounted above the frame inlet opening, the valve outlet opening being positioned above the valve member and above the frame inlet opening, the valve member pivoting away from the cushion to close the valve outlet opening, the valve outlet opening being provided on a forward part of the assembly.

In embodiments of this disclosure a mask assembly is provided which comprises:
a) a cushion with a proximal face contacting portion, a distal inlet aperture, and a side wall between the proximal face contacting portion and the distal inlet aperture;
b) a frame comprising at least one headgear connector and an inlet opening configured to connect to a source of pressurised gas; wherein
c) the cushion is mounted on the frame.

For example, the distal inlet aperture of the cushion may be removably connected to the inlet opening of the frame. The frame may be configured to overlap an exterior portion of the side wall of the cushion.

In embodiments of this disclosure a mask assembly is provided which comprises a cushion with a proximal face contacting portion, a distal inlet aperture, and a side wall between the proximal face contacting portion and the distal inlet aperture, and a frame comprising an inlet opening configured to connect to a source of pressurised gas.

The distal inlet aperture of the cushion may be removably connected to the inlet opening of the frame. The frame may comprise a pair of upper and a pair of lower headgear connectors. The cushion may comprise an uppermost portion, both pairs of upper and lower headgear connectors being below the cushion uppermost portion.

The mask assembly may further comprise a headgear assembly. The headgear assembly may comprise a strap assembly including at least a pair of opposing side straps, and/or a rear strap or panel, and/or a crown strap. The pair of opposing side straps may be a pair of opposing upper side straps; the headgear assembly further comprising a pair of opposing lower side straps. A free end of each of the upper straps may be coupled to the mask frame. A free end of each of the lower side straps may be coupled to the mask frame.

According to an aspect of this disclosure there is provided a mask assembly comprising a mask seal and a mask frame, the mask seal comprising a region configured to buckle in a single direction to define a roll of material that extends over the mask frame.

According to an aspect of this disclosure there is provided a mask assembly comprising a mask seal mounted on a mask frame, the mask frame comprising an elongate member extending over an apex of the mask seal, the mask seal comprising a region configured to buckle in a single direction to define a roll of material that extends over the elongate member.

According to an aspect of this disclosure there is provided a mask assembly comprising a mask seal mounted on a mask frame, the mask frame defining an upper frame portion and a lower frame portion and an opening therebetween, the mask seal being exposed through the opening, the mask seal comprising a region configured to buckle in a single direction to define a roll of material that extends over the upper frame portion.

According to an aspect of this disclosure there is provided a mask assembly comprising a mask seal and a mask frame, the mask seal comprising a region configured to buckle in a single direction to define a roll of material that extends over the mask frame, the mask frame comprising at least one headgear connector.

According to an aspect of this disclosure there is provided a mask assembly comprising
a) a cushion with a face contacting portion and a non-face contacting portion, a side wall extending between the face contacting portion and the non-face contacting portion, and
b) a frame comprising an inlet opening configured to connect to a source of pressurised gas; wherein
the cushion comprises an upper portion adjacent the nasal bridge of the user; the upper portion of the cushion comprising a rolling bridge portion which is pivotable towards or away from the nasal bridge of the user to overlap with the mask frame.

According to an aspect of this disclosure there is provided a mask assembly comprising a mask cushion and a mask frame, the mask seal comprising a proximal face-contacting portion and a distal non-face contacting portion, the mask cushion further comprising an upper portion and a lower portion, the upper portion comprising a rolling bridge portion and being pivotable relative to the lower portion, the upper portion comprising a region of reduced stiffness that is positioned between a first boundary, the first boundary being defined by a stiffness greater than that in the region of reduced stiffness, the second boundary being defined by a stiffness greater than that in the region of reduced stiffness, wherein when the first boundary is moved toward the second boundary, the region of reduced stiffness buckles to define a roll of material that increases in size away from the face-contacting portion as the first boundary continues to move toward the second boundary to facilitate movement of the rolling bridge portion of the mask cushion relative to the lower portion of the mask cushion,
wherein the mask frame is configured to overlay an exterior portion of the second boundary such that the roll of material extends over the mask frame.

The roll of material may be movable relative to the lower portion of the mask cushion to extend partially over an upper portion of the mask frame.

The roll of material may be movable relative to the lower portion of the mask cushion to extend fully over the upper portion of the mask frame.

The upper portion of the mask cushion may be pivotable relative to the lower portion of the mask cushion about a pivot axis, the pivot axis being substantially mid-way up the mask cushion, when the mask cushion is viewed from the side.

The roll of material may extend over a forward margin of the upper portion of the mask frame.

The region of reduced stiffness may buckle in a single direction to define the roll of material.

The frame may form part of the second boundary.

The frame may comprise at least one headgear connector and an inlet opening configured to connect to a source of pressurised gas; the cushion comprising an inlet aperture; wherein
the inlet aperture of the cushion is removably connected to the inlet opening of the frame; and
   the frame is configured to overlap an exterior portion of the side wall.

The frame may comprise any one or more of:
a) an elongate frame member extending across the upper portion of the cushion.
b) an upper frame portion;
c) opposed side frame portions;
d) a lower frame portion comprising the inlet opening configured to connect to a source of pressurised gas;
e) a frame aperture defined between the upper, side and lower frame portions, and configured such that the cushion is exposed through the frame aperture.

An inlet aperture of the cushion may form a connection with the frame by way of being connected to an inlet opening of the frame, the remainder of the cushion not being connected to the frame.

According to an aspect of this disclosure there is provided a mask assembly comprising:
a cushion with a proximal face contacting portion and a distal non-face contacting portion;
a side wall extending between the face contacting portion and the non-face contacting portion;
a mask frame, wherein the cushion module is mounted on the frame; wherein;
the mask frame comprising a pair of laterally opposed headgear connectors spaced part by a lateral dimension, wherein
the mask frame is configured to be laterally compliant so as to resiliently deform such that the lateral dimension between the headgear connectors can change when subject to lateral forces.

According to an aspect of this disclosure there is provided a mask assembly comprising:
a cushion with a proximal face contacting portion and a distal non-face contacting portion,
a side wall extending between the face contacting portion and the non-face contacting portion;
a mask frame, wherein the cushion module is mounted on the frame; wherein;
the mask frame comprising a pair of laterally opposed headgear connectors spaced part by a lateral dimension, wherein
the mask frame is configured to be longitudinally non-compliant in a longitudinal direction between the proximal face contacting portion and the distal non-face contacting portion, so as to resist deformation of the mask frame when subject to longitudinal forces.

According to an aspect of this disclosure there is provided a mask assembly comprising
a cushion with a proximal face contacting portion and a distal non-face contacting portion,
a side wall extending between the face contacting portion and the non-face contacting portion;
a mask frame, wherein the cushion module is mounted on the frame; the mask frame comprising a pair of laterally opposed lower headgear connectors, and a pair of laterally opposed upper headgear connectors; wherein;
   the frame is configured to deform under lateral forces to the extent that, when in a deformed condition:
a) elongate elements of the upper headgear connectors, which are inwardly inclined when viewed from the front in a rest condition of the mask assembly, are displaced to a position in which the elongate elements are substantially parallel; and/or
b) the lower headgear connectors are displaced to a position in which the lower headgear connectors are each aligned with a respective outer margin of an inlet opening of the frame; and/or
c) the lower headgear connectors are displaced to a position in which the lower headgear connectors are each aligned with a respective laterally outermost margin of a central portion of the frame; and/or
d) the lower headgear connectors are displaced to a position in which the lower headgear connectors are each aligned with a respective weakened region or living hinge adjacent a central portion of the frame; and/or
e) the lower headgear connectors are displaced to a position in which the lateral distance between the lower headgear connectors decreases by a distance in the range of 140 mm to 20 mm, or 140 to 60 mm, or 110 to 40 mm.

According to an aspect of this disclosure there is provided a mask assembly comprising:
a cushion with a face contacting portion and a non-face contacting portion,
a side wall extending between the face contacting portion and the non-face contacting portion, and
a frame configured to overlay an exterior portion of the side wall of the cushion; wherein the frame comprises:
a pair of headgear connectors, and
a lateral dimension defined between the pair of headgear connectors;
wherein the frame is configured to be laterally compliant so as to resiliently deform such that the lateral dimension can change when subject to lateral forces.

According to an aspect of this disclosure there is provided a mask assembly comprising:
a cushion with a proximal face contacting portion, a distal inlet aperture, and a side wall between the proximal face contacting portion and the distal inlet aperture, and
a frame comprising a pair of headgear connectors, a lateral dimension defined between the pair of headgear connectors; the distal inlet aperture of the cushion being connected to the frame;
the remainder of the cushion not being connected to the frame;
wherein the frame is configured to be laterally compliant so as to resiliently deform such that the lateral dimension can change when subject to lateral forces.

The frame may be configured to be longitudinally non-compliant in a longitudinal direction between the proximal face contacting portion and the distal non-face contacting portion, so as to resist deformation of the mask frame when subject to longitudinal forces.

The frame may be configured to be relatively laterally compliant and relatively longitudinally non-compliant.

The frame may comprise a plurality of frame regions, at least one frame region being weakened relative to at least one other frame region.

The plurality of frame regions may comprise at least one frame region which is relatively thin in one dimension to encourage bending of the frame about a first predetermined axis and relatively wide in another dimension to resist bending about a second predetermined axis.

The frame may comprise an upper portion which extends laterally across a portion of the mask assembly, the upper portion comprising a weakened region configured to allow bending of the upper portion.

The upper portion may be arcuate, wherein bending of the upper portion increases or decreases radius of arc.

At least one of the frame regions may comprise a living hinge.

A weakened frame region may be adjacent the inlet.

According to an aspect of this disclosure there is provided a mask assembly comprising:
a cushion with a proximal face contacting portion, a distal inlet aperture, and a side wall between the proximal face contacting portion and the distal inlet aperture, and
a frame comprising at least one headgear connector and an inlet opening configured to connect to a source of pressurised gas; wherein
the distal inlet aperture of the cushion is removably connected to the inlet opening of the frame, and
the frame is configured to overlap an exterior portion of the side wall.

The frame may comprise a lower portion, the inlet opening being provided in the lower portion, the lower portion overlapping with the cushion.

The frame may comprise an upper portion, the upper portion overlapping an upper portion of the cushion.

The frame may comprise any one or more of:
a) an elongate frame member extending across an upper portion of the cushion.
b) an upper frame portion;
c) opposed side frame portions;
d) a lower frame portion comprising the inlet opening configured to connect to a source of pressurised gas;
e) a frame aperture defined between the upper, side and lower frame portions, and configured such that the cushion is exposed through the frame aperture.

An inlet aperture of the cushion may form a connection with the frame by way of being connected to an inlet opening of the frame, the remainder of the cushion not being connected to the frame.

According to an aspect of this disclosure there is provided a mask assembly comprising a resilient deformable cushion, and a frame, wherein the frame comprises:
an upper frame portion;
opposed side frame portions;
a lower frame portion comprising an inlet opening configured to connect to a source of pressurised gas; wherein
a frame aperture is defined between the upper and lower frame portions, and configured such that the cushion is exposed through the frame aperture.

The upper frame portion may extend across an upper portion of the cushion.

The cushion may comprise a region configured to buckle in a single direction to define a roll of material that extends over the upper frame portion, the upper frame portion extending across the upper portion of the cushion adjacent the region configured to buckle. In another embodiment, the upper frame portion may extend across the upper portion of the cushion so as to be spaced below the region configured to buckle.

The upper frame portion may comprise a forward margin, the cushion being exposed below the forward margin.

At least one of the upper frame portion, side frame portions, and lower frame portion may comprise an elongate frame member.

The inlet opening may comprise a central axis, the central axis extending generally downwardly.

The central axis of the frame inlet opening may extend through the cushion inlet aperture and below the lower part of the frame.

The inlet aperture of the cushion may form a connection with the frame by way of being connected to an inlet opening of the frame, the remainder of the cushion not being connected to the frame.

According to an aspect of this disclosure there is provided a mask assembly comprising:
a cushion with a proximal face contacting portion, a distal inlet aperture, and a side wall between the proximal face contacting portion and the distal inlet aperture, and
a frame comprising at least one headgear connector and an inlet opening configured to connect to a source of pressurised gas,
the distal inlet aperture of the cushion forming a connection with the frame by way of being connected to the inlet opening of the frame, the remainder of the cushion not being connected to the frame.

According to an aspect of this disclosure there is provided a mask assembly comprising:
a cushion with a proximal face contacting portion, a distal inlet aperture, and a side wall between the proximal face contacting portion and the distal inlet aperture, and
a frame comprising at least one headgear connector and a central connection portion configured to connect to a source of pressurised gas;
the distal inlet aperture of the cushion forming a connection with the frame by way of being connected to the central connection portion of the frame, the remainder of the cushion not being connected to the frame.

The cushion may be cantilevered on the frame by way of the distal inlet aperture of the cushion being connected to the inlet opening and/or central connection portion of the frame.

The frame may be configured to overlap an exterior portion of the side wall of the cushion.

The frame may be configured to be laterally compliant so as to resiliently deform such that a lateral dimension of the mask assembly can change when subject to lateral forces.

The frame may comprise any one or more of:
a) an elongate frame member extending across an upper portion of the cushion.
b) an upper frame portion;
c) opposed side frame portions;
d) a lower frame portion comprising the inlet opening configured to connect to a source of pressurised gas;
e) a frame aperture defined between the upper, side and lower frame portions, and configured such that the cushion is exposed through the frame aperture.

According to an aspect of this disclosure there is provided a mask assembly comprising:
a cushion with a proximal face contacting portion, a distal inlet aperture, and a side wall between the proximal face contacting portion and the distal inlet aperture, and
a frame comprising at least one headgear connector and an inlet opening configured to connect to a source of pressurised gas; wherein
the inlet opening is provided at a lower part of the frame;
the distal inlet aperture of the cushion is removably connected to the inlet opening of the frame, and
the side wall comprises a proximal portion adjacent the face contacting portion and a distal portion distal from the face contacting portion, the frame being configured to overlap the proximal portion of the cushion side wall and to expose the distal portion of the cushion side wall.

The frame may comprise an upper frame portion that extends across an upper portion of the cushion.

The upper frame portion may comprise a forward margin, the cushion being exposed below the forward margin.

The inlet opening may comprise a central axis, the central axis extending generally downwardly.

The central axis of the frame inlet opening may extend through the cushion inlet aperture and below the lower part of the frame.

The frame may comprise any one or more of:
a) an elongate frame member extending across an upper portion of the cushion.
b) an upper frame portion;
c) opposed side frame portions;
d) a lower frame portion comprising the inlet opening configured to connect to a source of pressurised gas;
e) a frame aperture defined between the upper, side and lower frame portions, and configured such that the cushion is exposed through the frame aperture.

At least one of the upper frame portion, side frame portions and lower frame portion may comprise an elongate frame member.

According to an aspect of this disclosure there is provided a mask assembly comprising:
a cushion with:
   a proximal face contacting portion, a distal inlet aperture, and a side wall between the proximal face contacting portion and the distal inlet aperture, and
a frame comprising an inlet opening configured to connect to a source of pressurised gas; wherein
the distal inlet aperture of the cushion is removably connected to the inlet opening of the frame, and
the inlet opening is provided at a lower part of the frame; and
the frame comprises an elongate frame member extending up the side wall and across an upper portion of the cushion.

According to an aspect of this disclosure there is provided a mask assembly comprising:
a cushion with a proximal face contacting portion, a distal inlet aperture, and a side wall between the proximal face contacting portion and the distal inlet aperture; and
a frame comprising an inlet opening configured to connect to a source of pressurised gas; wherein
the distal inlet aperture of the cushion is removably connected to the inlet opening of the frame, and
the inlet opening is provided at a lower part of the frame; and
the frame comprises an elongate frame member extending across an upper portion of the cushion; the elongate frame member comprising an apex portion, the apex portion of the elongate frame member comprising an elongate narrowed portion.

The elongate frame member may comprise distal end portions adjacent the side wall, the apex portion being between the distal end portions, the elongate narrowed portion being between the distal end portions and the central apex portion.

The cushion may comprise a nasal bridge portion, the elongate frame member being spaced below the nasal bridge portion.

The elongate frame member may comprise a forward margin, the cushion being exposed in front of the forward margin.

The inlet opening may comprise a central axis, the central axis extending generally downwardly.

The central axis of the frame inlet opening may extend through the cushion inlet aperture and below the lower part of the frame.

The frame may comprise a frame aperture defined between the upper, side and lower frame portions, and configured such that the cushion is exposed through the frame aperture.

According to an aspect of this disclosure there is provided a mask assembly comprising:
a cushion with a proximal face contacting portion, a distal inlet aperture, and a side wall between the proximal face contacting portion and the distal inlet aperture; and
a frame comprising:
   at least one headgear connector;
   an inlet opening configured to connect to a source of pressurised gas;
   lateral arms on either side of the inlet opening that extend along the side wall towards the face contacting portion of the cushion; and
   an elongate frame member that extends over an upper portion of the cushion and connects the lateral extensions/arms.

The elongate frame member may be arcuate.

The cushion may comprise an apex portion, the elongate frame member extending at or adjacent the apex portion.

The lateral arms and the elongate frame member may be configured to define a frame aperture which exposes the cushion.

The frame aperture may expose the cushion above the inlet opening of the frame, above the lateral extensions/arms and below the elongate connecting member.

The upper frame portion may comprise a forward margin, the cushion being exposed forward of the forward margin.

The frame may comprise a lower portion, the inlet opening being provided in the lower portion, the inlet opening comprising a central axis, the central axis extending generally downwardly.

The central axis of the frame inlet opening may extend through the cushion inlet aperture and below the lower part of the frame.

An inlet aperture of the cushion may form a connection with the frame by way of being connected to an inlet opening of the frame, the lateral arms of the frame not being connected to the cushion.

According to an aspect of this disclosure there is provided a mask assembly comprising:
a cushion with a face contacting portion and a non-face contacting portion, a side wall extending between the face contacting portion and the non-face contacting portion; and
a frame comprising an inlet opening configured to connect to a source of pressurised gas; wherein
the mask frame comprises:
   an upper pair and a lower pair of headgear connectors onto which a respective headgear strap is configured to be mounted;
   the lower pair of headgear connectors being inclined relative to the elongate posts of the upper pair of elongate posts, such that the headgear straps are non-parallel when connected to the headgear connectors.

According to an aspect of this disclosure there is provided a mask assembly comprising:
a cushion with a face contacting portion and a non-face contacting portion;
a side wall extending between the face contacting portion and the non-face contacting portion; and
a frame comprising an inlet opening configured to connect to a source of pressurised gas; wherein
the mask frame comprises:
   an upper pair and a lower pair of headgear connectors, each headgear connector comprising an elongate post on which a respective headgear strap is configured to be mounted;
   the elongate posts of the lower pair of headgear connectors being inclined relative to the elongate posts of the upper pair of elongate posts.

The elongate posts of the lower pair of headgear connectors may be substantially vertical when the mask assembly is viewed from the front.

The elongate posts of the upper pair of headgear connectors may be inwardly inclined when the mask assembly is viewed from the front.

The elongate posts of the lower pair of headgear connectors may be substantially vertical when the mask assembly is viewed from the side.

The elongate posts of the upper pair of headgear connectors may be forwardly inclined when the mask assembly is viewed from the side.

The lower pair of headgear connectors may be at a widest part of the frame.

The widest part of the frame may comprise a lower portion of the frame, an upper portion of the frame being narrower than the lower portion of the frame, when the mask assembly is viewed from the front.

The elongate posts of the upper pair of headgear connectors may be at or below an upper portion of the cushion.

According to an aspect of this disclosure there is provided a mask assembly comprising:
a cushion with a face contacting portion and a non-face contacting portion, a side wall extending between the face contacting portion and the non-face contacting portion; and
a frame comprising an inlet opening configured to connect to a source of pressurised gas;
the cushion comprising a rolling bridge portion which is pivotable about a pivot axis relative to the remainder of the cushion towards or away from the user; wherein
the mask frame comprises:
   a pair of laterally opposed headgear connectors onto which respective headgear straps can be connected;
   each headgear connector being positioned such that the headgear straps overlap the pivot axis when the headgear straps are mounted on the headgear connectors.

The pair of laterally opposed headgear connectors may comprise an upper pair of headgear connectors, the mask assembly further comprising a lower pair of headgear connectors.

The elongate posts of the upper pair of headgear connectors may be at or below an upper portion of the cushion.

The pair of headgear connectors may be inwardly inclined when the mask assembly is viewed from the front.

The upper pair of headgear connectors may be forwardly inclined when the mask assembly is viewed from the side.

Each headgear connector may comprise an elongate post.

According to an aspect of this disclosure there is provided a mask assembly comprising:
a cushion with a face contacting portion and a non-face contacting portion, a side wall extending between the face contacting portion and the non-face contacting portion; and
a frame comprising an inlet opening configured to connect to a source of pressurised gas;
the inlet opening being provided at a lower part of the frame; wherein the mask frame comprises:
   opposed lateral arms extending laterally outwardly from the lower part of the frame, each lateral arm comprising a headgear connector onto which a respective headgear strap can be connected; wherein the lateral arms each extend laterally outwardly from the lower part of the frame in a direction substantially aligned with a primary force vector from the headgear straps when connected to the headgear connectors.

The inlet opening may comprise a central axis, the lateral arms extending away from the inlet opening at an angle inclined relative to the central axis.

The angle may be between 10 and 80°, or between 10 and 80°, or between 15 and 70°, or between 20 and 60°, or between 25 and 55°, or is substantially 45°.

The headgear connectors may be positioned sufficiently rearwardly on the frame to be adjacent the face contacting portion of the cushion.

The headgear connectors may be positioned sufficiently rearwardly to be aligned with a rolling bridge portion of the cushion.

The headgear connectors may be positioned nearer the rear of the mask assembly than the front of the mask assembly.

The cushion may comprise an inlet aperture, the inlet aperture forming a connection with the frame by way of being connected to an inlet opening of the frame, the lateral arms of the frame not being connected to the cushion.

According to an aspect of this disclosure there is provided a mask assembly comprising:
a cushion with a face contacting portion and a non-face contacting portion,
a side wall extending between the face contacting portion and the non-face contacting portion; and
a frame comprising an inlet opening configured to connect to a source of pressurised gas; wherein
the inlet opening is provided at a lower part of the frame,
the mask frame comprising a pair of opposed headgear connectors onto which respective headgear straps can be connected; wherein
the mask frame comprises at least one guide surface configured to guide the headgear straps, during connection to the headgear connectors, toward a respective headgear connector.

The guide surface may extend forwardly of the headgear connector.

The frame may comprise a central portion, the inlet opening being provided in the central portion, and lateral portions laterally outward of the central portion, the lateral portions each comprising a guide surface.

The guide surface may comprise a portion of a leading or forward edge of the frame.

Each headgear connector may be elongate, the or each guide surface being elongate, a longitudinal axis of each guide surface intersecting a longitudinal axis of a respective headgear connector.

The guide surface may comprise upper and lower guide surfaces that are relatively inclined to define a guide mouth converging on the headgear connector element

According to an aspect of this disclosure there is provided a mask assembly comprising
a cushion with a face contacting portion and a non-face contacting portion, a side wall extending between the face contacting portion and the non-face contacting portion; and
a frame comprising an inlet opening configured to connect to a source of pressurised gas;
   the inlet opening being provided at a lower part of the frame; wherein
   the inlet opening comprises a central axis, the central axis extending generally downwardly and below the lower part of the frame.

According to an aspect of this disclosure there is provided a mask assembly comprising:
a cushion with a face contacting portion and a non-face contacting portion;
a side wall extending between the face contacting portion and the non-face contacting portion; and
a frame comprising an inlet opening configured to connect to a source of pressurised gas;
the inlet opening being provided at a lower part of the frame,
the inlet opening having a central axis which extends generally vertically; wherein
the cushion comprises an inlet aperture, the central axis of the frame inlet opening extending through the cushion inlet aperture.

According to an aspect of this disclosure there is provided a mask assembly comprising:
a cushion with a face contacting portion and a non-face contacting portion:
a side wall extending between the face contacting portion and the non-face contacting portion; and
a frame comprising an inlet opening configured to connect to a source of pressurised gas;
the inlet opening is provided at a lower part of the frame, the inlet opening comprising a central axis; wherein
the assembly further comprises an anti-asphyxia valve comprising a valve member and a valve outlet opening, the valve being movable between a closed position in which the valve member closes the valve outlet opening, and an open position in which the valve member opens the valve outlet opening, the valve outlet opening comprising a valve outlet central axis,
the inlet opening central axis being substantially perpendicular to the valve outlet central axis.

According to an aspect of this disclosure there is provided a mask assembly comprising:
a cushion with a face contacting portion and a non-face contacting portion;
a side wall extending between the face contacting portion and the non-face contacting portion; and
a frame comprising an inlet opening configured to connect to a source of pressurised gas;
   the inlet opening is provided at a lower part of the frame; wherein
   the lower part of the frame comprises a pair of finger grips, the inlet opening being intermediate and adjacent the finger grips.

According to an aspect of this disclosure there is provided a mask assembly comprising:
a cushion with a face contacting portion and a non-face contacting portion;
a side wall extending between the face contacting portion and the non-face contacting portion; and
a frame comprising an inlet opening configured to connect to a source of pressurised gas; wherein
   the inlet opening is provided at a lower part of the frame, the mask assembly further comprising a vent hole and a diffuser configured to cover the vent hole.

The diffuser may be removably mounted on the mask assembly.

The diffuser may comprise a diffuser frame. The diffuser may be integral with the diffuser frame.

The diffuser may be configured to clip onto the frame.

The diffuser may be mounted on the frame. The diffuser may be integral with the frame. The diffuser may be mounted on a separate component, the separate component being mounted on the frame.

The mask assembly may comprise an array of vent holes.

The frame may comprise two opposed pairs of headgear connectors:
a) a lower pair of headgear connectors; and
b) an upper pair of headgear connectors.

The headgear connectors may all be located below a nasal bridge portion of the mask assembly.

The mask assembly may comprise a lower, central portion that is a different colour to the remainder of the frame and/or cushion.

The diffuser may be configured such that part of the colour of lower, central portion is exposed, when the diffuser is mounted on the mask assembly.

The frame may comprise a recess when viewed from the front, the diffuser being exposed through the recess.

The recess may comprise inclined side margins. The recess may be substantially ‘V’ shaped. The angle of the side margins of the recess may be substantially the same as the longitudinal axis of headgear straps of the mask assembly, when the headgear straps are connected to the headgear connectors.

According to an aspect of this disclosure there is provided a mask assembly comprising:
a cushion with a face contacting portion and a non-face contacting portion;
   a side wall extending between the face contacting portion and the non-face contacting portion; and
   a frame comprising an inlet opening configured to connect to a source of pressurised gas:
the inlet opening is provided at a lower part of the frame;
the cushion comprising an inlet aperture in the non-face contacting portion;
an anti-asphyxia valve being provided between the cushion inlet aperture and the frame inlet opening, and comprising a valve member and a valve outlet opening, the valve member being movable between a closed position in which the valve member closes the valve outlet opening, and an open position in which the valve member opens the valve outlet opening, the valve outlet opening comprising a valve outlet opening central axis; wherein
the valve member is pivotally mounted above the frame inlet opening, the valve outlet opening being positioned above the valve member and above the frame inlet opening, the valve member pivoting away from the cushion to close the valve outlet opening, the valve outlet opening being provided on a forward part of the assembly, the inlet opening central axis being substantially perpendicular to the valve outlet central axis.

According to another aspect of this disclosure there is provided a mask assembly comprising:
a cushion with a proximal face contacting portion, a distal inlet aperture; and a side wall between the proximal face contacting portion and the distal inlet aperture; and
a frame comprising:
   at least one headgear connector;
   a central portion, the central portion being substantially rigid;
   a pair of lateral arms, each arm extending away from a respective side of the central portion; the lateral arms being flexible along at least a portion of their length; and
   a connecting frame member that connects the lateral arms at a position distal from the central frame portion.

The central portion of the frame may comprise any one or more of:
a) an inlet opening configured to connect to a source of pressurised gas;
b) a bias vent hole;
c) a diffuser;
d) an anti-asphyxiation valve;
e) an outlet configured to fluidly couple with the distal inlet aperture of the cushion.

The connecting frame member may comprise an elongate band.

The connecting frame member may connect the lateral arms at a position above and behind the central portion.

The connecting frame member may extend over an upper portion of the cushion.

The connecting frame member may be integral with the lateral arms.

The connecting frame member may be flexible along at least a portion of its length.

The connecting frame member may comprise a central region, the central region being flexible.

The central region may be an apex region of the frame.

The cushion may comprise an upper portion adjacent the nasal bridge of the user; the upper portion of the cushion comprising a rolling bridge portion which is pivotable towards or away from the nasal bridge of the user to overlap with the central region of the connecting frame member.

The connecting frame member may comprise end regions, distal from the central region, the end regions being less flexible than the central region.

The central region may comprise a relatively thin and/or weakened structure, and the end regions comprise a relatively thick and/or reinforced structure.

The central region may comprise a first width, and the end regions comprise a second width, the second width being greater than the first width.

At least one headgear connector may comprise a pair of headgear connectors, the pair of headgear connectors being at or adjacent the end regions.

The central portion, lateral arms, and connecting frame member may together define a closed aperture of the frame.

The mask assembly may be arranged such that, from the side, an acute angle is formed between the lateral arms and the connecting frame member.

According to an aspect of this disclosure, a mask assembly may be provided comprising any one or more of the above features, wherein the cushion comprises an upper portion comprising a region of reduced stiffness that is positioned between a first boundary, the first boundary being defined by a stiffness greater than that in the region of reduced stiffness, the second boundary being defined by a stiffness greater than that in the region of reduced stiffness, wherein when the first boundary is moved toward the second boundary, the region of reduced stiffness buckles in a single direction to define a roll of material that increases in size away from the face-contacting flange or side as the first boundary continues to move toward the second boundary to facilitate movement of the rolling bridge portion of the mask seal relative to the lower portion of the mask seal.

In embodiments of this disclosure a mask assembly is provided which comprises:
a) a cushion with a proximal face contacting portion, a distal inlet aperture, and a side wall between the proximal face contacting portion and the distal inlet aperture;
b) a frame comprising at least one headgear connector and a central connection portion configured to connect to a source of pressurised gas; wherein the cushion is mounted on the central connection portion of the frame.

The frame may comprise a frame clip configured to removably mount the frame on another component of the mask assembly. The central connection portion may comprise the frame clip.

The frame clip may be configured to be mounted only on one component of the mask assembly. The frame clip may therefore be configured not to be mounted on any other component of the mask assembly.

The frame may be configured to be removed from the other components of the mask assembly, without disassembly of those other components.

The cushion of the mask assembly may be configured to be removably mounted on a component of the mask assembly. The mask assembly may be configured such that the cushion can be removed from the component of the mask assembly without removing the mask frame from the mask assembly. The mask assembly may be configured such that the cushion can be removed from the component of the mask assembly without removing any other component from the mask assembly.

The mask assembly may comprise an inlet connector configured to be connected to a gas delivery conduit to deliver breathable gas into the inlet connector and into the cushion, the frame clip being configured to be removably mounted on the inlet connector. The cushion may be configured to be removably mounted on the inlet connector. The inlet connector may comprise an inlet port configured to be connected to the gas delivery conduit. The inlet connector may comprise an outlet port configured to be connected to the cushion. A gas flow path may be defined between the inlet port and the outlet port, through the inlet connector.

The mask assembly may comprise a conduit connector cuff of a gas delivery conduit configured to deliver breathable gas into the cushion, the frame clip being configured to be removably mounted on the conduit connector cuff.

The frame clip may be removably mounted on both the inlet connector and the conduit connector cuff.

The frame clip may comprise opposed ends between which is defined a mouth configured to receive another part of the mask assembly to mount the frame clip on the mask assembly.

The frame clip may comprise a front wall, and side walls extending rearwardly from the front wall, each side wall comprising an opposed end, the opposed ends being spaced apart to define the mouth at the rear of the frame clip.

The frame clip may comprise a rear wall, and side walls extending forwardly from the rear wall, each side wall comprising an opposed end, the opposed ends being spaced apart to define the mouth at the front of the frame clip.

The inlet port of the inlet connector may comprise a central inlet axis.

The mask assembly may be configured such that the central inlet axis is substantially vertical when the mask assembly is viewed from the front.

The inlet port may be considered to occupy a plane, the plane of the inlet port being substantially horizontal, or inclined less than 30° from the horizontal, in normal use, when the mask assembly is viewed from the front.

The central inlet axis may extend generally downwardly, when the mask assembly is viewed from the side. The central inlet axis may be inclined less than 30° from the vertical, when viewed from the side.

The central inlet axis may be substantially vertical when the mask assembly is viewed from the side.

The frame clip may be configured to be mounted on the mask assembly in a mounting direction substantially orthogonal to the central inlet axis.

The frame clip may be configured to be mounted on the mask assembly in a mounting direction that intersects the central inlet axis.

The mounting direction may be from front to rear of the mask assembly. The mounting direction may be from rear to front of the mask assembly.

At least one portion of the frame clip may be resiliently deformable, and configured to resiliently deform during mounting of the frame on the mask assembly. Preferably the at least one portion of the frame clip is configured to resiliently deform such that that opposed ends of the frame clip may deform away from each other to increase the size of the mouth of the frame clip, during mounting of the frame clip on the mask assembly. The frame clip may resiliently expand during mounting of the mask frame onto the remainder of the mask assembly, and then contract when fully mounted on the mask assembly.

The frame clip may be configured to be spaced from the remainder of the frame, such that forces applied to the remainder of the frame are decoupled from the frame clip. This decoupling may assist in minimising, for example, forces applied to the headgear connector(s) via headgear tension from deforming the frame clip and inadvertently disconnecting the frame clip from the mask assembly. The frame clip may comprise opposed sides that extend away from the mask frame, the opposed sides being spaced from the lateral arms and/or headgear connectors of the frame. The lateral arms and/or headgear connectors of the frame may be configured to deflect towards and/or away from the mask frame, without deforming the frame clip.

The frame clip may extend from the central connection portion of the frame. The frame clip may extend only from the central connection portion, such that the remainder of the frame clip is spaced from the remainder of the mask frame.

The opposed ends of the frame clip may extend outwardly away from the central connection portion. In such a configuration the opposed ends may comprise elongate fingers that define guide surfaces configured to guide the mask assembly during assembly. The elongate fingers may function as grips for the user to assist in expanding the frame clip during assembly and disassembly.

The opposed ends of the frame clip may function as locking formations configured to engage with another component of the mask assembly to resist removal of the frame clip from the mask assembly.

The frame clip may comprise a single integral component. The frame clip may comprise a plurality of components. The frame clip may comprise a pair of frame clip components the front and rear of the frame clip each comprising a respective mouth. Each of the frame clip components may comprise an end of a lateral arm of the frame, the other end of each lateral arm comprising a headgear connector.

The frame clip may be configured to be mounted on, and received in, a corresponding recess on the mask assembly. The recess may be provided for example on the inlet connector or on the conduit connector cuff, or may be on both components. The recess may be bounded by a lower radially outwardly extending formation, such as an annular flange, to resist movement of the frame clip in a generally downward direction. The recess may be bounded by an upper radially outwardly extending formation, such as an annular flange, to resist movement of the frame clip in a generally upward direction.

The mask assembly may comprise one or more guide surfaces configured to engage the frame clip during mounting of the frame clip on the mask assembly to guide the frame clip into a fully mounted position on the mask assembly.

The one or more guide surfaces may comprise inclined surfaces. The guide surfaces may be inclined generally upwardly in the mounting direction. The guide surfaces may comprise upper and lower guide surfaces, the guide surfaces converging in the mounting direction.

The one or more guide surfaces may be provided on the inlet connector. The one or more guide surfaces may be alternatively or additionally provided on the conduit connector cuff.

The mask assembly may comprise an end stop configured to abut the frame clip to limit the amount by which the frame clip can be moved onto the mask assembly.

The frame clip may be configured to resist or prevent relative rotation between the mask frame and the remainder of the mask assembly.

The frame clip may comprise at least one substantially straight or flat wall portion. The frame clip may comprise a plurality of substantially straight or flat wall portions. The substantially straight of flat wall portion may be configured to engage with one or more corresponding straight or flat portions of the mask assembly.

The frame clip may comprise a side wall, the straight or flat wall portion comprising a portion of the side wall.

The frame clip may comprise a pair of laterally opposed straight or flat side wall portions.

The side wall portions may be substantially straight and parallel.

The side wall portions may be substantially straight and non-parallel. For example, the side wall portions may converge as the side wall portions extend away from the mouth.

The frame clip may comprise one or more internal walls that define an inlet opening of the frame, the opening being non-circular.

The frame clip may comprise a front or rear wall, the straight of flat wall portion comprise a portion of the front or rear wall.

The frame clip may comprise an engagement formation, configured to engage a corresponding engagement formation on the mask assembly. The engagement formations may comprise a combination of one or more recesses on one component configured to receive one or more protrusions on another component. The engagement formations may be located laterally of the frame clip. The engagement surfaces may be provided on, or located adjacent, the frame clip. The engagement formations may be located on an interior surface of the frame clip. The engagement formations may be provided on an exterior surface of the frame clip. The exterior surface may be provided on a tab that projects forwardly of the frame clip. The engagement formations may engage with an interference fit.

The mask assembly components may comprise:
a) a mask frame comprising at least one headgear connector; and
b) a cushion with a proximal face contacting portion, a distal inlet aperture, and a side wall between the proximal face contacting portion and the distal inlet aperture; and
c) an inlet connector configured to receive breathable gases from, and be connected to, a gas delivery conduit, the inlet connector being in fluid communication with the cushion.

The mask assembly may be configured such that the mask frame, and in particular the frame clip, is sandwiched between other components of the mask assembly.

The frame clip may be sandwiched between:
a) An exterior surface of the inlet connector and a front surface of the mask assembly;
b) An exterior surface of the inlet connector and an interior surface of the distal inlet aperture;
c) An exterior surface of the inlet connector and an interior surface of the cushion;
d) An exterior surface of the inlet connector and a front surface of the cushion.

The frame clip may be substantially flush with a front surface of the mask assembly. The frame clip may be spaced behind a front surface of the mask assembly.

The frame clip, when fully mounted on the mask assembly, may be connected to one of, or a combination of:
a) The cushion.
b) The distal aperture of the cushion.
c) The inlet connector.
d) The gas delivery conduit.

The cushion may be provided with a latching formation, such as a tooth, configured to engage with a latch on the mask frame, and when so engaged to retain the mask frame on the cushion.

The cushion may comprise a pair of laterally spaced apart teeth. The teeth may extend laterally.

The latch may be provided on the frame clip. The latch may comprise a button to release the latch from engaging the tooth. The button may comprise a tab on the frame clip, the tab being movable relative to the frame clip between latching and non-latching positions.

The mask frame may comprise a second latch configured to releasably engage the inlet connector.

The second latch may be provided on the tab such that the tab functions as a dual function button. A first, for example front part, of the tab, can be pressed to move each tab in a first direction relative to the inlet connector and cushion to disengage the tooth, and release the cushion from the mask frame. A second, for example rear, part of the tab can be moved in the opposed direction, to disengage the second latching formation from the inlet connector, and release the frame from the inlet connector.

The tab may provide a single button with which the user can:
a) release the cushion from the frame whilst retaining the frame on the inlet connector; or
b) release the inlet connector from the frame, whilst retaining the frame on the cushion.

According to an aspect of this disclosure there is provided a mask assembly comprising:
a) a mask frame comprising at least one headgear connector;
b) a cushion; and
c) an inlet connector configured to receive breathable gases from, and be connected to, a gas delivery conduit, the inlet connector being in fluid communication with the cushion.

The cushion may comprise any one or more of:
a) a proximal face contacting portion;
b) a distal inlet aperture;
c) a side wall between the proximal face contacting portion and the distal inlet aperture.

The mask frame may comprise a central connection portion; the inlet connector being mounted on the central connection portion.

The cushion may be mounted on the inlet connector and/or the frame so as to receive gas from the inlet connector.

The frame may be removable from the remainder of the mask assembly independently of the cushion and independently of the inlet connector.

The cushion may be removable from the remainder of the mask assembly independently of the frame and/or independently of the inlet connector.

The inlet connector may comprise an inlet port configured to be connected to the source of pressurised gas. The inlet port of the inlet connector may comprise a central inlet axis, The central inlet axis may be substantially vertical when the mask assembly is viewed from the front; and/or extends substantially downwardly when viewed from the side.

The mask frame may be removably mounted on a component of the mask assembly by moving the mask frame onto the component of the mask assembly in a mounting direction, the mounting direction intersecting, and preferably being orthogonal, to the central inlet axis.

The frame may comprise a frame clip configured to be removably mounted on the inlet connector to mount the central connection portion of the frame to the inlet connector.

The frame clip may be spaced from, and not in contact with, the remainder of the frame, such that forces applied to the remainder of the frame are decoupled from the frame clip.

The central connection portion may be relatively rigid. The remainder of the frame may be relatively flexible.

The frame may comprise lateral portions that extend away from the central connection portion, the lateral portions being relatively flexible.

The frame may be configured to be laterally resiliently deformable. The frame maybe configured to resist deformation in a direction from front to rear of the mask assembly, or vice versa.

The frame may be made from material which is different from the material of the cushion. The frame may be made from material which is relatively harder and/or less deformable than the cushion, and preferably is made from a plastics material.

The inlet connector may be made from material which is different from the material of the cushion. The inlet connector may be made from material which is relatively harder and/or less deformable than the cushion, and preferably is made from a plastics material.

The cushion may be formed from a relatively soft material. The cushion may be formed from a resiliently deformable material, and preferably is formed from silicone. The side walls of the cushion may be resiliently deformable.

A portion of the frame may overlay the cushion. Preferably a portion of the frame overlays the side walls of the cushion. A portion of the frame may overlay an upper portion of the cushion.

The frame may comprise lateral arms, and an upper portion comprising a connecting frame member which extends over an upper portion of the cushion between the lateral arms.

The connecting frame member may:
a) be integral with the lateral arms.
b) be flexible along at least a portion of its length.
c) comprise a central region, wherein the central region may be flexible, and/or may be an apex region of the frame.
d) comprise a central region and end regions, distal from the central region, the end regions being less flexible than the central region.
e) comprise a central region comprising a first width, and end regions comprising a second width, the second width being greater than the first width.

The cushion may comprise an upper portion adjacent the nasal bridge of the user; the upper portion of the cushion comprising a rolling bridge portion which is pivotable towards or away from the nasal bridge of the user to overlap with a central region of the connecting frame member.

Each side wall of the cushion may comprise a scalloped portion and a non-scalloped portion, the scalloped portion being a portion of the side wall that is recessed from the non-scalloped portion, such that the width of the cushion defined between the scalloped portions is narrower than the width of the cushion defined between the non-scalloped portions, when the cushion is viewed from the front, the scalloped portions being delineated from the non-scalloped portions by a respective scallop line that extends along the cushion side wall between the proximal and distal portions of the cushion.

The scalloped portions may each be provided on a lower portion of a respective cushion side wall.

A distal end of each scallop line may be positioned adjacent the diffuser.

Each scallop line may smoothly curve to the cushion proximal portion.

The scallop lines may be contiguous with side margins of the diffuser.

The scalloped portions may recess the cushion inwardly of the inlet connector to which the cushion is mounted.

The scalloped portions may expose portion of the rear of the inlet connector, the exposed portions providing finger grips for a user when removing the inlet connector from the cushion.

In embodiments of this disclosure a mask assembly is provided which comprises:
a) a cushion
b) an inlet connector configured to connect to a source of pressurised gas;
c) a frame comprising at least one headgear connector; the inlet connector being mounted on the frame; the cushion being mounted on the inlet connector so as to receive gas from the inlet connector; wherein
   the frame is removable from the inlet connector independently of the cushion.

The cushion may comprise a proximal face contacting portion, a distal inlet aperture, and a side wall between the proximal face contacting portion and the distal inlet aperture.

The frame may comprise a central connection portion, the inlet connector being mounted on the central connection portion.

The frame may comprise a frame clip configured to mount the frame onto another part of the mask assembly.

The frame clip may be configured to be removably mounted on the inlet connector.

In embodiments of this disclosure a mask assembly is provided which comprises:
a) a cushion with a proximal face contacting portion, a distal inlet aperture, and a side wall between the proximal face contacting portion and the distal inlet aperture;
b) an inlet connector comprising an inlet port configured to connect to a source of pressurised gas;
c) a frame comprising at least one headgear connector and a central connection portion; the inlet connector being mounted on the central connection portion; the cushion being mounted on the inlet connector and/or the frame so as to receive gas from the inlet connector; wherein
   the inlet port of the inlet connector comprises a central inlet axis, wherein the central inlet axis is substantially vertical when the mask assembly is viewed from the front; wherein the mask frame is removably mounted on the mask assembly by moving the mask frame onto the mask assembly in a mounting direction, the mounting direction being orthogonal to the opening axis.

In embodiments of this disclosure a mask assembly is provided which comprises:
a) a cushion with a proximal face contacting portion, a distal inlet aperture, and a side wall between the proximal face contacting portion and the distal inlet aperture;
b) an inlet connector comprising an inlet port configured to connect to a source of pressurised gas;
c) a frame comprising at least one headgear connector and a central connection portion; the central connection portion being mounted on the inlet connector; the cushion being mounted on the inlet connector and/or the frame so as to receive gas from the inlet connector; wherein
   the inlet port of the inlet connector comprises a central inlet axis, wherein the central inlet axis extends downwardly; wherein the mask frame is removably mounted on the inlet connector by moving the mask frame onto the inlet connector in a mounting direction, the mounting direction intersecting the central inlet axis.

The central inlet axis may be inclined relative to the vertical, when the mask assembly is viewed from the side.

The mounting direction may be orthogonal to the central inlet axis.

In embodiments of this disclosure a mask assembly is provided which comprises:
a) a cushion with a proximal face contacting portion, a distal inlet aperture, and a side wall between the proximal face contacting portion and the distal inlet aperture;
b) an inlet connector comprising an inlet port configured to connect to a source of pressurised gas;
c) a frame comprising at least one headgear connector and a central connection portion; the central connection portion being mounted on the inlet connector; the cushion being mounted on the inlet connector and/or the frame so as to receive gas from the inlet connector; wherein
   the inlet port of the inlet connector comprises a central inlet axis; wherein the frame is removably mounted on the inlet connector by moving the frame onto the inlet connector in a mounting direction, the mounting direction being orthogonal to the central inlet axis; wherein the cushion is removably mounted on the frame and/or the inlet connector.

The mask assembly may be configured such that:
a) The cushion may be removed from the inlet connector and/or frame without removing any other component of the mask assembly; and/or
b) The inlet connector may be removed from the frame and/or cushion without removing any other component of the mask assembly.

In embodiments of this disclosure a mask assembly is provided which comprises:
a) a cushion with a proximal face contacting portion, a distal inlet aperture, and a side wall between the proximal face contacting portion and the distal inlet aperture;
b) an inlet comprising an inlet connector comprising an inlet port configured to be connected to a conduit connector cuff configured to receive pressurised gas;
c) a frame comprising at least one headgear connector and a central connection portion; the inlet being mounted on the central connection portion; the cushion being mounted on the inlet and/or the frame so as to receive gas from the inlet; the at least one headgear connector being spaced from the central connection portion; wherein
   the frame comprises a frame clip configured to be removably mounted on the inlet to mount the central connection portion of the frame to the inlet; wherein the frame clip is spaced from, and not in contact with, the remainder of the frame, such that forces applied to the remainder of the frame are decoupled from the frame clip.

The frame clip may comprise opposed sides that extend away from the mask frame, the opposed sides being spaced from the headgear connectors of the frame.

The headgear connectors of the frame may be configured to deflect towards and/or away from the mask frame, without deforming the frame clip.

In embodiments of this disclosure a mask assembly is provided which comprises:
a) a cushion with a proximal face contacting portion, a distal inlet aperture, and a side wall between the proximal face contacting portion and the distal inlet aperture;
b) an inlet comprising an inlet connector comprising an inlet port configured to be connected to a conduit connector cuff configured to receive pressurised gas;
c) a frame comprising at least one headgear connector and a central connection portion; the inlet connector being mounted on the central connection portion; the cushion being mounted on the inlet connector and/or the frame so as to receive gas from the inlet connector; wherein
   the frame is removably mounted on the inlet by moving the frame onto the mask assembly in a mounting direction; wherein the mask assembly comprises at least one guide formation configured to engage the frame and guide the frame onto the mask assembly in the mounting direction.
In embodiments of this disclosure a mask assembly is provided which comprises:
a) a cushion with a proximal face contacting portion, a distal inlet aperture, and a side wall between the proximal face contacting portion and the distal inlet aperture;
b) an inlet comprising an inlet connector comprising an inlet port configured to be connected to a conduit connector cuff configured to receive pressurised gas;
c) a frame comprising at least one headgear connector and a central connection portion; the inlet being mounted on the central connection portion; the cushion being mounted on the inlet and/or the frame so as to receive gas from the inlet; wherein
   the frame comprises a frame clip, the frame clip being configured to be removably mounted on the inlet to mount the frame to the inlet, the frame clip comprising at least one alignment surface that engages with the mask assembly to resist relative rotation between the frame and mask assembly.

According to an aspect of this disclosure there is provided a conduit for transporting respiratory gases, the conduit comprising:
a conduit body comprising an inlet opening, an outlet opening, a gas flow passage extending between the inlet opening and the outlet opening, and a valve opening;
an AA valve located in the gas flow passage and configured to open and close the valve opening;
   wherein the AA valve is overmoulded to the conduit inside the gas flow passage;
wherein the inlet opening and the outlet opening are each configured to receive a respective mould tool to overmould the AA valve inside the gas flow passage;
wherein the inlet opening and the outlet opening are each configured such that the mould tools share a common draw direction.

According to an aspect of this disclosure there is provided a conduit for transporting respiratory gases, the conduit comprising:
a conduit body comprising an inlet opening, an outlet opening, and a gas flow passage extending between the inlet opening and the outlet opening, and a valve opening; and
an AA valve located in the gas flow passage and configured to open and close the valve opening;
wherein the AA valve is overmoulded to the conduit inside the gas flow passage;
wherein the mould material is delivered from only inside the conduit.

According to an aspect of this disclosure there is provided a conduit for transporting respiratory gases, the conduit comprising:
a conduit body comprising an inlet opening, an outlet opening, and a gas flow passage extending between the inlet opening and the outlet opening, and a valve opening; and
an AA valve located in the gas flow passage and configured to open and close the valve opening;
wherein the AA valve is overmoulded to the conduit in the gas flow passage using only two mould tools.

According to an aspect of this disclosure there is provided a conduit for transporting respiratory gases, comprising:
a conduit body comprising an inlet opening, an outlet opening, and a gas flow passage extending between the inlet opening and the outlet opening;
the conduit further comprising an AA valve located in the gas flow passage and configured to open and close a valve opening, and an overmould that comprises the AA valve and extends over a portion of the conduit body.

According to an aspect of this disclosure there is provided a conduit for transporting respiratory gases, comprising:
a conduit body comprising an inlet opening, an outlet opening, and a gas flow passage extending between the inlet opening and the outlet opening;
wherein the conduit body comprises a wall comprising a wall thickness defined between an outer surface and an inner surface of the conduit body;
an AA valve anchoring formation extending from the inner surface of the wall;
the anchoring formation comprising a through-bore configured to receive part of an AA valve to mount the AA valve in the gas flow passage;
wherein the through-bore is aligned with the gas flow passage.

The anchoring formation may extend substantially orthogonally from the inner surface of the wall.

The anchoring formation may extend from the inner surface of the wall so that the anchoring formation is inclined relative to the inner surface of the wall. The anchoring formation may comprise a first surface that faces the inlet opening, and a second surface that faces the outlet opening, at least one of the first and second surfaces being inclined relative to the inner surface of the wall.

According to an aspect of this disclosure there is provided a conduit for transporting respiratory gases, comprising:
a conduit body comprising an inlet opening, an outlet opening, and a gas flow passage extending between the inlet opening and the outlet opening;
the conduit body comprising:
a valve seat extending across the gas flow passage; a valve opening being provided in the valve seat, the valve opening having a valve opening central axis; and
a valve anchor comprising a through-bore extending through the valve seat and comprising a through-bore axis, the through-bore being configured to receive part of an AA valve to mount the AA valve in the gas flow passage, the through-bore axis being aligned with the valve opening central axis.

The conduit may comprise an AA valve located in the gas flow passage.

The AA valve may be overmoulded to the conduit.

According to an aspect of this disclosure there is provided a conduit for transporting respiratory gases, comprising:
a conduit body comprising an inlet opening, an outlet opening, and a gas flow passage extending between the inlet opening and the outlet opening;
the conduit body, inlet opening and outlet opening being configured such that a, straight, gas flow passage axis extends unimpeded through the conduit body between the centre of the inlet opening and the centre of the outlet opening;
the conduit further comprising an AA valve located in the gas flow passage and configured to open and close a valve opening, and an overmould that comprises the AA valve and extends over a portion of the conduit body.

According to an aspect of this disclosure there is provided a conduit for transporting respiratory gases, comprising:
a conduit body comprising an inlet opening, an outlet opening, and a gas flow passage extending between the inlet opening and the outlet opening;
wherein the conduit body comprises a wall comprising a wall thickness defined between an outer surface and an inner surface of the conduit body; the conduit body comprising an AA valve anchoring formation extending from the inner surface of the wall into the gas flow passage;
the conduit further comprising an AA valve located in the gas flow passage and configured to open and close the valve opening;
the conduit further comprising an overmould comprising the AA valve, the overmould extending over both an upstream portion of the anchoring formation, and a downstream portion of the anchoring formation.

The conduit may comprise a valve opening adjacent the AA valve anchoring formation.

The conduit may comprise a valve seat adjacent the AA valve anchoring formation.

The valve seat may extend fully across the gas flow passage.

The AA valve may comprise a valve flap pivotally movable within the gas flow passage about a pivot, at least a distal portion of the valve flap distal from the pivot being configured to overlap the valve seat.

A proximal portion of the valve flap, adjacent the pivot, may overlap the valve seat.

The AA valve anchoring formation may comprise at least one post, extending into the gas flow passage, the AA valve comprising a valve mount, the valve mount being overmoulded onto the post.

The inner surface of the wall may comprise an arcuate wall portion that curves into the gas flow path adjacent the AA valve anchoring formation.

The AA valve anchoring formation may be positioned between a wider conduit portion and a narrower conduit portion.

The AA valve anchoring formation may be positioned between first and second portions of the conduit body.

The inlet opening may comprise an inlet opening axis and the outlet opening may comprise an outlet opening axis, wherein the inlet opening axis and the outlet opening axis are not aligned.

The inlet opening axis and the outlet opening axis may be relatively inclined.

The inlet opening and the outlet opening may be different sizes.

The valve mount may comprise an upstanding flange that projects upwardly from valve mount. The flange may extend substantially across the width of the valve mount.

The valve mount may substantially triangular, when viewed from the side.

The valve mount may comprise an inclined surface adjacent the valve flap, the valve flap being joined to the valve mount by at least one strut that extends between the valve flap and the inclined surface. A plurality of struts may be provided. A gap may be provided between each pair of struts.

According to an aspect of this disclosure there is provided a conduit for carrying respiratory gases, the conduit comprising:
a conduit body comprising an inlet opening, an outlet opening, and a wall defining a gas flow passage extending between the inlet opening and the outlet opening; and
an AA valve located in the gas flow passage, the AA valve comprising a movable valve member;
the conduit body comprising a valve opening formed in the wall of the conduit body; the wall comprising a valve sealing surface surrounding the valve opening and against which the valve member can seal to close the valve opening;
the valve sealing surface comprising a non-planar portion against which a part of the valve member can seal.

The valve member may be pivotally movable about a pivot axis.

The valve member may have a length extending from a proximal valve portion adjacent the pivot axis to a distal valve portion distal the pivot axis.

The non-planar portion may extend along at least part of the length of the valve sealing surface, when viewed along the pivot axis, that is, in side profile, between the pivot axis and a distal margin of the valve opening.

The non-planar portion may have increased curvature at a proximal portion of the valve sealing surface, the proximal portion being between the pivot axis and a mid-point of the valve sealing surface.

The valve sealing surface may comprise a portion of reduced curvature, or no curvature at all, at a distal portion of the valve sealing surface, the distal portion being between the mid-point and the distal margin of the valve sealing surface.

The non-planar portion may extend along the side margins of the valve opening.

The non-planar portion may extend across a lower margin of the valve opening.

The non-planar portion may not extend across an upper margin of the valve opening.

The conduit may be such that, in cross section when viewed from the side, the valve sealing surface comprises:
a) an aerofoil shape;
b) an arcuate portion;
c) one or more bulbous portions that project into the conduit;
d) one or more planar portions;
e) one or more thicker portions;
f) one or more thinner portions.

A portion of the valve sealing surface nearer the pivot axis of the valve flap may project further into the conduit, than the portion of the valve sealing surface distal from the pivot axis.

The valve sealing surface may comprise an arcuate portion having an apex, wherein the apex is closer to the pivot axis of the valve flap, than to the distal margin of the valve opening.

The valve sealing surface may be entirely arcuate, that is, does not comprise any planar portion(s).

The valve sealing surface may comprise one or more non-planar portions and one or more planar portions.

At least one of the non-planar portions may be relatively thicker than at least one of the planar portions.

The valve opening may be positioned:
a) in the non-planar portion;
b) in a thicker portion of the valve sealing surface;
c) in a planar portion of the valve sealing surface;
d) in a thinner portion of the valve sealing surface;
e) to extend across one portion to another portion of the valve sealing surface.

The valve member may comprise a reinforcing formation configured to seal against the valve sealing surface. The reinforcing formation may comprise a thickness, the thickness being the distance by which the reinforcing formation extends away from the remainder of the valve flap. The thickness of the reinforcing formation may be constant along the length of the reinforcing formation. The thickness of the reinforcing formation may vary along the length of the reinforcing formation. The thickness of the reinforcing formation may be greater adjacent, or nearer, a pivot axis of the valve flap.

According to an aspect of this disclosure there is provided a cushion for a respiratory mask assembly, comprising a face contacting portion, and an inlet aperture having an aperture axis, the inlet aperture being configured to be connected to a connector for delivering respiratory gases to the cushion, the cushion comprising a peripheral seal extending around the periphery of the distal inlet aperture, the peripheral seal comprising a sealing rib that protrudes outwardly from the exterior of the cushion, generally in the direction of the aperture axis.

The distal inlet aperture may comprise a relatively rigid ring that is more rigid than the proximal face contacting portion, the rigid ring providing a connection between the cushion and the connector.

The rigid ring may be located inside the distal inlet aperture.

The rigid ring may comprise an integral part of the cushion.

The rigid ring may be overmoulded to the cushion.

The sealing rib may comprise part of the overmould.

The sealing rib may be adjacent the rigid ring.

The rigid ring may extend around the inside perimeter of the distal inlet aperture.

The rigid ring may not project forwardly from the distal inlet aperture, that is, the rigid ring is substantially flush with, or recessed from, the outer surface of the cushion.

The rigid ring may comprise a thickened or reinforced part of the cushion, formed from the same material as the cushion.

The rigid ring may be made from a plastics material.

The distal inlet aperture may be configured to be connected to the connector with an interference fit.

The rigid ring may comprise at least one connecting formation configured to connect with the connector.

According to an aspect of this disclosure there is provided a mask assembly comprising
a cushion; and
a frame, the cushion being mounted on the frame;
the frame comprising a pair of headgear connectors each comprising a mounting post; each mounting post comprising a width dimension;
the frame comprising a pair of elongate guide portions, each elongate guide portion extending along the frame to a respective mounting post and configured to guide a headgear connector clip along that elongate guide portion to the respective mounting post;
each elongate guide portion comprising a width dimension;
wherein the width dimension of each elongate guide portion is less than the width dimension of the mounting post.

Each elongate guide portion comprises a guide rail.

The guide rail may be substantially oblong in transverse cross section and comprises the width dimension and a height dimension, when viewed along a longitudinal axis of the guide rail.

The frame may comprise a pair of lateral arms extending outwardly and rearwardly from a central portion of the frame, wherein each elongate guide portion extends along a respective lateral arm of the frame.

Each lateral arm may have a width dimension, wherein the width dimension of the elongate guide portion is less than the width dimension of the lateral arm.

The mask assembly may comprise a headgear connector clip comprising a hook comprising a post receiving portion configured to receive the mounting post to connect the headgear connector clip to the frame.

The height dimension of the guide rails may be greater than a height dimension of the post receiving portion of the hook.

The hook of the headgear connector clip may comprise a mouth portion adjacent the post receiving portion and having a width dimension that is less than the width dimension of the post receiving portion.

The width dimension of each guide rail may be less than the width dimension of the mouth portion.

The width dimension of each guide rail may be less than the width dimension of the mouth portion of the headgear connector clip.

The headgear connector clip may be mountable on the headgear connector of the frame with an interference fit.

The cushion may comprise a proximal face contacting portion, a distal inlet aperture, and a side wall between the proximal face contacting portion and the distal inlet aperture.

The cushion may be releasably mounted on the frame.

According to an aspect of this disclosure there is provided a conduit for carrying respiratory gases, the conduit comprising:
a conduit body comprising an inlet opening, an outlet opening, and a gas flow passage extending between the inlet opening and the outlet opening; and
an AA valve located in the gas flow passage; and
a valve seat comprising a valve port;
the AA valve comprising a valve flap pivotally movable relative to the valve seat about a pivot axis and comprising a proximal portion proximal the pivot axis, a distal portion distal the pivot axis, and opposed side portions extending between the proximal and distal portions;
the valve flap being configured to pivot about the pivot axis to a closed condition in which the valve flap closes the valve port;
wherein, when the valve flap is in the closed condition, the proximal and distal portions of the valve flap overlap the valve seat; and the side portions of the valve flap do not overlap the valve seat.

The valve flap may be planar.

The conduit may be configured such that:
a) the distal portion of the valve flap is arcuate; and/or
b) the side portions of the valve flap are straight; and/or
c) the proximal portion of the valve flap is straight.

The conduit of any one of claims 60 to 62 wherein the side portions of the valve flap are spaced apart by a width dimension, the width dimension being less than or equal to the width of the valve port.

The valve flap may comprise any one or more of:
a) a reinforcing formation on a non-sealing surface of the valve flap; and/or
b) a reinforcing formation on a sealing surface of the valve flap.

The reinforcing formation may comprise a perimeter ridge extending around at least a portion of the perimeter of the valve flap.

The reinforcing formation may comprise a thickness, the thickness being the distance by which the reinforcing formation extends away from the remainder of the valve flap. The thickness of one or each reinforcing formation may be constant along the length of the reinforcing formation. The thickness of one or each reinforcing formation may vary along the length of the reinforcing formation. The thickness of one or each reinforcing formation may be greater adjacent, or nearer, a pivot axis of the valve flap.

According to an aspect of this disclosure there is provided a conduit comprising an anti-asphyxia valve, the valve comprising a valve seat comprising a valve port, and a valve flap configured to close the valve port, the valve flap comprising end portions that overlap the valve seat when the valve flap closes the valve port, and side portions between the end portions, wherein the side portions do not overlap the valve seat when the valve flap closes the valve port.

According to an aspect of this disclosure there is provided a mask assembly comprising an anti-asphyxia valve, the valve comprising a valve seat comprising a valve port, and a valve flap configured to close the valve port.

The AA valve may comprise an anti-inversion feature, configured to prevent or resist the valve flap from moving into the valve port. The anti-inversion feature may comprise any one or more of:
a) one or more lugs extending from the periphery of the valve port and terminating part way across the valve port; and/or
b) one or more ribs extending from the periphery of the valve port, all the way across the inlet port.

A plurality of lugs and/or ribs may be provided.

The or each rib or lug may form part of the surface against which the valve flap is moulded during overmoulding of the AA valve.

The valve flap may be configured to be biased to a closed position, that is, a position in which the valve flap closes the valve port. The valve flap may be configured to be inclined relative to a valve mount,, for example after moulding the AA valve in the mask assembly.

The valve flap may be smaller than the inlet port, such that when the valve flap closes the valve port, a gap is provided between the periphery of the valve flap and the periphery of the valve port.

The valve flap may be the same size as the valve port, such that when the valve flap closes the valve port, no gap is provided between the periphery of the valve flap and the periphery of the valve port.

The valve flap may be larger than the valve port, such that when the valve flap closes the valve port, the periphery of the valve port overlaps with the periphery of the valve port.

The valve flap may comprise a reduced thickness region. The reduced thickness region may be adjacent a valve mount of the AA valve. The reduced thickness region may be semi-circular when viewed in plan.

The valve flap may be connected to a valve mount by a thinned region. The thinned region may be of non-constant thickness across its width, that is, in a direction parallel with the pivot axis of the valve flap. The thinned region may comprise opposed side margins, the opposed side margins being thicker than the remainder of the thinned region. The opposed side margins may comprise side walls that are thicker than the thinned region. The thickness of the thinned region may taper from the side margins to a thinner central region between the side margins.

The valve flap may comprise a pull tab, used to pull the valve flap into the mask assembly during assembly. The pull tab may be integral with the valve flap. The pull tab may be removable from the valve flap.

The valve mount may be received in a slot. The slot may be provided in for example an inlet connector of the mask assembly. The slot and/or the valve mount may be configured to limit movement of the valve mount through the slot. The slot and/or the valve mount may comprise at least one tapered surface. The slot and/or the valve mount may become smaller along an insertion direction of the valve mount. The slot and/or valve mount may comprise at least one stepped surface.

The AA valve may be collapsible to allow the valve to be collapsed during assembly. The AA valve may be foldable.

According to an aspect of this disclosure there is provided a mask assembly comprising:
a) a mask frame comprising at least one headgear connector;
b) a cushion;
c) an inlet connector configured to receive breathable gases from, and be connected to, a gas delivery conduit, the inlet connector being in fluid communication with the cushion; and
d) a diffuser comprising a diffuser frame.

The diffuser frame and/or the inlet connector may be provided with a scalloped gripping region to allow a user to insert a finger into the gripping region to remove the diffuser frame from the inlet connector. The gripping region may be provided at a lower margin of the diffuser frame.

The diffuser frame may be slidably mountable on the inlet connector. A lower portion of the diffuser frame may comprise a tongue configured to be received in a corresponding slot of the inlet connector, when the diffuser frame is fully slidably mounted on the inlet connector. The diffuser frame may comprise one or more gripping portions to facilitate a user moving the diffuser frame relative to the inlet connector.

According to an aspect of this disclosure there is provided a mask assembly comprising:
a) a mask frame comprising at least one headgear connector;
b) a cushion; and
c) an inlet connector configured to receive breathable gases from, and be connected to, a gas delivery conduit, the inlet connector being in fluid communication with the cushion.

The frame and/or the inlet connector may comprise one or more engagement features configured to resist relative rotation between the frame and inlet connector.

The frame may comprise at least one female engagement feature. The inlet connector may comprise at least one male engagement feature.

The frame may comprise at least one male engagement feature. The inlet connector may comprise at least one female engagement feature.

Th engagement feature(s) may be partially spherical. The engagement features may be partially cylindrical.

A pair of engagement features may be provided on opposed sides of the inlet connector, and on opposed sides of the frame.

The male engagement features may protrude in a direction which extends radially outward of an inlet port of the inlet connector. The male engagement features may protrude in a direction aligned with the axis of the inlet port of the inlet connector.

The engagement features may comprise a recess at a forward portion of one of the inlet connector and frame clip, and at least one rib on a forward portion of the other of the inlet connector and frame clip, the rib being received in the recess. The recess may be oblong. The rib may extend in a direction parallel with the axis of the inlet port. A plurality of ribs may be provided. A plurality of recesses may be provided, each recess receiving a respective rib.

The engagement feature may comprise an aperture on a forward portion of one of the inlet connector and frame clip, and a pin on a forward portion of the other of the inlet connector and frame clip, the pin being received in the aperture.

The engagement feature may comprise a downwardly directed tab on the frame, and a cut-out on the inlet connector, the tab being received in the cut-out. The tab may be provided at the bottom of the frame. The cut-out may be provided at the bottom of the inlet connector. The tab and the cut-out may be oblong. The cut-out may be provided in a lowermost flange of the inlet connector.

The engagement feature may comprise one or more lugs on the inlet connector and one or more projections on the frame, each lug comprising a guide surface over which the projection passes during assembly of the inlet connector onto the frame, each lug further comprising an engagement surface against which the projection engages, when the inlet connector is connected to the frame. The guide surface may be inclined or arcuate. The engagement surface may be planar. The frame may deform outwardly as the lug passes over the guide surface before reverting to its original shape once the lug engages the engagement surface. A pair of lugs and a pair of projections may be provided.

The frame may comprise a guide rail along which the inlet connector slides during assembly. A pair of guide rails may be provided. The inlet connector may comprise one or more elongate channels each configured to receive a respective guide rail.

The frame may comprise one or more thinned portions configured to increase flexibility of the frame. A thinned portion may be provided between each lateral arm and the frame clip.

The inlet connector may comprise one or more cushion retainers configured to releasably engage with the cushion to connect the cushion to the inlet connector. A pair of cushion retainers may be provided, on opposed sides of the inlet connector. The cushion may comprise at least one formation configured to be engaged by the cushion retainers. The at least one formation may comprise a ridge on the cushion. Each cushion retainer may comprise a flexible finger. Each flexible finger may be elongate. Each flexible finger may comprise a mount configured to movably mount the flexible finger on the inlet connector. The mount may be part way along the flexible finger. The flexible finger may comprise a distal end adjacent the cushion and a proximal end spaced from the cushion. The proximal end may comprise a hook configured to engage the cushion. The distal end may comprise a user contact surface to enable the user to press the distal end to move the proximal end and disconnect the flexible finger from the cushion.

The cushion may comprise one or more tabs configured to be received in one or more corresponding slots on the inlet connector. A pair of tabs and a pair of slots may be provided. Each tab may comprise a hook to releasably engage the inlet connector, when the tab is received in the slot. The tabs may extend forwardly.

The inlet connector may comprise a thinned region comprising a recess, the cushion comprising a ridge configured to releasably engage the recess, the recess being disengaged from the ridge by the user moving the thinned region.

The cushion may comprise a blade seal extending around the periphery of the inlet aperture of the cushion. The blade seal may be configured to be rearwardly directed when in a rest condition, that is, inclined into the cushion away from the inlet aperture. The blade seal may be configured to invert during removal of the cushion from the inlet connector, that is, the blade seal may be configured to move from the rearwardly directed position to a forwardly directed position during removal. The cushion may comprise a rigid ring and the blade seal. The inlet connector may comprise a first peripheral groove, the blade seal being received in the first peripheral groove when the inlet connector and cushion are connected together. The inlet connector may comprise a second peripheral groove, the rigid ring of the cushion being received in the second peripheral groove when the inlet connector and cushion are connected together.

The inlet opening of the cushion may comprise a region of thickened material configured to engage with the outlet boss of the inlet connector. The region of thickened material may terminate in a chamfered peripheral surface, configured to engage with a corresponding chamfered surface of the outlet boss.

The cushion may comprise on or more reinforced regions, adjacent the inlet aperture. The reinforced regions may comprise internal ribs. The ribs may be spaced around the inlet aperture.

According to an aspect of this disclosure there is provided an inlet connector for a respiratory mask assembly, the inlet connector comprising:
an inlet opening configured to connect to a source of breathable gas;
bias vent holes configured to vent gas from the inlet connector;
an anti-asphyxia valve opening; and
   a diffuser that covers the anti-asphyxia valve opening and the bias vent holes;
   wherein the diffuser is mounted on the inlet connector; at least one vent being defined between the diffuser and the inlet connector, the vent forming a gases pathway between the bias vent holes and/or the AA valve opening, and the external environment.

The diffuser may comprise a body of diffuser material and a diffuser frame, the diffuser material being held on the diffuser frame, the diffuser frame being mounted on the inlet connector. The diffuser frame may form a perimeter around the periphery of the diffuser material.

The diffuser may be permanently mounted on the inlet connector.

The diffuser may be removably mounted on the inlet connector.

The diffuser frame may be substantially rigid.

The diffuser and the inlet connector may be configured such that a plurality of vents are defined therebetween.

At least one vent may be adjacent a lower portion of the diffuser. At least one vent may be adjacent a lower margin of the diffuser.

At least one vent may be adjacent an upper portion of the diffuser. At least one vent may be adjacent an upper margin of the diffuser.

At least one vent may be adjacent a side portion of the diffuser. At least one vent may be adjacent a side margin of the diffuser.

At least one vent may be adjacent the AA valve opening.

At least one vent may be adjacent the bias vent holes.

The bias vent holes may be provided in an upper portion of the inlet connector.

The AA valve opening may be provided in a lower portion of the inlet connector.

The bias vent holes may be positioned above the AA valve opening, when the inlet connector is viewed from the front.

At least one vent may have the same configuration as another vent.

At least one vent may have a different configuration from another vent.

The vent configuration may include any one or more of the following features:
a) vent opening size;
b) vent opening shape;
c) vent pathway length;
d) vent position;
e) vent pathway direction.

The inlet connector may comprise a forward wall. The bias vent holes may be formed in the forward wall. The anti-asphyxia valve opening may be formed in the forward wall.

The inlet opening may comprise an inlet opening axis, the forward wall being inclined relative to the inlet opening axis.

The forward wall may comprise upper and lower margins, and may be curved between the upper and lower margins.

The forward wall may comprise opposed side margins, and may be curved between the side margins.

The bias vent holes may be adjacent the anti-asphyxia valve opening. The bias vent holes may be above the anti-asphyxia valve opening, when the inlet connector is viewed from the front.

The inlet connector may comprise a central body, and an outer body depending from the central body. The outer body may comprise a flange extending outwardly from the central body. The flange may extend laterally outwardly from the central body. The central body may comprise opposed side portions when viewed from the front, the flange extending laterally outwardly from both side portions of the central body.

The bias flow vent holes and the anti-asphyxia valve opening may be provided in the central body. The diffuser, when mounted on the inlet connector, may overlap the outer body. The diffuser may be in contact with the central body, but spaced apart from the outer body.

At least one vent may be provided between the flange and the diffuser frame.

The inlet connector may comprise a locating/alignment feature configured to accommodate a corresponding locating/alignment feature of the diffuser. The inlet connector and/or the diffuser may comprise a plurality of locating/alignment features. At least one locating/alignment feature may comprise a notch configured to accommodate another locating/alignment feature. The notch may be configured to receive another locating/alignment feature. The other locating/alignment feature may comprise a corner configured to mate with the notch, to locate/align the inlet connector with the diffuser.

Where the inlet connector comprises a flange, the flange may comprise an outer margin, the outer margin comprising a locating/alignment feature. The locating/alignment feature of the flange may comprise a corner configured to mate with a locating/alignment notch of the diffuser.

The vent may be an opening defined by one or both of the inlet connector and the diffuser.

The opening may be defined by a channel formed by one or both of the inlet connector and the diffuser.

The inlet connector may comprise a recessed portion, the recessed portion configured to provide a clearance from the diffuser frame to at least partially define the vent.

The diffuser frame may comprise a recessed portion, the recessed portion configured to provide a clearance from the inlet connector to at least partially define the vent.

More than one recessed portion may be provided.

Both the diffuser frame and the inlet connector may comprise respective recessed portions.

The recessed portion may be elongate so as to define a channel between the inlet connector and the diffuser frame.

The recessed portion may comprise one or more indents.

The inlet connector may be configured to connect to a cushion.

The inlet connector may be configured to connect to a frame, the frame being configured to connect to headgear.

The inlet connector may comprise one or more tube connection features configured to connect to a breathing gases delivery tube.

The anti-asphyxia valve opening may comprise a plurality of openings.

The inlet connector may comprise a rib configured to extend across the anti-asphyxia valve opening. The rib may be configured to extend between upper and lower margins of the anti-asphyxia valve opening. The rib may be configured to project outwardly from the anti-asphyxia valve opening. The rib may be configured to engage the diffuser, to space at least part of the diffuser from the anti-asphyxia valve opening. The rib may be configured to space the diffuser material from the anti-asphyxia valve opening.

The inlet connector may comprise a rib adjacent the bias vent holes. The bias vent holes may be arranged in a bias vent hole array, the rib being configured to extend across the bias vent hole array. The rib may be configured to extend only partially across the bias vent hole array. The rib may be configured to extend in a direction between upper and lower margins of the bias vent hole array. The rib may be configured to project outwardly from the bias vent hole array. The rib may be configured to engage the diffuser, to space at least part of the diffuser from the bias vent hole array. The rib may be configured to space the diffuser material from the bias vent hole array.

The inlet connector may comprise a first rib adjacent the anti-asphyxia valve opening and a second rib adjacent the bias vent holes. The first and second ribs may be spaced apart to enable fluid communication therebetween.

The diffuser frame may comprise:
a) an arcuate upper margin;
b) a lower margin; and
c) a pair of side margins that extend between the upper and lower margins.

The lower margin may be arcuate.

The lower margin may be shorter than the upper margin.

The side margins may be straight.

The side margins may be inclined laterally outwardly from the lower margin to the upper margin.

The diffuser frame may widen laterally outwardly from the lower margin to the upper margin, when the diffuser frame is viewed from the front.

The diffuser material may comprise an outer periphery which is substantially the same shape as the shape of the outer periphery of the diffuser frame, when the diffuser is viewed from the front, the diffuser frame forming a perimeter around the periphery of the diffuser material.

The inlet connector may comprise an anti-asphyxia valve.

According to an aspect of this disclosure there is provided an inlet connector for a respiratory mask assembly, the inlet connector comprising:
an inlet opening configured to connect to a source of breathable gas;
bias vent holes configured to vent gas from the inlet connector;
an anti-asphyxia valve opening; and
a diffuser that covers the anti-asphyxia valve opening and the bias vent holes;
   wherein the diffuser is mounted on the inlet connector; a plurality of vents being defined between the diffuser and the inlet connector, each vent forming a respective gases pathway between the bias vent holes and/or the AA valve opening, and the external environment;
the plurality of vents comprising:
   a first vent defined between the diffuser and inlet connector adjacent the AA valve opening; and
   a second vent defined between the diffuser and the inlet connector adjacent the bias vent holes.

The diffuser may comprise a body of diffuser material and a diffuser frame, the diffuser material being held on the diffuser frame, the diffuser frame being mounted on the inlet connector. The diffuser frame may form a perimeter around the periphery of the diffuser material.

The diffuser may be permanently mounted on the inlet connector.

The diffuser frame may be removably mounted on the inlet connector.

The inlet connector may comprise a diffuser frame, the first and second vents being defined between the diffuser frame and the inlet connector.

The diffuser frame may be substantially rigid.

The first and second vents may be defined between the diffuser cover and the inlet connector.

A third vent may be defined between the diffuser and the inlet connector.

At least one vent may have the same configuration as another vent.

At least one vent may have a different configuration from another vent.

The vent configuration may include any one or more of the following features:
a) vent opening size;
b) vent opening shape;
c) vent pathway length;
d) vent position;
e) vent pathway direction.

At least one vent may be adjacent a lower portion of the diffuser. At least one vent may be adjacent a lower margin of the diffuser.

At least one vent may be adjacent an upper portion of the diffuser. At least one vent may be adjacent an upper margin of the diffuser.

At least one vent may be adjacent a side portion of the diffuser. At least one vent may be adjacent a side margin of the diffuser.

The inlet connector may comprise a forward wall. The bias vent holes may be formed in the forward wall. The anti-asphyxia valve opening may be formed in the forward wall.

The inlet opening may comprise an inlet opening axis, the forward wall being inclined relative to the inlet opening axis.

The forward wall may comprise upper and lower margins, and may be curved between the upper and lower margins.

The forward wall may comprise opposed side margins, and may be curved between the side margins.

The bias vent holes may be adjacent the anti-asphyxia valve opening. The bias vent holes may be above the anti-asphyxia valve opening, when the inlet connector is viewed from the front.

The inlet connector may comprise a central body, and an outer body depending from the central body. The outer body may comprise a flange extending outwardly from the central body. The flange may extend laterally outwardly from the central body. The central body may comprise opposed side portions when viewed from the front, the flange extending laterally outwardly from both side portions of the central body.

The bias flow vent holes and the anti-asphyxia valve opening may be provided in the central body. The diffuser, when mounted on the inlet connector, may overlap the outer body. The diffuser may be in contact with the central body, but spaced apart from the outer body.

At least one vent may be provided between the flange and the diffuser frame.

The inlet connector may comprise a locating/alignment feature configured to accommodate a corresponding locating/alignment feature of the diffuser. The inlet connector and/or the diffuser may comprise a plurality of locating/alignment features. At least one locating/alignment feature may comprise a notch configured to accommodate another locating/alignment feature. The notch may be configured to receive another locating/alignment feature. The other locating/alignment feature may comprise a corner configured to mate with the notch, to locate/align the inlet connector with the diffuser.

Where the inlet connector comprises a flange, the flange may comprise an outer margin, the outer margin comprising a locating/alignment feature. The locating/alignment feature of the flange may comprise a corner configured to mate with a locating/alignment notch of the diffuser.

The vent may be an opening defined by one or both of the inlet connector and the diffuser.

The opening may be defined by a channel formed by one or both of the inlet connector and the diffuser.

The inlet connector may comprise a recessed portion, the recessed portion configured to provide a clearance from the diffuser frame to at least partially define the vent.

The diffuser frame may comprise a recessed portion, the recessed portion configured to provide a clearance from the inlet connector to at least partially define the vent.

More than one recessed portion may be provided.

Both the diffuser frame and the inlet connector may comprise respective recessed portions.

The recessed portion may be elongate so as to define a channel between the inlet connector and the diffuser frame.

The recessed portion may comprise one or more indents.

The inlet connector may be configured to connect to a cushion.

The inlet connector may be configured to connect to a frame, the frame being configured to connect to headgear.

The inlet connector may comprise one or more tube connection features configured to connect to a breathing gases delivery tube.

According to an aspect of this disclosure there is provided an inlet connector for a respiratory mask assembly, the inlet connector comprising:
an inlet opening configured to connect to a source of breathable gas;
bias vent holes configured to vent gas from the inlet connector;
an anti-asphyxia valve opening;
a diffuser that covers the anti-asphyxia valve opening and the bias vent holes; and
a vent.

The diffuser may comprise a body of diffuser material and a diffuser frame, the diffuser material being held on the diffuser frame, the diffuser frame being mounted on the inlet connector. The diffuser frame may form a perimeter around the periphery of the diffuser material.

The diffuser may be permanently mounted on the inlet connector.

The diffuser frame may be removably mounted on the inlet connector.

The diffuser frame may be substantially rigid.

The diffuser frame and the inlet connector may be configured such that a at least one vent is defined therebetween. A plurality of vents may be defined therebetween.

At least one vent may be adjacent a lower portion of the diffuser frame. At least one vent may be adjacent a lower margin of the diffuser frame.

At least one vent may be adjacent an upper portion of the diffuser frame. At least one vent may be adjacent an upper margin of the diffuser frame.

At least one vent may be adjacent a side portion of the diffuser frame. At least one vent may be adjacent a side margin of the diffuser frame.

At least one vent may be adjacent the AA valve opening.

At least one vent may be adjacent the bias vent holes.

The bias vent holes may be provided in an upper portion of the inlet connector.

The AA valve opening may be provided in a lower portion of the inlet connector.

The bias vent holes may be positioned above the AA valve opening, when the inlet connector is viewed from the front.

At least one vent may have the same configuration as another vent.

At least one vent may have a different configuration from another vent.

The vent configuration may include any one or more of the following features:
a) vent opening size;
b) vent opening shape;
c) vent pathway length;
d) vent position;
e) vent pathway direction.

The inlet connector may comprise a forward wall. The bias vent holes may be formed in the forward wall. The anti-asphyxia valve opening may be formed in the forward wall.

The inlet opening may comprise an inlet opening axis, the forward wall being inclined relative to the inlet opening axis.

The forward wall may comprise upper and lower margins, and may be curved between the upper and lower margins.

The forward wall may comprise opposed side margins, and may be curved between the side margins.

The bias vent holes may be adjacent the anti-asphyxia valve opening. The bias vent holes may be above the anti-asphyxia valve opening, when the inlet connector is viewed from the front.

The inlet connector may comprise a central body, and an outer body depending from the central body. The outer body may comprise a flange extending outwardly from the central body. The flange may extend laterally outwardly from the central body. The central body may comprise opposed side portions when viewed from the front, the flange extending laterally outwardly from both side portions of the central body.

The bias flow vent holes and the anti-asphyxia valve opening may be provided in the central body. The diffuser, when mounted on the inlet connector, may overlap the outer body. The diffuser may be in contact with the central body, but spaced apart from the outer body.

At least one vent may be provided between the flange and the diffuser frame.

The inlet connector may comprise a locating/alignment feature configured to accommodate a corresponding locating/alignment feature of the diffuser. The inlet connector and/or the diffuser may comprise a plurality of locating/alignment features. At least one locating/alignment feature may comprise a notch configured to accommodate another locating/alignment feature. The notch may be configured to receive another locating/alignment feature. The other locating/alignment feature may comprise a corner configured to mate with the notch, to locate/align the inlet connector with the diffuser.

Where the inlet connector comprises a flange, the flange may comprise an outer margin, the outer margin comprising a locating/alignment feature. The locating/alignment feature of the flange may comprise a corner configured to mate with a locating/alignment notch of the diffuser.

The vent may be an opening defined by one or both of the inlet connector and the diffuser.

The opening may be defined by a channel formed by one or both of the inlet connector and the diffuser.

The inlet connector may comprise a recessed portion, the recessed portion configured to provide a clearance from the diffuser frame to at least partially define the vent.

The diffuser frame may comprise a recessed portion, the recessed portion configured to provide a clearance from the inlet connector to at least partially define the vent.

More than one recessed portion may be provided.

Both the diffuser frame and the inlet connector may comprise respective recessed portions.

The recessed portion may be elongate so as to define a channel between the inlet connector and the diffuser frame.

The recessed portion may comprise one or more indents.

The inlet connector may be configured to connect to a cushion.

The inlet connector may be configured to connect to a frame, the frame being configured to connect to headgear.

The inlet connector may comprise one or more tube connection features configured to connect to a breathing gases delivery tube.

The anti-asphyxia valve opening may comprise a plurality of openings.

The inlet connector may comprise a rib configured to extend across the anti-asphyxia valve opening. The rib may be configured to extend between upper and lower margins of the anti-asphyxia valve opening. The rib may be configured to project outwardly from the anti-asphyxia valve opening. The rib may be configured to engage the diffuser, to space at least part of the diffuser from the anti-asphyxia valve opening. The rib may be configured to space the diffuser material from the anti-asphyxia valve opening.

The inlet connector may comprise a rib adjacent the bias vent holes. The bias vent holes may be arranged in a bias vent hole array, the rib being configured to extend across the bias vent hole array. The rib may be configured to extend only partially across the bias vent hole array. The rib may be configured to extend in a direction between upper and lower margins of the bias vent hole array. The rib may be configured to project outwardly from the bias vent hole array. The rib may be configured to engage the diffuser, to space at least part of the diffuser from the bias vent hole array. The rib may be configured to space the diffuser material from the bias vent hole array.

The inlet connector may comprise a first rib adjacent the anti-asphyxia valve opening and a second rib adjacent the bias vent holes. The first and second ribs may be spaced apart to enable fluid communication therebetween.

The diffuser frame may comprise:
a) an arcuate upper margin;
b) a lower margin; and
c) a pair of side margins that extend between the upper and lower margins.

The lower margin may be arcuate.

The lower margin may be shorter than the upper margin.

The side margins may be straight.

The side margins may be inclined laterally outwardly from the lower margin to the upper margin.

The diffuser frame may widen laterally outwardly from the lower margin to the upper margin, when the diffuser frame is viewed from the front.

The diffuser material may comprise an outer periphery which is substantially the same shape as the shape of the outer periphery of the diffuser frame, when the diffuser is viewed from the front, the diffuser frame forming a perimeter around the periphery of the diffuser material.

The inlet connector may comprise an anti-asphyxia valve.

According to an aspect of this disclosure there is provided a respiratory mask kit comprising:
a) the mask assembly of any one of the preceding statements; and
b) headgear.

The headgear may comprise a strap assembly comprising a pair of opposing upper side straps, and a pair of opposing lower side straps; wherein the lower side straps each terminate in headgear connector elements configured to connect to headgear connectors on the frame of the mask assembly.

The respiratory mask kit may comprise any one or more of:
a) a tube connector for connecting a breathing gas delivery tube to the mask assembly;
b) a breathing gas delivery tube.

The breathing gas delivery tube may comprise:
a) a tube heater wire; and/or
b) a sensor wire; and/or
c) a connector cuff at an end of the tube.

### BRIEF DESCRIPTION OF THE DRAWINGS

Throughout the drawings, reference numbers can be reused to indicate general correspondence between reference elements. The drawings are provided to illustrate example embodiments described herein and are not intended to limit the scope of the disclosure.

FIG. 1 is a front and top perspective view of a mask assembly in accordance with this disclosure, and a breathing conduit and conduit connector.

FIG. 2 is an exploded view corresponding to FIG. 1.

FIG. 3 is an enlarged perspective view of the mask assembly of FIG. 1, additionally showing straps of a headgear connected to the mask assembly.

FIGS. 4a to 4c are perspective views of components of the mask assembly of FIG. 1, respectively the mask assembly in an assembled condition, a cushion, and a mask frame. In this embodiment the mask frame is connected to, or includes, an inlet connector.

FIGS. 5a to 5c are front views of components of the mask assembly of FIG. 1, respectively showing the mask assembly in an assembled condition, a cushion, and a mask frame. In this embodiment the mask frame is connected to, or includes, an inlet connector.

FIGS. 6a to 6c are side views of components of the mask assembly of FIG. 1, respectively showing the mask assembly in an assembled condition, a cushion, and a mask frame. In this embodiment the mask frame is connected to, or includes, an inlet connector.

FIGS. 7a to 7c are top views of components of the mask assembly of FIG. 1, respectively showing the mask assembly in an assembled condition, a cushion, and a mask frame. In this embodiment the mask frame is connected to, or includes, an inlet connector.

FIGS. 8a to 8c are bottom views of components of the mask assembly of FIG. 1, respectively showing the mask assembly in an assembled condition, a cushion, and a mask frame. In this embodiment the mask frame is connected to, or includes, an inlet connector.

FIGS. 9a to 9c are rear views of components of the mask assembly of FIG. 1, respectively showing the mask assembly in an assembled condition, a cushion, and a mask frame. In this embodiment the mask frame is connected to, or includes, an inlet connector.

FIG. 10 is an enlarged perspective view of the mask assembly of FIG. 1, showing an upper end of a breathing gas delivery conduit connected to the mask assembly.

FIG. 11 is an exploded perspective view from the rear of the mask assembly of FIG. 1.

FIG. 12a) to 12e) are perspective, top, rear, front, and side views of a mask frame of the mask assembly of FIG. 1, with the inlet connector omitted.

FIGS. 13a) and 13b) are front and top views respectively of the frame of FIG. 12, with arrows showing the direction of applied forces.

FIG. 14 is a rear view of the frame of FIG. 12, showing frame regions that have different resistance to bending.

FIG. 15 is an enlarged view of part of the frame of FIG. 14.

FIG. 16 is a top view of the frame of FIG. 12, connected to a breathing gas delivery conduit.

FIG. 17 is a perspective view corresponding to FIG. 16.

FIG. 18a) is a side view of a cushion of the mask assembly of FIG. 1, with FIG. 18b) being an enlarged view of the area shown in the box of FIG. 18a), with both figures showing an upper portion of the cushion in a first condition.

FIG. 19 is a view corresponding to FIG. 18, showing the upper portion of the cushion in a second condition in which the upper portion of the cushion is pivoted forwardly so as to extend over an upper part of the frame.

FIG. 20a) to 20c) are side views of the cushion, with the dashed lines of FIG. 20a showing the cushion in the second condition, and FIG. 20c showing the cushion in the second condition.

FIGS. 21a) and 21b) are perspective views showing the cushion in the first and second conditions.

FIG. 22a) is a top view of the frame of FIG. 12 with the dashed lines showing a deformed position of the frame when subject to lateral forces; whilst FIGS. 22b) and 22c) are front and top views with the dashed lines indicating a deformed position of the cushion when subject to lateral forces.

FIGS. 23a) and 23b) are top views of the mask assembly, with FIG. 23b) showing the mask assembly in a deformed position when subject to lateral forces.

FIGS. 24a) and 24b) are side views of the mask assembly of FIG. 1 connected to headgear having upper and lower side straps connected to the frame of the mask assembly, showing the angle of the upper and lower side straps relative to each other, and to the mask assembly.

FIGS. 25a) and 25b) are top views of the mask assembly of FIG. 1 connected to headgear having upper and lower side straps connected to the frame of the mask assembly, showing the angle of the upper side straps relative to each other, and to the mask assembly, and showing the angle of the lateral arms of the frame.

FIG. 26 is a perspective view of the mask assembly of FIG. 1 connected to headgear and a breathing gas delivery conduit, showing a diffuser of the mask assembly.

FIG. 27 is a perspective view of a mask assembly in accordance with this disclosure, with a modified frame.

FIG. 28 is an enlarged side view of a headgear connector of the frame of FIG. 27.

FIG. 29 is a perspective view of the frame of FIGS. 27 and 28 and an inlet connector attached to, mounted on, or comprising part of, the frame.

FIG. 30 is an enlarged perspective view of the frame and inlet connector of FIG. 29, with a diffuser partially removed.

FIG. 31 is a rear perspective view corresponding to FIG. 30, with the diffuser in place.

FIG. 32 is a front perspective view corresponding to FIG. 30, with the diffuser partially removed.

FIG. 33 is a rear perspective view corresponding to FIG. 30, with the diffuser partially removed.

FIG. 34 is an enlarged front view corresponding to FIG. 30, with the diffuser in place.

FIG. 35 is a front view of the mask assembly of FIG. 1, showing a position of an upper strap of a headgear connected to the mask assembly.

FIG. 36 is an enlarged rear view if the inside of the inlet connector of the mask assembly of FIG. 1, showing vent or bias holes and an anti-asphyxia valve.

FIG. 37 is an exploded rear perspective view showing the anti-asphyxia valve and conduit connector and breathing gas delivery conduit.

FIGS. 38a) and 38b) are enlarged views of the anti-asphyxia valve in closed and open positions respectively.

FIG. 39 is an enlarged side view corresponding to FIG. 37.

FIG. 40 is an exploded view of another frame and inlet connector of a mask assembly in accordance with this disclosure.

FIG. 41 is an exploded view of another frame and inlet connector of a mask assembly in accordance with this disclosure.

FIG. 42 is a front view of another frame and inlet connector of a mask assembly in accordance with this disclosure.

FIGS. 43 to 46 are views of another frame of a mask assembly in accordance with this disclosure, showing a means of mounting lateral arms of the frame to a central portion of the frame.

FIG. 47 is a perspective view of a mask assembly in accordance with this disclosure.

FIG. 48a) to 48d) are views of mask assemblies in accordance with this disclosure, showing different configurations of finger grips.

FIG. 49 is a side view of the mask assembly of FIG. 1 connected to a breathing gas delivery conduit, with the black outline showing the relative orientation of a prior art mask assembly.

FIG. 50 is an enlarged, part sectional view of a lower part of the mask assembly of FIG. 49.

FIG. 51a) to 51c) are respectively perspective, front and rear views of an inlet connector of a mask assembly in accordance with this disclosure.

FIG. 52 is an enlarged sectional side view of a mask assembly in accordance with this disclosure, with the arrows showing approximate directions of a breathing gas flow into and out from the mask assembly.

FIG. 53 is a rear view of a mask assembly in accordance with this disclosure.

FIG. 54 is a rear view of the mask assembly of FIG. 53, showing example dimensions.

FIG. 55 is a side view of the mask assembly of FIGS. 53 and 54, showing example dimensions.

FIG. 56 is a front view of a cushion of the mask assembly of FIGS. 53 to 55, showing example dimensions.

FIG. 57 is a side view of a cushion of the mask assembly of FIGS. 53 to 55, showing example dimensions.

FIG. 58a) is a front view of another mask assembly in accordance with this disclosure, comprising a different frame; whilst FIG. 58b) and c) are front and side views of the frame.

FIG. 59a) and b) are perspective and side views of a mask assembly in accordance with this disclosure comprising different headgear strap connectors, whilst FIG. 59c) and d) are perspective and side views of the headgear strap connectors.

FIG. 60a) to 60c) are front views of a mask assembly in accordance with this disclosure, comprising a different frame having an adjustable upper portion, with the upper portion in first, second and third conditions.

FIG. 61a) and b) are side and front views of the frame of FIG. 60.

FIG. 62a) and b) are perspective views of a mask assembly in accordance with this disclosure, each showing a frame having a modified upper portion. FIG. 62c) to e) are perspective, front and side views of the mask frame of the mask assembly of FIG. 62a).

FIG. 63a) and b) are views from underneath a mask assembly in accordance with this disclosure, showing a modified frame, and modified frame and cushion respectively, whilst FIG. 63c) and d) are front and rear views of the cushion and frame respectively.

FIG. 64a) is a front view of a mask assembly in accordance with this disclosure having a modified frame and cushion, and FIG. 64b) and c) are enlarged sectional side views of the frame and cushion, Where FIG. 64b) shows the frame ad cushion of FIG. 1, whilst FIG. 64c) shows the modified frame and cushion of FIG. 64.

FIG. 65 is a rear view of a modified cushion of a mask assembly in accordance with this disclosure.

FIG. 66a) to c) are perspective, front and side views of a modified cushion of a mask assembly in accordance with this disclosure, and FIG. 66d) to e) showing the modified cushion with the mask frame and headgear strap connectors.

FIG. 67a) is a view from below of a modified cushion of a mask assembly in accordance with this disclosure, where the modified cushion has internal laterally extending reinforcing structures, and FIG. 67b) and c) are sectional views of the modified cushion taken on lines A-A, and B-B of FIG. 67a).

FIG. 68a) is a view from below of a modified cushion of a mask assembly in accordance with this disclosure, where the modified cushion has internal vertically extending reinforcing structures, and FIG. 68b) and c) are sectional views of the modified cushion taken on lines A-A, and B-B of FIG. 67a).

FIG. 69 is a part sectional side view of a modified cushion of a mask assembly in accordance with this disclosure, where the modified cushion has internal reinforcing structures adjacent a rolling bridge portion of the cushion.

FIG. 70a) and c) are perspective views of a mask assembly in accordance with this disclosure, each showing a modified mask frame, with FIG. 70b) showing the modified mask frame.

FIG. 71a) and c) are perspective views of a mask assembly in accordance with this disclosure, each showing a modified mask frame, with FIG. 71b) showing the modified mask frame, FIG. 71d) being an enlarged view of part of the inlet connector, and FIG. 71e) being an enlarged perspective view from the rear of part of the modified mask frame.

FIG. 72a) and c) are perspective and exploded views of a mask assembly in accordance with this disclosure, each showing a modified mask frame, with FIG. 72b) showing the modified mask frame.

FIG. 73a), c) and f) are perspective, exploded and side views of a mask assembly in accordance with this disclosure, each showing a modified mask frame, with FIG. 73b) showing the modified mask frame, FIG. 73d) showing the modified mask frame and inlet connector, FIG. 73e) showing the modified mask frame and inlet conduit.

FIG. 74a), d) and e) are perspective, front and side views of a mask assembly in accordance with this disclosure, each showing a modified mask frame, with FIG. 74b) omitting the mask frame, FIG. 74c) showing the modified mask frame, and FIG. 74f) being an enlarged view of a lower part of the mask frame and inlet connector.

FIG. 75a), c), d) and e) are perspective, front and side views of a mask assembly in accordance with this disclosure, each showing a modified mask frame, with FIG. 75b) omitting the mask frame, FIG. 75f) being an enlarged view of part of the modified mask frame and inlet connector, and FIG. 75g) being an enlarged sectional view of a lower part of the mask frame and inlet connector.

FIG. 76a), b) are side and front views of a mask assembly in accordance with this disclosure, each showing a modified mask frame, with FIG. 76c) and d) being enlarged side and perspective views of part of the mask frame and inlet connector, FIG. 76e) showing a view from below of the modified mask frame and inlet connector, and FIG. 76f) being an enlarged view of a central part of the mask frame.

FIG. 77a), b) are side and front views of a mask assembly in accordance with this disclosure, each showing a modified mask frame, with FIG. 76c) and d) being enlarged side and sectional views of part of the mask frame and inlet connector, and FIG. 76e) showing a view from below of the modified mask frame and inlet connector.

FIG. 78a), b) are side and front views of a mask assembly in accordance with this disclosure, each showing a modified mask frame, with FIG. 78c) being a view from below of the mask frame, FIG. 78d) being an enlarged exploded perspective view of the mask frame and inlet connector, FIG. 78e) being an enlarged sectional view of part of the mask frame and inlet connector, and FIG. 78f) showing a view from below of the modified mask frame and inlet connector.

FIG. 79a), b) and c), are side, front and perspective views of a mask assembly in accordance with this disclosure, each showing a modified mask frame, with FIG. 79d), e) and f) being enlarged sectional side, perspective and rear views of the mask frame and inlet connector.

FIG. 80a) and b) are perspective and side views of a mask assembly in accordance with this disclosure, each showing a modified mask frame, with FIG. 80c), d) and e) being enlarged view of part of a lower part of the modified mask frame and inlet connector, FIG. 80f) and g) being enlarged views of the modified mask frame and inlet connector in assembled and partially disassembled conditions, and FIG. 80h) being an enlarged view from below of the mask frame and inlet connector. FIG. 80i) is a top view of the modified mask frame, and FIG. 80j) is an enlarged top view of part of the modified mask frame. FIG. 80k) is a schematic top view of the modified mask frame, with the dashed lines indicating example deflected positions of the lateral arms. FIG. 80l) is an exploded side view of the mask assembly of FIG. 80a) to 80k), with the diffuser mounted on the inlet, and FIG. 80m) is an exploded side view of the mask assembly of FIG. 80a) to 80k), with the diffuser removed from the inlet. FIG. 81 a) and b) are perspective and exploded views of a mask assembly in accordance with this disclosure with a modified mask frame.

FIG. 82 a) and b) are perspective and exploded views of a mask assembly in accordance with this disclosure with a modified mask frame, with FIG. 82c) being an enlarged side view of part of the modified mask frame.

FIG. 83a) to d) are schematic sectional side views showing different configurations of inlet connector mask frame and cushion.

FIG. 84a), b) are front and side views of a mask assembly in accordance with this disclosure, each showing a modified mask frame, with FIG. 84c) being a sectional side view of the mask assembly, and FIG. 84d) being a perspective of the mask frame.

FIG. 85a) is a front view of a modified mask frame and inlet connector of a mask assembly in accordance with this disclosure, FIG. 85b) is a front view of the mask frame, and FIG. 85c) is an enlarged side view of part of the mask frame and inlet connector.

FIG. 86a) is an exploded view of an inlet connector and part of a mask frame of a mask assembly in accordance with this disclosure, FIG. 86b) is an enlarged front view of a central part of the mask frame, and FIG. 86c) is an enlarged side view of part of the inlet connector and mask frame.

FIG. 87 is an exploded view of a mask frame and inlet connector of a mask assembly in accordance with this disclosure.

FIG. 88 is an exploded view of a mask frame and inlet connector of a mask assembly in accordance with this disclosure.

FIG. 89a), b) and c), are front, side and perspective views of a mask assembly in accordance with this disclosure, each showing a modified mask frame, with FIG. 89d), being an exploded view, and FIG. 89e) being an enlarged exploded view of the inlet connector and part of the mask assembly.

FIG. 90a), b) and c), are front, side and exploded views of a mask assembly in accordance with this disclosure.

FIG. 91a) is a perspective view of a mask assembly in accordance with this disclosure, FIG. 91b) is a perspective view of a cushion of the mask assembly, and FIG. 91c) is an enlarged sectional top view of the mask assembly and cushion.

FIG. 92 is a front view of a modified mask frame of a mask assembly in accordance with this disclosure.

FIG. 93 a) and b) are perspective and exploded perspective views of a mask assembly in accordance with this disclosure, with an integral mask frame and inlet connector.

FIG. 94 a) and b) are perspective and exploded perspective views of a mask assembly in accordance with this disclosure, with an integral mask frame and inlet connector.

FIG. 95 a) and b) are perspective and exploded perspective views of a mask assembly in accordance with this disclosure, with an integral mask frame and inlet connector.

FIG. 96 a) and b) are schematic sectional side and end views of a conduit in accordance with this disclosure.

FIG. 97 a) to c) are bottom, top and sectional side views of an inlet connector in accordance with this disclosure.

FIG. 98 a) to c) are sectional side, rear, bottom of the inlet connector of FIG. 97, and an anti-asphyxia (AA) valve. FIG. 98 d) is an enlarged view of a hinge region of the AA valve.

FIG. 99 a) and b) are sectional side views of an example mould tool that can be used to mould the inlet connector of FIG. 97.

FIG. 100a) and b) perspective and side cross sectional views of an AA valve in accordance with this disclosure.

FIG. 101 a) is a top view of the inlet connector of FIG. 96 and the AA valve of FIG. 99, with a flap of the AA valve closing a valve opening of the inlet connector. FIG. 101 b) is a similar view but with the valve flap shown translucent such that the opening of the inlet connector can be seen, with the overlap of the valve flap with the inlet connector around the opening being seen. FIG. 101 c) to e) are schematic plan views of the valve flap, valve opening, and valve flap closing the valve opening.

FIG. 102 a) is a schematic side view of an AA valve flap adjacent part of an inlet connector, showing, in exaggerated form, a tendency of the valve flap to deform such that there is a gap between the valve flap and the adjacent part of the inlet connector. FIG. 102 b) shows a similar view but showing more contact between the valve flap and the adjacent part of the inlet connector. FIG. 102 c) to e) are enlarged perspective sectional, and side sectional views of an inlet connector in accordance with this disclosure in which part of the inlet connector has a profile configures to minimise any gap between the inlet connector and the AA valve flap.

FIG. 103 a) to c) are perspective, side and front views of a mask assembly in accordance with this disclosure, comprising a modified frame. FIG. 103d) is a perspective view of the modified frame, FIG. 103e) is a perspective view of the modified frame and a head gear connector clip, FIG. 103f) is a perspective cutaway view of a lateral arm of the frame, FIG. 103g) is a schematic cross-sectional view showing the profile of the lateral arm, FIG. 103h) is an enlarged view of part of a headgear clip, FIG. 103i) is a side view of the headgear clip, and FIG. 103j) and k) are perspective views of the headgear clip.

FIG. 104 a) and b) are front and enlarged perspective views of a mask cushion in accordance with this disclosure, showing features of the inlet opening of the cushion. FIG. 104 c) to f) are enlarged sectional side views of the mask cushion and the inlet connector.

FIG. 105 a) to c) are perspective, side and front views of a mask assembly in accordance with this disclosure, comprising a modified diffuser.

FIG. 106 a) and b) are enlarged perspective and side views of an inlet connector and diffuser in accordance with this disclosure. FIG. 106c) is an enlarged sectional side view of an upper part of the inlet connector and diffuser. FIG. 106 d) to f) are perspective, rear and front views of a diffuser frame.

FIG. 107 a) and b) are front and cross-sectional perspective views of an inlet connector and diffuser in accordance with this disclosure.

FIG. 108 a) to c) are perspective, side and front views of a mask assembly in accordance with this disclosure, comprising a modified frame.

FIG. 109 is an enlarged view of an upper part of a modified frame in accordance with this disclosure.

FIG. 110 a) and b) are perspective and plan views of a modified frame in accordance with this disclosure, and FIG. 110 c) is a perspective view from the rear of the modified frame connected to an inlet connector.

FIG. 111 a) to c) are front, front and rear of a diffuser in accordance with this disclosure, FIG. 111 d) is a side view of an inlet connector on which the modified diffuser can be mounted, and FIG. 111 e) is a cross-sectional view from above of the inlet connector and modified diffuser.

FIG. 112 a) and b) are perspective views from the rear of a modified inlet connector and FIG. 112 c) is perspective view from the front of a modified cushion, in accordance with this disclosure.

FIG. 113 a) to b) are perspective views of an inlet connector and AA-valve in accordance with this disclosure, showing different configurations of anti-inversion features, and FIG. 113 e) to j) are schematic views of anti-inversion features.

FIG. 114a) is a rear perspective view of an inlet connector and AA-valve in accordance with this disclosure, and FIG. 114b) is an enlarged section side view, showing upper and lower mould tools.

FIG. 115 is a rear perspective view of an inlet connector and AA-valve in accordance with this disclosure.

FIG. 116a) is a part sectional perspective view of an inlet connector and AA-valve in accordance with this disclosure, FIG. 116b) a sectional side view, and FIG. 116c) a rear view.

FIG. 117a) is a part sectional perspective view of an inlet connector and AA-valve in accordance with this disclosure, FIG. 117b) an enlarged plan view, and FIG. 117c) a schematic plan view of a valve flap and valve seat of the AA-valve.

FIG. 118a) is a part sectional perspective view of an inlet connector and AA-valve in accordance with this disclosure, FIG. 118b) is an enlarged sectional side view showing upper and lower mould tools, prior to overmoulding of the AA-valve.

FIG. 119a) is a perspective view from the rear of an inlet connector and AA-valve in accordance with this disclosure, FIG. 119b) a sectional plan view, and FIG. 119c) a schematic plan view of a valve flap and valve seat of the AA-valve.

FIG. 120 is a perspective view from the rear of an inlet connector and AA-valve in accordance with this disclosure. FIG. 121a) is a perspective view of an AA valve in accordance with this disclosure, FIG. 121b) is an enlarged side view of a hinge portion of the AA-valve, and FIG. 121c) is an enlarged sectional view of the hinge portion.

FIG. 122a) is a perspective view of an AA valve in accordance with this disclosure, FIG. 122b) is an enlarged part sectional end view of a hinge portion of the AA-valve.

FIG. 123 shows the following, in accordance with this disclosure:
a) a rear perspective view of an inlet connector;
b) a view from below of the inlet connector;
c) a perspective view of an AA-valve configured to be used with the inlet connector;
d) a plan view of the AA-valve;
e) a rear view of the AA-valve;
f) a side view of the AA-valve;
g) a perspective view of the AA-valve showing a removable pull tab; and
h) is an enlarged part sectional perspective view of the AA-valve in the inlet connector.

FIG. 124a) is a perspective view from above of an AA-valve in accordance with this disclosure, showing a removable pull tab, FIG. 124b) is an enlarged part sectional perspective view of the AA-valve in the inlet connector; and FIG. 124c) is a perspective view from the rear of the AA-valve.

FIG. 125 shows the following, in accordance with this disclosure:
a) a rear perspective view of an inlet connector and AA-valve, with the valve flap in a first condition;
b) a rear perspective view of the inlet connector and AA-valve, with the valve flap in a second condition;
c) a plan view of the AA-valve;
d) a perspective view of the inlet connector with the AA-valve folded prior to mounting in the inlet connector; and
e) a perspective view of the inlet connector with the AA-valve folded during mounting in the inlet connector.

FIG. 126a) is a plan view of an AA-valve in accordance with this disclosure, and FIG. 126b) is a perspective view of the inlet connector with the AA-valve folded prior to mounting in the inlet connector.

FIG. 127 is an enlarged sectional side view of an inlet connector and AA-valve in accordance with this disclosure.

FIG. 128 is a perspective view of a mask assembly in accordance with this disclosure, with a removable diffuser.

FIG. 129 is a perspective view of a mask assembly in accordance with this disclosure, with a removable diffuser.

FIG. 130a) is an enlarged perspective view of part of a frame clip of a frame in accordance with this disclosure, FIG. 130b) is a view from below of the frame clip connected to an inlet connector, and FIG. 130c) is an exploded perspective view of the frame and inlet connector prior to connection.

FIG. 131a) is a view from below of a frame clip of a frame in accordance with this disclosure; FIG. 131b) is an enlarged side view of part of an inlet connector, and FIG. 131c) is a view from below of the frame clip connected to the inlet connector.

FIG. 132a) is a perspective view of an inlet connector in accordance with this disclosure, and FIG. 132b) is a view from below of a frame clip connected to the inlet connector.

FIG. 133a) is an enlarged perspective view of part of a frame clip of a frame in accordance with this disclosure; FIG. 133b) is an enlarged side view of part of an inlet connector, and FIG. 133c) is a view from below of the frame clip connected to the inlet connector.

FIG. 134a) is an enlarged perspective view of part of a frame clip of a frame in accordance with this disclosure; FIG. 134b) is an enlarged side view of part of an inlet connector, and FIG. 134c) is an enlarged view from the side of the frame clip connected to the inlet connector.

FIG. 135a) is an enlarged perspective view of part of a frame clip of a frame in accordance with this disclosure; FIG. 135b) is an enlarged side view of part of an inlet connector.

FIG. 136a) is an enlarged perspective view of part of a frame clip of a frame in accordance with this disclosure; FIG. 136b) is a perspective view of part of an inlet connector, and FIG. 136c) is a view from below of the frame clip connected to the inlet connector.

FIG. 137a) is an enlarged perspective view of part of a frame clip of a frame in accordance with this disclosure; FIG. 137b) is perspective view of an inlet connector, and FIG. 137c) is a perspective view of the frame clip connected to the inlet connector.

FIG. 138a) is an enlarged perspective view of a frame and frame clip in accordance with this disclosure; FIG. 138b) is a perspective view of part of an inlet connector, and FIG. 138c) is a perspective view of the frame clip connected to the inlet connector showing an anti-rotation feature; and FIG. 138d) is an enlarged perspective view of the frame clip connected to the inlet connector showing a modified anti-rotation feature.

FIG. 139a) is an enlarged rear view of part of a frame clip of a frame in accordance with this disclosure; FIG. 139b) is perspective view of an inlet connector; FIG. 139c) is a perspective view from above of the frame clip connected to the inlet connector; and FIG. 139d) is an enlarged view from above of part of the frame clip and inlet connector.

FIG. 140a) is an enlarged perspective view from the front of a lower part of an inlet connector in accordance with this disclosure; and FIG. 140b) is an enlarged view from above of the inlet connector connected to a frame.

FIG. 141a) is a perspective view from the rear of a frame in accordance with this disclosure; FIG. 141b) is a perspective view of an inlet connector, and FIG. 141c) is a view from underneath of the frame connected to the inlet connector.

FIG. 142a) is an enlarged perspective view of part of a frame clip of a frame in accordance with this disclosure; FIG. 142b) is perspective view of an inlet connector; and FIG. 142c) is a perspective view of the frame clip connected to the inlet connector.

FIG. 143a) is a perspective view from the front of a frame in accordance with this disclosure; FIG. 143b) is a plan view of the frame; and FIG. 143c) is an enlarged perspective view of part of a frame clip of the frame.

FIG. 144a) is a sectional side view of an inlet connector and cushion in accordance with this disclosure; and FIG. 144b) is an enlarged side view.

FIG. 145a) is a perspective view from the front of an inlet connector and cushion in accordance with this disclosure; and FIG. 145b) is a sectional view from above.

FIG. 146a) is a perspective view from the front of an inlet connector and cushion in accordance with this disclosure; and FIG. 146b) is a sectional view from above.

FIG. 147a) is a perspective view from the rear of an inlet connector and cushion in accordance with this disclosure; and FIG. 147b) is a sectional view from above.

FIG. 148a) is an exploded view from the front of an inlet connector and cushion in accordance with this disclosure; FIG. 148b) is a perspective view from the rear of the inlet connector and cushion; and FIG. 145b) is a sectional view from above.

FIG. 149 is an enlarged sectional side view of a connection region of an inlet connector and cushion in accordance with this disclosure.

FIG. 150 is an enlarged sectional side view of a connection region of an inlet connector and cushion in accordance with this disclosure.

FIG. 151a) is a sectional side view of an inlet connector and cushion in accordance with this disclosure; and FIG. 151b) is an enlarged sectional side view of a connection region of the inlet connector and cushion.

FIG. 152a) is a part sectional rear view of an inlet connector and cushion in accordance with this disclosure; and FIG. 152b) is an enlarged sectional side view of a connection region of the inlet connector and cushion.

FIG. 153 is an enlarged sectional side view of a connection region of an inlet connector and cushion in accordance with this disclosure.

FIG. 154a) to f) are front, rear, side, rear perspective top and bottom views of a diffuser frame of an inlet connector in accordance with this disclosure.

FIG. 155a) to e) are front perspective, front, rear, rear from above, and side views of an inlet connector in accordance with this disclosure.

FIG. 156a) is an exploded side view of the diffuser frame and inlet connector of FIGS. 154 and 155, with FIG. 156 b) and c) being front and side views with the diffuser frame mounted on the inlet connector.

FIG. 157a) is a front view of the diffuser frame and inlet connector of FIGS. 154 and 155, showing section lines B-B, C-C and D-D. FIG. 157b) is section view taken along line B-B of FIG. 157a), FIG. 157c) is section view taken along line C-C of FIG. 157a), and FIG. 157d) is section view taken along line D-D of FIG. 157a).

FIG. 158a) is a side view of the inlet connector of FIG. 155, showing section line A-A. FIG. 158b) and c) are respectively sectional and enlarged sectional views taken along line A-A of FIG. 158a). FIG. 158d) is a rear view of part of the inlet connector and diffuser frame of FIGS. 154 and 155. FIG. 158e) is a front view of part of the inlet connector with the diffuser frame removed. FIG. 158f) is another sectional view from the rear of the inlet connector. FIG. 158g) is a perspective view from underneath of the inlet connector. FIG. 158h) is a plan view of the inlet connector.

FIG. 159a) is a side view of the diffuser frame and inlet connector of FIGS. 154 and 155 showing section line E-E. FIG. 159b) and c) are sectional views taken along line E-E of FIG. 159a), with FIG. 159b) showing the diffuser frame mounted on the inlet connector.

FIG. 160a) and b) are schematic sectional side and end views of a conduit in accordance with this disclosure.

FIG. 161a) to e) are top perspective, side, bottom perspective, sectional top perspective, sectional side views of an aa-valve in accordance with this disclosure.

FIG. 161f) is an enlarged sectional side view of the aa-valve mounted in an inlet connector in accordance with this disclosure.

FIG. 162a) and b) are front perspective and front views of a cushion in accordance with this disclosure. FIG. 162c) is an enlarged rear perspective view of the cushion of FIG. 162a), connected to an inlet connector. FIG. 162d) is an enlarged view from underneath of the cushion of FIG. 162a) connected to an inlet connector.

FIG. 163a) to c) are front perspective, front and side views of a mask assembly in accordance with this disclosure, comprising the inlet connector and diffuser frame of FIGS. 154 and 155, and the cushion of FIG. 162.

### DETAILED DESCRIPTION

Embodiments of mask assemblies, components and methods of assembly and manufacture will now be described with reference to the accompanying figures, wherein like numerals refer to like or similar elements throughout. Although several embodiments, examples and illustrations are disclosed below, it will be understood by those of ordinary skill in the art that the disclosures described herein extend beyond the specifically disclosed embodiments, examples and illustrations, and can include other uses of the disclosures and obvious modifications and equivalents thereof. The terminology used in the description presented herein is not intended to be interpreted in any limited or restrictive manner simply because it is being used in conjunction with a detailed description of certain specific embodiments of the disclosures. In addition, embodiments of the disclosures can comprise several novel features and no single feature is solely responsible for its desirable attributes or is essential to practicing the disclosures herein described.

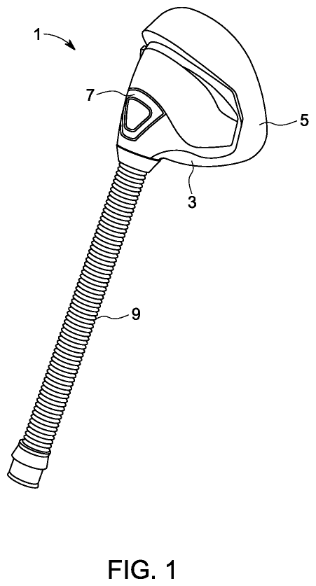

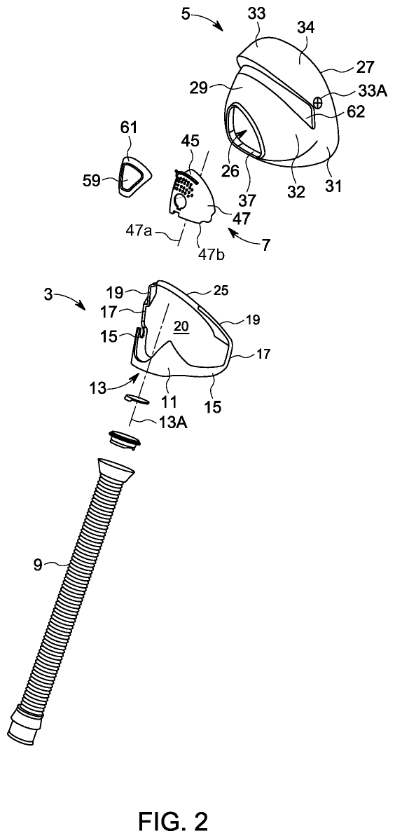

With reference initially to FIGS. 1 and 2, a mask assembly 1 is provided comprising a frame 3, a cushion 5, and an inlet connector 7. The mask assembly 1 is connected to a breathing gas delivery conduit 9 via the inlet connector 7.

The mask assembly is configured to be relatively compact, and in particular to minimise the height of the mask assembly from top to bottom, and the depth of the mask assembly from front to back.

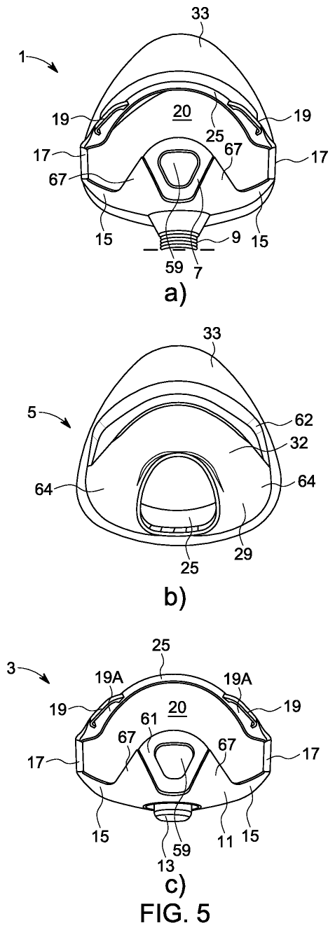

The mask assembly 1, when viewed from the front, comprises an elongate base, inclined sides inclining inwardly from bottom to top, the inclined sides meeting at an apex portion. The apex portion is arcuate, and comprises rounded intersections with the inclined sides. The elongate base also comprises rounded intersections with the inclined sides. When viewed from the front (for example, in FIG. 5) the cushion 5 has an identical or similar shape to the frame 3. The outermost periphery of the frame 3 is substantially contained within the outermost periphery of the cushion 5. The frame 3 comprises headgear connectors for connection to headgear straps. In the embodiments described herein, the headgear connectors provide a four-point connection to headgear.

The inlet connector 7 is multi-functional, and comprises all the gas flow features required for the mask assembly 1 to function. The inlet connector 7 comprises the connection to the breathing gas delivery conduit 9, the breathing gas flow path from the conduit 9 to the interior cavity of the cushion 5, bias flow vent holes, an anti-asphyxia valve, and a diffuser. The inlet connector 7 occupies, despite all of these features, a relatively small proportion of the frontal area of the mask assembly 1. For example, the maximum width of the inlet connector 7 may be between a third and a half of the maximum width of the frame 3. The maximum height of the inlet connector 7 may be around half of the maximum height of the frame 3.

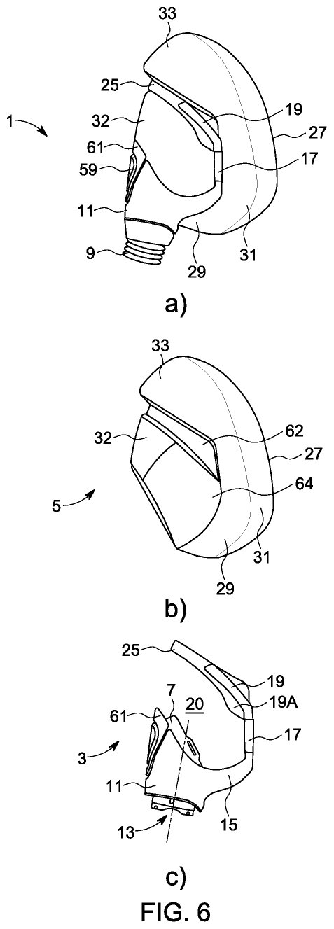

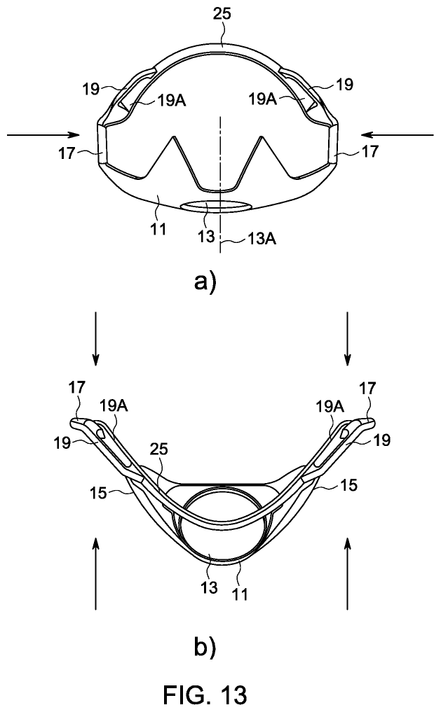

The frame 3 comprises a lower, central portion 11 comprising an inlet opening 13 configured to receive an inlet part, such as an inlet boss 47, of the inlet connector 7. The inlet opening 13 comprises an inlet opening axis 13A (see FIGS. 2, 6, and 13a) which is configured to extend generally downwardly in use of the mask assembly 1, such that the inlet opening 13 is formed in a base of the frame 3. The inlet opening 13 comprises an interior surface that is substantially smooth and flat, to assist in guiding inlet connector 7 into the inlet opening 13, during assembly or reassembly. The inlet opening axis 13A is inclined downwardly and towards the rear of the mask assembly when viewed from the side, see FIG. 6. The inlet opening axis 13A is substantially vertical when viewed from the front, see FIG. 13a).

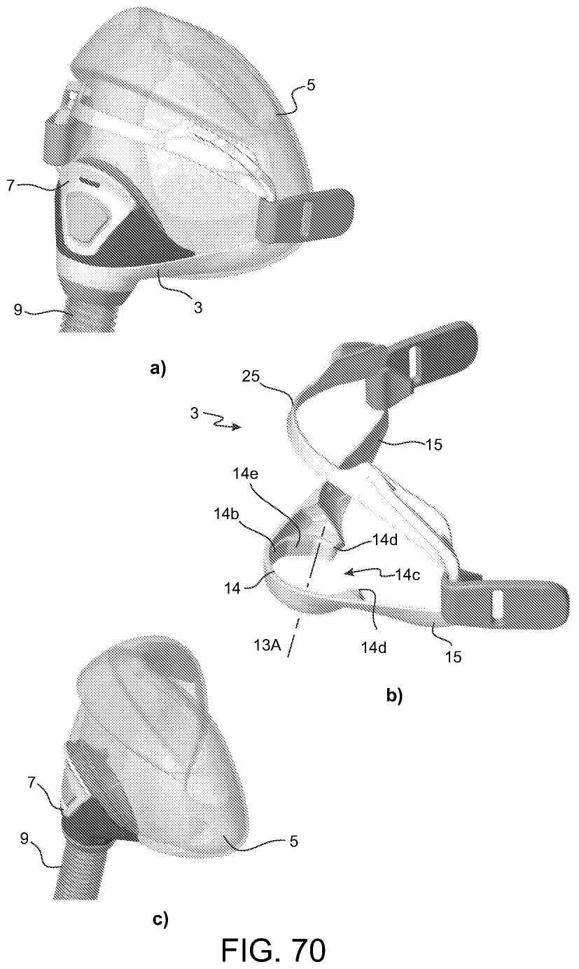

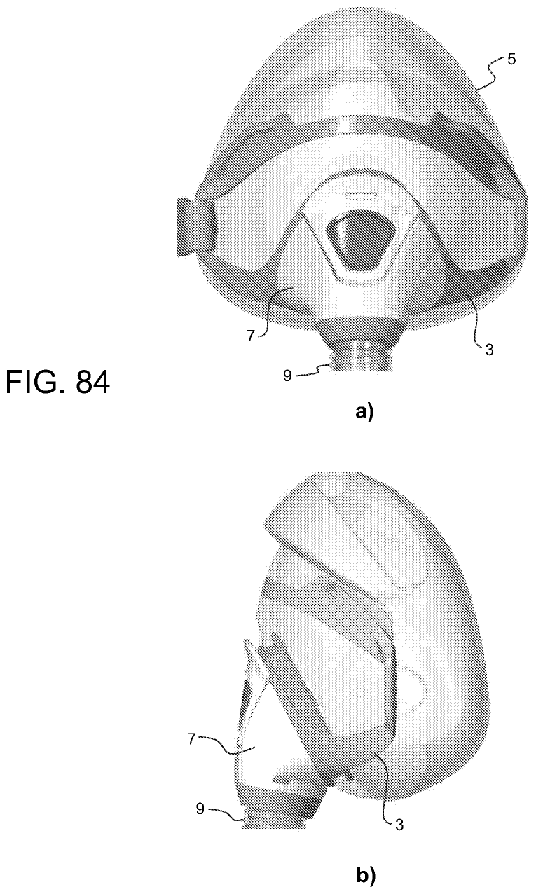

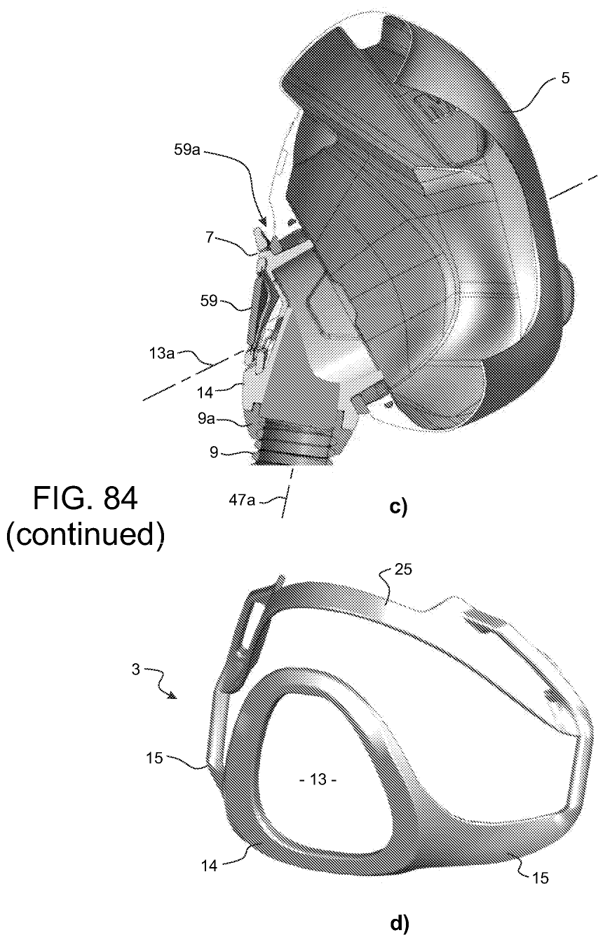

The inlet opening axis 13A may be substantially aligned with an inlet axis 47A of the inlet boss 47 of the inlet connector 7, see for example the embodiment of FIGS. 2 and 70. In other embodiments, the inlet opening axis 13A may not be aligned with, and may intersect, the inlet axis 47A, see for example the embodiment of FIG. 84. The gas flow path from the conduit 9 to the cushion 5 extends through the inlet connector 7.

Lateral arms 15 extend, as elongate side frame elements, outwardly from the sides of the lower, central portion 11 in a generally horizontal direction, for example see FIG. 5. The lateral arms 15 are inclined upwardly relative to the inlet opening axis 13A, when the frame 3 is viewed from the front and side, see for example FIGS. 5a), 6a), and 13a). The lateral arms 15 may each twist along their length, that is about a longitudinal axis extending along each arm 15. Such twisting can help enable the lower headgear connectors 19 to be correctly orientated relative to the remainder of the frame 3. The central portion 11, and the opening 13 provided in the central portion 11, are therefore intermediate the pair of lateral arms 15, at a lowermost part of the mask frame 3.

The ends of the lateral arms 15 terminate in a pair of lower headgear connectors 17.

The frame 3 further comprises a pair of upper headgear connectors 19, above the lower headgear connectors 17.

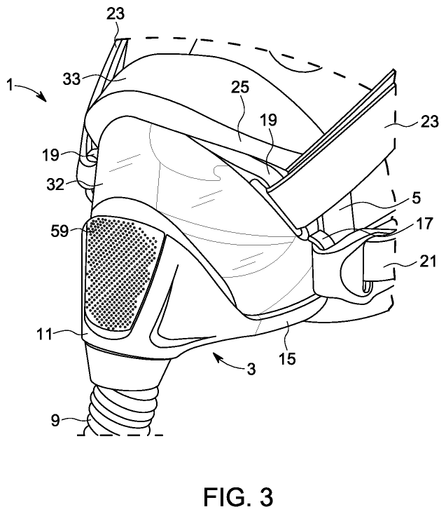

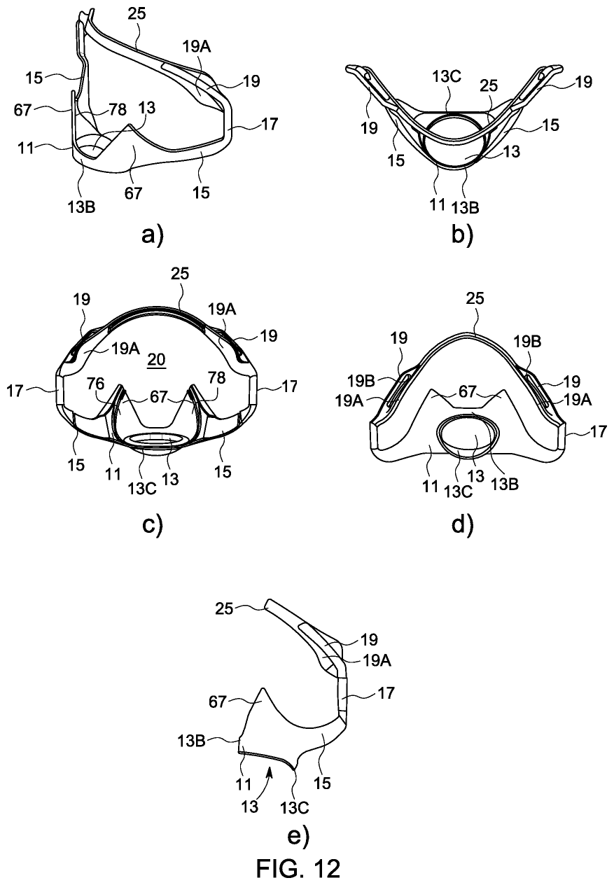

The frame 3 therefore comprises a pair of laterally spaced lower headgear connectors 17 and a pair of laterally spaced upper headgear connectors 19. As can be seen in FIG. 3, each headgear connector 17, 19 is configured to be connected to a respective headgear strap 21, 23. The upper ends of the pair of upper headgear connectors 19 are connected via end regions of a connecting frame member in the form of an upper frame portion 25 comprising an arcuate elongate frame element that comprises the upper most part of the frame 3. The central portion 11, lateral arms 15, headgear connectors 17, 19 and upper frame portion 25 form a frame ring inside of which is defined a closed, frame aperture 20 through which the cushion 5 is exposed. The central portion 11 is relatively rigid. The lateral arms 15 and the upper frame portion 25 are relatively flexible. As can be seen in FIG. 12e), the connecting frame member is inclined forwardly, such that when viewed from the side, an acute angle is formed between the lateral arms and the connecting frame member. For example, a longitudinal axis of the connecting frame member may be inclined at an acute angle relative to a longitudinal axis of the adjacent lateral arm. The acute angle may be between 10° and 80°, or between 20° and 65°, and in some embodiments is substantially 45°. As can be seen in FIG. 6, the connecting frame member connects the lateral arms 15 at a position above and behind the central portion 11. The connecting frame member extends over an upper portion of the cushion. The connecting frame member is integral with the lateral arms 15.

The central portion 11 is relatively tall when the frame 3 is viewed from the front. The central portion 11 tapers into the lateral arms 15 which are relatively narrow. Likewise the upper frame portion 25 is relatively narrow and forms a strip or band extending across the cushion 5.

The central portion 11 has a greater resistance to deformation and/or bending, as compared to the lateral arms 15 and upper frame portion 25. It is envisaged that the central portion 11 may be configured not to deform when side sleeping. In contrast the lateral arms 15 and upper frame portion 25 may deform relatively easily during side sleeping. The relative rigidity of the central portion 11 assists in maintaining a proper connection with the inlet connector 7.

The connecting frame member is flexible along at least a portion of its length, and in particular the connecting frame member comprises a central region being an apex of the connecting frame member, see FIG. 5, the central region being relatively flexible. The connecting frame member comprises end regions, distal from the central region, the end regions being less flexible than the central region. The end regions comprise the upper pair of headgear connectors 19. The central region comprises a relatively thin and/or weakened structure, and the end regions comprise a relatively thick and/or reinforced structure, for example at enlarged regions 19A of the frame 3.

Each headgear connector 17, 19 in this embodiment is elongate. The lower pair of headgear connectors 17 are each in the form of an elongate element comprising a mounting post. The lower headgear straps 21 may be mounted to the mounting posts via a suitable headgear clip that comprises a hook that receives the mounting post. Alternatively, each lower headgear connector 17 can comprise a hook, the lower headgear strap 21 comprising a post that is received in the hook.

The upper pair of headgear connectors 19 each comprise an elongate element around which each upper headgear strap 23 may be looped and secured to itself using a suitable securing component such as a hook and loop fastener, or a buckle for example.

The mounting posts of the lower pair of headgear connectors 17 are substantially parallel, and extend substantially vertically when the mask assembly 1 is viewed from the front and from the side in use. The mounting posts are inclined forwardly relative to the inlet opening axis 13A. When viewed from the front, the longitudinal axes of the elongate posts are parallel with that inlet opening axis 13A.

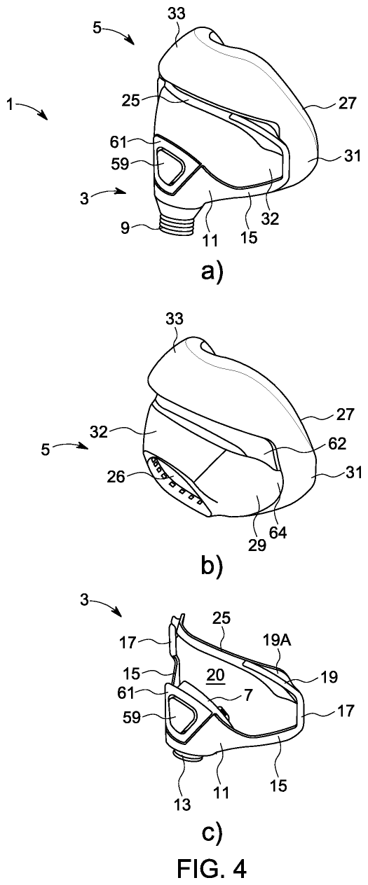

The elongate elements of the upper pair of headgear connectors 19 are inclined relative to the mounting posts of the lower pair of headgear connectors 17. The elongate elements of the upper pair of headgear connectors 19 are also inclined inwardly towards one another when the mask assembly 1 is viewed from the front. Further the elongate elements of the upper pair of headgear connectors 19 are inclined forwardly when the mask assembly 1 is viewed from the side in use. The elongate elements of the upper pair of headgear connectors 19 are inclined inwardly relative to the central axis 13A of the inlet opening 13, when the mask assembly 1 is viewed from the front. The elongate elements of the upper pair of headgear connectors 19 are inclined forwardly such that they intersect the inlet opening axis 13A, when the mask assembly 1 is viewed from the side, for example as shown in FIGS. 4a) and 6a).

The elongate elements of the upper pair of headgear connectors 19 may be inclined forwardly so as to define an acute angle relative to the lateral arms 15. For example, a longitudinal axis of an elongate element of the upper pair of headgear connectors 19 may be inclined at an acute angle relative to a longitudinal axis of the adjacent lateral arm 15. The acute angle may be between 10° and 80°, or between 20° and 65°, and in some embodiments is substantially 45°.

In this embodiment the lower headgear connectors 17 comprise the rearmost part of the frame 3, when the mask assembly 1 is viewed from the side. The lower headgear connectors 17 also comprise the outermost part of the frame 3 when viewed from the front.

As noted above, and as can be seen in FIGS. 5 and 6, the frame 3 further comprises an enlarged region 19A extending behind the elongate element of each upper headgear connector 19, such that a slot 19B is defined between each enlarged region 19A and the respective elongate element. The cushion 5 defines an elongate recess 62 which extends across the cushion 5. The upper portion 25 of the frame 3 rests in the recess 62. The distal ends of the recess 62 are shaped to match the shape of, and receive, the enlarged region 19A. the enlarged regions 19A are connected via the upper frame portion 25.

The recess 62 can comprises a thickened region of the cushion 5.

The enlarged regions 19A each provide an enlarged surface area that overlays the sides of the cushion 5, as compared to the upper frame portion 25, providing additional support to the cushion 5 in those regions.

The combination of the elongate element of the upper headgear connectors 19 and the adjacent enlarged regions 19A, increases the rigidity of the frame 3 in those regions, and resists bending of the upper headgear connectors 19. The enlarged regions 19A also act to ensure there is enough space/clearance for the upper headgear straps 23 to attach to the upper headgear connector 19, without interfering with the protruding, forward edge of the rolling bridge portion 33. The central region of the upper frame portion 25 is relatively thin, as compared to the relatively wide enlarged regions 19A. The enlarged regions 19A comprise end regions of the upper frame portion 25, that extend between, and connect, the distal ends of the lateral arms 15.

The frame 3 is made from material such as a plastics material which is relatively harder and less deformable than the cushion 5, such that the frame 3 provides support to the cushion 5, and in particular to resist outward movement of the cushion 5. The frame 3 is structured to permit the frame to resiliently deform in a lateral direction, but to resist deformation in the front to back direction. This will be described further below.

The inlet connector 7 may be made from material which is relatively harder and less deformable than the cushion 5, and preferably is made from a plastics material.

The frame 3 and/or the inlet connector 7, may be made from a plastics material comprising, solely or in combination:
a) Nylon (Nylon 12, 11, 6);
b) TPE-PEBA (Pebax® 7033, 7233);
c) Polypropylene;
d) Polycarbonate; and/or
e) Co-polyester.

The cushion 5 is configured to be mounted on the frame 3, and in this embodiment to be removably mounted on the frame 3. Mounting features can be provided to ensure the cushion 5 can be mounted on the frame 3. In the described embodiments, the cushion 5 is mounted on the frame 3 via the inlet connector 7.

The cushion 5 may be mounted on the frame 3 via engagement between the inlet opening 13 of the frame 3, with the inlet connector 7. The engagement may be provided by any one or more of:
a) frictional engagement between the inlet opening 13 of the frame 3;
b) an interference fit between the inlet opening 13 of the frame 3;
c) one or more clips on the inlet opening 13 or frame 3 engaging with one or more recesses on the other of the inlet opening 13 and frame 3;
d) one or more protrusions on the inlet opening 13 or frame 3 engaging with one or more apertures or slots on the other of the inlet opening 13 and frame 3;
e) adhesive; and/or
f) overmoulding.

The cushion 5 is formed from a relatively soft, resiliently deformable material such as silicone. The cushion 5 defines an internal cavity that receives breathing gases from the breathing gas delivery conduit 9 via the inlet connector 7. The cushion 5 is provided with an inlet aperture 26.

The cushion 5 comprises a proximal face contacting portion 27, a distal non face contacting portion 29, and a side wall 31 extending between the face contacting portion 27 and the non-face contacting portion 29. The inlet aperture 25 is provided in the distal non-face contacting portion 29.

At least part of the side wall 31 is transparent 32 so as to define a clear region of the cushion 5 where the inside of the cushion 5 can be seen from outside the cushion 5.

This clear region 32 is arcuate when viewed from the front and extends upwardly from one lower headgear connector 17, across the top of the inlet connector 7, and downwardly to the other lower headgear connector 17.

An upper portion of the cushion 5 comprises a rolling bridge portion 33 which is pivotable relative to a lower portion 35 of the cushion 5, between a rest position in which the rolling bridge portion 33 is relatively rearward, to an in use position in which the rolling bridge portion 33 pivots forwardly such that part of the rolling bridge portion 33 buckles to provide a roll of material that extends over the mask frame 3. The rolling bridge portion 33 pivots about a pivot axis 33A. The pivotal movement of the rolling bridge portion 33 enables the upper part of the cushion to better accommodate, and seal against, differently sized noses.

The rolling bridge portion 33 further comprises an arcuate reinforcing band 34, which extends over the apex over the rolling bridge portion 33, and down each side of the cushion to the pivot axis 33A.

The rolling bridge portion 33 comprises a region of reduced stiffness 93 that is positioned between a first boundary 94, the first boundary 94 being defined by a stiffness greater than that in the region of reduced stiffness 93, and a second boundary 96, the second boundary being defined by a stiffness greater than that in the region of reduced stiffness. In this example, the first boundary 94 is adjacent the reinforcing band 34. The second boundary 96 is at the forward margin of the rolling bridge portion 33. When the first boundary 94 is moved toward the second boundary 96, the region of reduced stiffness 93 buckles to define a roll of material that increases in size away from the face-contacting portion of the cushion 5 as the first boundary 94 continues to move toward the second boundary 96 to facilitate movement of the rolling bridge portion 33 of the cushion 5 relative to the lower portion of the cushion 5.

The inlet aperture 25 is defined in the distal non-face contacting portion 29 of the cushion 5, and in particular in a lower, forwardmost portion of the cushion 5. The inlet aperture 25 is larger than the inlet opening 13 in the frame 3. The inlet aperture 25 may be non-circular, may be taller than it is wide, and in this embodiment comprise a substantially planar base and inwardly inclined sides.

The inlet aperture 25 is defined in this example by a relatively rigid ring 37 that comprises an integral part of, or is a separate component which is secured to the distal non-face contacting portion 29 of the cushion 5, for example via adhesive or overmoulding or the like. The rigid ring 37 may be a separate component formed of a material which is more rigid than the relatively soft cushion 5. The rigid ring may comprise a thickened or reinforced part of the cushion 5, formed from the same material as the cushion 5.

The rigid ring 37 may be provided with connecting formations 39 configured to allow the rigid ring 37 to be connected to the inlet connector 7.

The rigid ring 3 may be made from a plastics material comprising, solely or in combination:
a) Nylon (Nylon 12, 11, 6)
b) TPE-PEBA (Pebax® 7033, 7233)
c) Polypropylene
d) Polycarbonate
e) Co-polyester

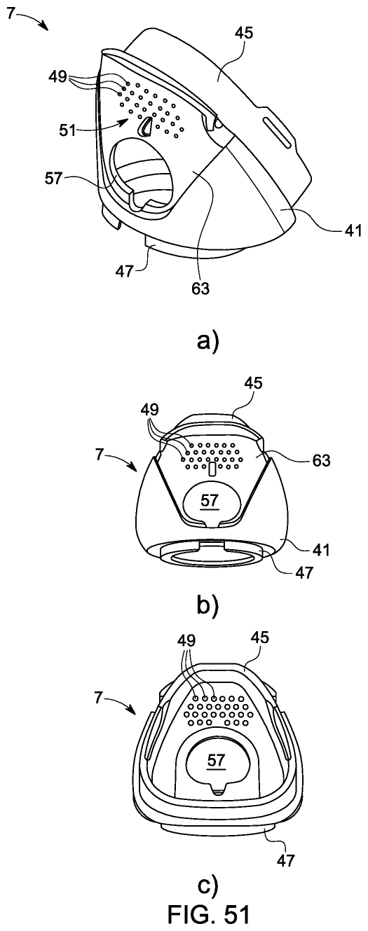

The inlet connector 7, which can best be seen in FIG. 51, comprises a connector body 41 comprising a proximal, rearwardly directed outlet boss 45 and a distal, lower inlet boss 47. The outlet boss 45 is configured to be received in, and to be connected to or mounted on the rigid ring 37 of the cushion 5. The inlet boss 47 is configured to be received in, and to be connected to or mounted on the inlet opening of the frame 3. When so positioned, the inlet connector body 41 forms a breathing gases flow path between the breathing gases delivery conduit 9 and the cavity inside the cushion 5.

As will be described in embodiments below, the inlet connector 7 may be:
a) removably connected to the frame 3;
b) removably connected to the cushion 5;
c) permanently connected to the frame 3;
d) permanently connected to the cushion 5;
e) integrally formed with the frame 3;
f) integrally formed with the cushion 5.

With reference to FIG. 51, the inlet connector 7 advantageously combines a number of gas flow features into a single, relatively compact, component.

In this embodiment the inlet connector 7 comprises:
a) a rearwardly directed outlet boss 45 for connection to the cushion 5, the outlet boss 45 comprising an outlet port 45a that is in fluid communication with the cushion cavity;
b) a downwardly directed inlet boss 47 for connection to the frame 3, the inlet boss 47 comprising an inlet port 47b configured to receive breathable gas from the conduit 9, and comprising a central inlet axis 47a;
c) one or more bias vent holes 49. In this embodiment a plurality of vent holes 49 are provided in a single vent hole array 51;
d) an anti-asphyxia valve 53. In this embodiment the valve 53 comprises a valve body 54, and a valve member 55 movably mounted adjacent the inlet boss 47, and an inlet valve opening 57 above the inlet boss 47. The valve member 55 is movable between a closed position in which the valve member 55 closes the valve opening 57, and an open position in which the valve member 55 opens the opening instead closing the passage through the inlet boss 47. The valve member 55 comprises a planar valve flap;
e) a diffuser 59. Diffuser 59 comprises a body of diffuser material that covers the vent holes 49, so as to diffuse any gases flow from those vent holes 49.

The diffuser material may be permanently mounted on the inlet connector 7, for example via adhesive or overmoulding, or could be removably mounted on the inlet connector 7, for example via a rigid diffuser frame 61 that clips onto the inlet connector 7. The frame 61 may comprises a cover that substantially covers the diffuser material.

The inlet connector 7 has a base and inclined sides, when viewed from the front. The outlet boss 45 matches the size and shape of the rigid ring 37 of the cushion 5. The inlet connector 7 comprises an inclined, forward wall 63 that extends between the outlet boss 45 and inlet boss 47, as can be seen in FIG. 51a). The vent holes 49 and valve opening 57 are provided on the forward wall 63. The plane of the forward wall 63 is substantially parallel with the inlet opening axis 13A of the inlet opening 13 of the frame 3. When mounted on the rigid ring 37, the forward wall 63 has substantially the same profile as the non-face contacting portion 29 and the side wall 31 of the cushion 5.

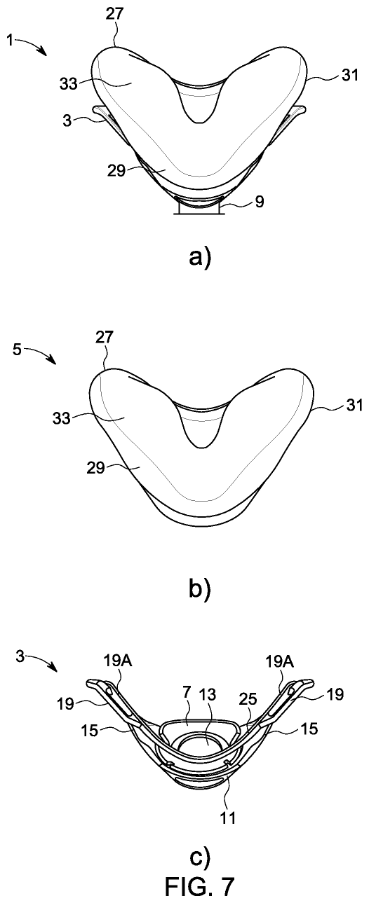

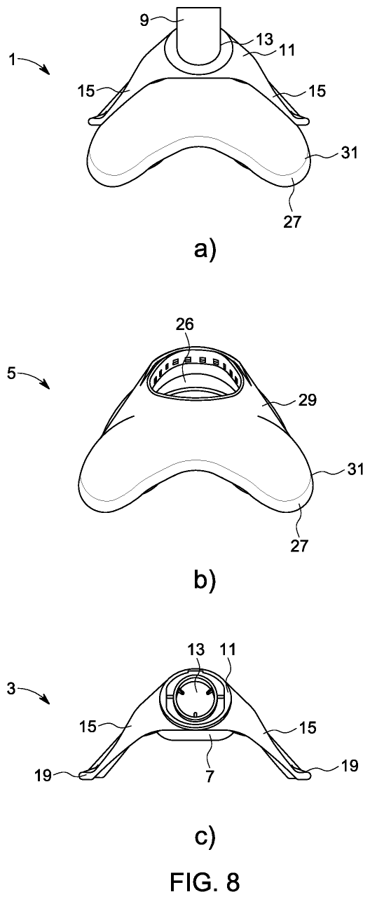

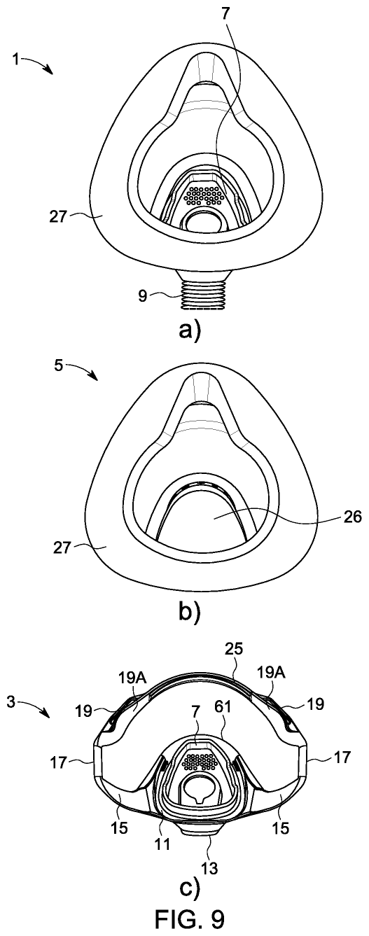

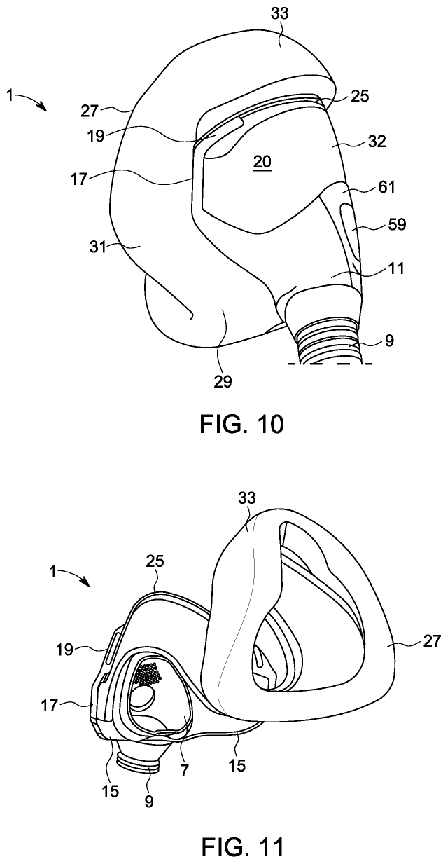

Referring additionally to FIGS. 4 to 11, further aspects of the mask assembly 1 in accordance with this disclosure are now described.

The cushion 5 is removably mounted on the frame 3. In this embodiment, the cushion 5 is mounted on the frame 3 via a connection between the central portion 11 of the frame 3, and the distal non-face contacting portion 29 of the cushion 5. The connection is provided by the rigid ring 37 of the cushion 5 being connected (permanently or removably) to the outlet boss 45 of the inlet connector 7, and by the inlet boss 47 of the inlet connector 7 being connected to the inlet opening 13 of the frame 3, as described above.

In this embodiment the cushion 5 is removably mounted on the frame 3 solely by the connection between the distal non-face contacting portion 29 of the cushion 5 and the central portion 11 of the frame 3. The remainder of the cushion 5 is not connected to the remainder of the frame 3. The cushion 5 may therefore be considered to be cantilevered to the frame 3, with the fulcrum of the cantilever being the connection at the central portion 11 of the frame 3, with the remainder of the cushion 5 being free to move relative to the frame 3.

However, the reminder of the frame 3 that is not connected to the cushion 5, namely the lateral arms 15, lower and upper pairs of headgear connectors 17, 19, and the upper frame portion 25, support the cushion 5, and in particular resist the cushion 5 deflecting or inflating outwardly. This is achieved by the remainder of the frame 3 overlaying the cushion 5, and in particular overlaying the side wall 31 and upper portion of the cushion 5, below the rolling bridge portion 33.

This configuration enables the mask assembly 1 to be relatively flexible in a lateral direction. In particular the lower centre of the mask assembly, including the central portion of frame 11, is relatively inflexible, but the lateral regions and upper centre of the mask assembly 1, including the lateral arms 15 and upper frame portion 25, are relatively flexible, enabling the lateral regions of the mask assembly 1 to be relatively easily elastically deformed when side sleeping for example.

Portions of the cushion 5 may be recessed so as to receive, partially receive or partially engage portions of the frame 3. For example, the upper portion of the cushion 5, below the rolling bridge portion 33, comprises elongate recess 62 which extends laterally across the upper portion of the cushion 5, and in which the upper portion 25 of the frame 3 is received. The side walls of the recess 62 engage the margins of the upper portion 25 of the frame 3. Likewise, the side wall 31 of the cushion 5 comprises generally vertically extending side recesses 64 that engage the lower and/or upper headgear connectors 17, 19 of the frame 3.

The frame 3 substantially overlays the cushion 5. However, the frame comprises frame portions that are relatively thin, in particular the lateral arms 15 and the upper frame portion 25. Consequently, the frame 3 defines a relatively large frame aperture 20 through which the cushion 5 is exposed, and in particular through which the non-face contacting portion 29 and the side wall 31 are exposed. The frame aperture 20 forms a window, through which the cushion 5 is exposed.

The frame aperture 20 corresponds to the transparent part 32 of the cushion 5 such that the transparent part 32 of the cushion is exposed via the frame aperture 20. This enables the inside of the cushion 5, and therefore part of the user's face, especially the nose, to be seen from outside the mask assembly 1, when the mask assembly 1 is worn by the user and in use. This can have the effect of significantly reducing the visual impact of the mask assembly 1 when worn, increasing compliance of use by the user, and/or minimising any psychologically adverse effect of having to wear such a mask assembly 1.

As can best be seen in FIGS. 5 and 12, the frame aperture 20 is arcuate when the mask assembly 1 is viewed from the front. The central lower part of the mask assembly 1 is provided by the inlet connector 7 which covers the tip of the nose of the user. The mouth of the user is covered by the central portion 11 of the frame 3. The central portion 11 is wider than the remainder of the frame 3 and comprises two generally upwardly directed triangular wings 67 that provide support to the central part of the cushion 5, and strengthen the intersection of the lateral arms 15 with the central portion 15 of the frame 3. The wings 67 provide rigidity to the central portion 15 of the frame 3. The wings 67 defined between them a ‘V’ or ‘U’ shaped recess in which the forward wall 63 of the inlet connector 7 is received.

With reference to FIGS. 1, 5a) and 6a), the size and configuration of the inlet connector 7 further minimises the visual impact of the mask assembly 1 in that the inlet connector 7 comprises a number of different components and functions into a relatively small connector body 41, thus freeing up the remainder of the frame 3 and cushion 5 to not need to provide such components and functions. An inlet connector 7 having such multiple functions assists in providing a relatively large frame aperture 20.

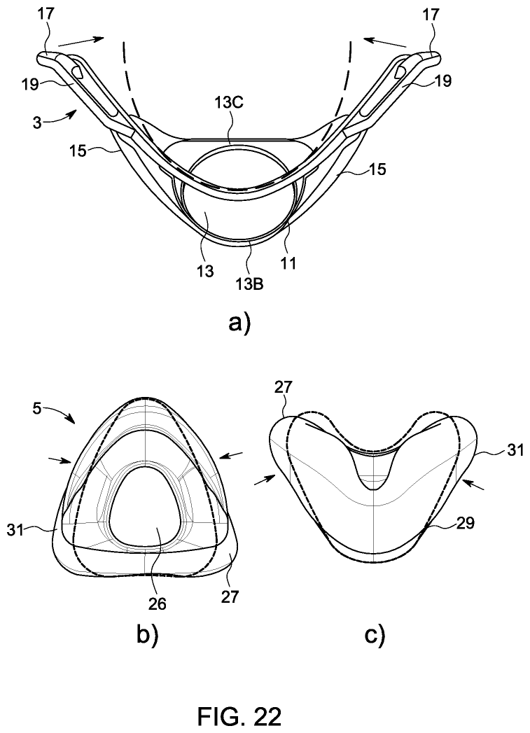

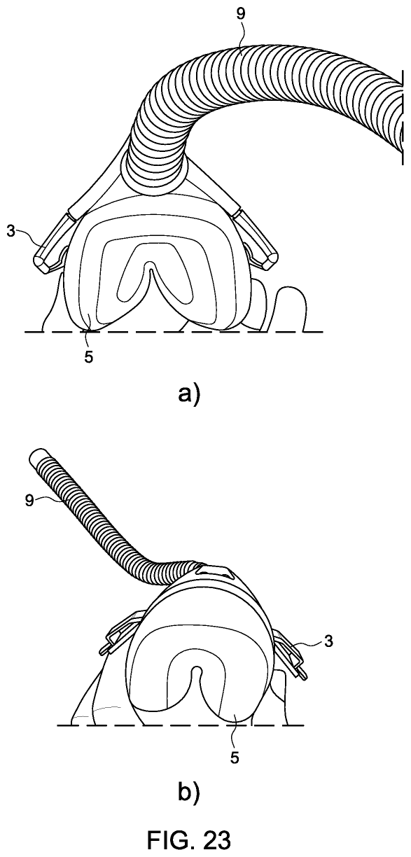

Referring to FIGS. 13 to 15, 22 and 23, one aspect of the mask assembly 1 in accordance with this disclosure is the ability for the frame 3 and cushion 5 to resiliently deform in a lateral direction, as shown by the arrows in FIG. 13a). Another aspect of the mask assembly 1 in accordance with this disclosure is the ability of the frame 3 and cushion 5 to resist deformation in a front to back (or vice versa) direction, as shown by the arrows in FIG. 13b).

In particular the frame 3 is configured to be relatively laterally resiliently deformable (that is relatively flexible across the user's face), but to be relatively non-deformable in a front to back direction (that is relatively rigid in a direction into the user's face).

The frame 3 is configured to do this by:
a) comprising a central portion 11 being relatively wide, relatively thick, and comprising a relatively large amount of material, and by the lateral arms 15 and upper frame portion 25 being relatively narrow, relatively thin, and comprising a relatively small amount of material; and/or
b) being connected to the cushion 5 only at the central portion 11; and/or
c) having a frame opening 13 which is reinforced and/or braced, as compared to the lateral arms 15. For example, the front and rear margins 13B, 13C of the frame opening 13 extend laterally across the central portion 13 and can be considered to form struts that function to brace the frame opening 13 against lateral compression.
d) comprising one or more reinforced regions; and/or
e) comprising one or more weakened regions.

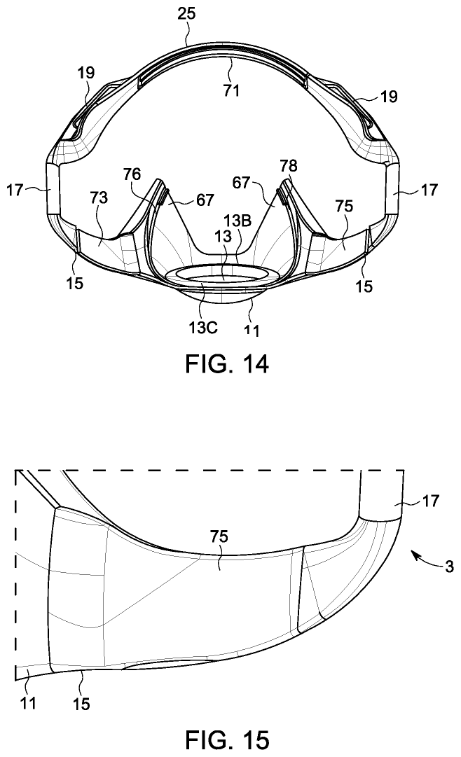

For example, with reference to FIG. 14, three weakened regions 71, 73, 75 are shown, region 71 being provided by the relatively thin, arcuate upper portion 25 of the frame, regions 73, 75 being regions of thinned material, where the central portion 11 transitions into the lateral arms 15. Region 71 comprises a central region of the elongate frame portion 25. Region 71 comprises a thinner band of material than the widened regions 19A. The weakened regions 71, 73,75 are more flexible than the adjacent regions.

With reference to FIGS. 12 and 14, the sides of the central portion 13, either side of the frame opening 13 are reinforced with an arcuate, upwardly extending ridge or thickened portion 76, 78. These thickened portions join the front and rear margins 13B, 13C to increase the rigidity of the central portion 13 of the frame 3.

Referring to FIG. 27, a discrete hinge 77 may be provided at one or more regions of the frame 3, for example where the central portion 11 transitions into the lateral arms 15. The hinge 77 may comprise a living hinge.

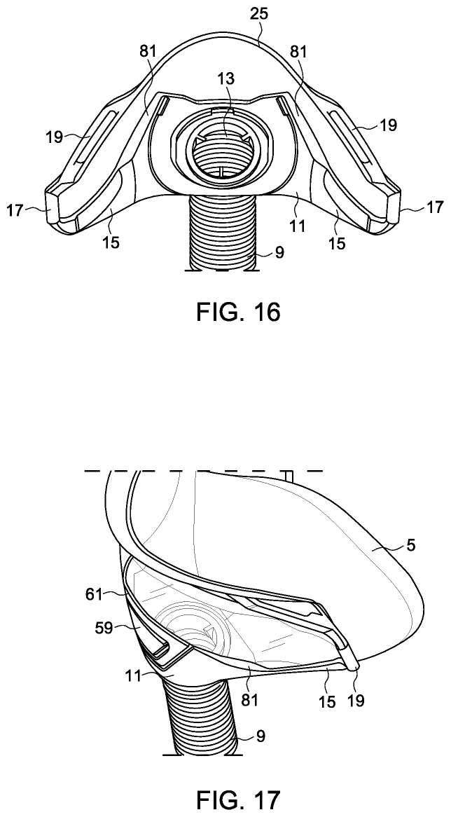

Referring to FIGS. 16, 17 and 26, the mask assembly 1 comprises a pair of laterally spaced finger grips 81 configured to facilitate the user gripping the mask assembly with two fingers.

The finger grips 81 are below the centre of the mask assembly 1 when viewed from the front, above the inlet opening 13 in the frame, and adjacent the inlet connector 7, in particular adjacent the vent holes 49 and the diffuser 59.

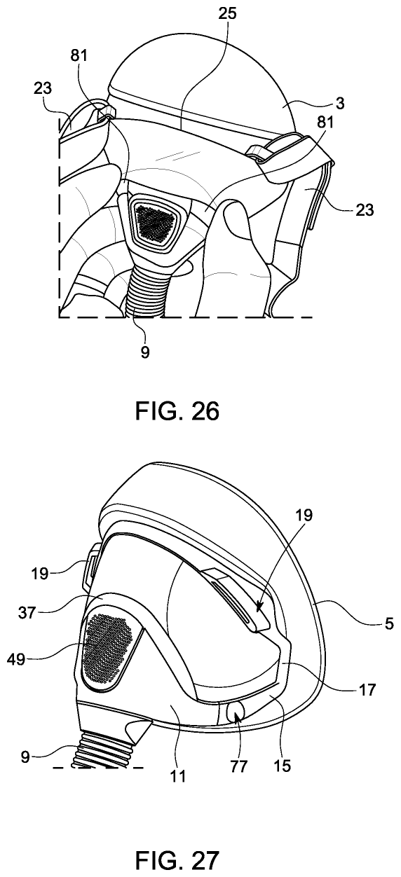

In the embodiment of FIGS. 16 and 17, the finger grips 81 are provided at the upper margins of the wings 67 of the central portion 11 of the frame 3, these upper margins comprising a thickened edge of the frame 3 to provide a larger surface for the fingers to grip, for example when detaching the cushion 5 from the frame 3. The frame 3 deflects inwardly to allow the user's fingers to grip this edge, as shown in FIG. 26.

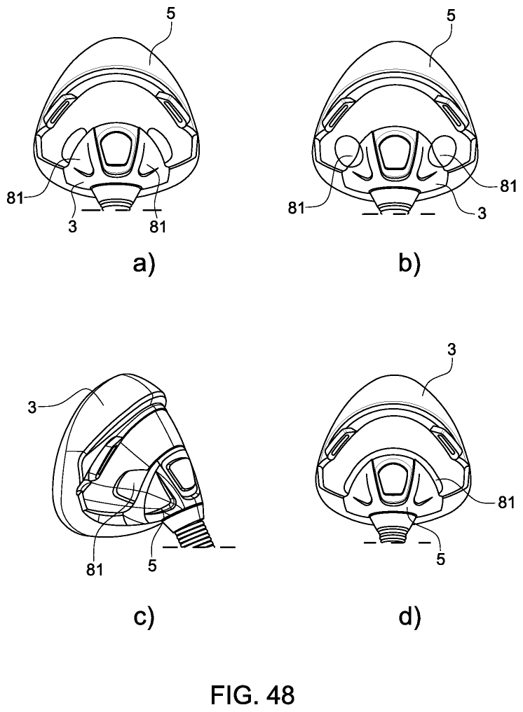

FIG. 48 shows alternative embodiments of finger grips 81. FIG. 48a shows finger grips 81 each provided by a recessed region formed in the inlet connector 7 and cushion 5. FIG. 48b) shows finger grips 81 formed by indented region of the lateral margins of the wings of the frame 3. FIG. 48c) is similar to FIG. 48a) but with deeper recesses in the frame 3 and cushion 5. FIG. 48d) shows a finger grip 81 comprising an elongate, arcuate recess in the cushion extending around the sides and top of the central portion 11 of the frame 3.

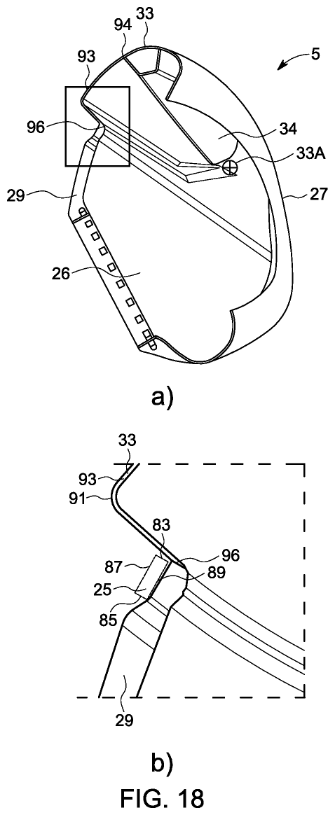

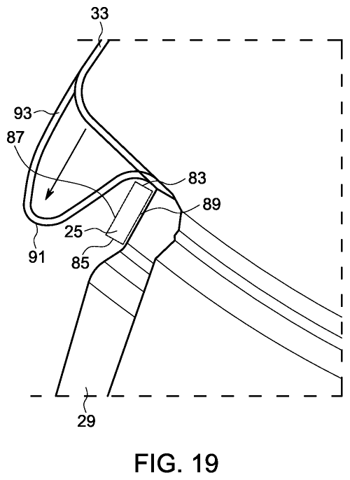

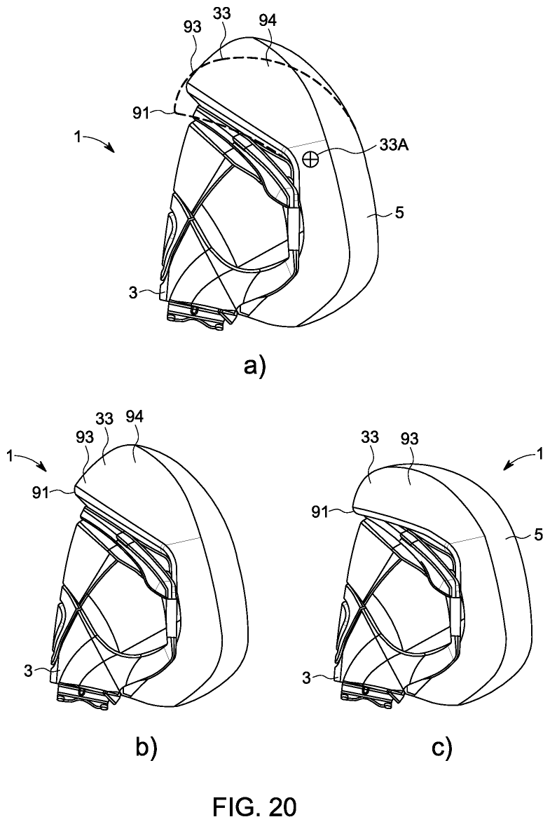

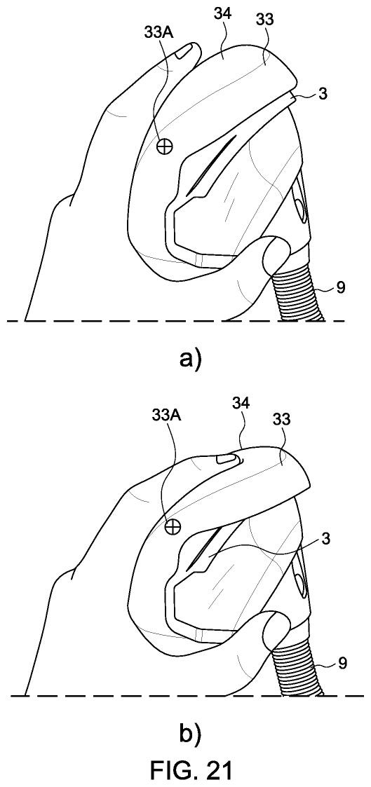

Referring to FIGS. 18 to 21, these figures show the rolling bridge 33 of the cushion 5 in more detail. FIGS. 18 and 19 show an embodiment for the upper portion 25 of the frame 3. In this embodiment the elongate frame member of the upper portion 25 of the frame 3 is of rectangular cross section and comprises upper and lower walls 83, 85 and forward and rear walls 87, 89. The rear wall 89 rests against the forward surface of the cushion 5. The upper wall 83 is positioned underneath the forward part of the rolling bridge 33. The upper portion 25 of the frame may have other cross sections, for example quadrilateral, circular, elliptical, or triangular. Whichever shape cross section is provided will have a forwardmost margin, and an uppermost margin, over which the rolling bridge portion 33 can roll.

An upper portion of the cushion 5 comprises a rolling bridge portion 33 which is pivotable relative to a lower portion 35 of the cushion 5, between a rest position in which the rolling bridge portion 33 is relatively rearward, to an in use position in which the rolling bridge portion 33 pivots forwardly such that part of the rolling bridge portion 33 buckles to provide a roll of material that extends over the mask frame 3. The rolling bridge portion 33 pivots about a pivot axis 33A.

As shown in FIGS. 19, 20 and 21, a forward margin of the rolling bridge portion comprises a bend 91 and a stiffer region (e.g., a region of thicker cross section) adjacent to a region of reduced stiffness 93 help to initiate rolling of the region of reduced stiffness 93. In other words, a controlled buckling of the region of reduced stiffness 93 occurs with the assistance of the adjacent stiffer portions. In addition, positioning an edge of the relatively more rigid upper frame portion 25 adjacent to the bend 91 further helps to induce rolling in the region of reduced stiffness 93. In some configurations, the region of reduced stiffness 93 is bounded by a first boundary 94, and a second boundary 96, wherein the first boundary 94 and the second boundary 96 have an increased stiffness relative to the region of reduced stiffness. The first boundary 94 may defined by or alongside the thickened band 34 extending over the rolling bridge portion 33, while the second boundary is defined by or alongside the bend 91. In some configurations, the second boundary 96 can be defined by or alongside an edge of the more rigid upper frame portion 25. In some configurations, the second boundary 96 can be defined along a portion of the cushion 5 positioned between the mask frame 3 and the region of reduced stiffness 93.

As the rolling bridge 33 of the cushion 5 is displaced about the pivot axis 33A, the roll increases in size. In other words, as the first boundary 94 initially moves toward the second boundary 96, a roll is formed in the cushion 5. As the first boundary 94 continues to move toward the second boundary 96, the roll continues to increase in size. Thus, in the illustrated embodiment, the roll defined in the rolling bridge 33 starts at nothing and progressively increases during displacement. Preferably, the rolling between the first boundary 94 and the second boundary 96 creates a single bend or inflection between the first boundary 94 and the second boundary 96. The single bend results in legs approaching the bend location that increase in size as the first boundary 94 moves toward the second boundary 96. In other words, the rolling created by movement of the first boundary 94 toward the second boundary 96 preferably does not result in a fan-folding appearance such as a pleated configuration.

The rolling bridge 33 is configured to move forwardly from a rest condition where the rolling bridge 33 is not pivoted about pivot axis 33A, as shown in FIGS. 18, 20b) and 21a). The rolling bridge 33 can roll forwardly, about pivot axis 33A to a fully pivoted condition where the rolling bridge 33 extends over the upper and forward walls 83, 87 of the upper frame portion 25. This can be seen with reference to FIGS. 18b), 19, 20a) and c), and 21b). As can best be seen with reference to FIG. 19, the forward portion 33A of the rolling bridge 33 rolls forwardly and downwardly when such that when in the fully pivoted condition, the forward portion 33B extends over and substantially encloses the upper frame portion 25, and in particular the forward wall 87 of the upper frame portion 25.

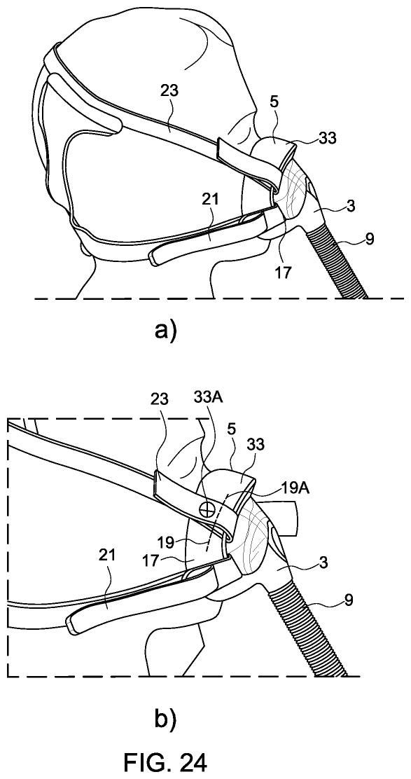

Referring to FIGS. 24 and 35 the headgear straps 21, 23 are shown connected to respective headgear connectors 17, 19 on one side of the mask assembly 1. The upper headgear strap 23 extends from upper headgear connector 19 in a direction in which the upper headgear strap 23 overlaps the pivot axis 33A of the rolling bridge 33. The longitudinal axis 23A of the upper headgear strap 23 may intersect with the pivot axis 33A. As can best be seen in FIG. 24b), the elongate element of the upper headgear connector 19 has an axis 19A. A line which perpendicularly bisects the first axis 19A intersects with the pivot axis 33A of the rolling bridge portion 33. In other words a line through the centre of the headgear connector intersects the rolling bridge axis 33A, when the mask assembly 1 is viewed from the side.

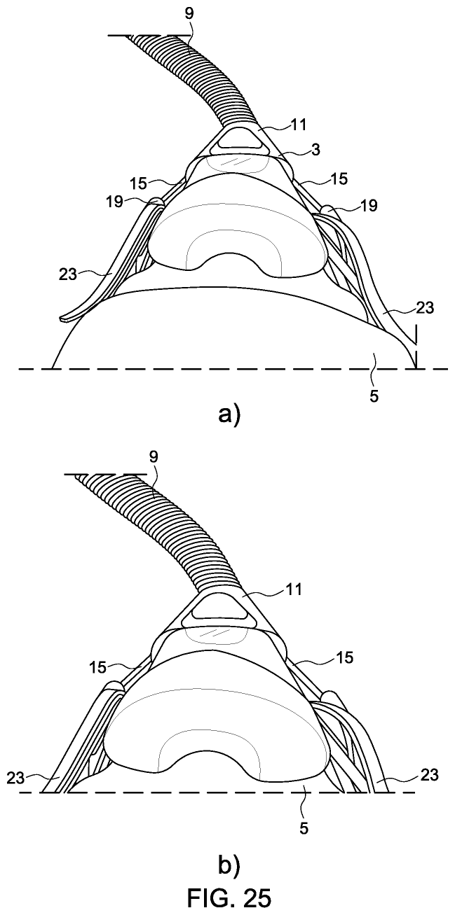

Referring to FIGS. 25 and 35, the headgear straps 21, 23 extend outwardly from the lateral arms 15 of the mask frame 3 such that the longitudinal axis of the headgear straps 21, 23 is aligned with the longitudinal axis 15A of the arms 15, when the mask assembly 1 is viewed from above. In other words the angle of the arms is substantially aligned with a direction of tensile force applies by the straps 21, 23, when the straps are connected to the headgear connectors 17, 19. This facilitates the frame 3 holding the cushion 5 to the face effectively, and reduces a tendency for the frame 3 to bend due to the force applied by the straps. This curved shape allows the headgear connectors 17, 19 to be located further back from the front of the mask assembly 1 and thus closer to the face. This allows the user, when side sleeping, to turn their face further into the pillow, without being obstructed by a rigid frame. In the embodiment illustrated, the lower headgear connectors 17 are the rearmost part of the frame 3, when the frame 3 is viewed from the side.

When the cushion 5 is mounted on the frame 3, the lateral arms 15 may be spaced away from the sides of the cushion 5 so that there is a gap or clearance therebetween.

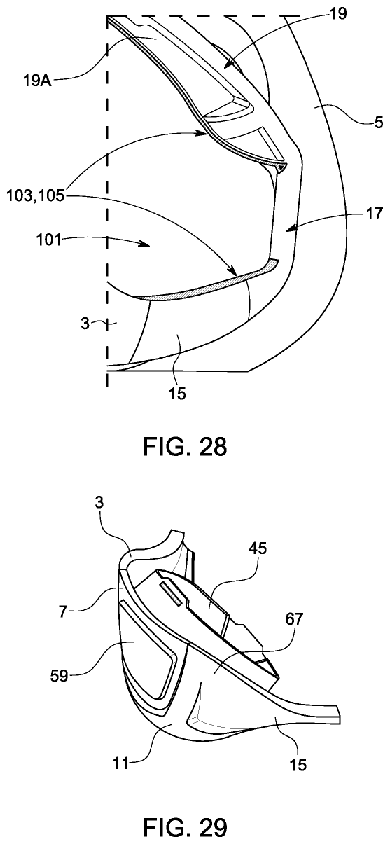

Referring now to FIG. 28, the lower headgear connectors 17 are located in a guide mouth 101 defined by a lower guide surface 103 being the top surface of a portion of the lateral arms 15, and an upper guide surface 105 being the underside of a portion of the upper portion 25 of the frame, adjacent the upper headgear connectors 19. The guide surfaces 103, 105 are downwardly and upwardly inclined respectively away from the headgear connectors 17 so that the distal ends of the guide surfaces 103, 105, are spaced further vertically apart than the length of the post of each headgear connector 17. The guide surfaces 103, 105 thus define a guide mouth 101 into which the headgear strap 21 can be positioned and pulled rearwardly, to facilitate engagement of the headgear strap 21 with a lower headgear connector 17. The headgear strap 21 may comprise a clip comprising a hook that is guided, along guide surfaces 103, 105 onto the headgear connector 17 as the headgear strap is pulled rearwardly. The guide surfaces 103, 105 thus facilitate quick, reliable and easy connection of the lower headgear strap 17 to a lower headgear connector 17.

In the embodiment shown in the figures the lower guide surface 103 comprises an arcuate top surface of the lateral arms 15. The top surface may be rounded or planar. The upper guide surface 105 comprises an arcuate underside of the enlarged region 19A of the frame 3.

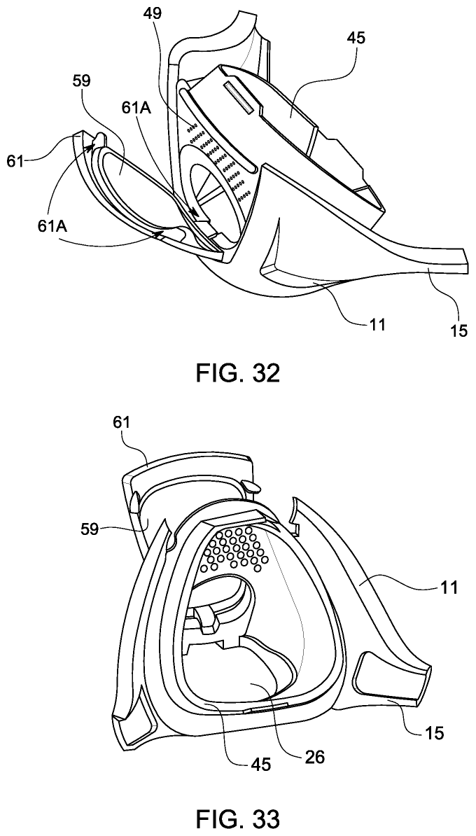

Referring now to FIGS. 29 to 33, some further detail of the inlet connector 7 are shown. The bias vent holes 49 are provided in an array on the inclined forward wall 63 of the inlet connector 7. The bias vent holes 49 help circulate air and ensure that expired carbon dioxide is not rebreathed by the patient. As noted above, a diffuser 59 may be provided to cover the bias vent holes 49. The diffuser 59 diffuses gases flow from the bias vent holes 49, minimising any unwanted jetting effects and noise caused by the bias flow.

The diffuser 59 may be removably or permanently mounted on the inlet connector 7 via a diffuser frame 61 to which the diffuser material 59 is mounted, for example via adhesive, overmoulding, hot welding or the like. The diffuser frame 61 may be removably mounted onto the inlet connector 7 via connection formations 61A on one or both of the frame 61 and inlet connector 7. The diffuser frame 61 may alternatively be permanently or removably mounted on the central portion 11 of the frame 3. When removably mounted, the diffuser frame 61 may be provided with an outwardly protruding finger tab 61B to facilitate the user applying a force to the diffuser frame 61 to remove the diffuser frame 61 from the mask assembly 1.

Additionally or alternatively, a cut-out may be provided on the diffuser frame 61 or the forward wall 62 of the inlet connector 7, to create a touch point for a user's finger.

The inner margins of the wings 67 of frame 3 between them define a recess 67A in which the diffuser frame 61 is received. The margins of the recess 67A are substantially straight and inwardly inclined, and facilitate correct alignment of the diffuser frame 61 with the frame 3, and alignment of any connection formations 61A.

The connection formations 61A may comprise a tab at a lower margin of the diffuser frame 61 that is inserted into a recess at the bottom or recess 67A. The diffuser frame 61 can be pivoted forwardly via engagement of the tab with the recess, such that an upper part of the diffuser frame 61 clips onto, or engages with, the inlet connector 7 above the array of vent holes 49 via one or more further connections formations 61A.

When the diffuser frame 61 is mounted in the recess 67A, a space 67B may be provided between the margins of the diffuser frame 61 and the recess 67A. Space 67B therefore comprises a channel, for example approximately 0.5 mm wide around the perimeter of the diffuser frame 61, which provides a small tolerance to the fit of the diffuser frame 61 in the recess 67A.

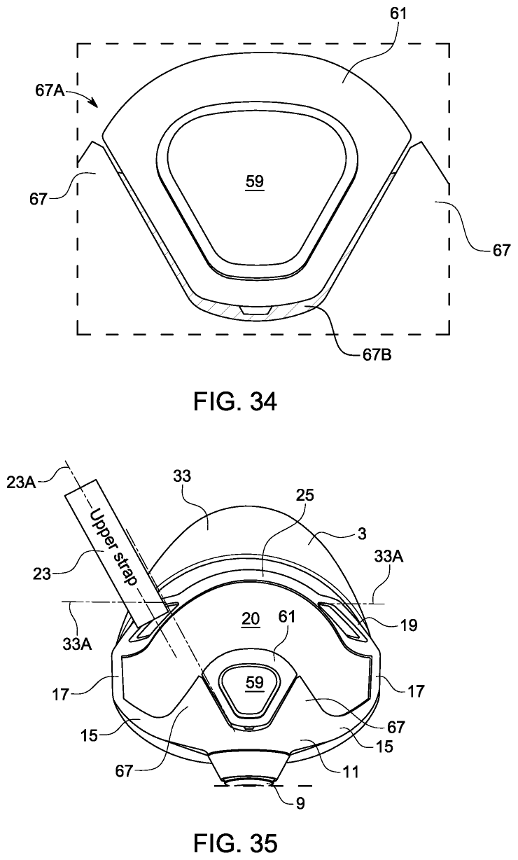

As can be seen in FIG. 34, the inlet connector 7 may be of a different colour from the diffuser frame 61, and/or the frame 3. For example, the inlet connector 7 may be blue, and the diffuser frame 61 grey or white. Consequently, when the diffuser frame 61 is mounted on the inlet connector 7, the colour of the inlet connector 7 can be viewed through the space 67B.

As can be seen in FIGS. 34 and 35, the recess 67A comprises opposed outer side margins that are substantially straight and which are inclined so as to converge at a base of the recess 67A. The recess 67A can be considered to be substantially V shaped when the mask assembly 1 is viewed from the front. When the upper headgear straps 23 are connected to the upper headgear connectors 19, the longitudinal axis of each upper headgear straps 23 is parallel with a respective side margin of the recess 67A. An edge margin of each upper headgear strap 23 may be substantially aligned with a respect side margin of the recess 67A. The upper headgear straps 23, the recess 67A, and the diffuser frame 61, each have a shape with similarly inclined, straight, side margins, giving the mask assembly 1 a distinctive appearance when viewed from the front.

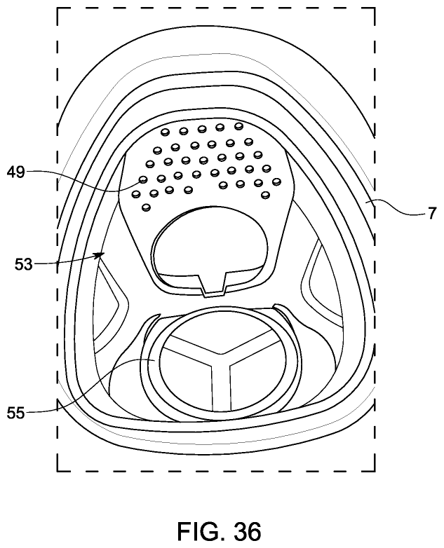

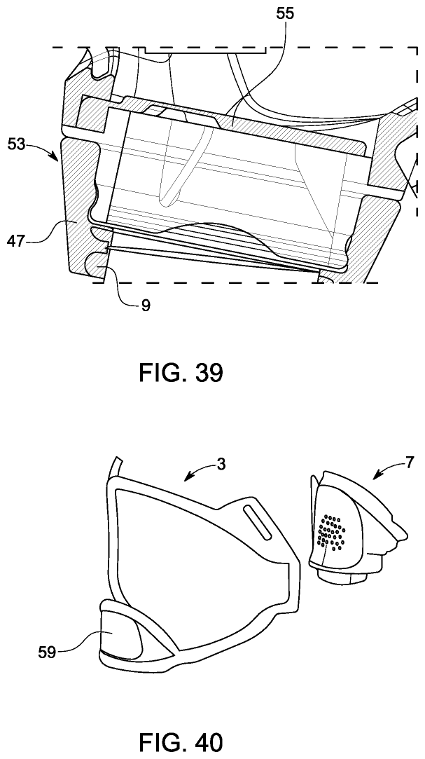

Referring now to FIGS. 36-39 the anti-asphyxia valve 53 can be seen in more detail. The valve opening 57 acts as a non-return valve (NRV). When a positive pressure is applied, the valve member 55 is open and covers valve opening 57. When a negative pressure arises, the valve member 55 covers the gas inlet to prevent backflow and provide a large leak for exhaled gas through the valve opening 57.

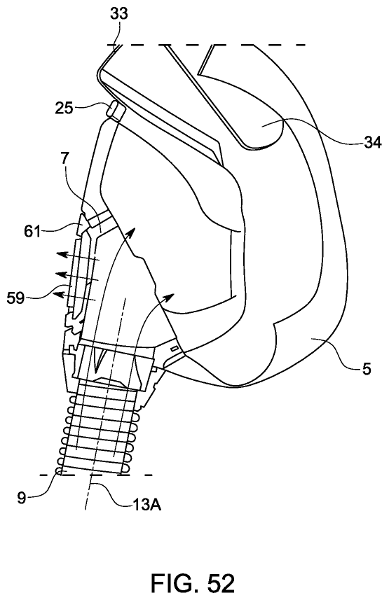

In this embodiment the valve member 55 is movably mounted adjacent the inlet boss 47, at a forward part of the inlet connector 7. The valve member 55 pivots upwardly and forwardly towards the forward wall 53, away from the outlet boss 45. The valve member 55 is movable between a closed position on which the valve member 55 closes the valve opening 57, and an open position in which the valve member 55 opens the opening (instead closing the passage through the inlet boss 47). When in the closed position, because the valve member 55 pivots to a position substantially parallel with the forward wall 63 of the inlet connector, the valve member 55 is out of the inlet flow path through the inlet connector 7, between the inlet boss 47 and outlet boss 45. This allows the outlet opening defined by outlet boss 45 to be relatively large, and to be positioned relative to inlet boss 47 such that the inlet flow path through the inlet connector 7 is substantially straight. Thus, flow path can be seen via the arrows in FIG. 52. Because of this configuration, a substantial part of the inlet flow can be delivered directly into the cavity inside the cushion 5, without having to be deflected or turn a corner.

Further, the positioning of the anti-asphyxia valve 53 in this way, also enables the inlet connector 7 to be relatively small and discrete, and to occupy a relatively small proportion of the frontal area of the mask assembly 1. This minimises the visual impact of the inlet connector 7, vents holes 49, and the anti-asphyxia valve 53, and enables a relatively large frame aperture 20 to be provided. Having a multi-function inlet connector 7 further facilitates providing these benefits.

Such an inlet connector 7 is also relatively compact when viewed from the side or front, in a vertical direction, i.e. the inlet connector 7 extends a smaller distance below the cushion 5. The entre mask assembly 1 can therefore be more compact in a vertical direction.

In this embodiment the central axis through the inlet boss 47, and frame inlet opening 13 is substantially perpendicular to the axes through the vent holes 49. In other words, the direction of the inlet gases flow is substantially perpendicular to the direction of the bias vent flow, as indicated by the arrows in FIG. 52.

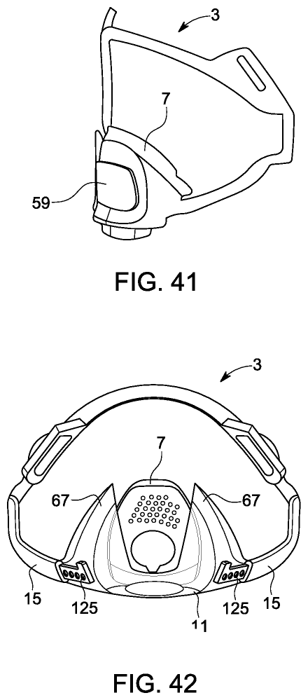

Referring to FIGS. 40 and 41, alternative inlet connectors 7 are provided. These inlet connectors 7 can have any of the features described above, for example the outlet boss 45, inlet boss 47, vent holes 49, anti-asphyxia valve 53, and diffuser 59.

The inlet connector 7 may be a separate component, or could be integrally formed with the frame 3.

The diffuser 59 may be mounted on, or integrally formed with, the frame 3, or the inlet connector 7.

FIG. 40 shows an embodiment where the frame 3 and diffuser 59 are integral, with the inlet connector 7 a separate component.

FIG. 41 shows an embodiment where the frame 3, inlet connector 7, and diffuser 59 are integral, and form a single component.

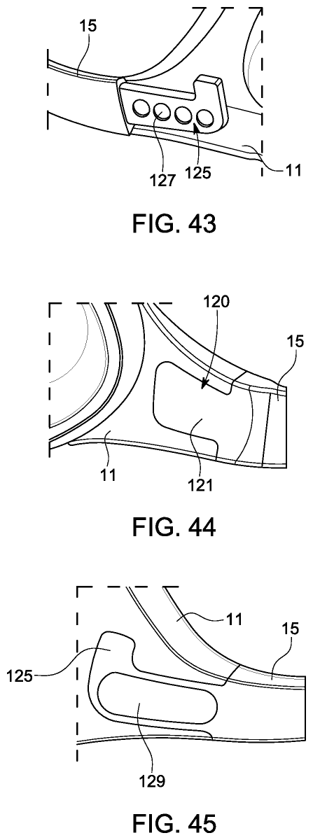

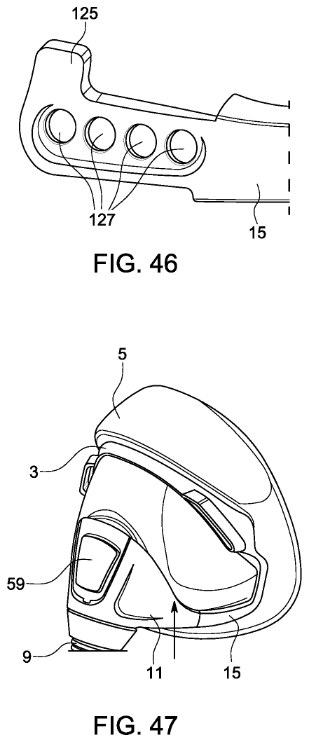

In the above embodiments, the lateral arms 15 are integrally formed with, and part of, the frame 3. Referring now to FIGS. 42 to 47, the lateral arms 15 and frame 3 may be separate components. This allows the frame 3 to be made of a different material from the lateral arms 15 for example. FIG. 44 shows a dove tail connection 120 between the dove tailed ends 121 of the lateral arms 15 and a corresponding slot 123 in the lower portion of the frame 3. FIGS. 42, 43, 45 and 46 show barbed ends 125 of the lateral arms 15 that are received in corresponding shaped slots 127 in the lower portion of the frame 3. The barbed ends 125 may then be overmoulded 129 to the central frame portion 15. The ends of the arms 15 may be provided with apertures 129 through which overmould material can flow during manufacturing. These examples help ensure that the connection between the arms 15 and frame 3 is sufficient to withstand the tensile forces exerted by the headgear straps 21, 23 on the frame 3 in use.

Referring now to FIGS. 3, 49 and 52 the configuration of the frame 3, cushion 5 and inlet connector 7 is such that the breathing gas inlet conduit 9 extends generally below the mask assembly 1. That is, when viewed from the side, the inlet conduit 9 extends downwardly from the mask assembly 1, and upper end of the conduit 9 does not project substantively forwardly. The upper end of the conduit does no project forwardly of the forward margin of the mask assembly 1, that forward margin being defined by the central portion 13 of the frame 3.

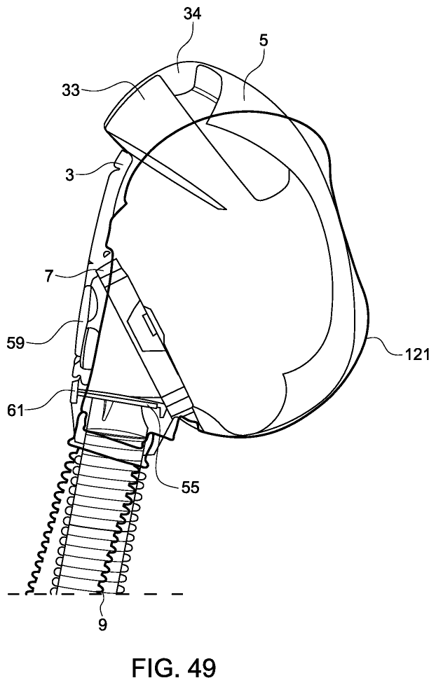

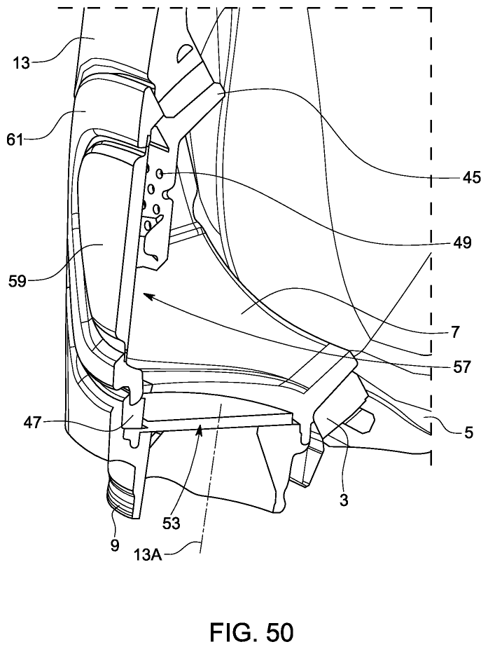

A comparison between the angle of the conduit 9 between the current mask assembly 1 and a prior art mask assembly 121 can be seen in FIG. 49. In normal use, with a user standing or sitting, the mounting posts of the lower headgear connectors 17 are substantially vertical. The base of the central portion 13 of the frame 3 is substantially planar, the inlet opening 13 being slightly inclined. Likewise, the inlet boss 47 of the inlet connector 7 is also slightly inclined. Consequently, the connection between the conduit 9 and the inlet connector 7 is such that the conduit 9 hangs below the mask assembly 1, when viewed from the side. The upper end of the conduit 9 is within the forwardmost margin of the mask assembly 1. This configuration minimises any forward, outward, protrusion of the inlet connector 7 and conduit 9 in us use, that could contact a pillow or bed when the patient is side sleeping.

When viewed from the front, the maximum vertical dimension of the frame 3 is less than the maximum vertical dimension of the cushion 5. In particular the rolling bridge portion 33 of the cushion 5 projects above the uppermost part of the frame 3.

When viewed from the front the maximum lateral dimension of the frame 3 is substantially the same as the maximum lateral dimension of the cushion. The outer margin of the lower most headgear connectors 17 is substantially aligned with the outer margin of the side wall 31 of the cushion 5.

When viewed from the front, the shape of the outer periphery of the frame 3 substantially matches the shape of the outer periphery of the cushion 5. In this embodiment, the cushion and frame comprise a substantially planar base and inwardly inclined sides. The lower part of the mask assembly 1 is relatively wide, the sides of the mask assembly 1 are inwardly inclined towards a relatively narrow apex portion.

When viewed from the front, the outer periphery of the frame 3 is substantially within the outer periphery of the cushion 5.

When viewed from the side, the outer periphery of the lateral arms 15 and upper frame portion 25 are within the outer periphery of the cushion 5.

The ratio of the height of the inlet connector 7 to the height of the frame 3 may be between 0.5 to 0.75, and is preferably approximately 0.67.

The ratio of the width of the central portion 13 of the frame 3 to the width of the frame 3 may be between 0.45 and 0.65, and is preferably approximately 0.57.

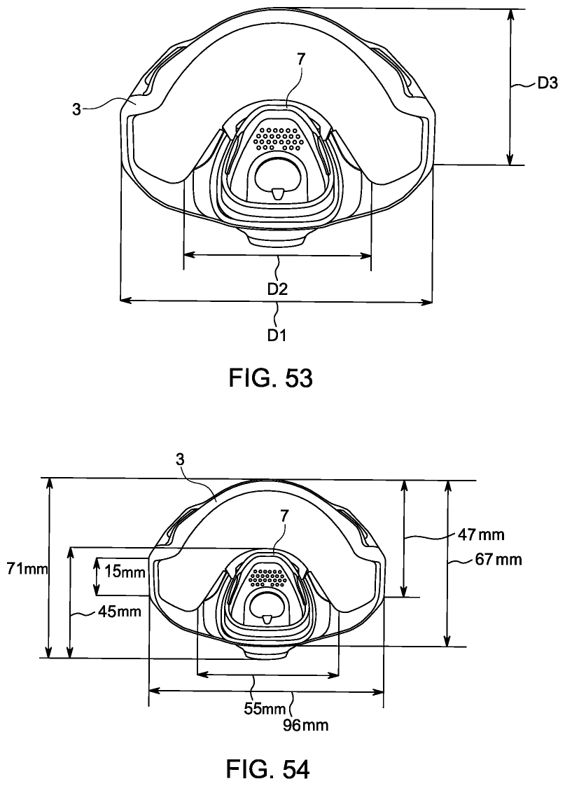

With reference to FIGS. 53 to 57 some example dimensions of a mask assembly 1 in accordance with this disclosure are shown.

Referring initially to FIG. 53, the overall width D1 of the frame 3, from one lower headgear connector 17 to the other, may be approximately 100 mm, and preferably 96 mm. The overall width D2 of the central portion 13 of the frame 3 may be approximately 55 mm. A vertical distance between the bottom of the lower headgear connectors 17 and the apex of the frame 3 (at the top of elongate frame portion 25) may be approximately 50 mm, and preferably 47 mm.

The frame 3 may be configured such that the maximum lateral deformation of the frame 3 is equal to the difference between D1 and D2, that is, the frame 3 can deform sufficiently that the lower headgear connectors 17 can move to a position aligned with the outer margin of the lower portion 13 of the frame 3.

The frame 3 may be configured to be able to resiliently deform in a lateral direction from a resting condition, to a deformed condition in which the frame width:
a) equals the width of the central portion 13 of the frame 3; or
b) is greater than the width of the central portion 13 of the frame 3; or
c) is less than the width of the central portion 13 of the frame 3.

The frame 3 may be configured such that the maximum lateral deformation of the frame is substantially equal to D1, that is, the frame 3 can deform sufficiently that the lower headgear connectors 17 can move to a position aligned with the centre of the lower portion 13 of the frame 3, that is, aligned with the axis 13A.

In embodiments in accordance with this disclosure, D1 may be between 60-140 mm, 60-96 mm, 60-100 mm, 60-110 mm, 70-100 mm, 80-110 mm, 90-100mm, less than 110 mm or less than 140 mm.

In embodiments in accordance with this disclosure, D2 may be between 30-70mm, 40-70 mm, 40-55 mm, 40-60 mm, 50-70 mm, less than 60 mm, or less than 70 mm.

In embodiments in accordance with this disclosure, D3 may be between 30-80 mm, 30-50 mm, 35-50 mm, 40-50 mm, 40-60 mm, 40-80 mm, less than 50 mm, less than 60 mm, or less than 80 mm.

With reference to FIGS. 54 and 55, some possible dimensions for a mask assembly 1 in accordance with this disclosure, can include any one or more of:
a) frame height from inlet boss 47, when viewed from the front: 71 mm;
b) frame height from inlet opening 13, when viewed from the front: 67 mm;
c) frame width: 96 mm;
d) frame depth, when viewed from the side wit the elongate posts of the lower headgear connectors 17 vertical: 45 mm;
e) width of central portion 15: 55mm;
f) inlet opening 13: 24 mm high by 26 mm wide;
g) frame aperture 20: 25 mm wide, length from one lateral arm 15 to the other: 140 mm;
h) height of inlet connector 7 from inlet boss 47: 33mm;
i) length of mounting post of lower headgear connectors 17: 15mm;
j) inlet connector height from inlet boss 47: 45mm.
With reference to FIG. 55, some possible angles for a mask assembly 1 in accordance with this disclosure, can included any one or more of:
a) angle of elongate element of upper headgear connector 19 relative to angle of mounting post of lower headgear connector 17, when viewed from the side: 45°;
b) angle of upper margin of frame 3 relative to the mounting post of lower headgear connectors 17, when viewed from the side: 60°.

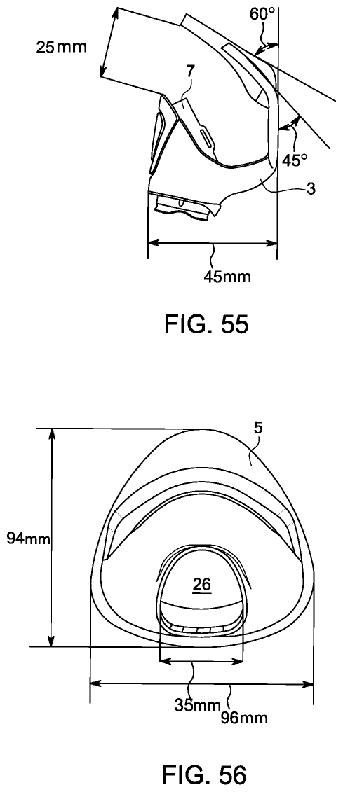

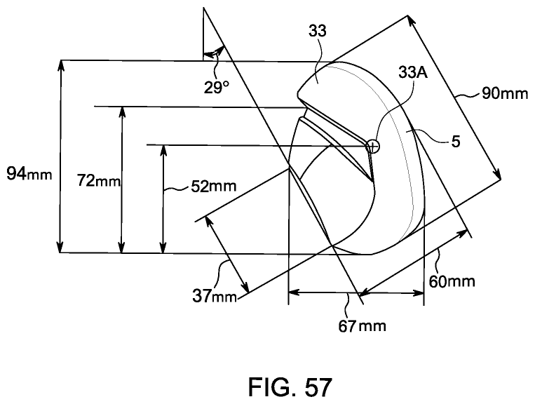

With reference to FIGS. 56 and 57, some possible dimensions for a mask assembly 1 in accordance with this disclosure, include:
a) cushion height when looking in direction along axis of inlet opening 25: 90 mm;
b) cushion height when viewed from the front: 94 mm c) cushion width: 96 mm;
d) cushion depth front to back, in a direction perpendicular to axis of inlet boss 47: 60 mm;
e) inlet aperture 25: 37 mm high, 35 mm wide;
f) height of pivot axis 33A from base of cushion 5 when viewed from the side: 52 mm.

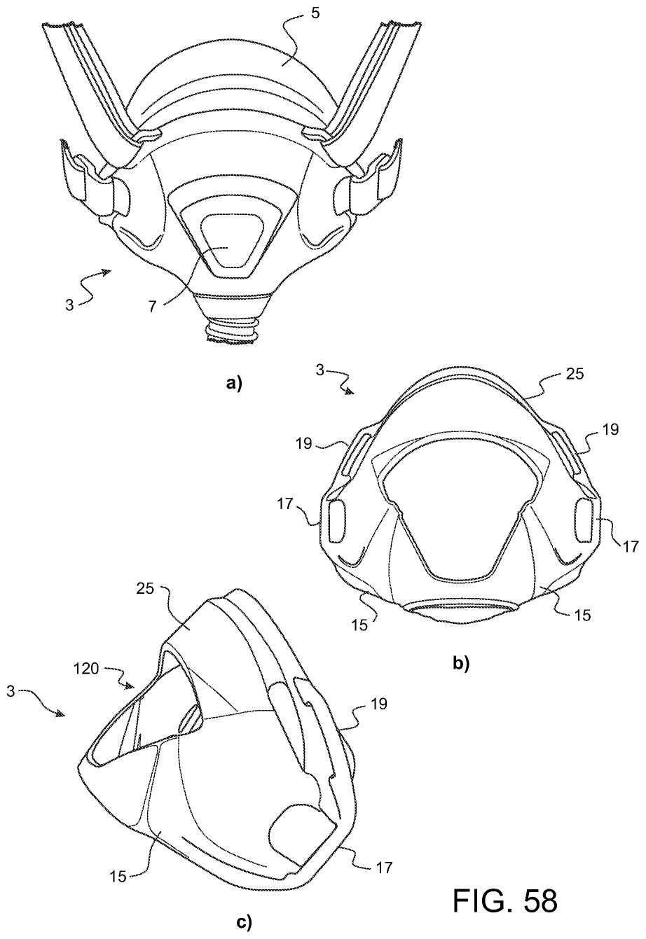

With reference to FIG. 58, a mask assembly comprises a modified frame 3. In this embodiment, features of the frame 3 are wider as compared to earlier described embodiments. The frame features have an increased contact area with the cushion as compared to earlier examples. The wider features provide increased support to the cushion 5, to assist in preventing or reducing leaks from the cushion 5, seal inversions and/or excessive ballooning of the cushion 5. The features of the frame 3 that are widened in this example include the lateral arms 15 and the upper portion 25.

The arms 15 are significantly widened so that the distal end of each arm 15 extends to a position adjacent both the lower and the upper headgear connectors 17, 19. The upper margin of each arm 15 is inclined upwardly from the frame opening 13 and terminates adjacent upper headgear connector 19. The lower margin of each arm 15 extends as per earlier embodiments to the lower end of lower headgear connector 17.

The upper portion 25 is also significantly wider than in earlier embodiments, such that the lower forward margin of the upper portion 25 is arcuate and defines the upper margin of the opening in which inlet connector 7 is received. The lower forward margin of the upper portion 25 is in contact with and engages the upper margin of the inlet connector 7. In this embodiment the frame 2 does not comprise window 20, instead comprising a trapezoidal opening 120 configured to receive and engage with the inlet connector 7.

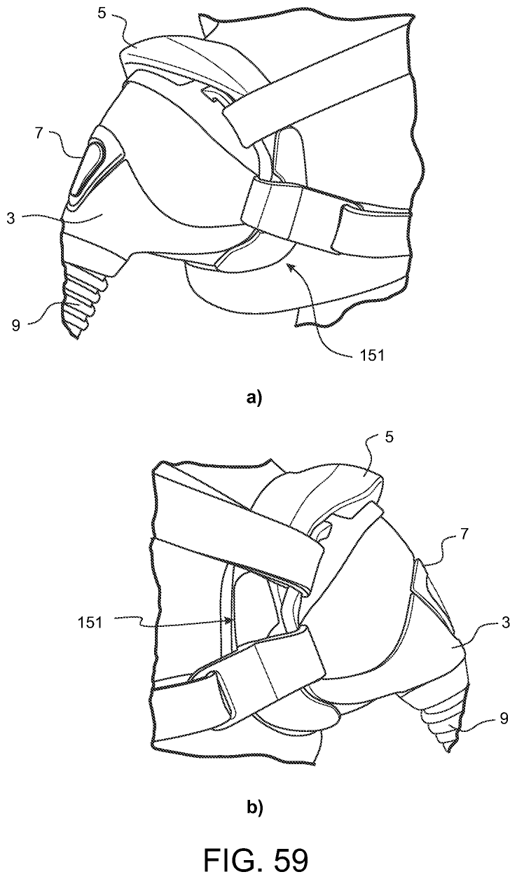

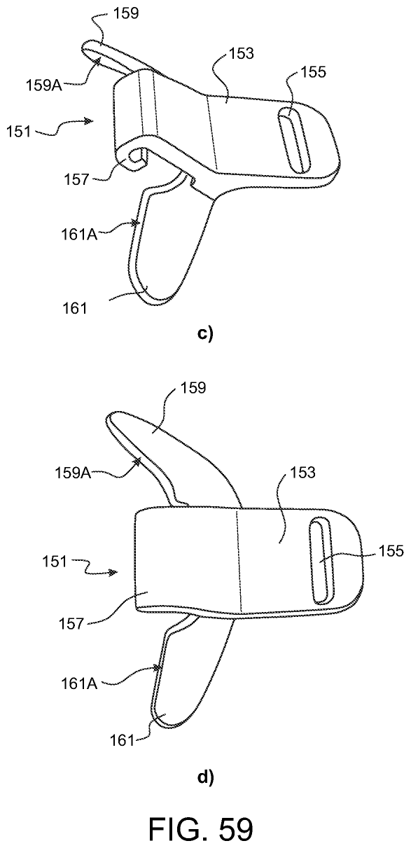

With reference to FIG. 59, a mask assembly comprises headgear clips 151 configured to connect the headgear straps to the frame 3. In one example, a pair of headgear clips 151 are provided to connect the lower headgear straps 21 to the lower headgear connectors 17 on the frame 3.

Each headgear clip 151 comprises an extension configured to support the side wall 31 of the cushion 5 to assist in reducing or preventing cushion blow out.

Each headgear clip 151 comprises an elongate body 153 one end of which comprises a slot 155 to receive the lower headgear strap 21, and the other end of which comprises a hook 157 to receive the post of the lower headgear connector 17. Each headgear clip 151 further comprises a pair of upper and lower extensions 159, 161 that extend away from the body 153. The extensions 159, 161 in this example are each shaped to match the shape of the side wall 31 of the cushion 5, and each comprise a support surface 159A, 161A against which the cushion side wall 31 can rest. Each support surface 159A, 161A limits lateral expansion of the cushion side wall 31, to help resist cushion blow out.

The lower extension 159 curves downwardly and inwardly to match the shape of a lower portion of the cushion side wall 31, the distal end of the lower extension 159 terminating below the lateral frame arm 15. The upper extension 161 curves upwardly and inwardly to match the shape of a side portion of the cushion side wall 31, the distal end of the upper extension 161 terminating adjacent upper headgear strap 23 and adjacent upper headgear connector 19.

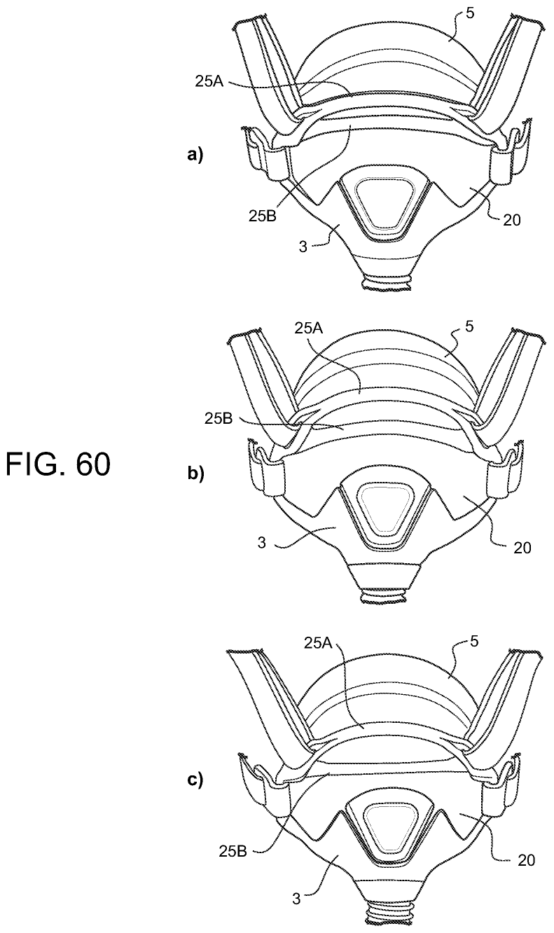

With reference to FIG. 60, a mask assembly comprises a modified frame 3. In particular, upper portion 25 comprises a pair of arcuate elongate frame elements 25A, 25B that are vertically spaced apart. Frame element 25A comprises the upper most part of the frame 3. Frame element 25B defines the upper margin of the closed, frame aperture 20 through which the cushion 5 is exposed.

The frame elements 25A, 25B may be configured to be fixed in spaced apart relationship relative to frame 3. The spacing of the frame elements 25A, 25B, and the provision of more than one frame element 25A, 25B, can provide additional support to the cushion 5. This may help pull the cushion 5 towards the face, rather than pulling cushion 5 upwardly.

The frame elements 25A, 25B may configured such that the space between the frame elements 25A, 25B can be adjusted to adjust the support provided to the cushion 5 by the frame elements 25A, 25B. The spacing may be adjusted, for example, between a closed position in which the frame elements 25A, 25B are relatively close together, for example with reference to FIG. 60A, to an intermediate position in which the frame elements 25A, 25B are spaced apart for example with reference to FIG. 60B, to a spaced position in which the frame elements 25A, 25B are relatively spaced apart for example with reference to FIG. 60C. By adjusting the spacing between the frame elements 25A, 25B, the support provided by the upper frame portion 25 to the cushion 5 may be adjusted.

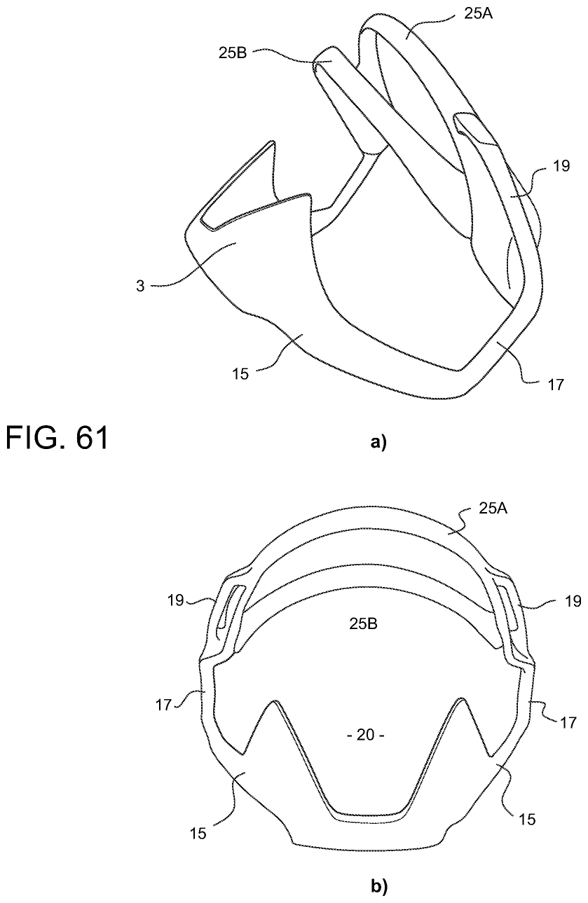

With additional reference to FIG. 61, the upper frame element 25A may extend between upper headgear connectors 19 as per earlier embodiments. The lower frame element 25B may be movable relative to the fixed upper frame element 25A and the remainder of the frame 3. The lower frame element 25B may be a separate component that is movably mounted to frame 3, for example by way of a pivotal mount 163. The lower frame element 25B may be integral with the frame 3 but comprise one or more regions that allow the lower frame element 25B to move relative to the upper frame element 25A, for example regions of predetermined weakness, one or more living hinges.

In another example, the lower frame element 25B may be fixed relative to frame 3, with upper frame element 25A being movable. In a further example, both frame elements 25A, 25B may be movable relative to frame 3.

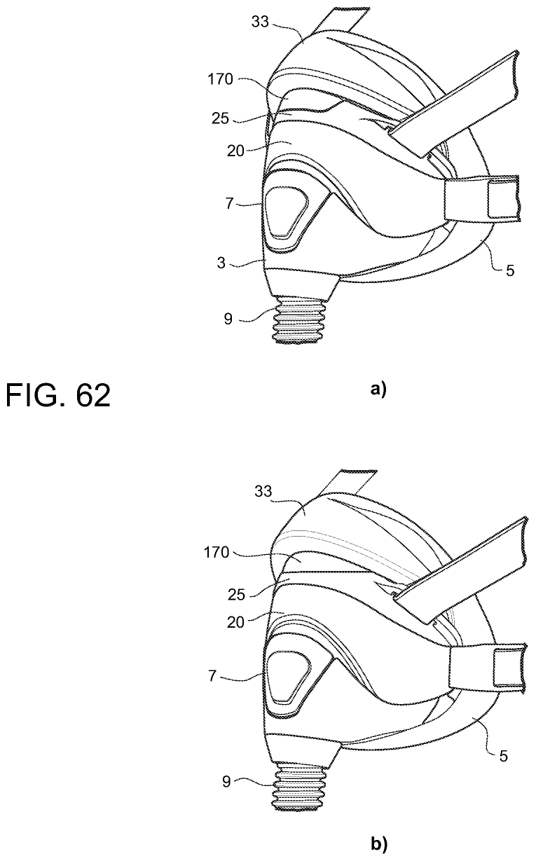

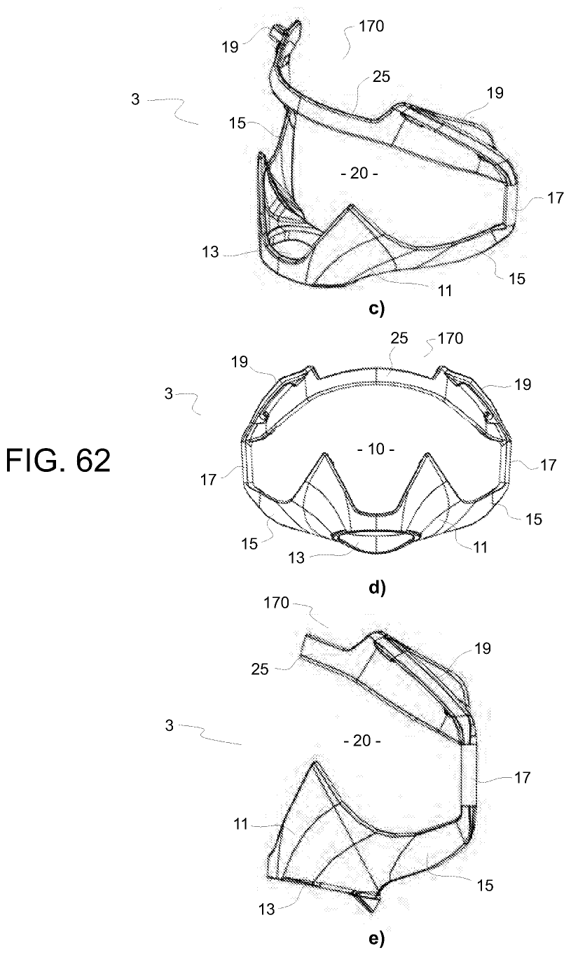

With reference to FIG. 62, a mask assembly comprises a modified frame 3. In particular, frame 3 comprises an upper portion 25 which is spaced below the forward margin of the nasal bridge region 33 of the cushion 5. The upper portion 25 may be spaced below the uppermost margin of the frame 3. In this position, the upper portion 25 of the frame pushes the cushion 5 towards the face more effectively, and can create a more equal load distribution on the cushion 5 that improves the seal with the patient's face.

Furthermore, when the frame 3 is experiencing a horizontal compressive force (for example as shown in FIG. 13a), frame upper portion 25 is less likely to lift away from the cushion 5.

With reference to FIG. 62a) and c) to e), the distal ends of the upper portion 25 narrows from a wider frame region adjacent the upper end of each upper headgear connector 19 to a narrower central apex, to define a space 170 between the forward margin of the nasal bridge region 33 of the cushion 5 and the upper margin of the upper portion 25 of the frame 3. The upper portion 25 thus comprises a narrowed region. As can be seen with reference to FIG. 62d), the upper margin of the narrowed region is positioned below the uppermost margin of the frame 3. In this embodiment, space 170 is defined by a cut-out portion of the central apex of the upper portion 25.

With reference to FIG. 62b) the upper portion 25 extends from the upper end of each upper headgear connector 19 across the mask assembly and smoothly tapers to a narrowed region, such that the upper portion 25 is spaced 170 below the forward margin of the nasal bridge region 33 of the cushion 5. Again, the upper margin of the narrowed region is below the uppermost margin of the frame 3.

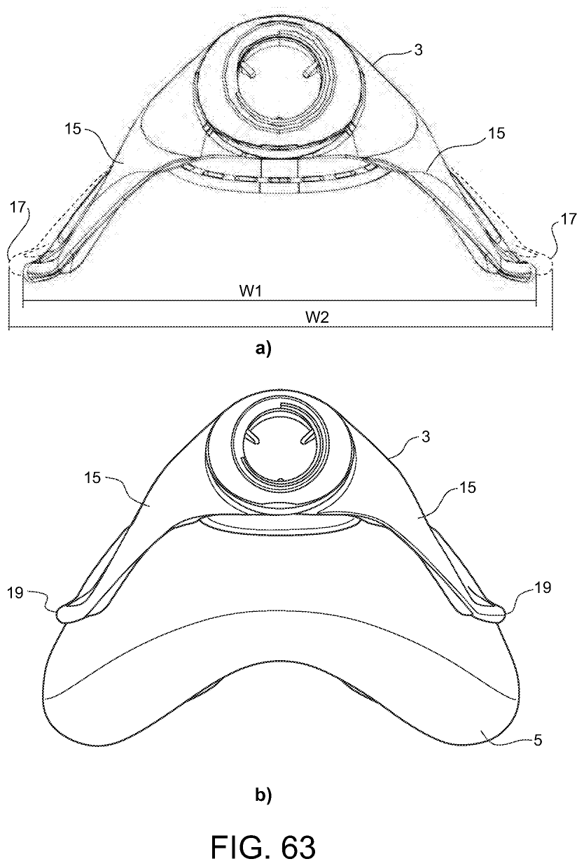

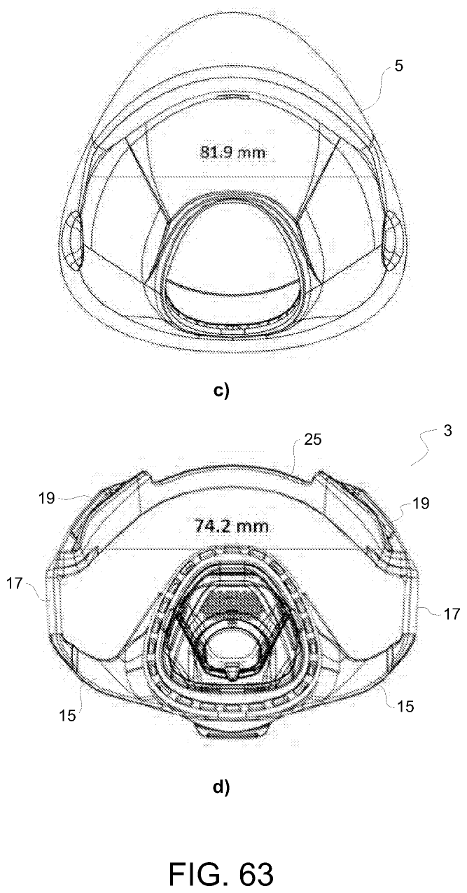

With reference to FIG. 63, a mask assembly comprises a modified frame 3. In particular, frame 3 is configured to deform the cushion 5 when the cushion 5 is mounted on the frame 3. The frame 3 squeezes the cushion 5, which can improve the seal between the cushion 5 and the patient's face, particularly at the patient's nose.

The frame 3 in this embodiment may therefore be narrower than the frame 3 of earlier embodiments. With reference to FIG. 63a) a frame 3 in accordance with this embodiment with a narrower width W1 is shown superimposed on a frame 3 as per previous embodiments with a wider width W2.

The frame 3 of this embodiment comprises lateral arms 15 which define a distance between them which is less than the corresponding dimension of the cushion 5. In particular the distance between the cushion engaging surfaces of the lateral arms 15 is less than the distance between the side walls 31 of the cushion 5. With reference to FIG. 63, example lateral dimensions of the cushion 5 and between the frame arms 15 are 82 mm and 74 mm respectively. In this example the distance between the frame arms 15 is around 90% of the lateral dimension of the cushion 5. In other examples the distance between the frame arms 15 may be between 80 and 97% of the lateral dimension of the cushion, or between 85 and 95%.

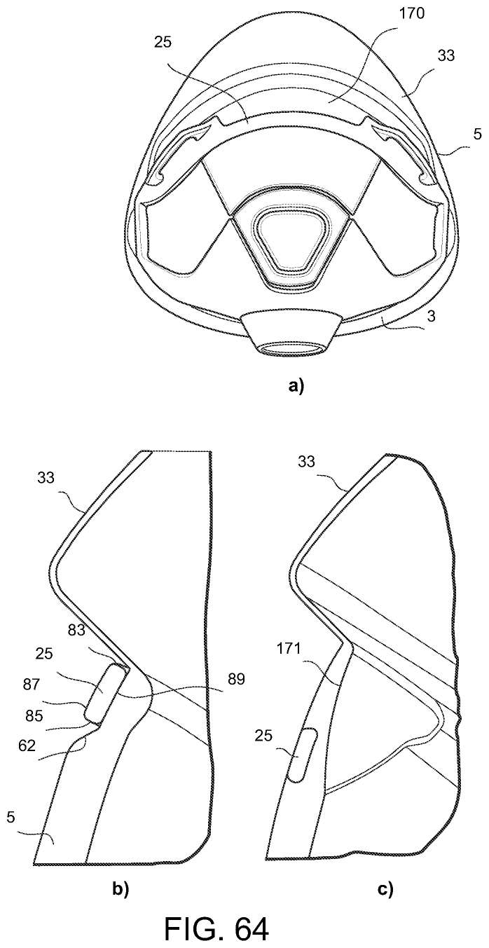

With reference to FIG. 64, a mask assembly comprises a modified frame 3 and cushion 5.

Referring to FIG. 64b), and earlier FIGS. 18 to 21, the elongate frame member of the upper portion 25 of the frame 3 of earlier embodiments is of rectangular cross section and comprises upper and lower walls 83, 85 and forward and rear walls 87, 89. The rear wall 89 rests against the forward surface of the cushion 5. The upper wall 83 is positioned underneath the forward part of the rolling bridge 33. The upper portion 25 of the frame 3 rests in recess 62 across the front of the cushion 5.

In the current embodiment, with reference to FIG. 64a) and c), the upper portion 25 of the frame 3 rests on the adjacent surface of the front wall of the cushion 5. As the cushion 5 and frame 3 are assembled together, the more rigid frame 3 deforms the resiliently deformable cushion 5. In particular, the upper portion 25 of the frame 3 deforms the adjacent surface of the cushion 5 such that the upper portion 25 rests on the front wall of the cushion 5. The upper portion 25 is mounted on the cushion 5 with an interference fit, as can be seen with reference to FIG. 64a) and c). The upper portion 25 thus resiliently deforms the cushion 5 during assembly, the deformation assisting in retaining the cushion 5 on the upper portion 25 of the frame 3. When so retained, the outer surface of the upper portion 25 may be flush with the outer surfaces of the cushion 5 adjacent the recess 62.

In the modified embodiment, as described above with reference to FIG. 62, the upper portion 25 of the frame 3 presses against the upper front surface of the cushion 5 at a position spaced 170 below the rolling bridge portion 33 of the cushion 5, and in particular below the second boundary 96 at the forward margin of the rolling bridge portion 33. This spacing 170 allows more clearance for the rolling bridge portion 33 to roll forwardly in use. The pressing of the upper portion 25 against the cushion 5 deforms the cushion 5 such that the upper portion 25 squeezes the cushion 5 in an interference fit.

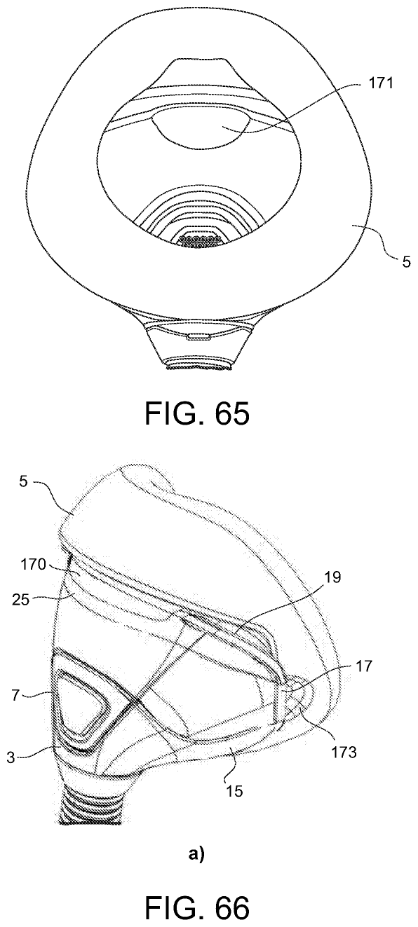

Referring to FIGS. 64c) and 65, the cushion 5 in this embodiment is modified to comprise a recessed region 171 to provide greater clearance between the interior surface of the cushion 5 and the patient's nose. The recessed region 171 is located centrally, on the interior of the cushion 5, directly below, but adjacent to, the rolling bridge portion, and in particular directly below the second boundary 96.

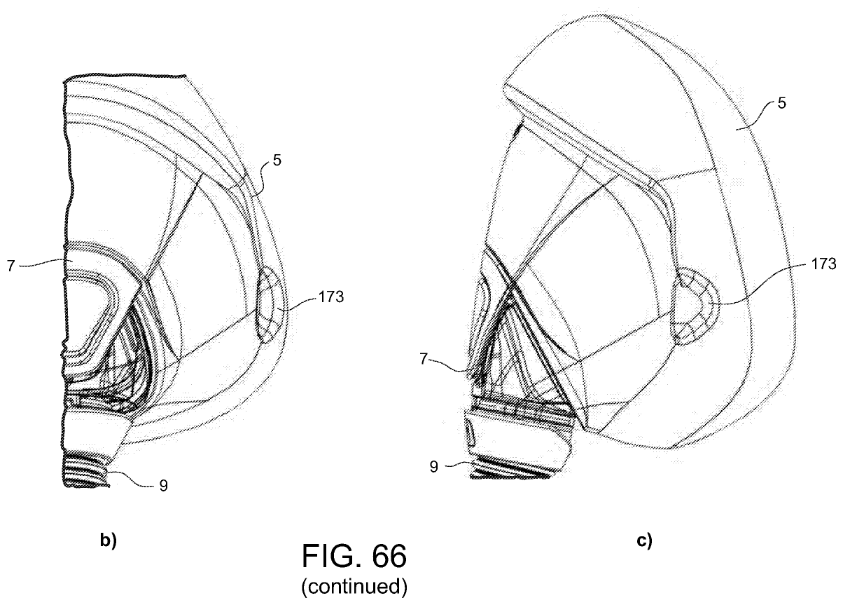

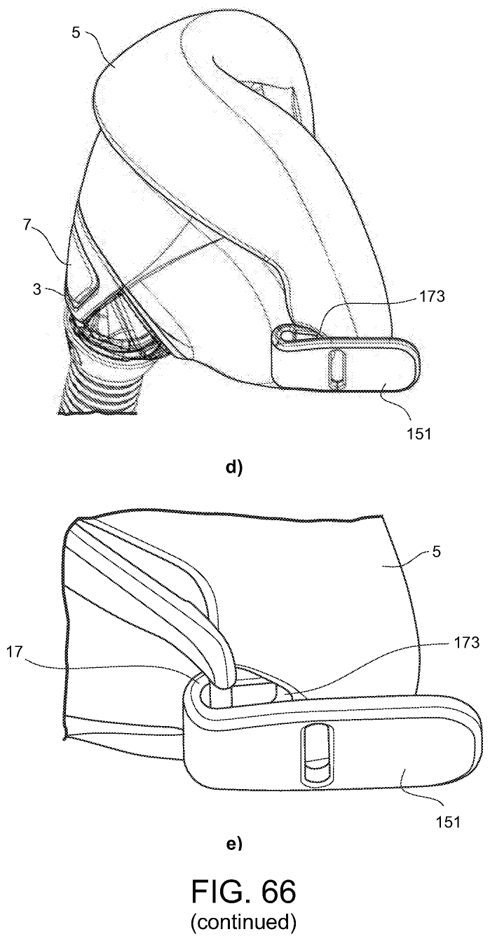

Referring to FIG. 66, a mask assembly comprises a modified cushion 5. In particular the region of the side wall 31 of the cushion 5 adjacent each lower headgear connector 17 of frame 3 is indented 173. Each indent 173 is located adjacent a respective lower headgear connector 17, and extends rearwardly. As can be seen in FIG. 66d) and e), each indent 173 allows clearance between the hook portion of a headgear clip 175 and the side wall 31 of the cushion 5. Each indent 173 may allow the headgear clip 175 to rotate about the post of lower headgear connector 17, without contacting the cushion 5.

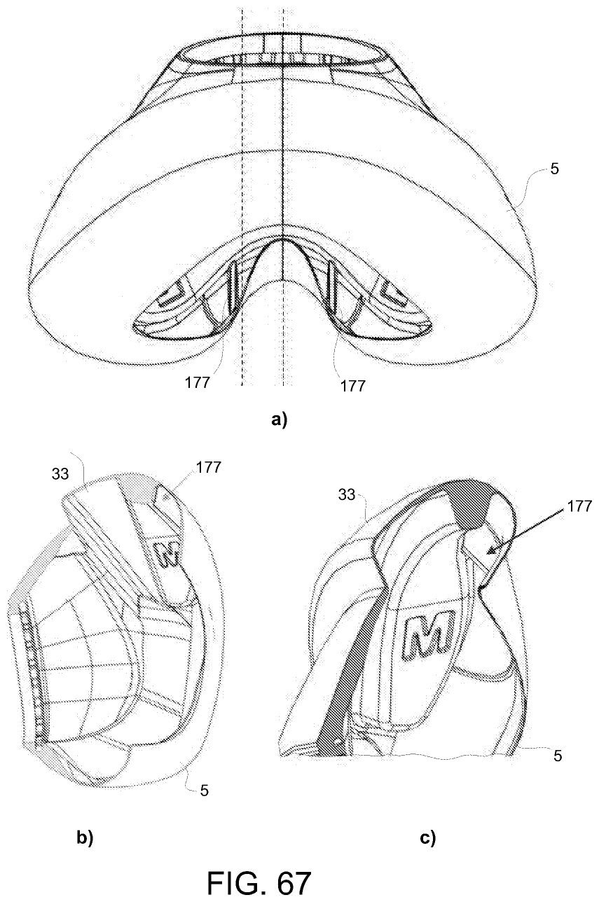

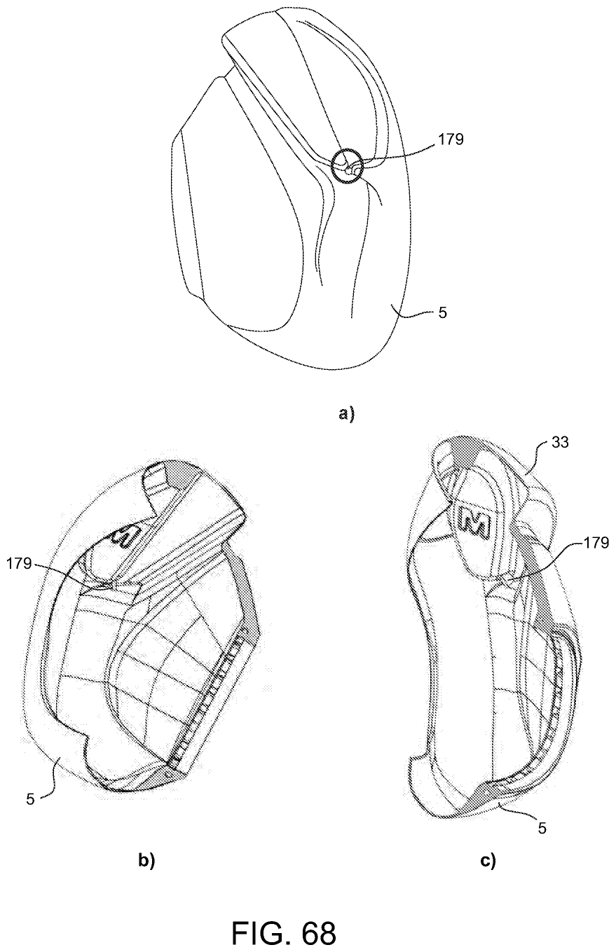

Referring to FIGS. 67 and 68, a mask assembly comprises a modified cushion 5. In this embodiment the cushion 5 is provided with one or more support ribs, internally of the cushion 5, to resist deformation of the cushion 5.

With reference to FIG. 67, pair of laterally spaced apart, generally laterally extending inclined support ribs 177 are provided, one each side of the vertical axis of the cushion 5. These laterally extending support ribs 177 are located in an upper portion of the cushion 5, adjacent the rolling bridge portion 33. Each rib 177 may be integrally formed with the cushion, from the same cushion material.

With reference to FIG. 68, a pair of laterally spaced apart, generally vertically extending support ribs 179 are provided, each rib 179 being at or adjacent the pivot axis 33A of the rolling bridge portion 33 of the cushion 5. These vertically extending support ribs 179 can limit how much the nose portion of the cushion 5 balloons under pressure, reducing the likelihood of eye leaks.

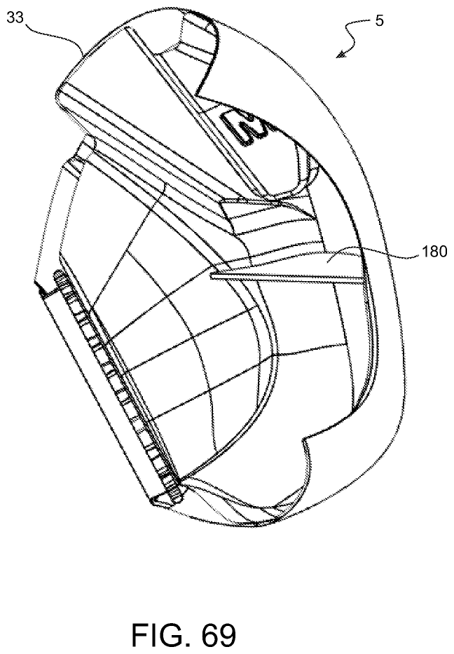

With reference to FIG. 69, a pair of laterally spaced apart support ribs 180 are provided, each rib 180 being adjacent but below the pivot axis 33A of the rolling bridge portion 33 of the cushion 5. These ribs provide support and reinforcement adjacent the area of the cushion 5 that pivots during movement of the rolling bridge portion 33.

Mask assemblies can be cleaned regularly by the patient. Some disassembly of the mask assembly may be required for this process.

It may be advantageous to separate the cushion 5 for individual cleaning. The cushion 5 may require more frequent cleaning than other components of the mask assembly. At least some of the above described embodiments allow for the cushion 5 to be disconnected independently from the remainder of the mask assembly, to enable this.

It may be advantageous to separate the headgear for individual cleaning. It may be advantageous to be able to remove the headgear without altering the strap connections with the mask frame 3, such that re-sizing is not required when next donning the headgear. In some embodiments, the upper headgear straps 23 are fastened around the frame 3, for example as shown in FIG. 24. As such, it may be advantageous to have the ability to remove the headgear and frame 3 together without needing to remove the inlet connector 7 and/or conduit 9 first.

The following embodiments are configured to provide the ability to selectively remove one or more components of the mask assembly, such as the headgear and/or frame 3, without disassembly of other components.

Referring to FIG. 70, a mask assembly comprises a modified mask frame 3. In this embodiment the frame 3 is removably connected to the inlet connector 7 and can be removed from the other components of the mask assembly, namely the inlet connector 7, cushion 5 and conduit 9, without disassembly of those other components.

In this embodiment the central portion 11 of the frame 3 comprises a frame clip 14 that is configured to mount the frame 3 onto the inlet 7. The central portion 11 is therefore a central connection portion of the frame 3. The opening 13 in the central connection portion of the frame 3 is defined by the internal walls of the frame clip 14, the central connection portion circumscribing the inlet opening. In this embodiment the frame clip 14 comprises front 14a and side 14b walls that extends around the front and sides of the opening 13. The side walls 14b terminate towards the rear to leave a mouth 14c at the rear of the opening 13, defined between opposed spaced apart ends 14d of the side walls 14b of the frame clip 14, such that the opening 13 is not bound completely by the frame clip 14.

When viewed from above, along the central axis of the opening 13, the frame clip 14 is omega shaped. The mouth 14d allows the side walls 14b of the frame clip 14 to resiliently deform during assembly of the inlet connector 7 on the frame 3. The frame clip 14 resiliently expands during mounting of the mask frame 3 onto the remainder of the mask assembly, and then contracts around the inlet connector 7 when the inlet connector 7, or at least part of the inlet boss 47, is fully received in the opening 13. The frame 3 is not connected to the inlet connector 7 and cushion 5 at any other location, other than at the frame clip 14, namely at the central connection portion. In use, the headgear provides tension through the frame 5, encouraging the clip 14 to remain secured to the inlet connector 7. Consequently, the frame 3 can be removed, as a single component, from the remainder of the mask assembly, by expanding the frame clip 14, increasing the size of the opening 13, such that the frame clip 14 disengages the inlet connector 7.

The frame clip 14 may comprise one or more anti-rotation and/or guide surfaces configured to resist rotation of the frame 3 relative to the mask assembly. For example, the front and/or side walls 14a, 14b of the frame clip 14 may be straight or flat, or comprise one or more straight or flat portions. This may help resist or prevent relative rotation between the mask frame 3 and the inlet connector 7. The frame clip 14 in this embodiment, comprises at least one non-circular wall portion 14e that engages the inlet connector 7. In this embodiment the opening 13 comprises a plurality of substantially straight side wall portions 14e of the side walls 14b that engage with corresponding straight portions of the inlet connector 7.

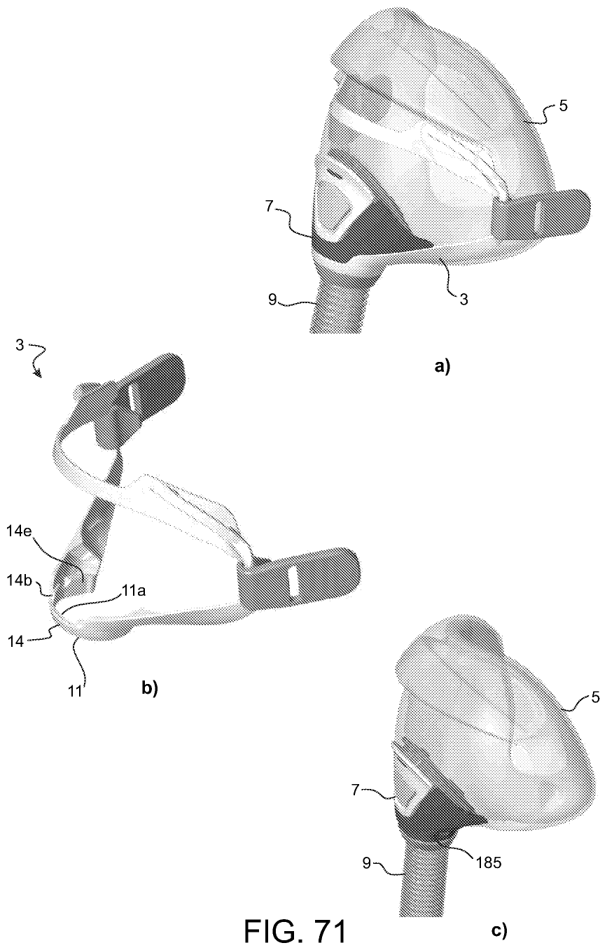

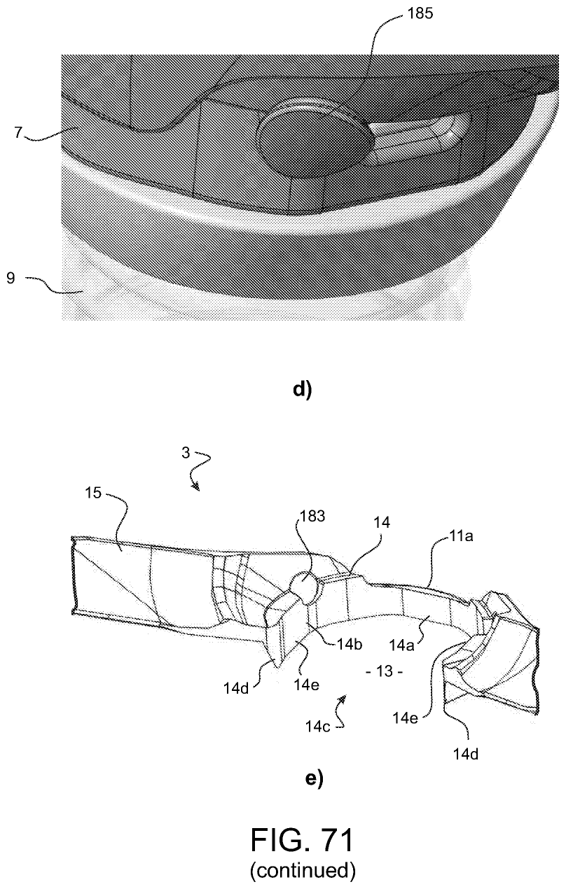

Referring to FIG. 71, the modified mask frame 3 is similar to that of FIG. 70. However, in this embodiment the front part of the central portion 11 of the frame 3 is recessed 11a, when viewed from the front, such that a portion of the central portion 11 adjacent the frame clip 14 is relatively thinner than the remainder of the central portion 11. This may enable the front portion of the frame 3 to be more flexible, for example to allow the frame clip 14 to more easily deform during assembly and disassembly with the inlet connector 7.

With further reference to FIG. 71, the frame 3 and the inlet connector 7 are provided with corresponding engagement features that engage with one another when the frame 3 is mounted on the inlet connector 7. In this embodiment the frame 3 comprises a female engagement feature 183 that receives a male engagement feature in the form of a lug 185 on the inlet connector 7. In this embodiment that engagement features are positioned laterally of the central portion 11 of the frame 3. When engaged, the engagement features 183, 185 help retain the inlet connector 7 on the frame 3, and help resist relative rotation between the inlet connector 7 and the frame 3.

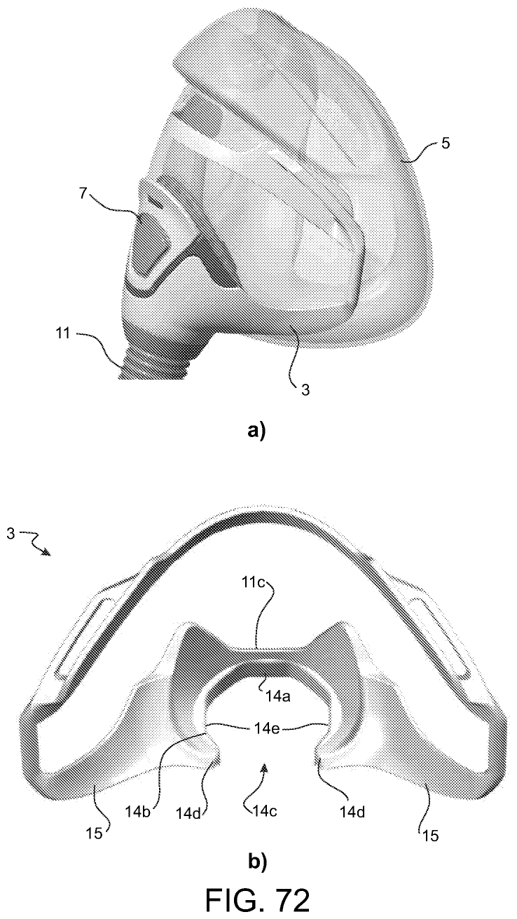

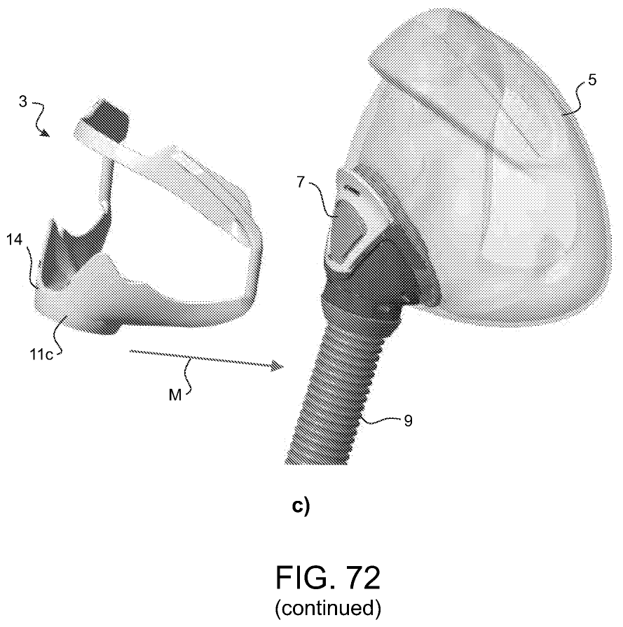

Referring to FIG. 72, the modified mask frame 3 is similar to that of FIG. 70. However, in this embodiment the front part of the central portion 11 has an increased height front wall 11c, when viewed from the front, such that a portion of the central portion 11 adjacent the frame clip 14 is relatively thicker than the reminder of the frame clip 14. This may enable the front portion of the frame 3 to be more rigid, for example to resist the frame clip 14 from deforming. Arrow M indicates a mounting direction in which the frame 3 is pushed onto the inlet connector 7.

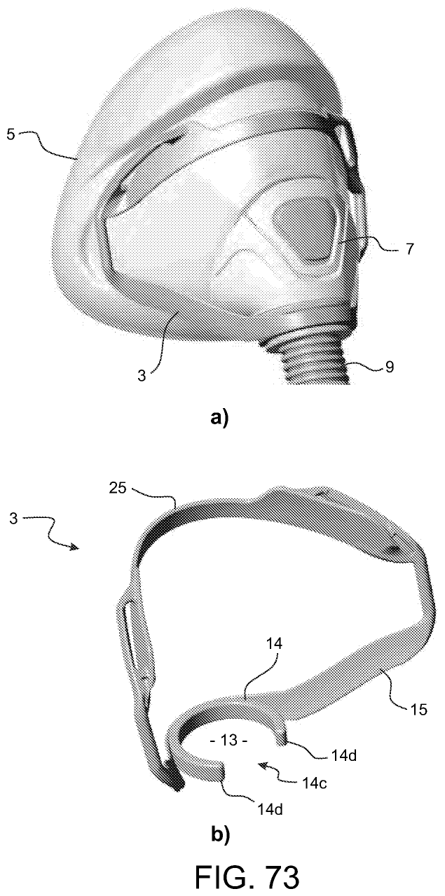

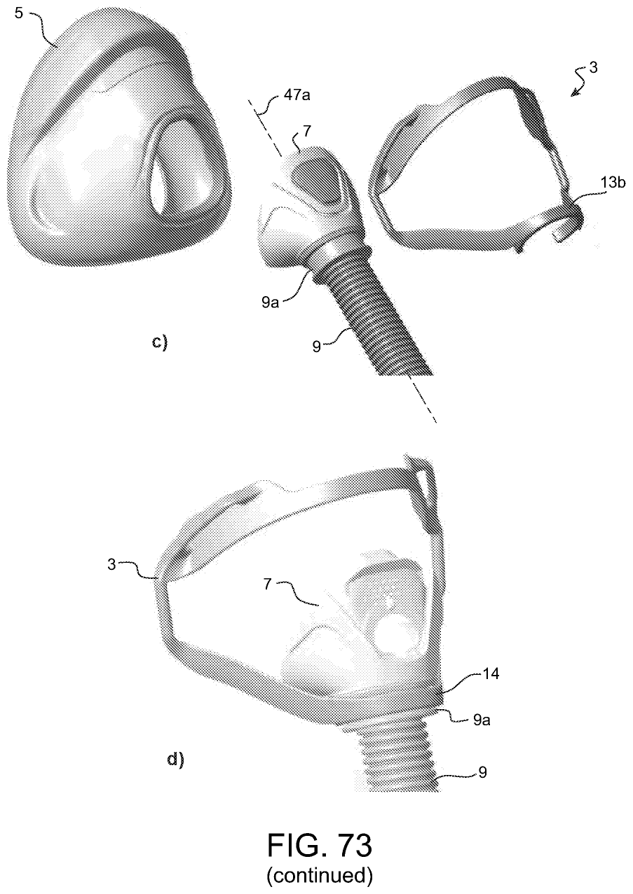

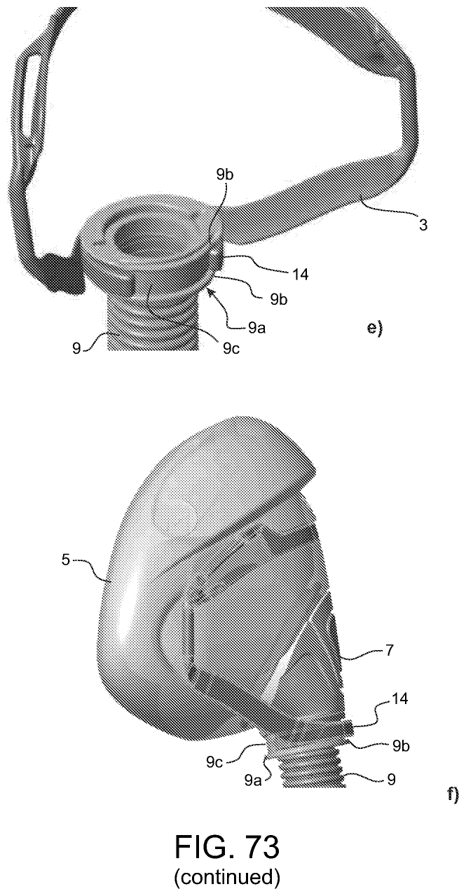

Referring to FIG. 73, in this embodiment the mask frame 3 is removably mounted on a conduit connector cuff 9a at the end of the inlet conduit 9, instead of being mounted on the inlet connector 7. In this embodiment, the mask frame 3 is only connected to the conduit connector cuff 9a, and is not connected to the remainder of the mask assembly.

In this embodiment the frame clip 14 is similar to the frame clip 14 described with reference to FIG. 70, and comprises a mouth 14c at the rear of the frame clip 14, defined between opposed spaced apart ends 14d of the frame clip 14, such that the opening 13 is not fully bound by the frame clip 14. The frame clip 14 resiliently deforms outwardly to expand the mouth 14c as the spaced part ends 14d engage the conduit connector cuff 9a, and then return to their rest position to contract the mouth 14c to connect the mask frame 3 to the conduit connector cuff 9a.

The conduit connector cuff 9a may comprise a frame location feature configured to locate and/or guide the frame 3 into the desired position on the conduit connector cuff 9a.

Referring to FIG. 73c), e) and f), the conduit connector cuff 9a comprises a pair of spaced apart frame location features in the form of longitudinally spaced apart circumferential flanges 9b between which is defined a recess 9c that receives the mask frame clip 14. The flanges 9b resist movement of the mask frame 3 in a direction along the axis of conduit 9.

The frame clip 14 extends away from the frame's lateral arms 15 to partially extend around the rear of conduit connector cuff 9a. The frame clip 14, at least at the sides, is laterally spaced from the remainder of the frame 3, in in particular from the lateral arms 15. The frame clip 14 is therefore decoupled from the remainder of the frame 3 in the sense that forces exerted onto the frame via the headgear are decoupled from the frame clip 14. This decoupling of forces assists in preventing unintentional disconnection of the frame 3 from the conduit connector cuff 9a in situations where the sides of the frame 3 are pushed forwards or away from each other, as this deformation is less likely to affect the connection at the frame clip 14.

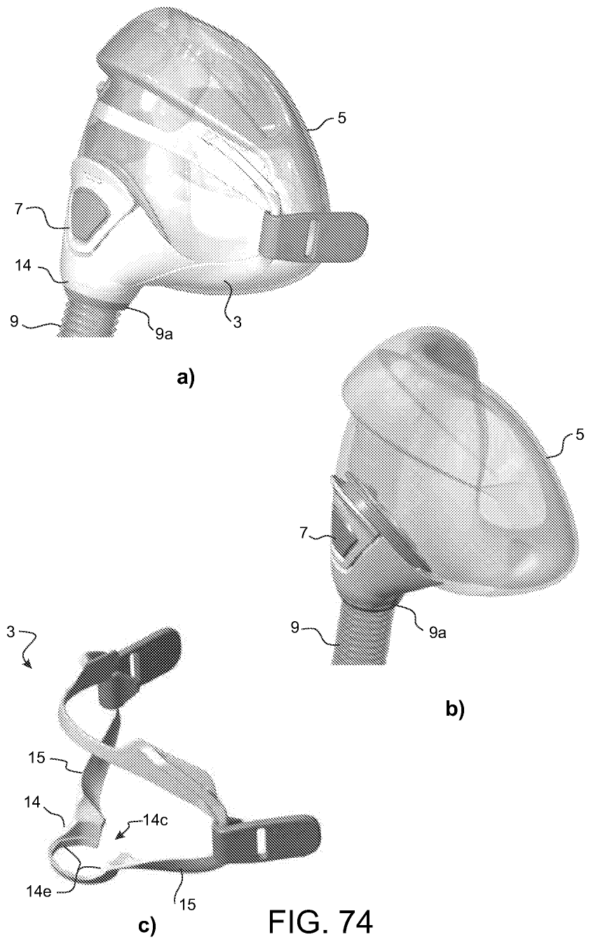

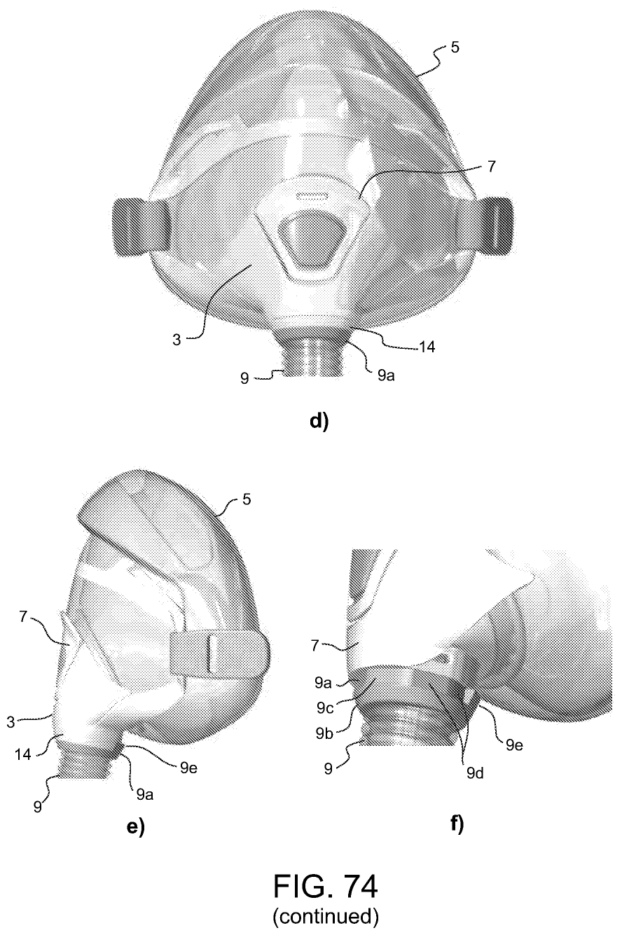

Referring to FIG. 74, the modified mask frame 3 is similar to that of FIG. 73 and comprises a mouth 14c at the rear of the frame clip 14, defined between opposed spaced apart ends 14d of the frame clip 14, such that the opening 13 is not closed by the frame clip 14. However, in this embodiment, the frame 3 connects with both the inlet connector 7 and the conduit connector cuff 9a of the conduit 9.

The internal walls of the frame clip 14 are shaped to resist or prevent relative rotation between the mask frame 3 and the inlet connector 7, and between the mask frame 3 and the conduit connector cuff 9a. In this embodiment the frame clip 14 comprises a plurality of substantially straight portions 14e of side walls 14b at the sides of the frame clip 14. The conduit connector cuff 9a comprises a corresponding plurality of substantially straight wall portions 9d. When the mask is connected to the conduit connector cuff 9a the wall portions 14e engage the wall portions 9d to resist relative rotation.

In this embodiment, the straight wall portions 14e of the frame clip 14 extend upwardly from the frame clip 14 and overlap with side portions 7a of the inlet connector 7. The wall portions 14e extend to some extent around the rear of the inlet connector 7 such that the wall portions 14e connect with the inlet connector 7. The frame 3 therefore engages the front, sides, and some of the rear of the inlet connector 7 and the conduit connector cuff 9a.

The conduit connector cuff 9a comprises an end stop 9e against which the frame clip 14 abuts when the frame 3 is fully connected to the conduit connector cuff 9a, to limit by how much the mask frame 3 can be pushed onto the conduit connector cuff 9a.

In this embodiment the conduit connector cuff 9a comprises a single, lower circumferential flange 9b, generally upward movement of the frame 3 being limited by the lowermost surface of the inlet connector 7. Recess 9c is therefore defined between the lower flange 9b and the lowermost surface of the inlet connector 7.

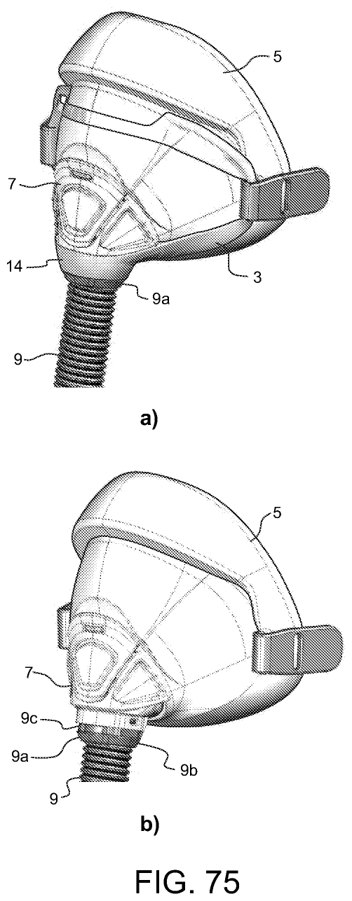

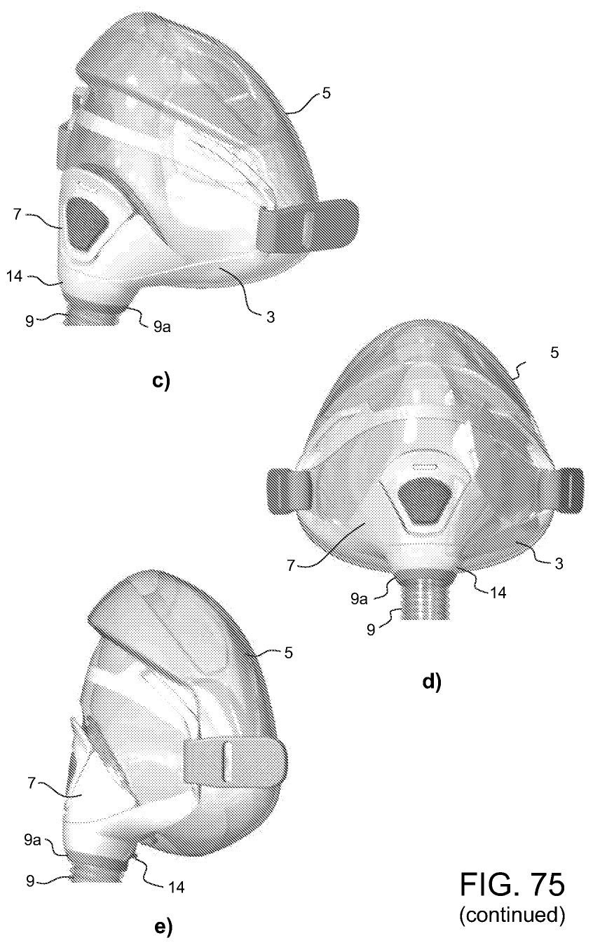

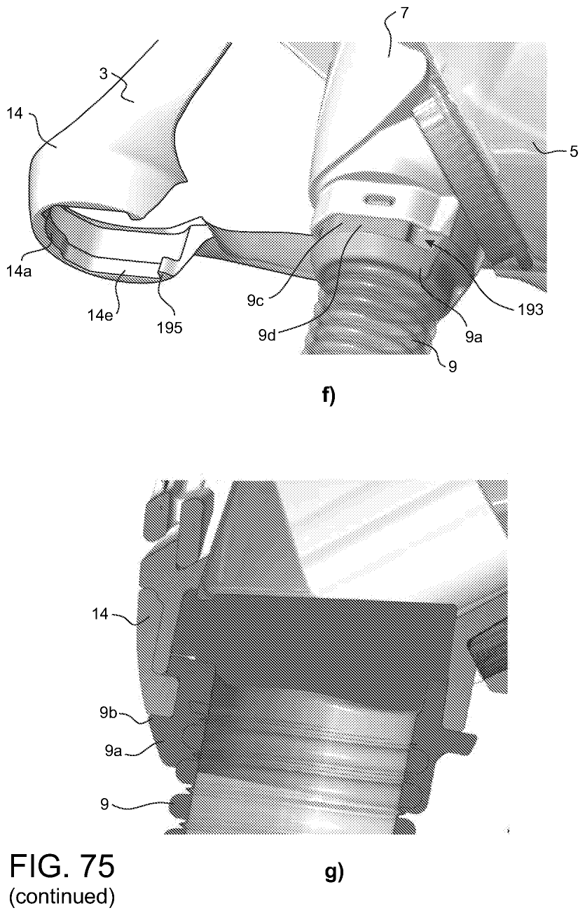

Referring now to FIG. 75, an embodiment is shown which is similar to that of FIG. 74, and which comprises a frame 3 that connects to both the inlet connector 7 and the conduit connector cuff 9a.

In this embodiment the recess 9c on the conduit connector cuff 9a is deeper than in the FIG. 74 embodiment. Consequently, the frame clip 14 that fits into and connects with the recess 9c, can be thicker. The thicker frame clip 14 has an increased stiffness that helps prevent the frame 3 from unintentional disconnection when the front of the frame 3 is rotated up or down, for example due to tensile forces from the headgear, or movement of the mask assembly by the user. The thicker frame clip 14 in this embodiment comprises an annular rib 14f that projects away from the remainder of the mask frame 3, into the opening 13.

With further reference to FIG. 75, the frame 3 and the conduit connector cuff 9A are provided with corresponding engagement features that engage with one another when the frame 3 is mounted on the conduit connector cuff 9a. In this embodiment the conduit connector cuff 9a comprises a female engagement feature 193 that receives a male engagement feature in the form of a lug 195 on the opening 13. In this embodiment the engagement features are positioned laterally of the central portion 11 of the frame 3. When engaged, the engagement features 193, 195 help retain the frame 3 on the conduit connector cuff 9a, and help resist relative rotation between the frame 3 and the conduit connector cuff 9a.

With further reference to FIG. 75, the frame clip 14 comprises substantially straight, parallel, opposed side portions 14e that engage with corresponding substantially straight side surfaces 9d of the conduit connector cuff 9a. The engagement between the side portions 14e and surfaces 9d resists rotation of the mask frame 3 relative to the conduit 9.

In the embodiments of FIGS. 70 to 75, the portion of the mask frame 3 around the opening 13 that fits into and connects with the recess 9c, can be considered to be a resiliently deformable frame clip 14, that clips onto the inlet connector 7 and/or the conduit connector cuff 9A. In these embodiments, the frame clip 14 releasably clips onto the inlet connector 7 and/or the conduit connector cuff 9A.

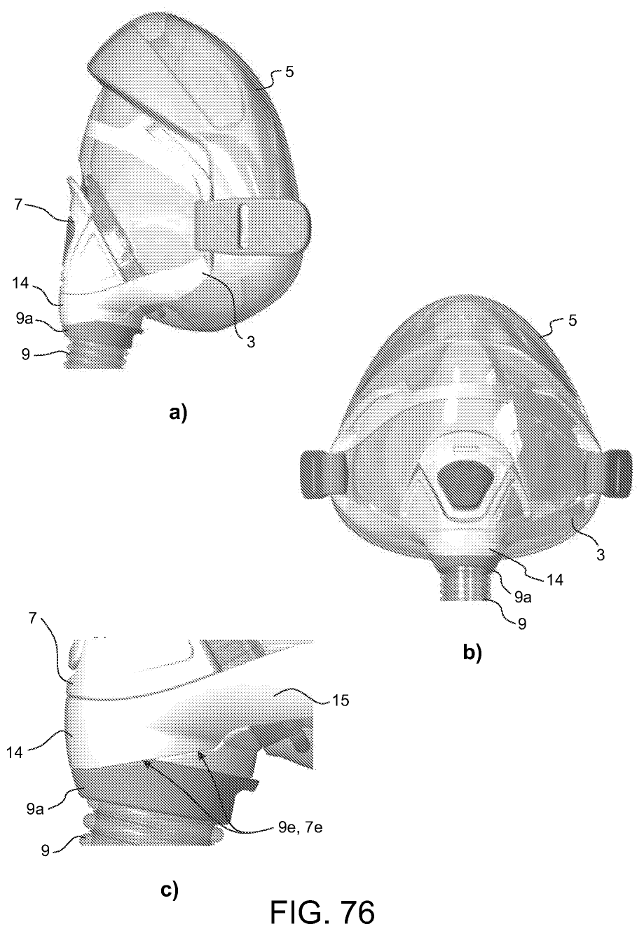

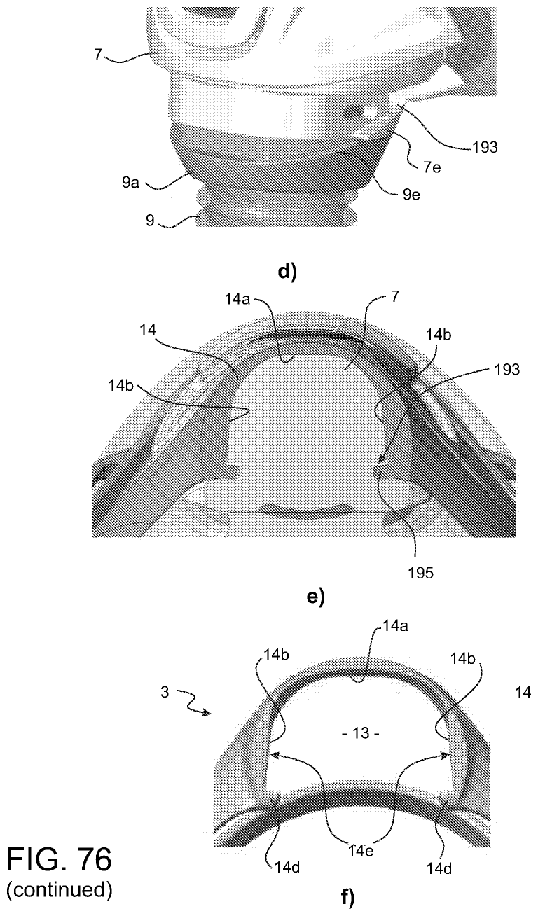

Referring now to FIG. 76, an embodiment is shown which is similar to that of FIG. 74, and which comprises a frame 3 that connects to both the inlet connector 7 and the conduit connector cuff 9a.

In this embodiment the side portions 9e of the recess 9c in the conduit connector cuff 9a are upwardly inclined towards the lower part of the inlet connector 7. The inclined side portions 9e lead to upwardly inclined side portions 7E on the inlet connector 7. The side portions 9e, 7e together define smooth, upwardly inclined guide surfaces that guide the frame clip 14 into the desired position to connect with the inlet connector 7 and the conduit connector cuff 9a, as the mask frame 3 is pushed onto these components from the front of the mask assembly.

In this embodiment, the female engagement feature 193 is provided on the inlet connector 7 rather than on the inlet conduit connector 9a. The engagement features 193, 195 help retain the frame 3 on the inlet connector 7, and help resist relative rotation between the frame 3 and the inlet connector 7, once the frame 3 has been fully pushed onto the inlet connector 7.

As can best be seen in FIG. 76e) and f), the frame clip 14, when viewed from above or below comprises substantially straight side walls 14b. The side walls 14b are angled towards one another as they extend away from the mouth 14c. Consequently, as the frame clip 14 is pushed onto the inlet connector 7 and conduit connector cuff 9a, increasing force is required to fully mount the frame clip 14 onto the inlet connector 7 and conduit connector cuff 9a.

The ends 14d of frame clip 14 define fingers that project from the ends of the frame clip 14 radially inwardly towards one another. The fingers 14d are configured to engage the rear of the inlet connector 7, to retain the frame clip 14 on the inlet connector 7, as shown in FIG. 76e). To disconnect the components, the fingers 14d are moved outwardly, away from one other, to so that they no longer engage the rear of the inlet connector 7.

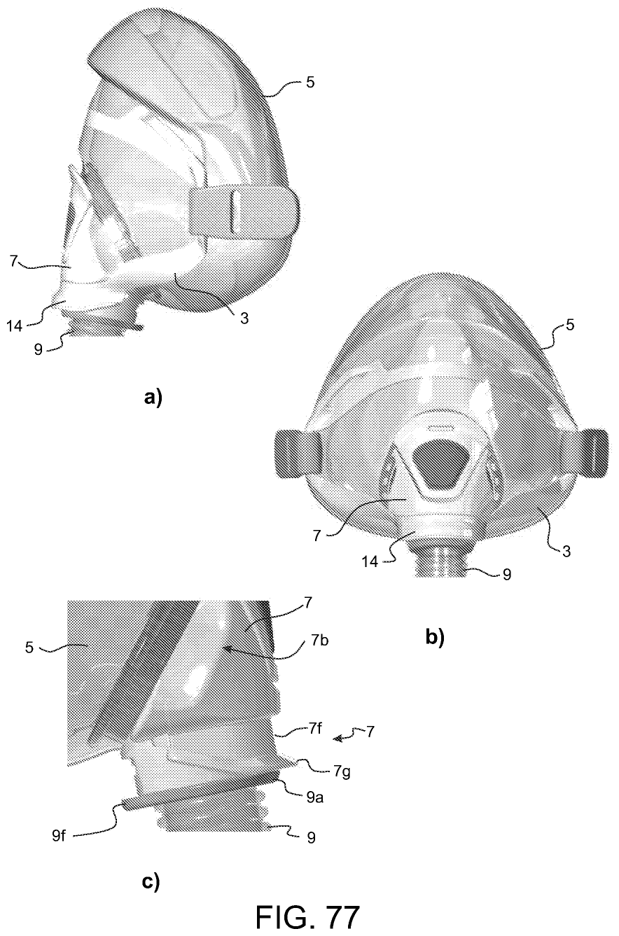

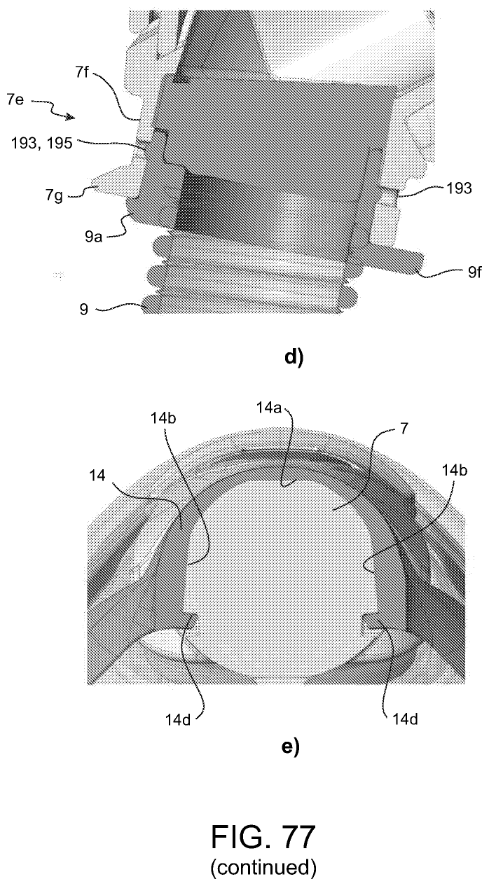

Referring now to FIG. 77, an embodiment is shown which comprises a frame 3 that connects only to the inlet connector 7, and not to the conduit connector cuff 9A.

In this embodiment, the lower part of the inlet connector 7 comprises a skirt 7e that extends downwardly over the front and sides of the conduit connector cuff 9a. The skirt 7e comprises a recess 7f configured to received and engage with the frame clip 14 of the mask frame 3, and a guide flange 7g that projects outwardly from the skirt 7e below the recess 7f. The sides of the guide flange 7g are inclined upwardly from front to rear, to guide the mask frame 3 upwardly as it is pushed onto the inlet connector 7. The inclined recess 7f and inclined flange 7g together provide smooth uninterrupted surfaces for the frame clip 14 to slide over and engage with.

Additionally, any forward rotation of the mask frame 3 will be less likely to disconnect the conduit connector cuff 9a unintentionally.

The conduit connector cuff 9a comprises a rear release tab 9f that projects from a rear part of the cuff 9a, behind the inlet connector 7. This enables the user to grip and release the conduit 9 from the mask assembly, by pivoting the rear of the conduit connector cuff 9a away from the inlet connector 7.

The inlet connector 7 further comprises recessed hand grip portions 7b either side of the diffuser, and above the skirt 7e.

In this embodiment the engagement features 193, 195 are located at the front and rear of the frame clip 14 and conduit connector cuff 9a, so that the sides of the frame clip 14 and cuff 9a are smooth and therefore allow less resistance to pushing of the frame clip 14 onto the cuff 9a,

Similar to the frame clip 14 of FIG. 76, the side walls 14b are inwardly inclined away from mouth 14c, and the fingers 14d engage the rear of the inlet connector 7.

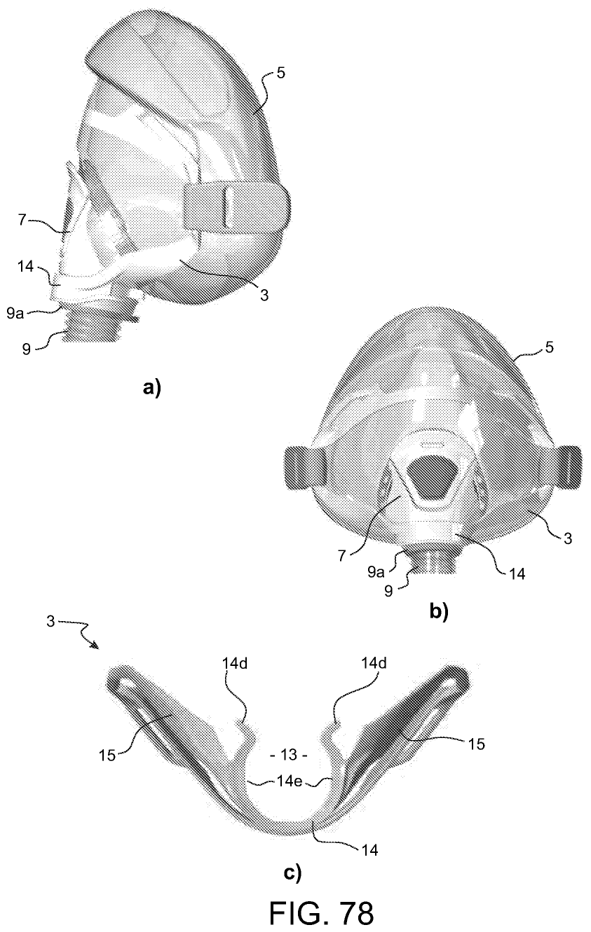

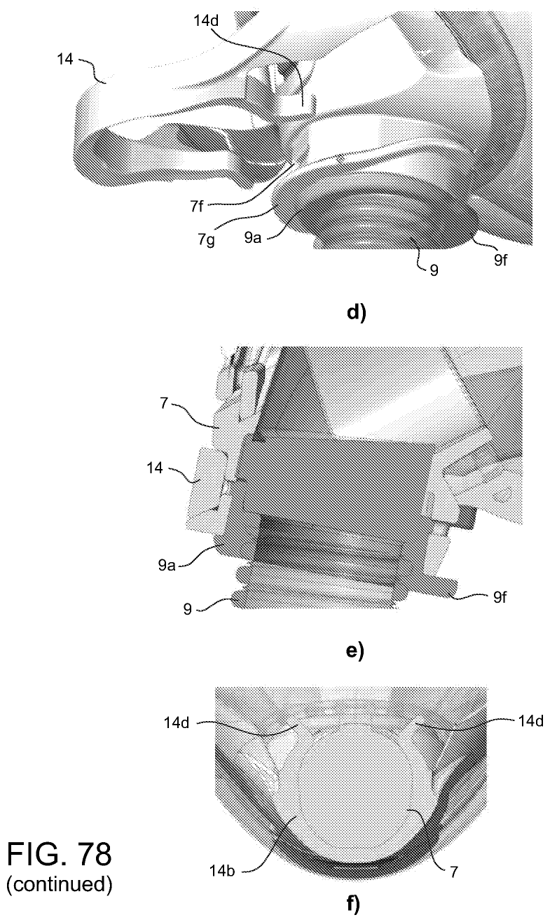

Referring now to FIG. 78, an embodiment is shown which has similar features to that of FIG. 77, in which the mask frame 3 connects only to the inlet connector 7.

In this embodiment, the frame clip 14 of the mask frame 3 that fits into and connects with the inlet connector recess 7F clips onto the inlet connector 7.

The frame clip 14 comprises front and side walls 14a, 14b, that extend partially around the rear of the opening 13, leaving a mouth 14c at the rear that initially receives the inlet connector 7. As the mask frame 3 is further pushed onto the inlet connector 7 from the front, the frame clip 14 resiliently deforms, to allow the inlet connector 7 to be fully received in, and to engage with, the frame clip 14.

In this embodiment, the frame clip ends 14d comprise fingers that project from the ends of the frame clip 14 radially outwardly away from the side walls 14b. The fingers 14d are therefore angled away from each other when viewed along opening axis 13a, to comprise guide surfaces that together define mouth 14c. At the start of assembly, the guide surfaces of the fingers 14d engage the inlet connector 7. Further movement of the mask frame 3 towards the inlet connector 7 further engages the fingers 14d which are forced to deform outwardly to increase the size of the mouth 14c to allow the inlet connector 7 to be fully received in the frame clip 14. When so received, the fingers 14d snap back to reduce the size of the mouth, engage the inlet connector 7 and retain the inlet connector 7 on the mask frame 3.

The fingers 14d are spaced apart from the frame 3 and in particular are spaced from the lateral arms of the frame 3. The fingers 14d are therefore decoupled from the lateral arms 15 of the frame 3, such that the lateral arms 15 of the frame 3 can be moved (for example under headgear tension) without opening and disconnecting the frame clip 14 from the inlet connector 7.

Referring to FIG. 78a) and e) the frame clip 14 is relatively tall, when viewed from the front and back, and relatively deep, when viewed in cross section, increasing the rigidity, and the resistance to deformation, of the frame clip 14. Providing a frame clip 14 with increased dimensions can also provide a more solid and reassuring feel to the user.

Referring to FIG. 78d) the inlet connector flange 7g extends around the rear of the inlet connector 7, so as to engage the fingers 14d of the frame clip 14.

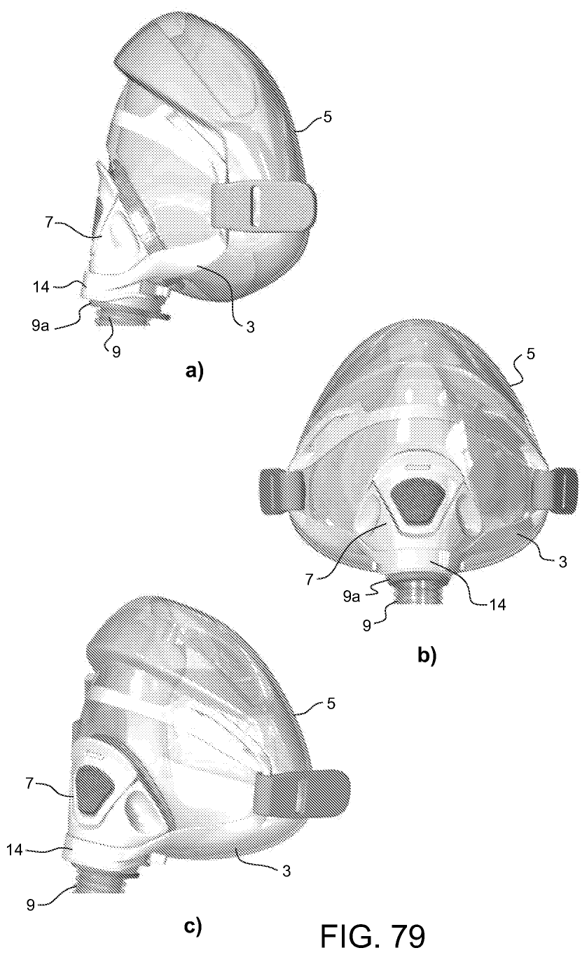

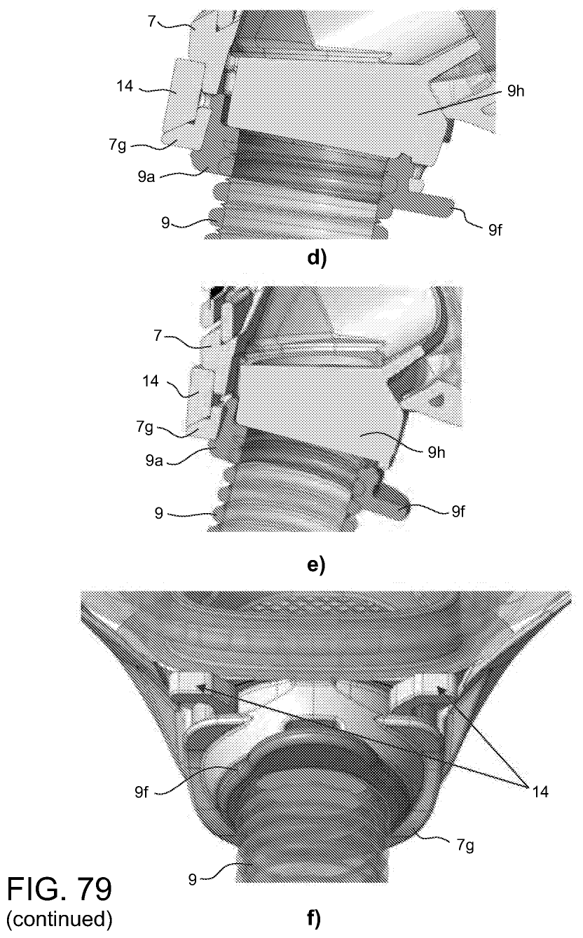

Referring now to FIG. 79, an embodiment is shown which has similar features to that of FIGS. 77 and 78, in which the mask frame 3 connects only to the inlet connector 7.

With reference to FIG. 79d) the uppermost rear portion of the conduit connector cuff 9A is downwardly inclined 9h towards the rear of the cuff 9a. This allows clearance for the cuff 9a to be more easily disconnected from the inlet connector 7 when the user presses down on the release tab 9e.

With particular reference to FIG. 79f), the fingers 14d extend further away from the opening 13 to provide release fingers that can be pushed apart to release the frame clip 14 from the inlet connector 7.

With particular reference to FIG. 79a) and b) the grip portions of the inlet connector 7 are deeper than those described above.

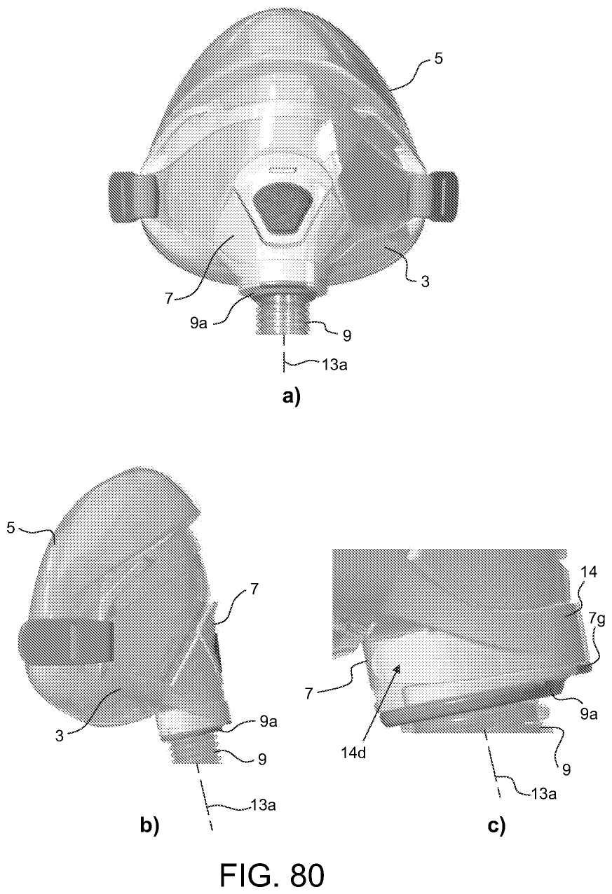

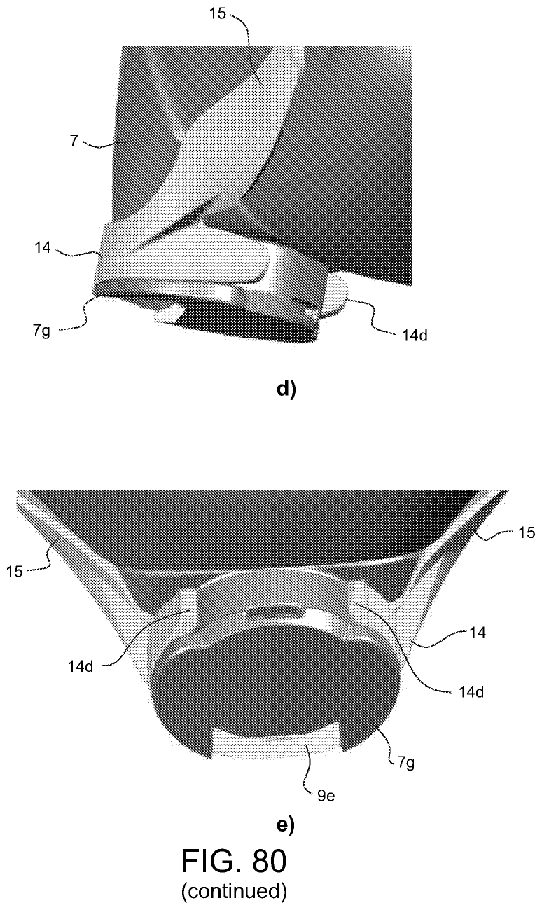

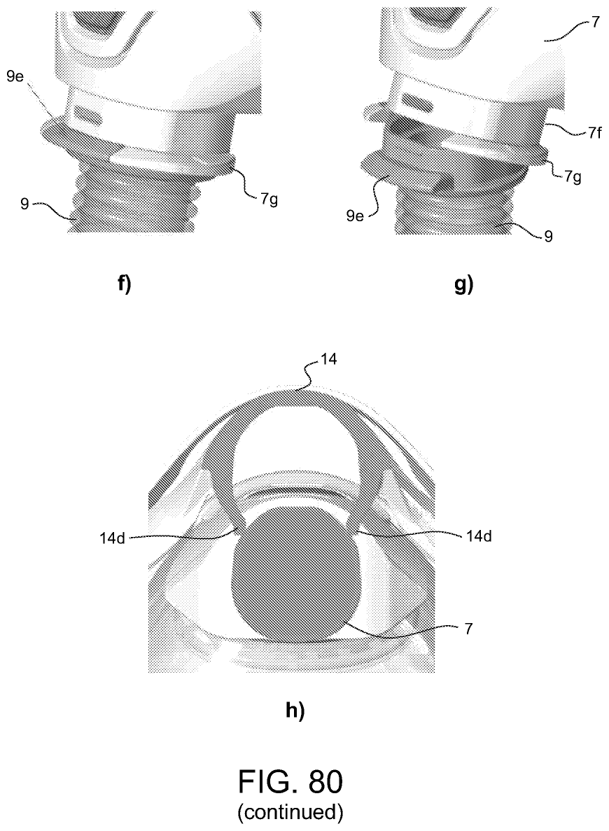

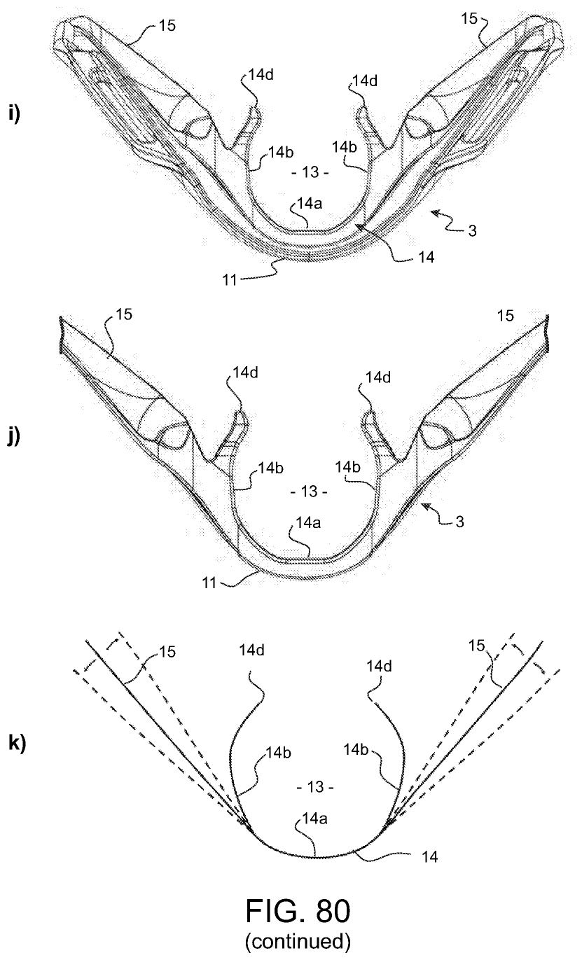

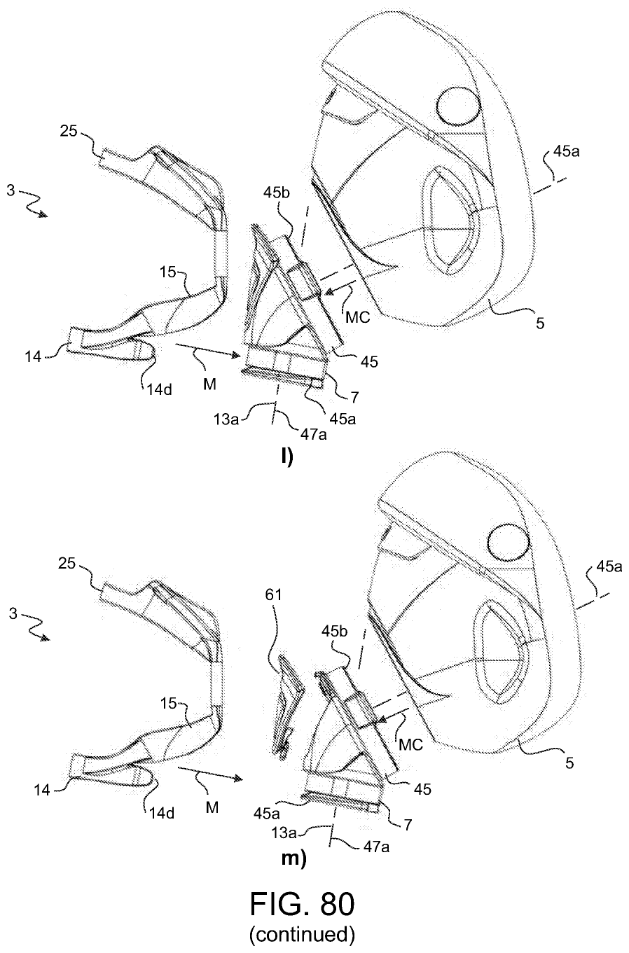

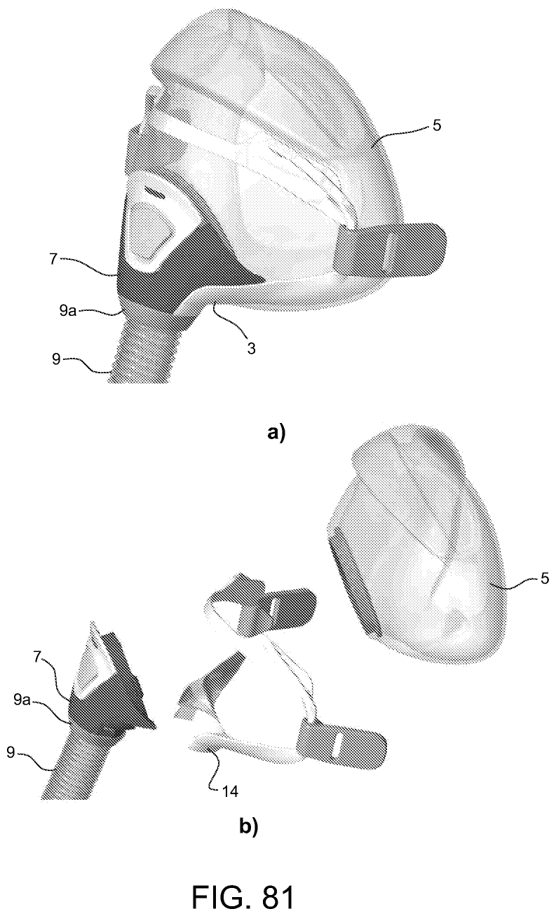

Referring now to FIG. 80, an embodiment is shown which has similar features to that of FIG. 77-79, in which the mask frame 3 connects only to the inlet connector 7.

In this embodiment, the lower part of the inlet connector 7 comprises the mounting recess 7F which is partly defined by the lower flange 7g at the sides and rear of the inlet connector 7. The lower flange 7g does not extend around the front of the inlet connector 7, leaving a gap 7h. In this embodiment, the conduit connector cuff 9a release tab 9e is located at the front of the cuff 9a and is dimensioned to be received in the gap 7h and when so received to define the front of the recess 7f. With reference to FIG. 80d) and e), to release the conduit 9 from the inlet connector 7, the release tab 9e is pushed downwardly, such that the front part of the conduit connector cuff 9a rotates away from the inlet connector 7.

As can best be seen in FIG. 80c) and f), the fingers 14d of the frame clip 14 are relatively short, and only project a short distance outwardly away from the remainder of the frame clip 14. The fingers 14d also extend only a short distance along the sides to the rear of the inlet connector 7, when the frame 3 is mounted on the inlet connector 7. Consequently, the flange 7g need only extend a relatively short distance along the sides to the rear of the inlet connector 7. The rear of the inlet connector 7 has no flange, as can be seen in FIG. 80e),

The shape of the interior walls 14a, 14b of the frame clip 14 closely correspond to the exterior profile, when viewed from above, of the inlet connector 7. This combined with the relatively short fingers 14d, provides the mask assembly with a relatively smooth, clean look, with minimal regions in the connection between the frame 3 and inlet connector 7 that are angular or project significantly outwardly.

Referring to FIG. 80i) and j), the frame clip side walls 14b are substantially straight, as is a central portion of front wall 14a, to resist rotation of the frame clip 14 relative to the mask assembly.

Referring to FIG. 80k), the frame clip 14 is to some extent decoupled from the lateral arms 15, such that the lateral arms 15 may deflect, for example to the positions shown in dashed line, without substantially affecting the mounting of the frame clip 14 on the inlet connector 7. For example, the lateral arms 15 may be temporarily deflected outwardly, away from the face, when for example the user is pushing the mask assembly away from their face to talk, or when adjusting the mask, or disconnecting the headgear. The lateral arms 15 may be pushed inwards for example when the headgear is being connected to the mask assembly. The fingers 14d of the frame clip 14 are substantially decoupled from the lateral arms, and therefore the geometry of the frame clip 14 is relatively unaffected by deformation of the mask frame 3.

Referring to FIG. 80l) and m), the relative orientations of the mask frame 3, cushion 5, and inlet connector 7 can be seen. These figures also shows the mounting direction M of the mask frame 3 onto the inlet connector 7, in a direction which is inclined but generally horizontal, in a rearward direction from front to rear of the mask assembly, in a direction substantially orthogonal to the to the central inlet axis 47a of the inlet port 47b of inlet boss 47. In this embodiment the mounting direction M is also substantially orthogonal to the opening axis 13a. In this embodiment the central inlet axis 47a is aligned with the opening axis 13a. The cushion 5 is mounted on to the rear of the inlet connector 7 in a generally forward direction MC from rear to front of the mask assembly, and in a downwardly inclined direction. The central outlet axis 45A of the outlet port 45b of the outlet boss 45 of the inlet connector 7 is inclined relative to the central inlet axis 47a, for example at an angle of between 30 and 60 degrees. In this embodiment the central axis 45A of the outlet boss 45 is also inclined relative to the opening axis 13A.

Referring now to FIG. 81, an embodiment is shown in which the mask frame 3 connects only to the inlet connector 7. In this embodiment the mask frame 3 comprises a frame clip 14 that connects to the rear, rather than the front, of the inlet connector 7.

The frame clip 14 is reversed from that described with reference to FIGS. 77 to 80 in that the mouth 14c is located at the front of the frame clip 14 rather than the rear. Consequently, in this embodiment, inlet connector 7 is pushed onto the mask frame 3 from the front so that the inlet connector 7 is initially received in the mouth 14c and then pushed rearwardly into the opening 13 and onto the frame clip 14. The cushion 5 is then pushed onto the rear of the inlet connector 7, through the frame 3 that is already connected to the inlet connector 7. In this embodiment, the cushion 5 can first be removed, before removal of the frame 3 from the inlet connector 7.

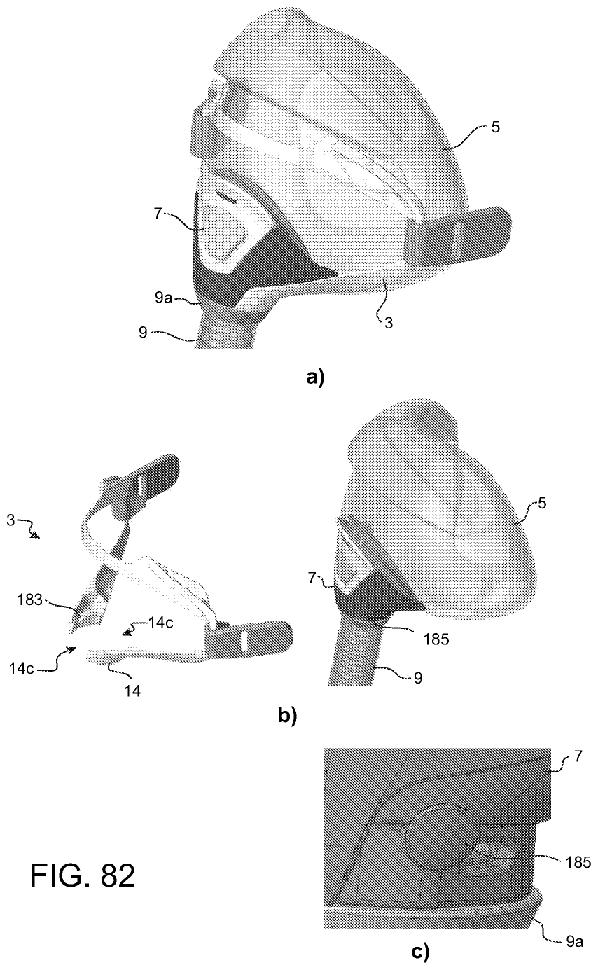

Referring now to FIG. 82, an embodiment is shown in which the mask frame 3 connects only to the inlet connector 7. In this embodiment the mask frame 3 comprises a frame clip 14 that connects to the rear of the inlet connector 7.

The frame clip 14 comprises a pair of opposed side walls 14b, but no front or rear portion. The frame clip 14 thus comprises a mouth 14c at the front and rear, such that the frame clip 14 only partially defines the opening 13. Each side wall 14b extends from a forward end of a respective lateral frame arm 15.

With further reference to FIG. 82 b) and c), the frame 3 and the inlet connector 7 are provided with corresponding engagement features that engage with one another when the frame 3 is mounted on the inlet connector 7. In this embodiment the frame 3 comprises a female engagement feature 183 that receives a male engagement feature in the form of a lug 185 on the inlet connector 7. In this embodiment that engagement features are positioned laterally of the central portion 11 of the frame 3. The female engagement features 183 are provided on the frame clip side portions 13F. When engaged, the engagement features 183, 185 help retain the inlet connector 7 on the frame 3, and help resist relative rotation between the inlet connector 7 and the frame 3.

It is envisaged that the mask frame 3, cushion 5, and inlet connector can be configured to be connected together such that the mask frame 3 is sandwiched between the cushion 5 and the inlet connector 7.

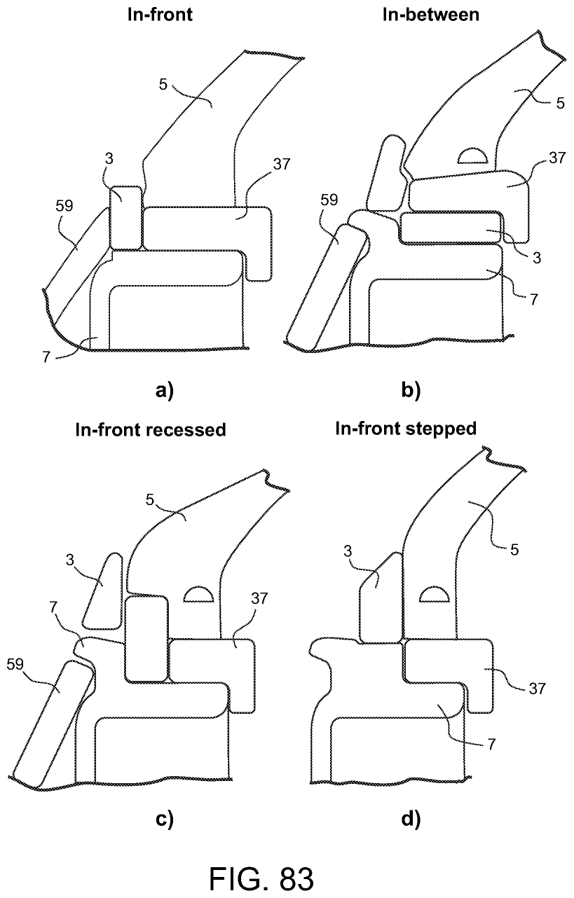

With reference to FIG. 83 four examples of such a sandwiched connection are shown schematically.

In the example of FIG. 83a), the frame 3, and specifically the front portion of the frame clip 14 is located at the front surface of the mask assembly, and is sandwiched between the inlet 7 below, and the cushion rigid ring 37 and cushion 5 to the rear and above. The upper portion of the diffuser 59 abuts the front surface of the frame 3. The mask frame 3 is flush with the front surface of the mask assembly, namely the diffuser 59 and the cushion 5.

In the example of FIG. 83b), the frame 3, is recessed behind the front surface of the mask assembly, and is sandwiched between the inlet 7 below, and the cushion rigid ring 37 to the rear and above.

In the example of FIG. 83c), the frame 3, is recessed behind the front surface of the mask assembly, and is sandwiched between the inlet 7 below, the cushion rigid ring 37 to the rear, and the cushion 5 above.

In the example of FIG. 83d), the frame 3, and specifically the front portion of the opening 13 is located at the front surface of the mask assembly, and is sandwiched between the inlet 7 below, and the cushion rigid ring 37 and cushion 5 to the rear. The upper portion of the diffuser 59 abuts the front surface of the frame 3. The mask frame 3 is stepped back from the front surface of the mask assembly such that the inlet connector 7 projects forwardly of the mask frame 3.

In these embodiments, the cushion 5 must be removed before disconnecting the frame 3 and headgear.

In these embodiments the frame 3 is not exposed to, and does not form part of, the flow path of breathable gases into the mask assembly. This provides an advantage that the frame 3 requires less cleaning than if the frame 3 were exposed to the flow path.

Referring now to FIG. 84, an embodiment is provided in which the frame 3 is sandwiched between the cushion 5 and rigid ring 37 at the rear, and the inlet connector 7 and diffuser 59 at the front. The upper part of the central connection portion at the frame opening 13 is stepped back from the diffuser 59, when the mask assembly is viewed from the side. In this embodiment the frame clip 14 fits onto and around the exterior of the front of the inlet connector 7, and is sandwiched between the cushion and the inlet connector 7/diffuser 59. As can best be seen in FIG. 84c), this provides a recess 59a behind the upper part of the diffuser frame 61 which allows the user to more easily grip and remove the diffuser frame without interference from the mask frame 3.

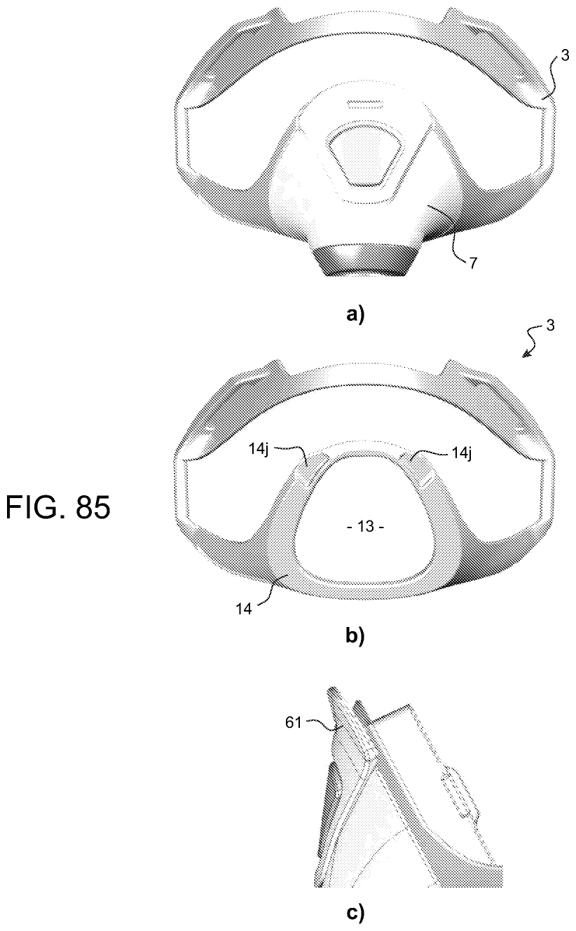

Referring to FIG. 85, a mask assembly is provided with similar features to that of FIG. 84. In this embodiment, the front portion of the mask frame 3 towards the top of the frame clip 14 is provided with two laterally spaced apart cut-outs 14j which ensure that the mask frame 3 does not interfere with the diffuser frame 61. The upper margin of the frame clip 14 is also relatively thin and relatively straight, to space the upper margin of the frame clip 14 from the diffuser frame 61.

It may be desirable to hold the frame 3 when disconnecting the cushion 5. In such a situation, it would be undesirable for the frame 3 to disconnect from the inlet connector 7 at the same time. It can therefore be desirable to ensure the frame 3 connects to the inlet connector 7 relatively securely, such that the connection resists disconnection.

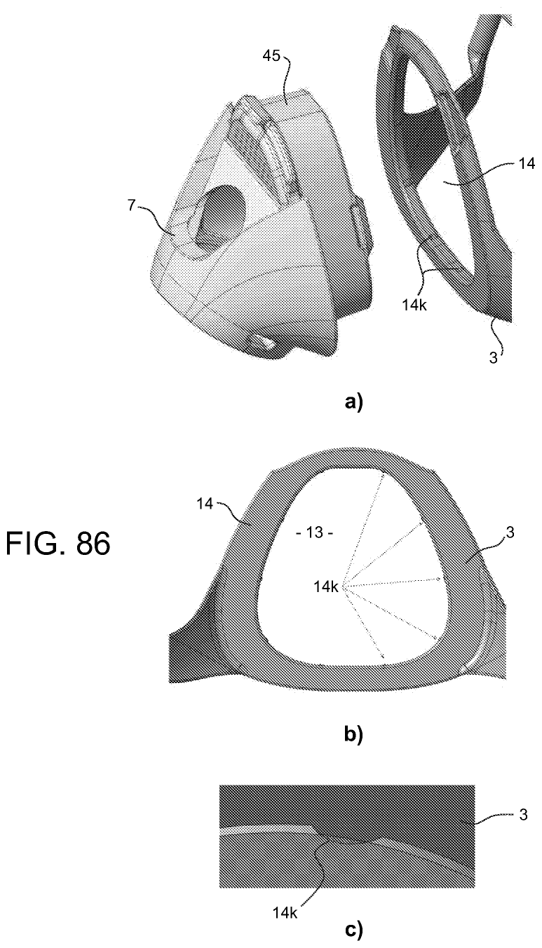

Referring to FIG. 86, a mask assembly is provided in which the inner walls 14a, 14b of the frame clip 14, which fit around and connect to the outlet boss 45 of the inlet connector 7, are provided with at least one protrusion 14k that engages the outlet boss 45. In the embodiment of FIG. 86, a plurality of protrusions 14k are provided, spaced around the interior walls 14a, 14b of the frame clip 14. Each protrusion 14k is in the form of a small bump that is dimensioned to provide an interference fit with the outlet boss 45, as can best be seen in FIG. 86c).

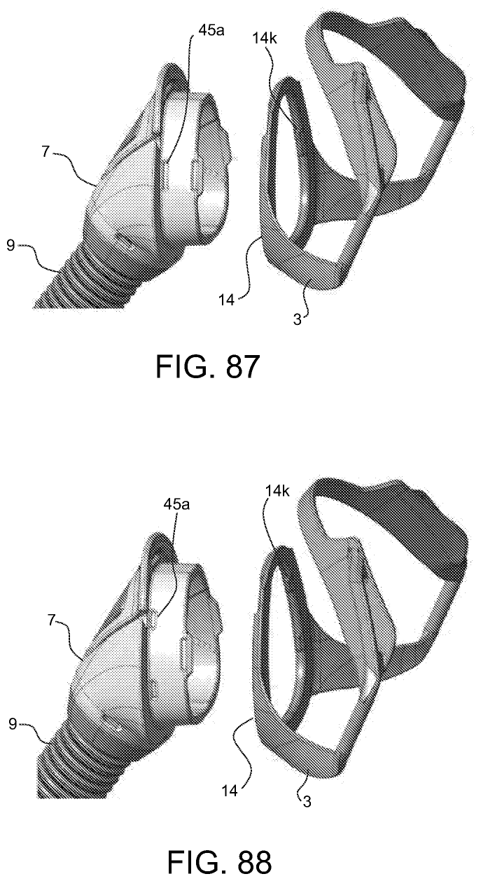

Referring to FIGS. 87 and 88, a mask assembly is provided with similar features to that of FIG. 86. In this embodiment, the frame clip 14 is provided with a smaller number of larger protrusions 14k. In this embodiment, the larger protrusions 14k fit into and engage with corresponding recesses 45a on the inlet boss.

In the embodiment of FIG. 87, the frame clip 14 is provided with a pair of laterally spaced apart elongate protrusions 14k that fit into respective elongate recesses 45a on the outlet boss 45. In the embodiment of FIG. 88, the frame clip 14 is provided with two opposed pairs of elongate protrusions 14k that fit into respective pairs of elongate recesses 45a on the outlet boss 45.

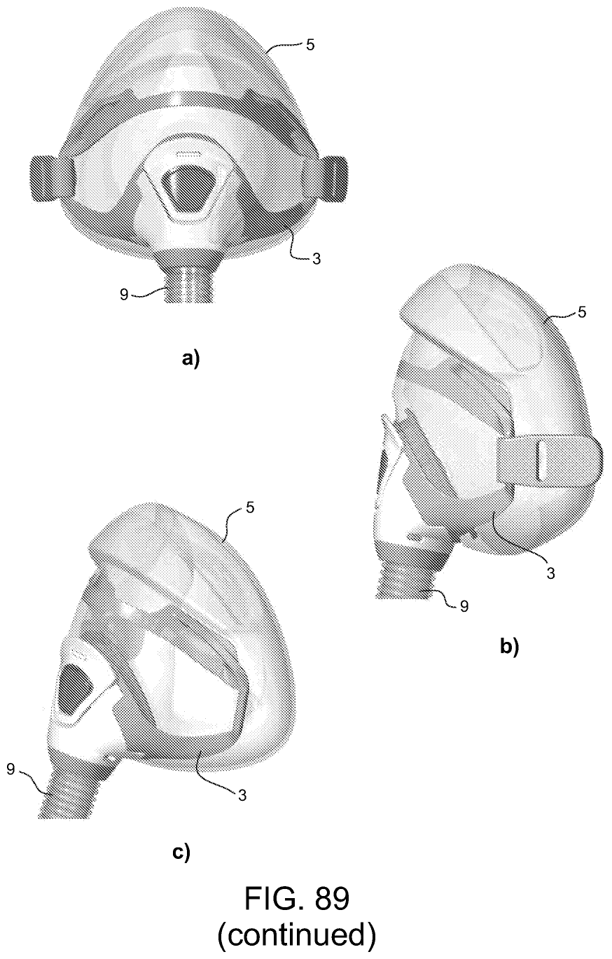

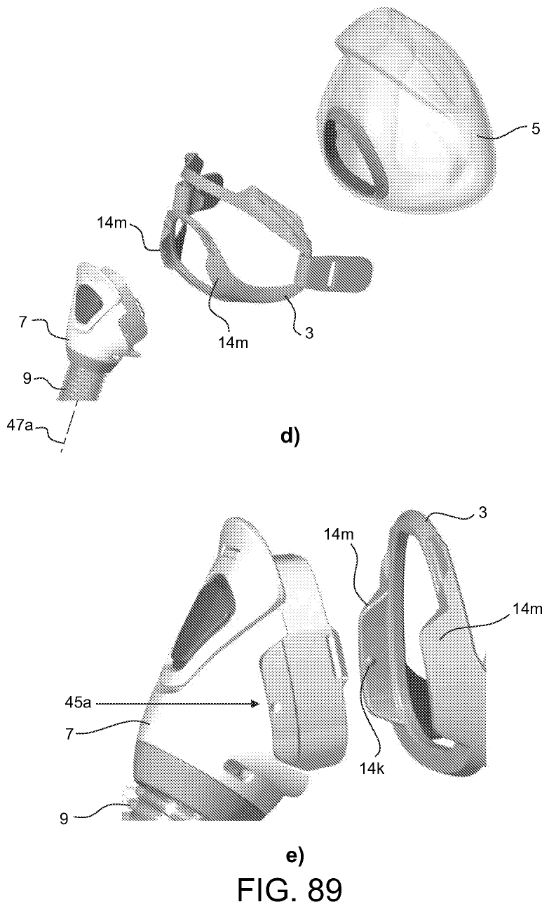

Referring to FIG. 89, a mask assembly is provided with similar features to those of FIGS. 86-88. In this embodiment, the side walls 14b of the frame clip 14 comprises forwardly directed tabs 14m that fit into corresponding cutouts 7j at the sides of the inlet connector 7. Each tab 14m comprises a protrusion 14k that engages a corresponding recess 7k in each cutout 7j to help retain the mask frame 3 on the inlet connector 7.

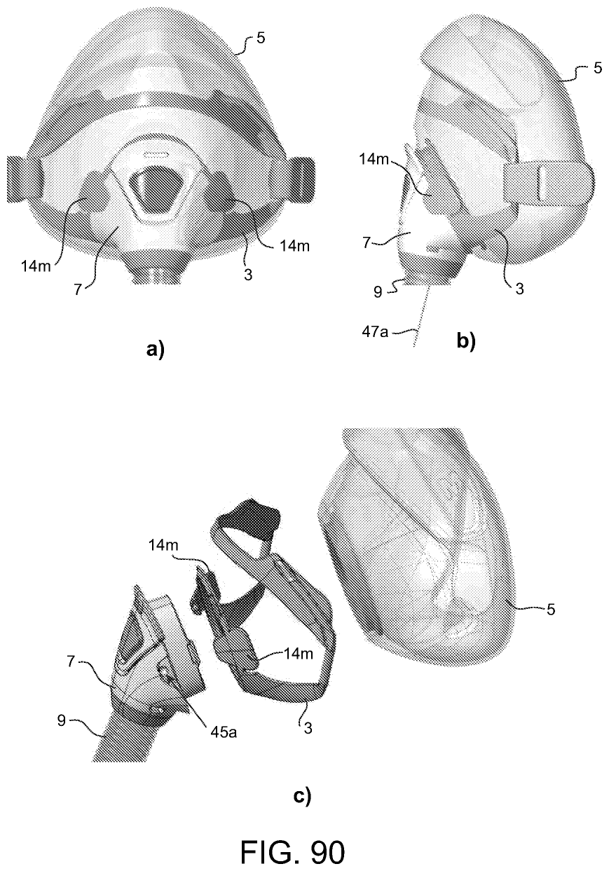

Referring to FIG. 90, a mask assembly is provided with similar features to that of FIG. 89. In this embodiment, each tab 14m comprises a rearwardly directed extension 14n that can be gripped by a user to help release the frame 3 from the inlet connector 7.

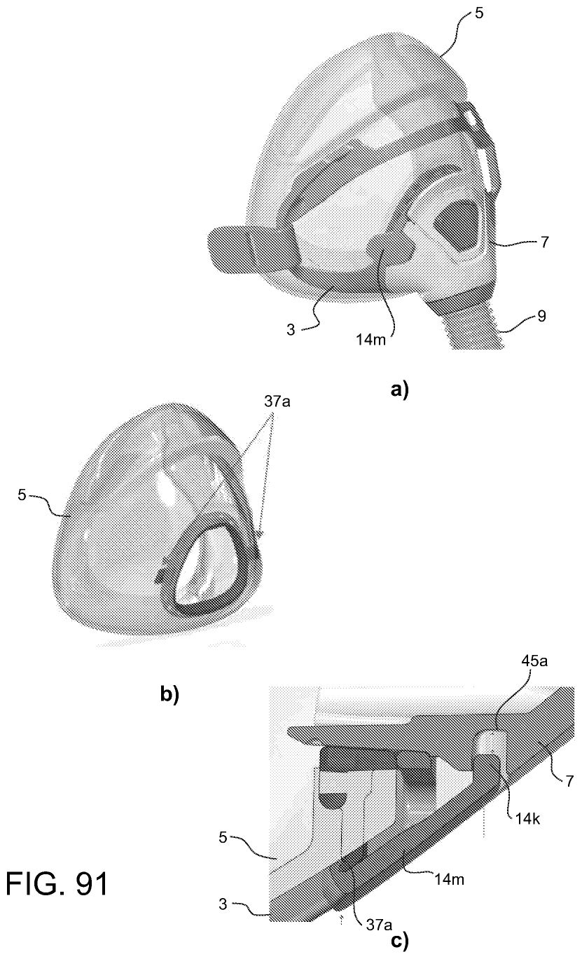

Referring now to FIG. 91, a mask assembly is provided with similar features to that of FIG. 90. As can best be seen from FIG. 91b), the rigid ring 37 of the cushion 5 is provided with a pair of laterally spaced apart, laterally extending teeth 37a. The rear of the rearwardly directed extension 14n of each tab 14m comprises a hook 14p each of which engages a respective tooth 37a, when the frame 3 is mounted on the cushion 5. Each tab 14m now functions as a dual function button. A front part of each tab 14m can be pressed to pivot each tab 14m in a first direction relative to the inlet connector 7 and cushion 5. When pivoted in this first direction, the hook 14p of each tab 14m disengages the tooth 37a, and releases the cushion 5 from the mask frame 3. When each tab 14m is pivoted in the opposed direction, by pressing a rear part of each tab 14m, the protrusions 14k on the tabs 14m disengage from their respective recesses 7k on the inlet connector, thus releasing the frame 3 from the inlet connector 7. Each tab 14m therefore provides a single button with which the user can:
a) release the cushion from the frame 3 whilst retaining the frame 3 on the inlet connector; or
b) or release the inlet connector 7 from the frame 3, whilst retaining the frame 3 on the cushion 5.

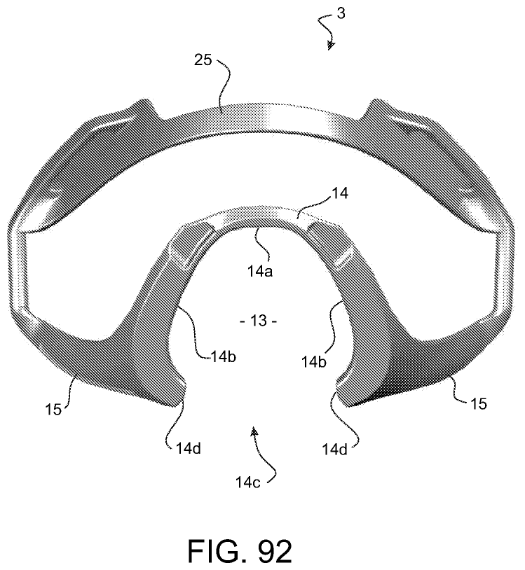

Referring to FIG. 92, a frame 3 of a mask assembly is provided comprising a frame clip 14. The frame clip 14 defines a mouth 14c at the bottom of the frame clip 14 that receives the top of the inlet connector 7, when the frame clip 14 is pushed substantially vertically downwardly onto the inlet connector 7. When viewed from the front the frame clip 14 comprises a wider lower portion, inclined side portions, and a narrower upper portion. The ends 14d extend inwardly such that the mouth 14c is smaller than the widest part of the frame clip 14. Consequently, as the frame clip 14 is pushed onto the inlet connector 7, the ends 14d resiliently deform in a laterally outward direction until the inlet connector 7 is fully received in the opening 13. The ends 14d of the frame clip 14 then snap back to their rest position, resisting removal of the frame 3 from the inlet connector 7.

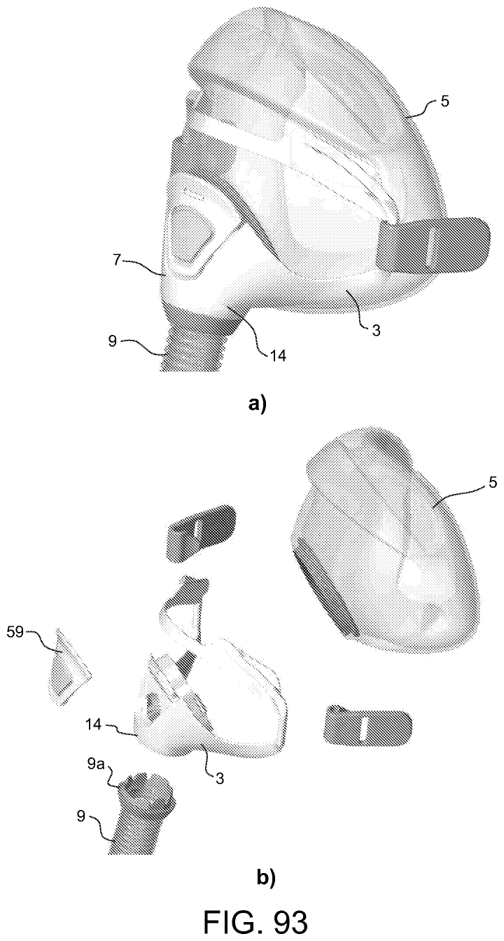

Referring to FIG. 93, a mask assembly is provided in which the frame 3 and inlet connector 7 are integral and therefore comprise a single component. Reducing the number of components of the mask assembly may reduce manufacturing and/or assembly costs, as well as simplifying use as less components must be assembled.

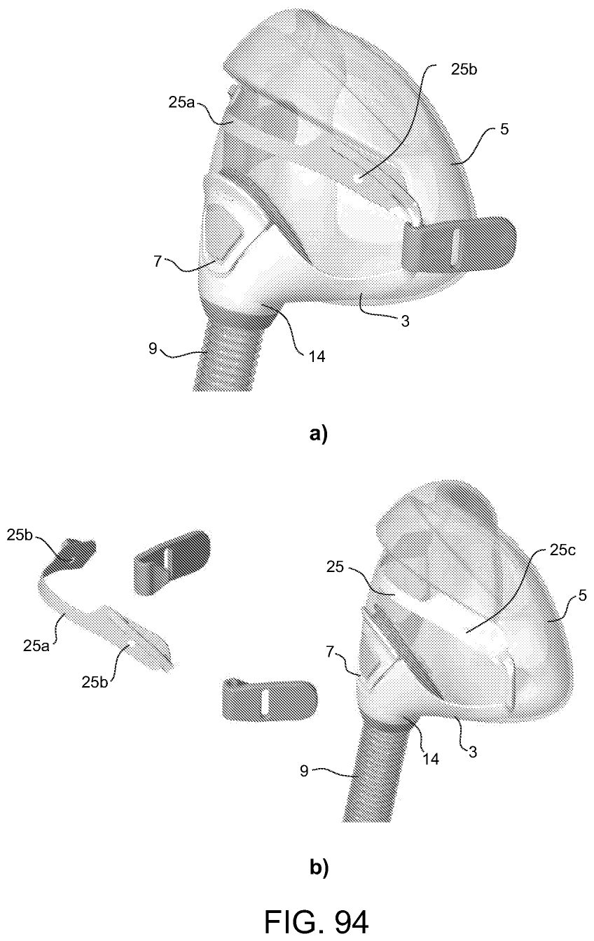

Referring to FIG. 94, a mask assembly is provided with similar features to that of FIG. 93. The frame 3 and inlet connector 7 are integral. In this embodiment, a headgear connector yoke 25A is removably mounted on the upper portion 25 of frame 3. In this embodiment the upper headgear connectors 19 are provided on the yoke 25A, rather than on the upper portion 25. The yoke 25A is provided with a plurality of apertures 25b configured to received corresponding lugs 25c on the upper portion 25. The engagement of the apertures 25b and lugs 25c locates and retains the yoke 25a on the upper portion 25 of the frame 3. Because the yoke 25a can be detached from the frame 3, the headgear can be removed without removing the cushion 5 and frame 3.

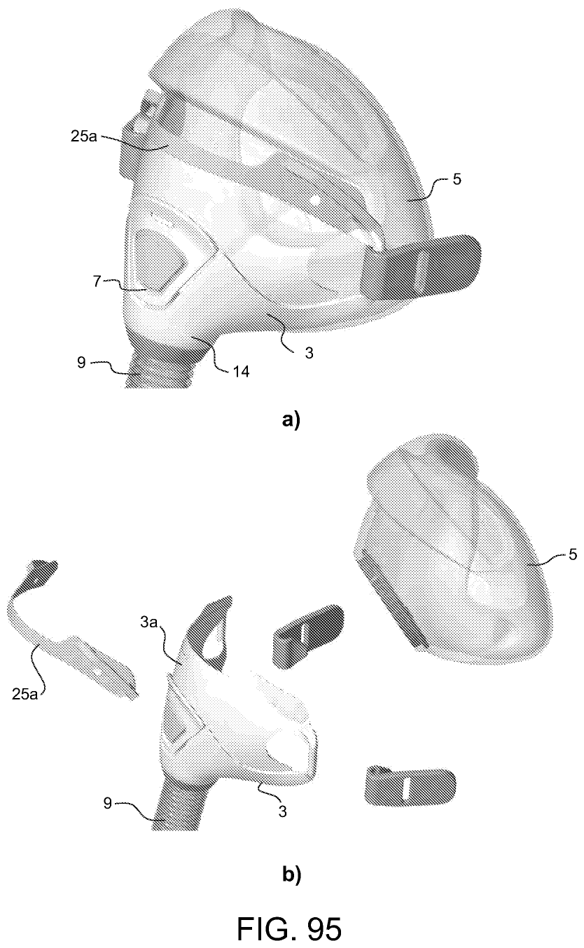

Referring to FIG. 95, a mask assembly is provided with similar features to that of FIGS. 93 and 94, and includes a headgear connector yoke 25A. In this embodiment, the window 20 between the lower part of the frame 3 and inlet connector 7, and the upper portion 25 of the frame 3, is omitted. Instead, the frame 3 further comprises an extended portion 3a that extends across the frame 3, from one lateral arm 15 to the other, above the inlet connector 7. The material of the extended portion 3a can add stability and rigidity to the mask frame 3.

In embodiments of this disclosure the mask assembly comprises a cushion 5 with a proximal face contacting portion, a distal inlet aperture, and a side wall between the proximal face contacting portion and the distal inlet aperture. A frame 3 is provided comprising at least one headgear connector and an inlet opening 13 configured to connect to a source of pressurised gas. The cushion 5 is mounted on the frame 3. For example, the distal inlet aperture of the cushion 5 may be removably mounted to the central connection portion of the frame 3 adjacent the inlet opening 13 of the frame 3. The frame 3 is configured to overlap an exterior portion of the side wall of the cushion 5.

In embodiments of this disclosure the mask assembly may comprise a cushion 5 with a proximal face contacting portion, a distal inlet aperture, and a side wall between the proximal face contacting portion and the distal inlet aperture, and a frame 3 comprising an inlet opening 13 configured to connect to a source of pressurised gas. The distal inlet aperture of the cushion 5 is removably connected to the central connection portion of the frame 3. The frame 3 comprises a pair of upper and a pair of lower headgear connectors. The cushion 5 comprises an uppermost portion, both pairs of upper and lower headgear connectors being below the cushion uppermost portion.

The frame 3 comprises a lower, central portion 11 which comprises a central connection portion configured to be removably connected to the cushion 5.

In embodiments of this disclosure the mask assembly 1 provides advantages when side sleeping. In particular, the mask assembly 1 is configured such that the lateral part of the mask assembly 1 that is nearest the bed or pillow can readily deform, whilst the lateral part of the mask assembly 1 furthest from the bed or pillow does not deform. The lateral deformation accommodates movement of the user's head, and to accommodates lateral forces applied by the bed or pillow to the lateral part of the mask assembly.

The deformation of the lateral part of the mask assembly 1 is preferably configured as elastic deformation, such that the lateral part of the mask assembly 1 reverts to its original shape once the deformation forces are removed. The cushion 5, plus the lateral arms 15 and upper frame portion 25 of the frame 3 can all be configured to elastically deform.

The frame 3, or at least portions thereof, can be configured to add only a relatively small amount of resistance to lateral deformation of the cushion 5, when lateral forces are applied to the mask assembly 1. The lateral arms 15 and upper frame portion 25 of the frame 3 can be so configured.

The frame 3 may be configured to deform under lateral forces to the extent that, when in a deformed condition:
a) the elongate elements of the upper headgear connectors 19, which are inwardly inclined when viewed from the front in a rest condition of the mask assembly 1, are displaced to a position in which the elongate elements are substantially parallel;
b) the lower headgear connectors 17 are displaced to a position in which the lower headgear connectors 17 are each aligned with a respective outer margin of the inlet opening 13 of the frame 3;
c) The lower headgear connectors 17 are displaced to a position in which the lower headgear connectors 17 are each aligned with a respective laterally outermost margin of the central portion 11 of the frame 3;
d) the lower headgear connectors 17 are displaced to a position in which the lower headgear connectors 17 are each aligned with a respective weakened region or living hinge between the lateral arms 15 and central portion 11 of the frame 3;
e) the lower headgear connectors 17 are displaced to a position in which the lateral distance between the lower headgear connectors decreases by a distance in the range of 140 mm to 20 mm, or 140 to 60 mm, or 110 to 40 mm.

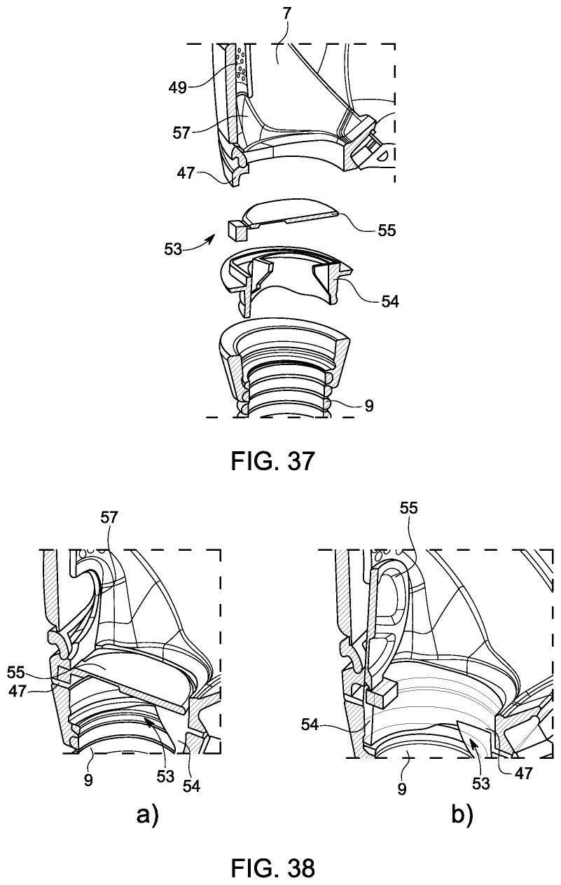

As described above with reference to FIG. 37, an AA valve 53 is provided comprising a valve member in the form of a valve flap 55 that is integral with, and pivotal relative to, a valve mount 55B.

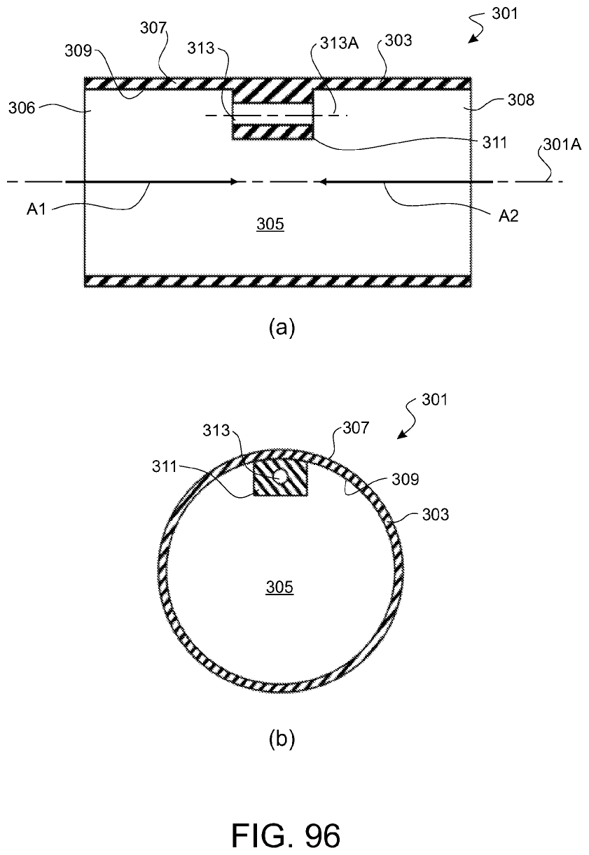

Referring to FIG. 96, a respiratory apparatus flow path component is shown schematically in the form of a conduit 301, comprising an outer wall 303 defining a lumen 305 being a gases flow path through the conduit 301, extending between an inlet 306 and an outlet 308 of the conduit 301. The outer wall 303 comprises an outer surface 307 and an inner surface 309.

An AA valve anchoring formation in the form of anchoring tab 311 is provided inside the gases flow path, extending radially inwardly at least partially across the gases flow path. In this example, the anchoring tab 311 extends from the inner surface 309. Anchoring tab 311 is provided with a through bore 313 which is an open bore, each end of which is exposed to the gases flow path. The through bore 313 extends along the conduit 301 such that a central axis 313A of the bore 313 is aligned with a central axis 301A of the conduit 301.

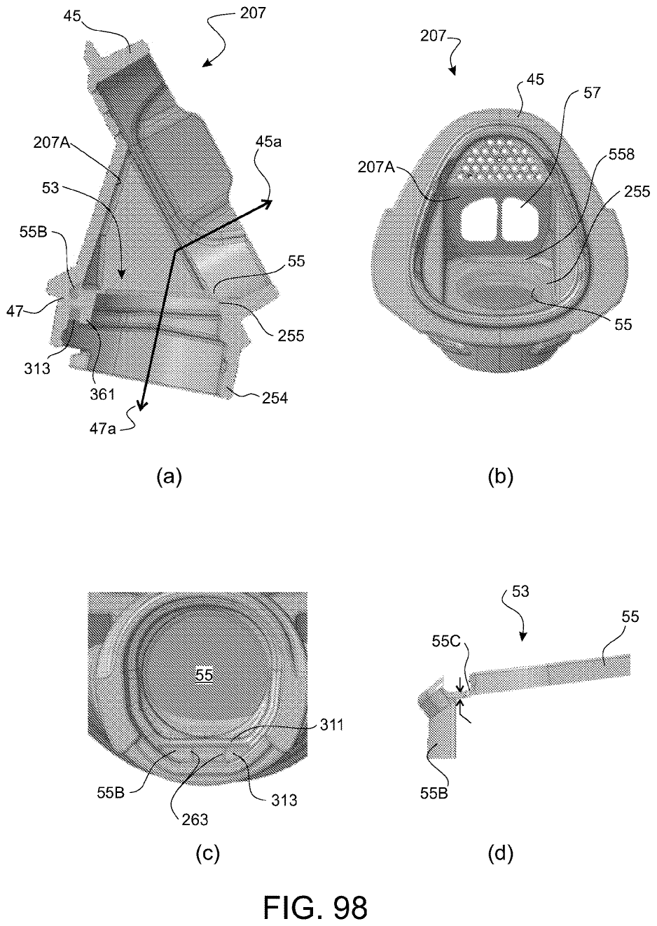

With reference to FIG. 98, the through bore 313 is configured to at least partially receive valve mount 55B of AA valve 53, to locate the AA valve 53 inside the conduit 301, such that the AA valve 53 can open and close the gases flow path.

The through bore 313, the shape and orientation of the gases flow path through the conduit 301, and the shape, size, relative position and orientation of the inlet 306 and outlet 308, are configured such that the mount of the AA valve 53 can be overmoulded inside the conduit 301 using a mould tool comprising only two mould tool halves. This is enabled by the alignment of the through bore 313 with the central axis 301A of the conduit 301. The first mould tool half is inserted into the conduit 301 via inlet 306 in the direction of arrow A1. The second, opposing mould tool half is inserted into the conduit 301 via outlet 308 in the direction of arrow A2. Both mould tool halves insertion directions are aligned, such that the mould tool halves share a common draw direction.

Further, because the central axis 313A of through bore 313 is aligned with the central axis 301A of the conduit 301, and with the draw direction of the mould tool halves, one of the mould tool halves can be provided with a shutoff formation to shut off one end of the through bore 313. Consequently, the valve mount of the AA valve can be moulded in the through bore 313, without mould material flowing all of the way through the through bore 313.

In this configuration, the entire AA valve, that is, the valve mount, the hinge, and the valve flap, can be moulded in situ inside the conduit 301, using a mould tool as described above, namely comprising only two mould tool halves and a single draw direction.

The mould tool halves enable the AA valve 53 to be overmoulded onto the conduit 301.

Conduit 301 could comprise any flow path component of a respiratory apparatus through which respiratory gases flow, and in which an AA valve could be provided. Conduit 301 could therefore comprise a length of flexible or rigid conduit, a conduit connector, a connector elbow, or an inlet connector 7 as described above.

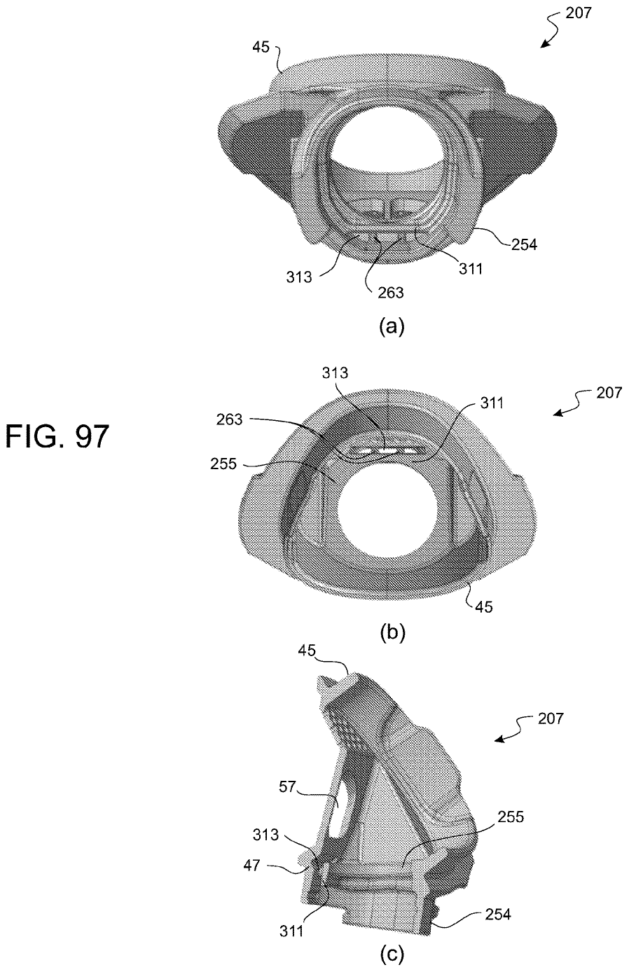

Referring to FIG. 97, a conduit 301 is provided comprising an inlet connector 207 in which the above described valve body 54 is integrally formed with the inlet connector 207, such that a separate process to secure the valve body 54 to the inlet connector 207 is not required. Providing the inlet connector 207 and valve body 54 as a single component also reduces the risk of misalignment between inlet connector 207 and valve body 54 during assembly.

Inlet connector 207 comprises comprises:
a) a rearwardly directed outlet boss 45 for connection to the cushion 5, the outlet boss 45 comprising an outlet port 45a that is in fluid communication with the cushion cavity;
b) a downwardly directed inlet boss 47 for connection to the frame 3, the inlet boss 47 comprising an inlet port 47b configured to receive breathable gas from the conduit 9, and comprising a central inlet axis 47a;
c) one or more bias vent holes 49. In this embodiment a plurality of vent holes 49 are provided in a single vent hole array 51;
d) an anti-asphyxia valve 53. In this embodiment the valve 53 comprises a valve body 54, and a valve member 55 movably mounted adjacent the inlet boss 47, and an inlet valve opening 57 above the inlet boss 47. The valve member 55 is movable between a closed position in which the valve member 55 closes the valve opening 57, and an open position in which the valve member 55 opens the opening 57, instead closing the passage through the inlet boss 47. The valve member 55 comprises a planar valve flap 55;
e) a diffuser 59. Diffuser 59 comprises a body of diffuser material that covers the vent holes 49, so as to diffuse any gases flow from those vent holes 49. The diffuser material may be permanently mounted on the inlet connector 207, for example via adhesive or overmoulding, or could be removably mounted on the inlet connector 207, for example via a rigid diffuser frame 61 that clips onto the inlet connector 207.

The inlet connector 207 has a base and inclined sides, when viewed from the front. The outlet boss 45 matches the size and shape of the rigid ring 37 of the cushion 5. The inlet connector 207 comprises an inclined, forward wall 207A that extends between the outlet boss 45 and inlet boss 47. The vent holes 49 and valve opening 57 are provided on the forward wall 207A. The plane of the forward wall 207A is substantially parallel with the inlet opening axis 13A of the inlet opening 13 of the frame 3. When mounted on the rigid ring 37, the forward wall 207A has substantially the same profile as the non-face contacting portion 29 and the side wall 31 of the cushion 5.

Inlet connector 207 comprises an integral valve body 254 that extends from the bottom of the inlet connector 207 away from the inlet boss 47. The features of the previously described valve body 54 are integrally formed, by the integral valve body 254 at the inlet boss 47. Integral valve body 254 namely comprises the features to enable the valve body 254 to connect to and form a gas flow path with the conduit 9, and the features to enable the AA valve 53 to be located in the inlet connector 207.

The integral valve body 254 comprises a planar valve seat 255 in which is formed the inlet port 47b, the inlet port 47b being circular in this example. In use, inlet port 47b is in fluid communication with the conduit 9. A forward margin of the valve seat 255 is terminated by an AA valve anchoring formation 311 comprising a downwardly directed anchoring tab that is spaced from the front wall 207A of the inlet connector 207 to define a through-bore 313 between the front wall 207A of the inlet connector 207 and the valve seat 255.

One or more posts 263 extend across the through-bore 313.

The through-bore 313 extends through the valve body 254 from above to below the valve seat 255.

Referring to FIG. 98, the AA valve 53 is shown inside the inlet connector 207. The valve flap 55 is planar and sits on the valve seat 255 to cover and close the inlet port 47b. The valve mount 55B is located in the through-bore 313. The valve flap 55 is connected to the valve mount 55B by a thinned region 55C that allows the valve flap 55 to pivot relative to the valve mount 55B, so that the valve flap 55 can pivot away from the valve seat 255 and inlet opening 47b, to a position adjacent the valve opening 57.

The valve flap 55 is movable from a first configuration in which the valve flap 55 rests on the valve seat 255, to a third configuration in which the valve flap 55 is adjacent the valve opening 57, via a second configuration in which the valve transitions between the first and third configurations.

In this example, the AA valve 53 is moulded inside the inlet connector 207. The inlet connector 207 and valve body 254 are first formed as a single, integral component. The AA valve 53 is subsequently moulded inside the inlet connector 207, with the valve mount 55B being formed (by the moulding process) inside the slot 313 to retain the AA valve 53 in the desired position.

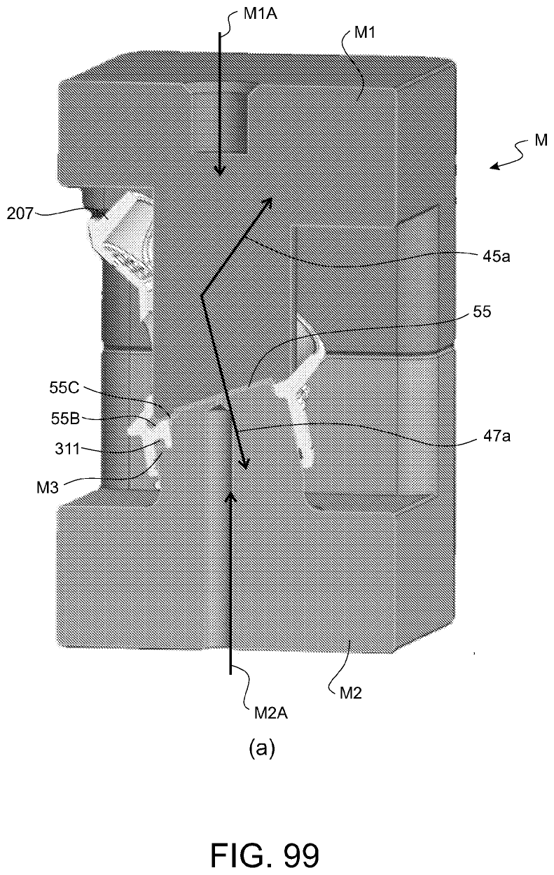

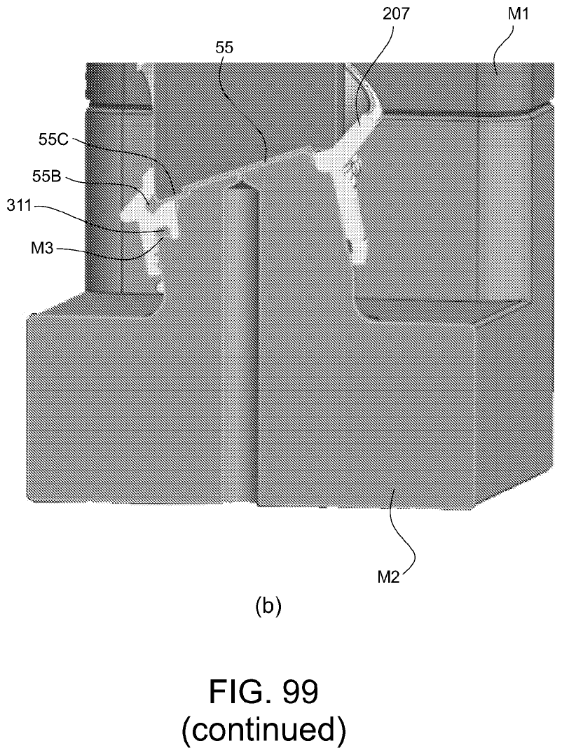

As can be seen in for example FIG. 98 and FIG. 99, inlet axis 47a of inlet port 47b of inlet boss 47 extends into the inlet connector 207 through inlet port 47b, and central outlet axis 45a extends into the inlet connector 207 through outlet opening 45b of outlet boss 45.

With reference to FIG. 99, the relative positioning and inclination of inlet axis 47a and outlet axis 45a, and the size and relative positioning on inlet port 47b and outlet opening 45b are such that a mould tool M can be used comprising only two mould halves M1, M2. Mould tool M1 can be moved along a mould direction axis MIA through outlet opening 45b, and mould tool M1 can be moved along a mould direction axis M2A through inlet port 47b. Mould direction axes M1A, M2A are the same. Such that mould halves M1, M2 share a common draw direction. Consequently, the inlet connector 207 is configured such that an upper mould half M1 and a lower mould half M2 can be inserted into the inlet connector 207 and can meet inside the inlet connector 207. Where the mould halves M1, M2 meet is where the AA valve 53 is moulded.

When the two mould halves M1 and M2 are fully inserted into the inlet connector 207, as shown in FIG. 98, the valve flap 55, the valve mount 55B and the thinned region 55C are all moulded as a single AA valve component. The valve mount 55B is moulded inside the slot 313, with the mould material flowing around the posts 263 to retain and secure the valve mount 55A in the slot 313.

The slot 313 is a through slot and thus exposed to both the upper and the lower mould halves M1, M2. The lower mould half M2 comprises a mould shut-off feature M3 which shuts off the underside of the slot 313 during moulding. Consequently, the AA valve 53 can be moulded as a single component, to the valve body 254 using a single moulding process using only two mould halves M1, M2. The moulding process is an overmoulding process.

In this example, the inlet opening axis 47A and the outlet opening axis 45A, are not aligned. However, a straight line can extend, unimpeded, between the centre of the inlet port 47B and centre of the outlet opening 45B.

In this example, the inlet opening axis 47A and the outlet opening axis 45A are relatively inclined.

In this example, the inlet port 47B and the outlet opening 45B are different sizes. A larger size allows insertion of a relatively large mould tool in the same draw direction as an opposing mould tool of a smaller size.

The embodiments of FIGS. 95 to 102 comprise an AA valve 53 that is moulded or formed directly inside a single component, namely the conduit comprising the inlet connector 207. In some examples, the component receiving the AA valve 53 may be the inlet connector of a mask as is described above.

Alternatively, the AA valve 53 may be located in any other fluid flow path component, such as for example a conduit connector cuff, or the gas delivery conduit itself, or in a conduit connector.

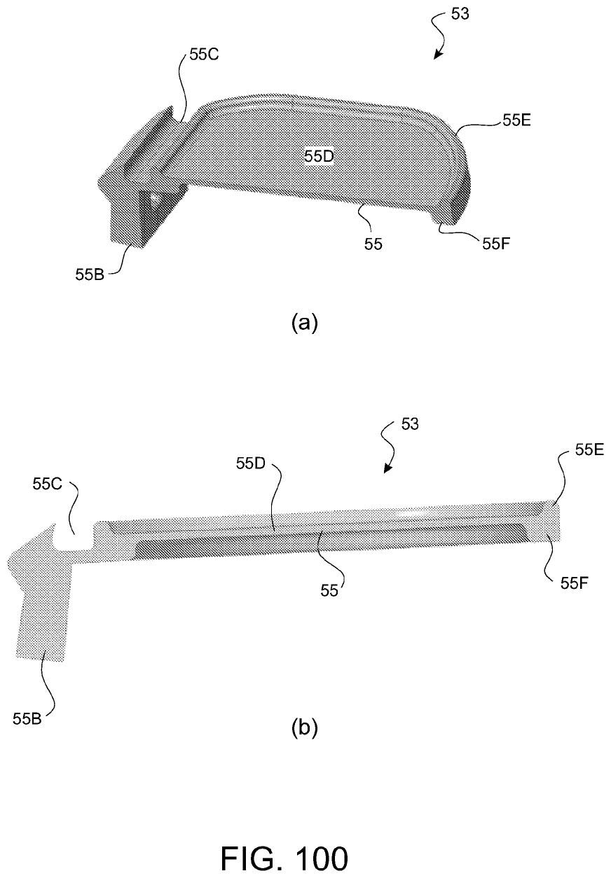

Referring now to FIG. 100, an example structure of an AA valve 53 in accordance with this disclosure can be seen.

The valve flap 55 is substantially planar and comprises a relatively thin central region 55D comprising an upper planar surface bounded by a thicker peripheral upper rim 55E and a lower planar surface bounded by a thicker peripheral lower rim 55F.

The upper rim 55E improves the seal with the valve opening 57, when the AA valve 53 is open. The upper rim 55E contacts the front wall 207A of the inlet connector 207, but the upper planar surface does not. This assists in minimising the risk of static holding the flap 55 in the open position even when the respiratory gases flow from the conduit 9 has stopped. The lower rim 55F improves the seal with the valve seat 255 around the inlet port 47B. The thicker upper and lower rims 55E, 55F, also help prevent, or at least minimise, tears and damage to the valve flap 55, and help provide stiffness to the valve flap 55 to prevent or minimise deformation in use.

The thinned region 55C between the valve flap 55 and valve base 55B is of relatively thin, constant thickness, that allows the valve flap 55 to open and close by allowing the valve flap 55 to pivot relative to the valve base 55B. The thinned region 55C is of constant cross section along its length, which allows for an even load distribution and avoids, or at least minimises, stress concentrations that might cause tears.

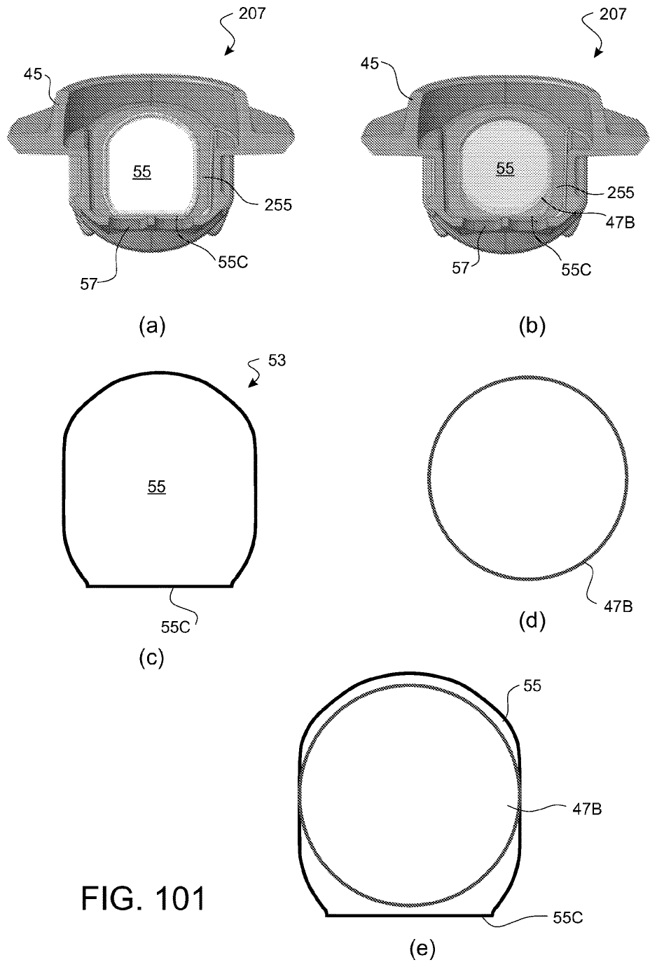

Referring now to FIG. 101, the relative position of the AA valve 53 and the inlet port 47B can be seen.

The inlet port 47B is substantially circular in this example. The valve flap 55A has a different shape to the inlet port 47B, and in this example is not circular. The valve flap 55 is shaped such that the flap 55 fully covers the inlet port 47B when the AA valve 53 is closed, meaning that leak through the inlet port 47B is minimised. The valve flap 55 comprises a straight margin adjacent the thinned region 55C, that is, at a portion of the valve flap proximal the pivot axis, straight side margins, and an arcuate distal end portion.

As can best be seen with reference to FIG. 101 e), the valve flap 55 is generally elongate in a direction extending way from the valve pivot, and extends beyond the inlet port 47B and overlaps with the valve seat 255 at the distal end portion, and overlaps with the valve seat 255 at the proximal portion of the flap 55, that is, there is overlap at positions distal from the thinned region 55C, and adjacent the thinned region 55C. This means that under normal breathing pressures, the valve flap 55 is unlikely to be inverted (and to be pushed downwards through the inlet port 47B towards the conduit 9).

The presence of diffuser 59, and in particular diffuser material over the bias holes 49 and valve opening 57, may be a reason for the inclusion of the valve seat 255 as opposed to a fully floating valve flap 55. The pressure drop created by the diffuser 59 means the valve flap 55 is more likely to be pushed downwards by breathing pressures. The valve seat 255 helps prevent the valve flap 55 from moving through the inlet port 47b.

At the side margins of the valve flap 55, the flap 55 is approximately as wide as the inlet port 47B, in that the side margins of the valve flap 55 contact the side margins of the inlet port 47B, but do not leave an air gap between the side margins of the valve flap 55 and the side margins of the inlet port 47B. The side margins of the valve flap 55 do not overlap with the side margins of the inlet port 47B. The side margins are spaced apart by a width dimension of the flap, the width dimension being less than or equal to the width of the inlet port 47b.

This lack of overlapping regions at the side margins of the flap 55 (combined with the relatively thin central region 55D), means that should the flap 55 be manually or otherwise pushed down towards the conduit 9 and become stuck, the minimum pressure delivered by the respiratory apparatus when providing a therapeutic gases flow is sufficient to push the flap 55 back up through the inlet port 47b and into the desired operating position where the valve flap 55 pivots away from the inlet port 47B to a position adjacent the valve opening 57. The minimum pressure could, in a typical CPAP apparatus, be around 4 cmH2O. The valve 53 can be configured, according to the minimum pressure delivered by the apparatus, to be pushed back through the inlet port 47b.

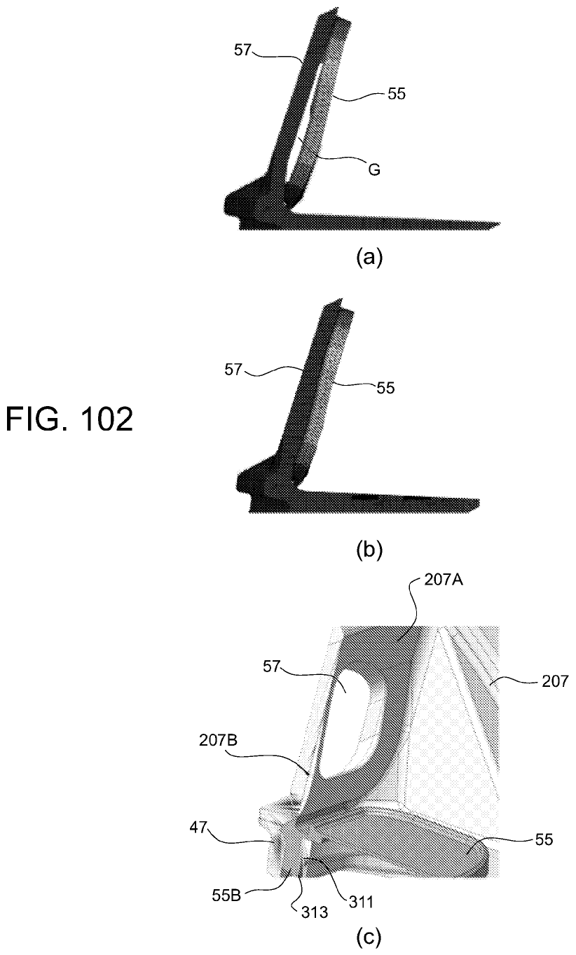

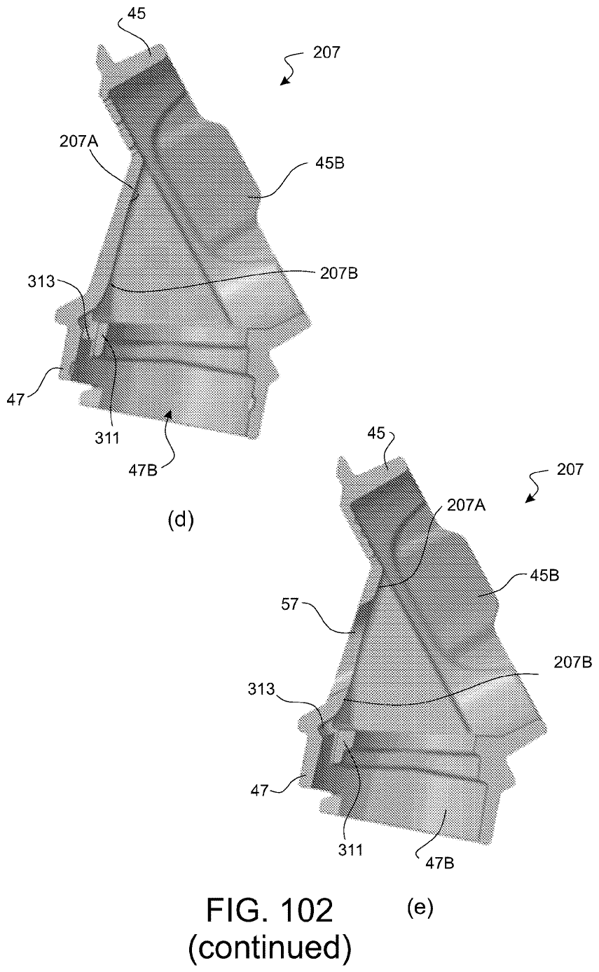

With reference to FIG. 102 a) an AA valve flap 55 is shown schematically in which the valve flap 55 becomes non-planar in use, that is, the valve flap 55 deforms to some degree along its length, as it pivots towards the valve opening 57. This deformation can mean that the upper surface of the valve flap 55 does not seal against the front wall 207A around the valve opening 57 as effectively as intended. This is due to a gap G forming between the valve flap 55 and the valve port 57.

With reference to FIG. 102 b), a more desirable configuration is shown in which the valve flap 55 is in better contact with the valve opening 57, such that there is no gap G, or gap G is at least minimised.

Reduction of gap G can be provided by a valve flap 55 in accordance with this disclosure in which the valve flap 55 is configured to minimise deformation, for example by using the thicker upper and/or lower peripheral rims 55E, 55F.

With reference to FIG. 102 c) to d), gap G can also be minimised by configuring the shape of the inner surface of the front wall 207A of the inlet connector 207, at valve opening 57, to match the shape of the upper surfaces of the valve flap 55 to some extent, and in particular to match the shape that the valve flap 55a has a tendency to adopt, in use, when viewed from the side, that is along the pivot axis of the valve flap 55.

The valve flap 55 can have a tendency to curve along its length, when viewed along its pivot axis (that is, in side profile), between the pivot axis and the distal margin of the valve flap 55.

The valve flap 55 can have a tendency to adopt an increased curvature at a proximal portion of the valve flap 55, that is, the portion between the pivot axis and a mid-point of the valve flap 55.

The valve flap 55 can have a tendency to adopt reduced curvature, or no curvature at all, at a distal portion of the valve flap 55, that is, the portion between the mid-point and the distal margin of the valve flap 55a.

These general tendencies can be seen in FIG. 102 a). The lower portion of the valve flap 55 tends to curve away from the valve sealing surface 207A adjacent the valve opening 57 (i.e. the front wall 207A of the inlet connector 207 in this example), whereas the upper portion of the valve flap 55 tends to curve towards the sealing surface 207A, or indeed to remain planar.

The front wall 207A of the inlet connector 207, and in particular the inner surface of that front wall 207A that forms the valve sealing surface with the upper rim 55E of the valve flap 55A, comprises at least a non-planar portion 207B having a shaped, non-planar profile when viewed in cross-section, such that the shape of the non-planar portion 207B in cross section to some extent mimics the shape that the sealing surface of the valve flap 55 tends to adopt in use, when respiratory gases are flowing through the conduit 9 into the inlet connector 7.

In cross section when viewed from the side in a direction along the pivot axis of the AA valve 53, the non-planar portion 207B may therefore comprise:
a) an aerofoil shape;
b) one or more arcuate valve sealing surfaces;
c) one or more bulbous portions that project into the inlet connector 207;
d) one or more planar portions;
e) one or more thicker portions;
f) one or more thinner portions.

Preferably the non-planar portion 207B is nearer the pivot axis than the distal margin of the valve opening 57.

Preferably, the portion of the front wall 207A nearer the pivot axis of the valve flap 55 projects further into the inlet connector 7, than the portion of the front wall 207A distal from the pivot axis.

The non-planar portion 207A is arcuate and comprises an apex, wherein the apex is closer to the pivot axis of the valve flap 55, than to the distal margin of the valve flap 55.

The inner surface of the front wall 207A may be entirely non-planar, that is, may not comprise any planar portion(s) between a position adjacent the pivot axis of the AA valve 53, and the distal margin of the valve opening 57.

The inner surface of the front wall 207A may comprise one or more non-planar portions and one or more planar portions.

The non-planar portion 207B may be relatively thicker, and the planar portion may be relatively thinner.

The valve opening 57 may be positioned, at least partially:
a) in the non-planar portion 207B;
b) in the thicker portion;
c) in a planar portion;
d) in a thinner portion;
e) to extend across one portion to another portion of the valve sealing surface.

The non-planar portion 207B extends along the side margins of the valve opening 57, and across a lower margin of the valve opening 57 (the lower margin being nearer the pivot axis of the AA valve 53, than a distal margin of the valve opening 57). The non-planar portion 207B in this example does not extend across an upper margin of the valve opening 57. The non-planar portion 207B therefore extends across the width of the valve sealing surface of the front wall 207A, such that the valve flap 55 seals against the non-planar portion 207B at the bottom and side margins of the valve opening 57.

The shaped profile of the inner wall 207A helps to ensure a seal between the inner wall 207A and the valve flap 55A along the entire length of the valve flap 55a, that is, from adjacent the pivot axis all the way to the distal margin of the valve flap 55a. This creates an improved seal, and minimises or eliminates any leakage gap G. The seal can be more consistent and controllable. The non-planar portion 207B does not necessarily have to extend to the distal margin of the valve opening 57, that is the entire length of the valve flap 55 does not necessarily need to seal against the non-planar portion 207B. The non-planar portion 207B extends as far as is necessary to mirror the portion or portions of the valve flap 55 that have a tendency to deform.

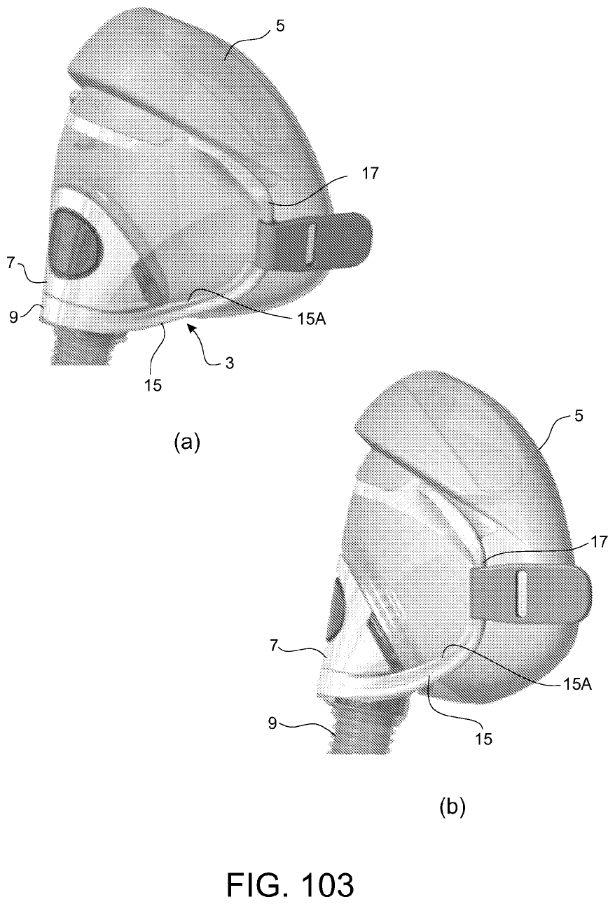

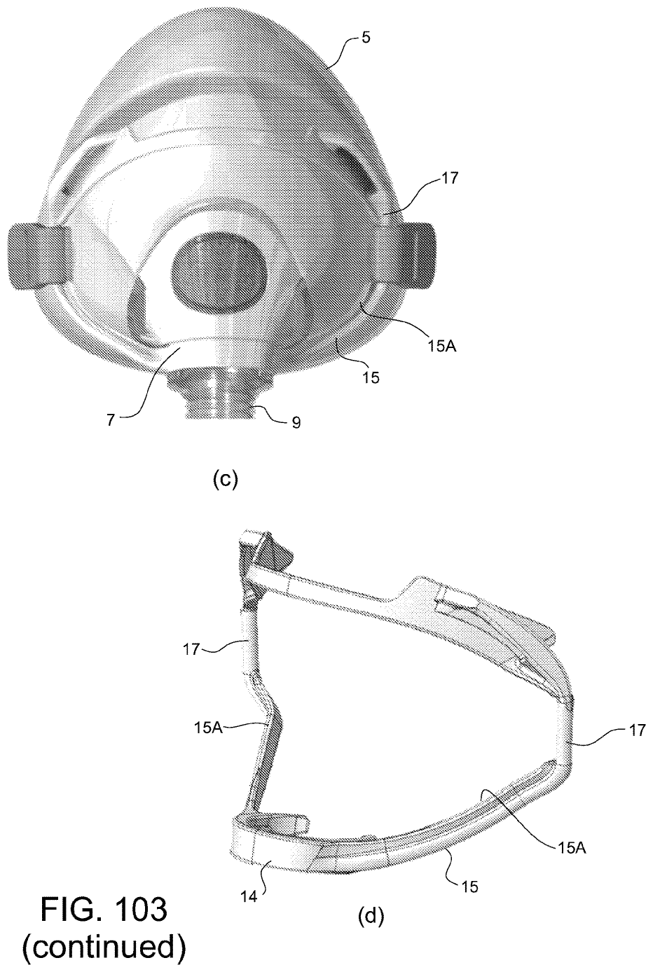

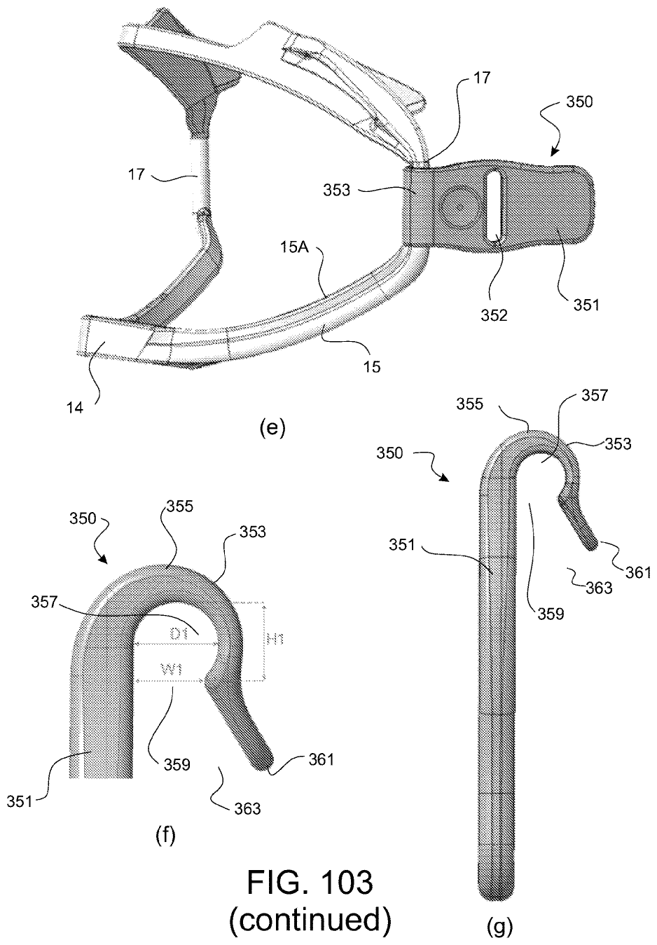

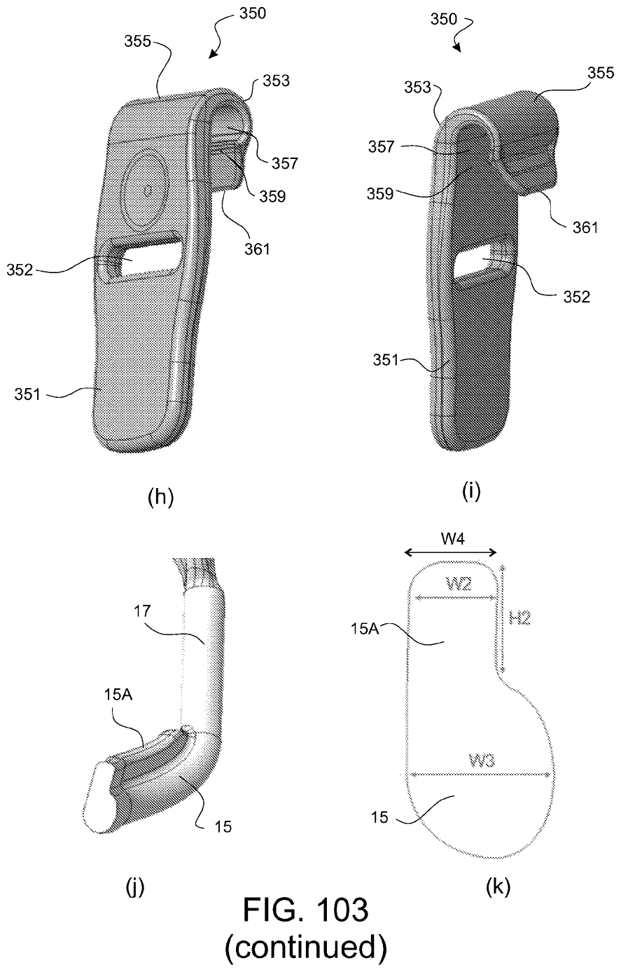

Referring now to FIG. 103, a modified frame 3 in accordance with this disclosure comprises lateral arms 15 that comprise elongate rails 15A. Each rail 15A extends part way along the length of a respective lateral arm 15, from a position adjacent the central portion of the frame 3, to a position adjacent the lower headgear connectors 17.

Each rail 15A provides an elongate guiding feature when connecting the lower headgear connector clips 350 to the lower headgear connectors 17. The user can loosely hook the headgear connector clips 350 over the rails 15A, and then slide the headgear connector clips 350 along the rails until the headgear connector clips 350 clip onto respective lower headgear connectors 17. Thus, the user can be relatively inaccurate in initially positioning the headgear connector clips 350 onto the lateral arms 15, and the guide rails 15A guide the headgear connector clips 350 along the arms 15 to the lower headgear connectors 17.

The rails 15A also add rigidity to the lateral arms 15, but are relatively narrow to allow the frame flexibility previously described. This flexibility ensures that the added rigidity provided by the rail 15A does not cause the frame 3 to be unintentionally disconnected (from the cushion 5 or inlet connector 7/207), when the lower headgear clip 350 is removed.

With reference to FIG. 103f) to i), headgear connector clip 350 comprises an elongate body 351. A headgear strap receiving slot 362 is provided part way along the elongate body 351.

One end of the elongate body 351 comprises a hook 353. Hook 353 comprises an arcuate end portion 355 that curves away from the end of the elongate body 351 to define a post receiving portion 357 having a maximum width D1. The post receiving portion 355 curves inwardly back towards the elongate body 351 to define a narrower, mouth portion 359 having a width W1, W1 being less than D1. The post receiving portion 355 has a height H1 extending from the narrower portion 359. A guide portion 361 extends outwardly from mouth portion 359, and is inclined away from elongate body 351 to define a lead-in 363 that leads to mouth portion 359.

With reference to FIG. 103 j) and k), each rail 15A comprises a thinned, oblong cross section region that extends along the top of the wider, more rounded cross section lower lateral arm 15. Each rail 15A comprises a width W2 which is less than the width W3 of the lateral arm 15, when viewed in transverse cross-section. Each rail 15A has a height H2, extending from the intersection between the arm 15 and rail 15A, and the distal, upper margin of the rail 15A.

The headgear connector clip 350 attaches to the lower headgear mount 17 of frame 3, with the mounting post of headgear connector 17 being receiving in the post receiving portion 355.

The maximum width D1 of post receiving portion 355 is the same as the diameter of the mounting post of lower headgear connector 17. The width W of the mouth portion 359 of headgear connector 350 is less than the diameter of the mounting post of lower headgear connector 17, such that the post receiving portion 355 deforms as the headgear connector clip 350 is pushed or pulled onto the mounting post, the lead-in 363 assisting in guiding the mounting post into the post receiving portion 355. Once the mounting post is fully received in the post receiving portion 355, the post receiving portion 355 reverts to its original size, to retain the mounting post in the post receiving portion 355, with the mouth portion 359 resisting movement of the mounting post out from the post receiving portion 355.

As explained above, the rails 15A function as a lead-in for the headgear connector clips 350 to guide the headgear connector clips 350 along the arms 15 and onto the lower headgear connectors 17. It is desirable to minimise the likelihood of the headgear connector clips 350 becoming stuck on a respective arm 15, and/or to keep friction between the headgear connector clips 350 and the rails 15A relatively low. Consequently, it may be desirable for the width W3 of the lateral arms 15 to be less than the width W1 of mouth portion 359 of headgear connector clip 350, such that the headgear connector clip 350 cannot become stuck on the largest width W3 of the lateral arm 15.

It may be desirable that:
a) the height H2 of rails 15A is greater than height H1 of the post receiving portion 355; and/or
b) the width W2 of the rails 15A is less than width W1 of mouth portion 359.

This can assist in ensuring that the narrowest width W1 of mouth portion 359 on the headgear connector clip 350 cannot extend below the rail 15A to the wider section W3 of arm 15, and cannot become stuck on rail width W2.

Depending on the angle the headgear connector clip 350 is applied relative to the frame 3, in some examples, only the bottom margin of the headgear clip 350 may contact the rail (for example if applied at the angle shown in FIG. 103e).

Therefore, it may be desirable that the width W4 of the top of the rails 15A is less than width W1 of narrower portion 359 of headgear connector clip 350, such that the headgear connector clip 350 does not become stuck on the rail 15A when applied at an angle (that is, the longitudinal axis of the headgear connector clip 350 is not aligned with the axis of the lateral arm 15).

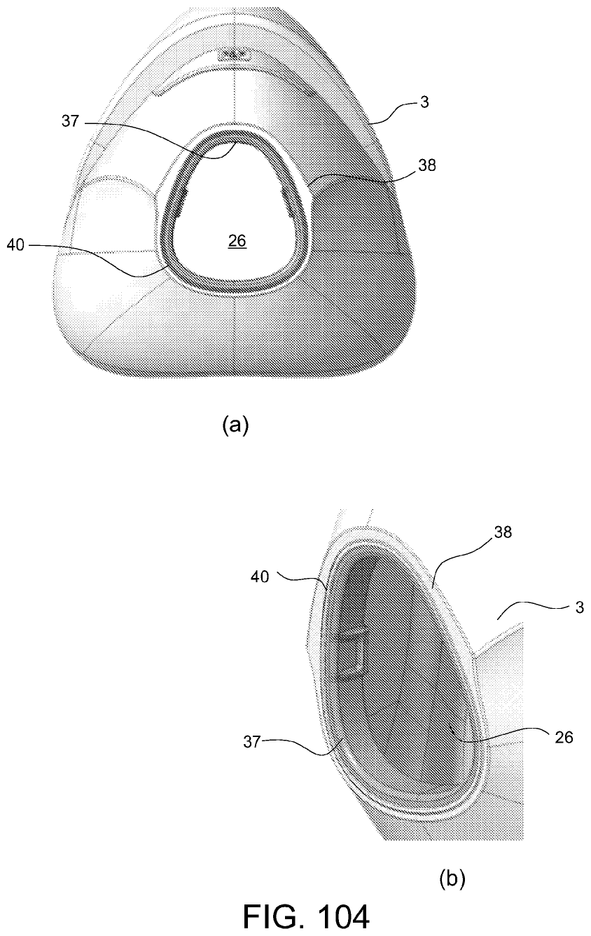

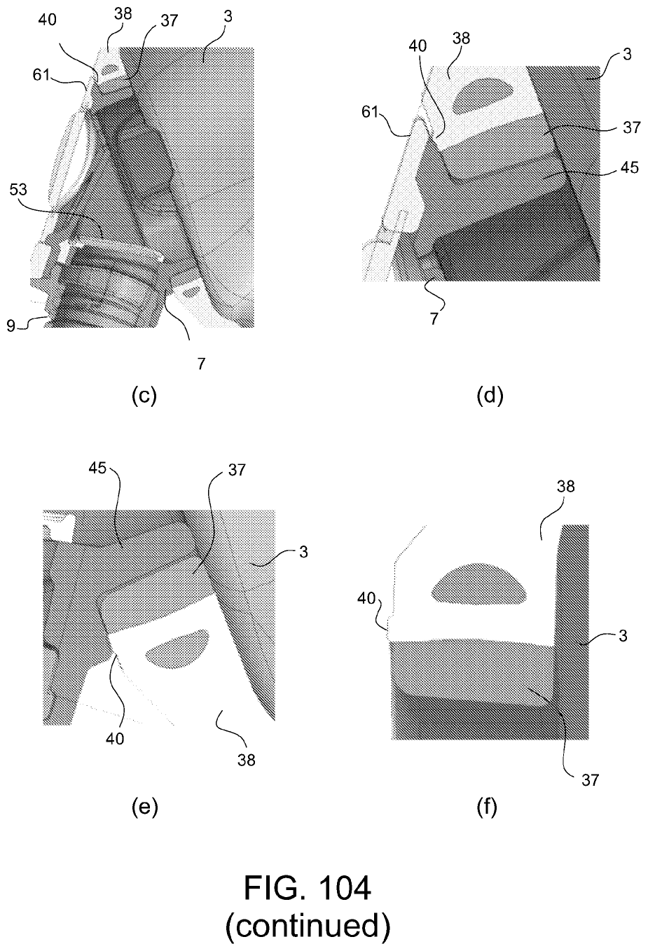

Referring now to FIG. 104, as described above, cushion 5 is provided with a rigid ring 37, around the inlet opening 26, that provides a connection between the cushion 5 and the outlet boss 45 of the inlet connector 7. The rigid ring 37 may be overmoulded to the cushion 5, in the earlier described embodiments.

Referring to FIG. 104, the cushion 5 comprises overmould material 38 that attaches the relatively rigid ring 37 to the cushion 5 at the inlet opening 26. In this example, a forward portion of the overmould material 38 comprises a continuous rib seal 40 that extends around the inlet opening 26. The rib seal 40 projects away from the front of the cushion 5, forwardly away from the rigid ring 37 in a direction along the axis of the inlet opening 26. In other words, the rib seal 40 projects axially outwardly from the cushion 5. The rib seal 40 extends around the outside of the cushion inlet opening 26, externally of the cushion 5.

The rib seal 40 comprises a generally flat front sealing face 40A, which can best be seen in FIG. 104 f). When the cushion 5 is mounted on the inlet connector 7, sealing face 40a contacts a rear face 45D inside outlet boss 45 of inlet connector 7, as can best be seen in FIG. 104 c) to e). When so mounted, the rib seal 40 is compressed, so as to seal against the rear face 45D, to provide a seal between the cushion 5 and the inlet connector 7.

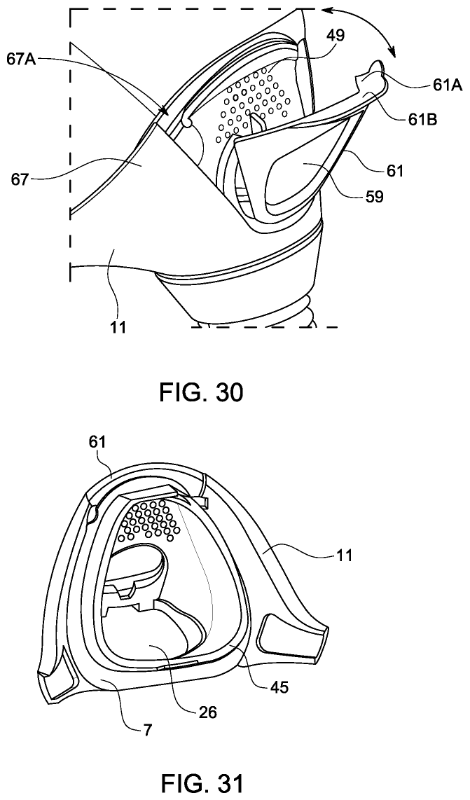

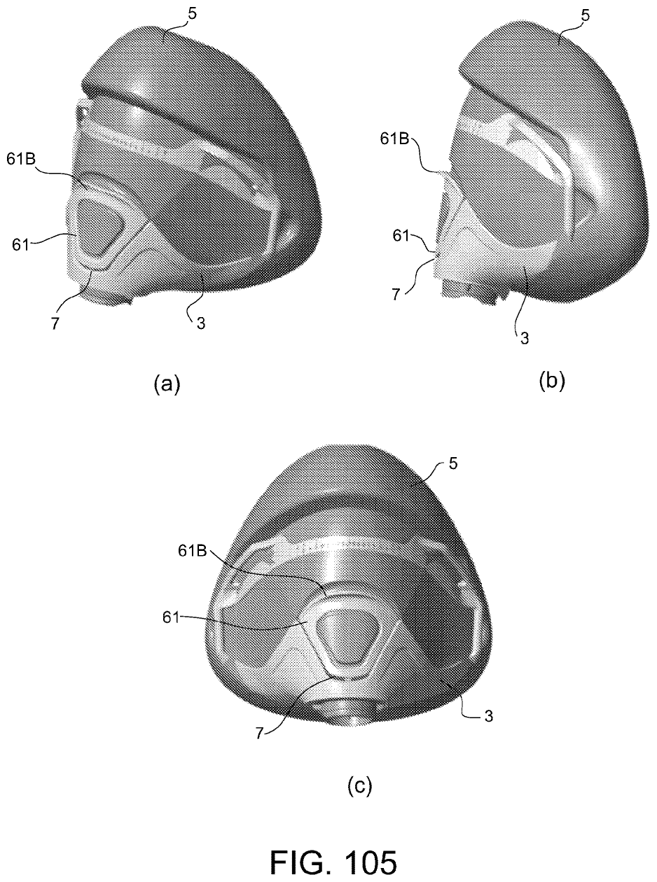

Referring now to FIG. 105, the diffuser frame 61 is similar to that described with reference to FIG. 30, but wherein the outwardly protruding finger tab 61B is longer than that shown in FIG. 30, and extends further across the top margin of the diffuser frame 61. The cushion 5 comprises a corresponding elongate recess 66 on the front face of the cushion 5 behind the finger tab 61B. The finger tab 61B is therefore spaced from the recess 66 to allow a user to more easily grip the finger tab 61B to pull the upper portion of the diffuser frame 61 away from the frame 3 to remove the diffuser 59.

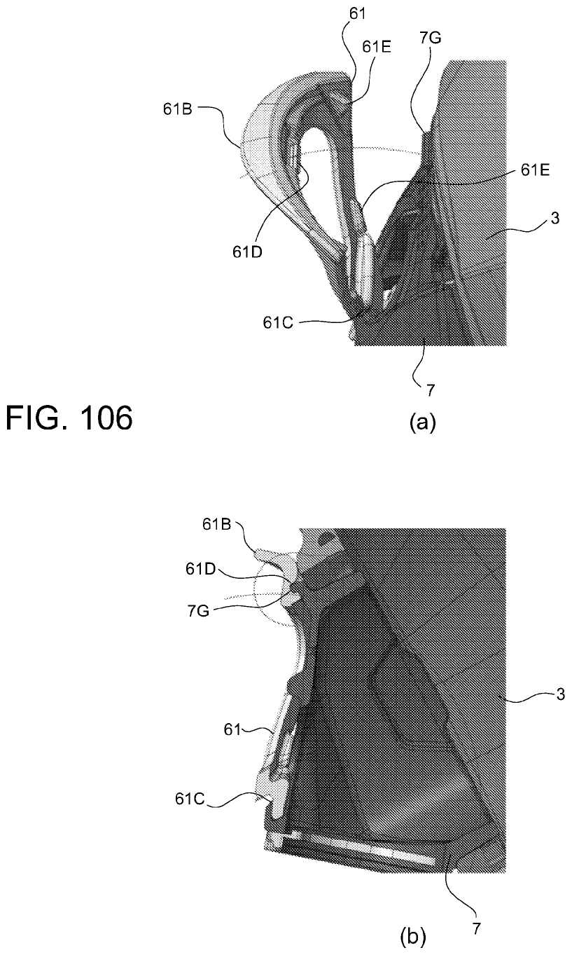

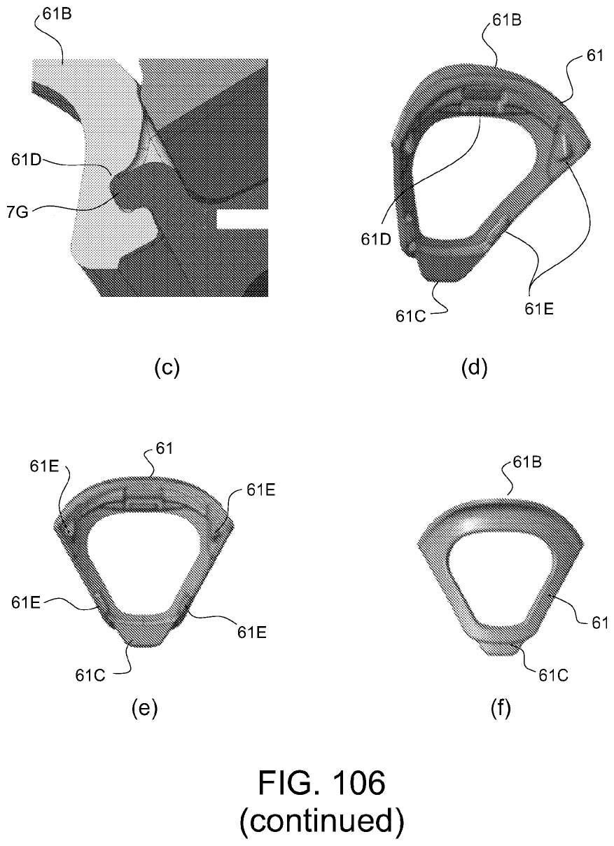

Referring now to FIG. 106, the diffuser frame 61 in this example comprises connection features, to connect the diffuser frame 61 to the frame 3, that are located centrally at the top and bottom of the diffuser frame 61, that is, in a position that corresponds to the likely location of a force applied by the user to release the diffuser frame 61 from the frame 3.

Diffuser frame 61 in this example therefore comprises a lower, central mounting tongue 61C that is received in a lower portion of the frame 3. Diffuser frame 61 further comprise an upper mounting feature comprising a recess 61D that clips onto a corresponding protrusion 7G above the valve opening 57, the protrusion 7G protruding from the inlet connector 7. The user can mount the diffuser 59 onto the inlet connector 7 by inserting the tongue 61B into a lower part of the frame 3, pivoting the diffuser 59 toward the inlet connector 7 about the tongue 61B, and then pressing the recess 61B onto the protrusion 7G. Protrusion 7G is received in recess 61B with an interference fit.

The diffuser frame 61 may further comprise one or more locating tabs 61E that are received in corresponding slots in the inlet connector 7, to help align and retain the diffuser frame 61 in the desired position on the inlet connector 7.

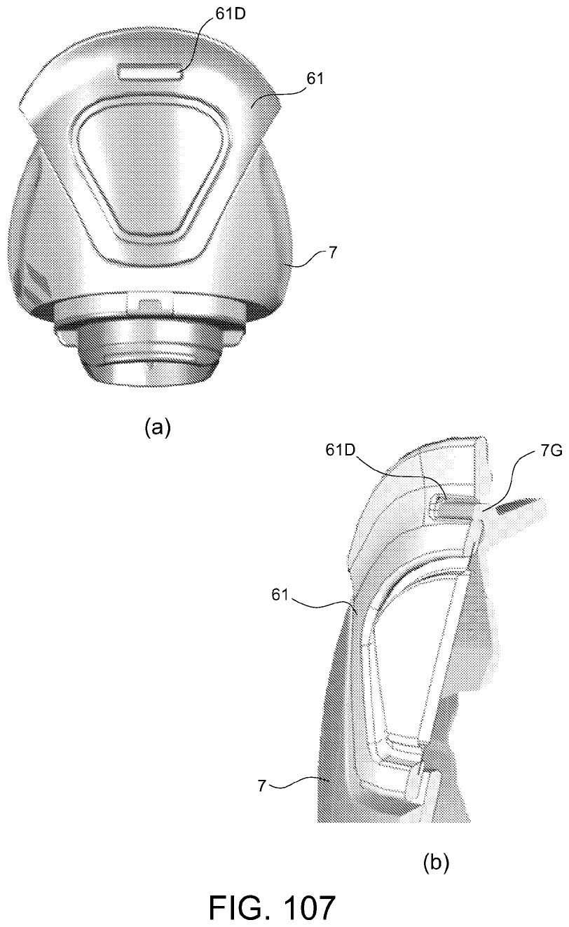

Referring now to FIG. 107, the recess 61D of the diffuser frame 61 may comprise a through recess 61D that extends through the diffuser frame 61 so as to comprise a small window, through which the user can see the protrusion 7G of the frame 3, when the protrusion 7G is received in the recess 61D. This provides the user with a visual indicator that the diffuser frame 61D is fully and correctly mounted on the frame 3 and inlet connector 7.

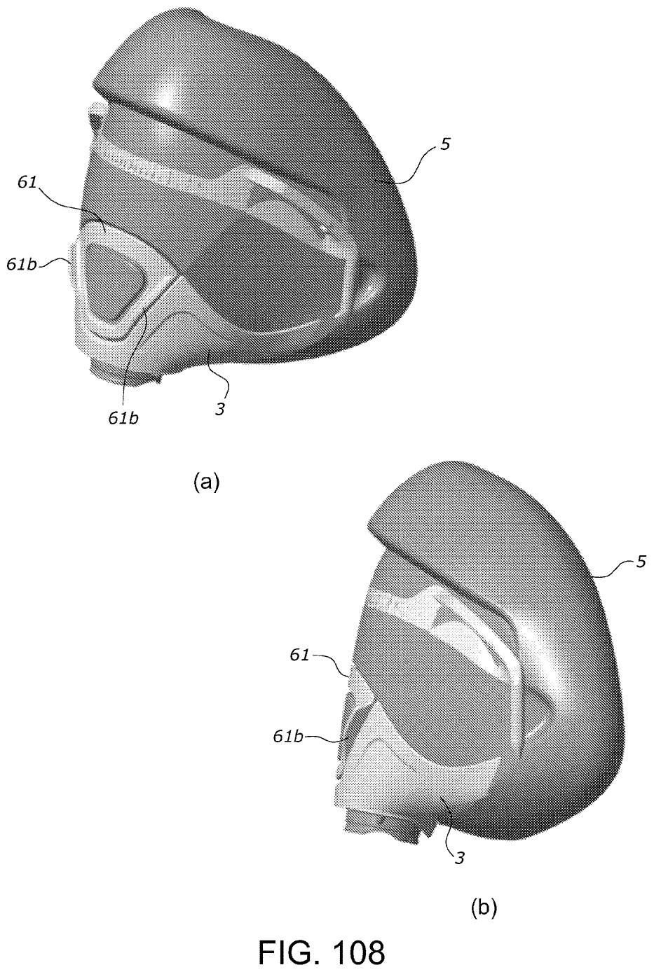

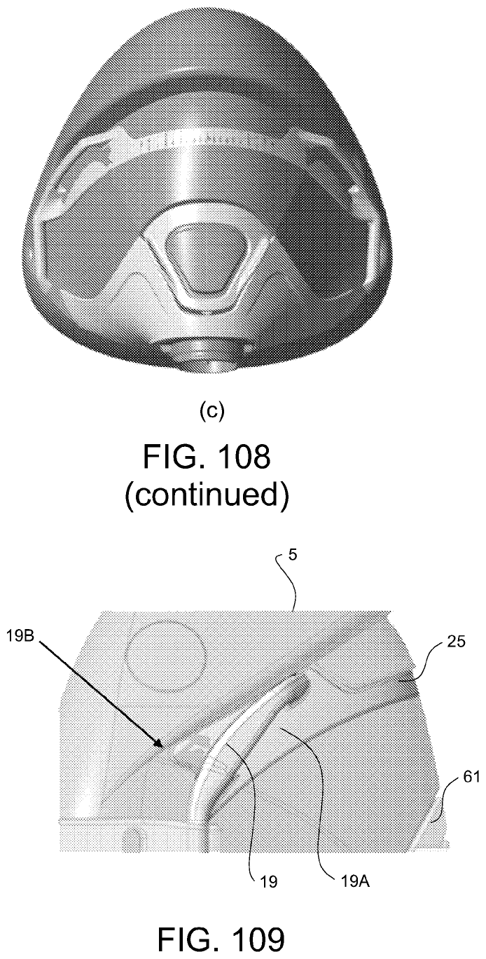

Referring now to FIG. 108, in this example the diffuser frame 61 comprises a pair of laterally opposed finger tabs 61B, each finger tab 61B extending along a respective side region of the diffuser frame 61B. In this example, the diffuser frame 61 may be mounted on one side of the frame 3 first, and then mounted on the other side of the frame 3.

Referring now to FIG. 109, the central region of the upper portion of frame 3 comprises a relatively thin and/or weakened structure, and the end regions comprise a relatively thick and/or reinforced structure at enlarged regions 19A. The enlarged regions 19A in this example are extended over those shown with reference to FIG. 5, to provide support ears 19B that extend generally rearwardly to provide additional support to the cushion 5 at or near the hinge of the rolling bridge region.

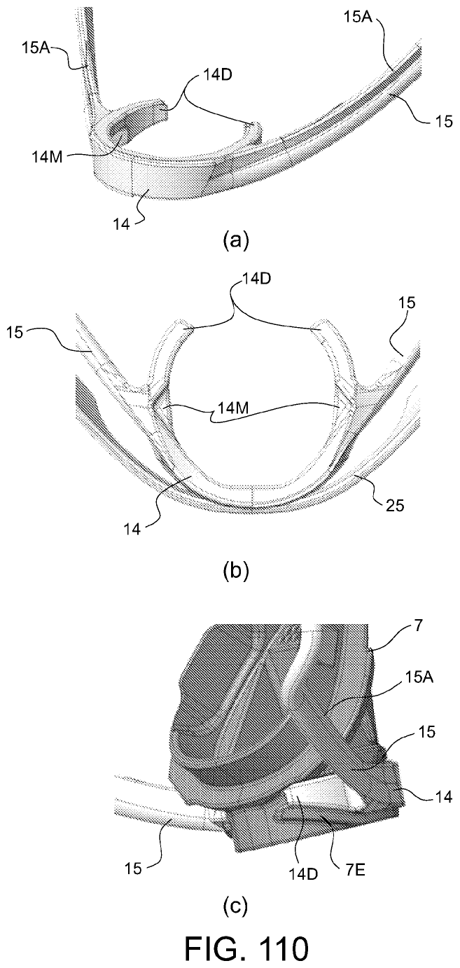

Referring to FIG. 110, frame 3 comprises a frame clip 14 similar to that of FIG. 80. In this example, the underside of the frame clip 14 is provided with a pair of opposed cut-outs 14m, at the region of the frame clip where the fingers 14d extend from the forward part of the frame clip 14. The cut-outs 14M prevent or minimise the occurrence of heat sink marks, that might otherwise occur from the moulding process.

Also referring to FIG. 110, the fingers 14d in this embodiment are tapered along their length, so that the distal ends of the fingers 14d are narrower, when viewed from the side, than the parts of the fingers 14d proximal the remainder of the frame clip 14. The skirt 7e is also tapered, to engage the tapered fingers 14e, the engagement resisting relative rotation between the inlet connector 7 and the frame clip 14 of the frame 3. The frame clip 14 therefore comprises one or more anti-rotation and/or guide surfaces configured to resist rotation of the frame 3 relative to the mask assembly.

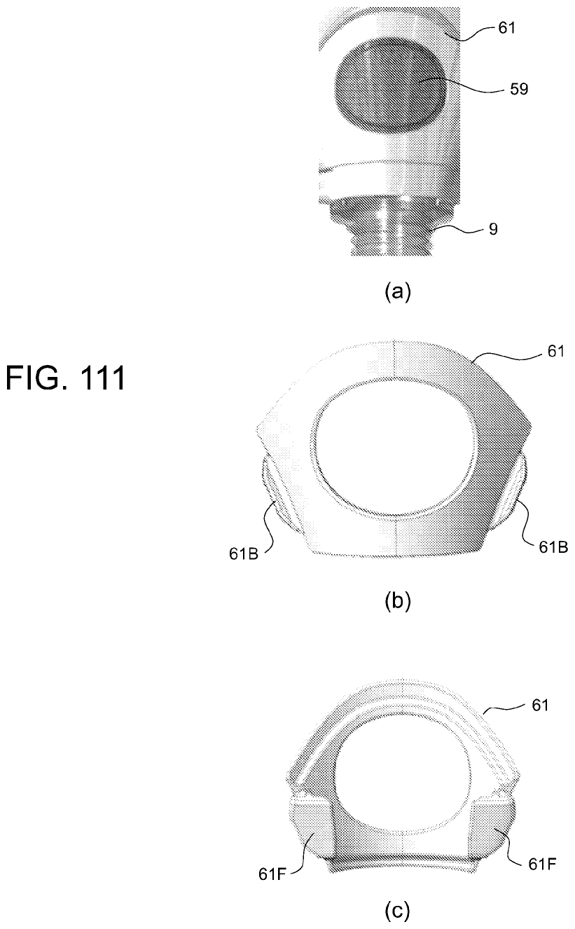

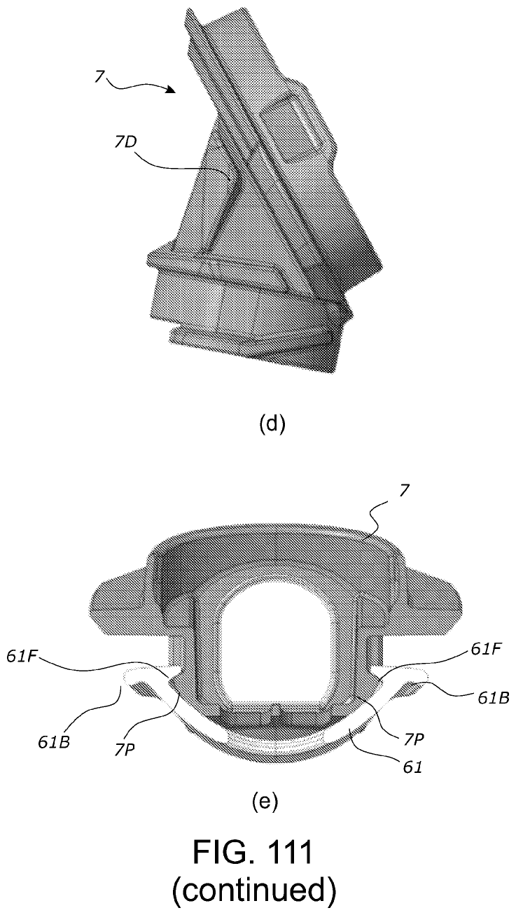

Referring now to FIG. 111, the diffuser 59 in this embodiment is substantially elliptical when viewed from the front, and is larger than the diffusers 59 described with reference to FIGS. 1 to 110. Similar to the diffuser frame 61 of FIG. 108, the diffuser frame 61 of FIG. 111 comprises a pair of laterally opposed finger tabs 61B, each finger tab 61B extending along a respective side region of the diffuser frame 61B. Adjacent each finger tab 61B, is a respective clip 61F that releasably engages respective protrusions 7P on the sides of the front of the inlet connector 7. Gripping each finger tab 61B enables the user to deform the frame clip 61 to disengage the clips 61F from the respective protrusions 7P.

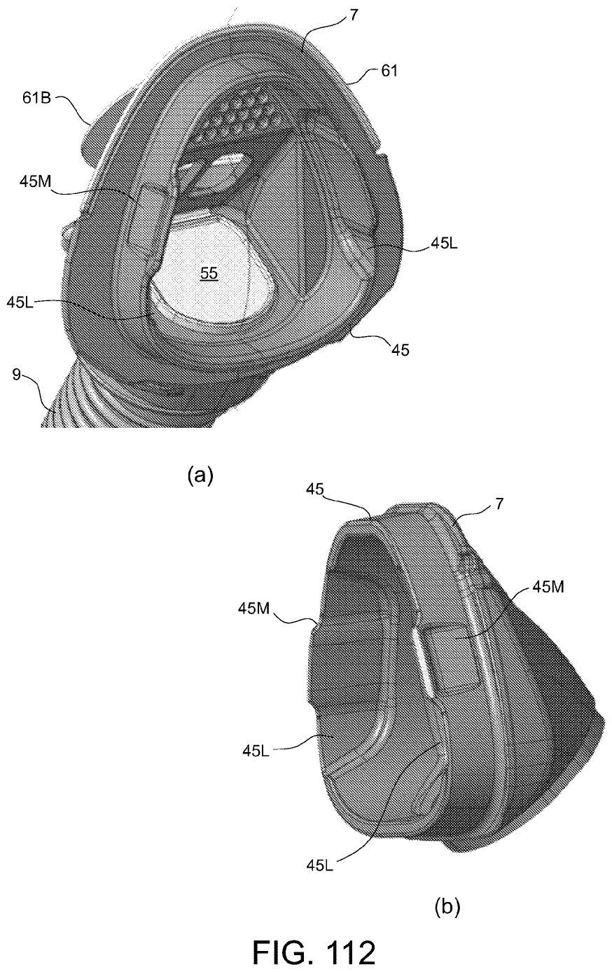

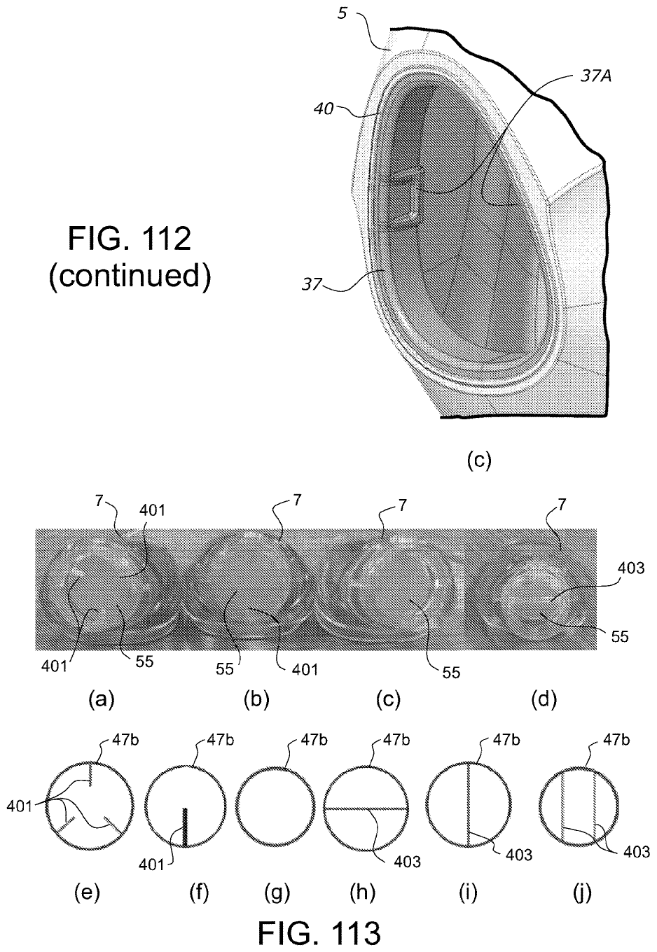

Referring to FIG. 112, a modified connection between the cushion 5 and the inlet connector 7 is shown. The outlet boss 45 of the inlet connector 7 comprises a pair of opposed thinned regions 45L on the internal surface of the outlet boss 45. These thinned regions 45L allow for increased flexibility of the outlet boss 45 to allow the outlet boss 45 to flex during connection to the cushion 5.

On the external surface of the outlet boss 47 are two cutouts 45M. These cutouts 45M receive respective protrusions 37A formed on the rigid ring 37 of the cushion 5, to connect the cushion 5 to the inlet connector 7. During assembly, the cutouts 45M deform radially inwardly to receive and engage with the protrusions 37A of the cushion 5 with an interference fit.

Referring now to FIG. 113, the inlet port 47b of AA valve 53 is provided with one or more anti-inversion features configured to resist the valve flap 55 from inverting through the inlet port 47b in the direction of the inlet conduit 9, i.e. to help ensure the valve flap 55 remains on or above the valve seat 255.

Each anti-inversion feature comprises at least one rib or lug that extends partially or fully across the inlet port 47b in a radial direction from the periphery of the inlet port 47b. In some cases the rib or lug extends through the central axis of the inlet port 47b. A plurality of ribs and/or lugs can be provided.

Examples of such anti-inversion features include:
a) FIG. 113a) and e) show an AA valve comprising three, equispaced lugs 401 that each extend from the periphery of the inlet port 47b towards the central axis of the inlet port 47b, each lug 401 terminating part way across the inlet port 47b.
b) FIG. 113b) and f) show an AA valve comprising a single lug 401 that extends from the periphery of the inlet port 47b towards the central axis of the inlet port, terminating at the central axis of the inlet port 47b.
c) FIG. 113c) and g) show an AA valve that does not comprise any anti-inversion feature.
d) FIG. 113d) and h) show an AA valve comprising a single rib 403 that extends all the way across the inlet port 47b from side to side, through the central axis of the inlet port 47b.
e) FIG. 113i) shows an AA valve comprising a single rib 403 that extends all the way across the inlet port 47b from front to back, through the central axis of the inlet port 47b.
f) FIG. 113j) shows a plurality of ribs 403, in this case a pair of ribs 403 that extend all the way across the inlet port 47b from front to back, each rib 403 being spaced from the other, and also spaced from the central axis of the inlet port 47b.

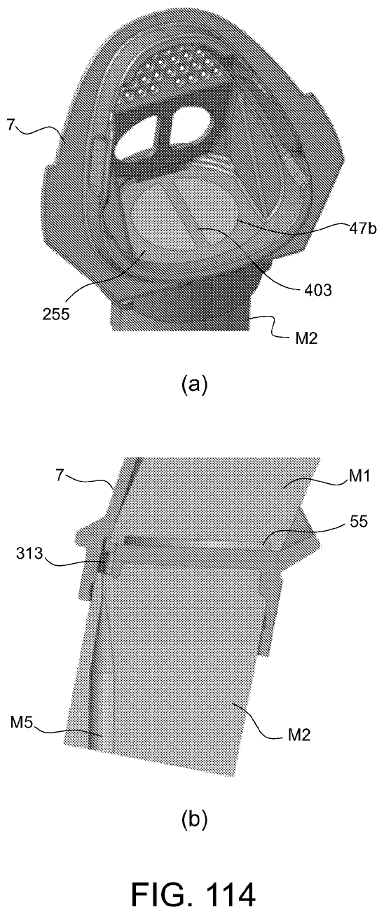

Referring now to FIG. 114 a single rib 403 is provided as per the example of FIG. 113i above. The rib 403 forms part of the surface against which the valve flap is moulded during overmoulding of the AA valve 53.

With reference to FIG. 114b) mould tool M can be used, comprising two mould halves M1, M2, as described above with reference to FIG. 99. The lower mould half M2 comprises a central recess that receives the rib 403 when the lower mould half M2 is inserted into the inlet connector 7 as part of the AA valve overmoulding process.

In this embodiment, the moulding material fills the AA valve cavity (defined between the upper and lower mould halves M1, M2) from the AA valve mount slot 313, as shown by the mould runner M5 formed in lower mould half M2.

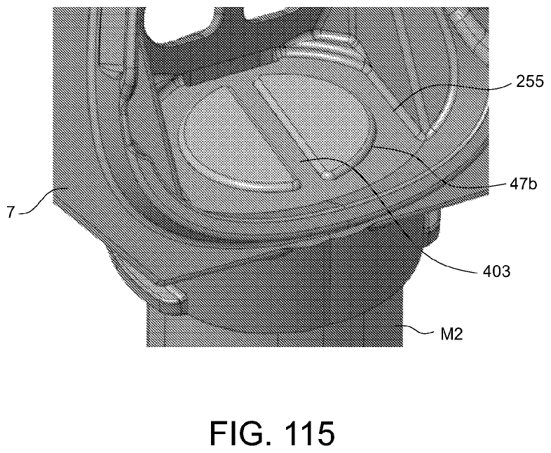

Referring to FIG. 115, a modified lower mould tool half M2 is configured to project through the inlet port 47b such that the upper surface of the mould half M2 projects above the valve seat 255 and rib 403. This can help prevent flash (extra material that may leak between adjacent surfaces) that could potentially stick the valve flap 55 to the valve seat 255 and prevent valve flap movement, or reduce or prevent the sealing of the valve flap 55.

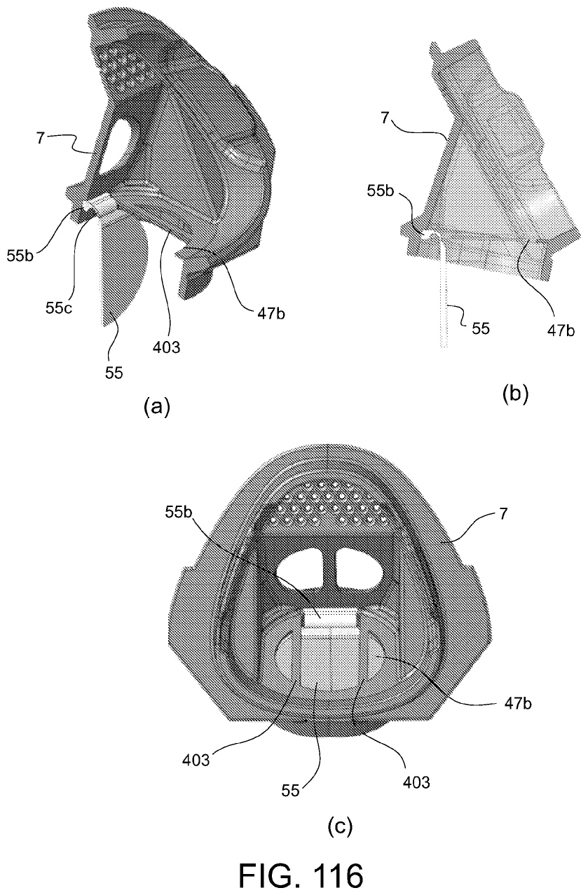

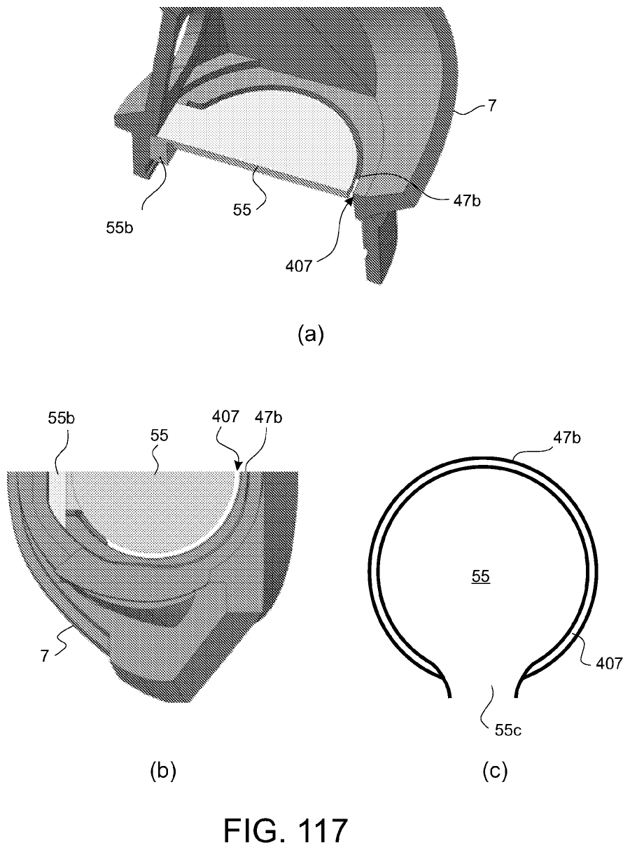

Referring to FIG. 116, a pair of spaced apart ribs 403 are provided, extending forward to back, similar to the embodiment of FIG. 113j).

In this case the AA valve 53 is overmoulded inside of the inlet 7, similarly to the process described above with reference to FIG. 99. In this embodiment, the lower mould tool half M2 forms the entire cavity for the valve flap 55 during the moulding process, the upper mould tool half M1 shutting off the valve mount 55B. Once moulded, the valve flap 55 is then pushed upwardly into the inlet connector 7, a described further below.

The valve flap 55 is moulded, in the inlet connector 7, to a pre-assembly condition in which the valve flap 55 hangs down below the valve mount 55, and in this example is moulded so as to be substantially orthogonal to the valve mount 55B, i.e. in a post-moulding condition at rest, the valve flap 55 hangs downwardly from the valve mount 55B. The valve flap 55 is therefore moulded so as to be biased or preloaded to this position.

Once moulded, the valve flap 55 initially hangs down below the valve seat 255. During assembly, the valve flap 55 is then pushed upwardly through the space between the two ribs 403 so that the valve flap 55 rests on top of the ribs 403 and is substantially aligned (i.e. co-planar) with the valve mount 55B. The valve flap 55 is preloaded as described above, so as to be biased downwardly against the ribs 403, so that the valve flap 55 is biased to rest on the ribs 403 and close the inlet port 47b, such that the valve flap 55 closes the inlet port 47b when there is no pressure differential in the inlet 7.

The valve 53 as described above with reference to FIG. 101 may have alternative shapes and/or configurations.

With reference to FIG. 117, in this embodiment the valve flap 55 is slightly smaller than the valve opening 47b, such that the valve flap 55 does not overlap with the valve seat 255 (other than at the valve mount 55B).

With reference to FIG. 117c), the periphery of the valve flap 55 is spaced from the valve opening 47b such that there is a small gap 407 between the two components, the gap 407 extending around the entire periphery of the valve flap 55 (other than at the valve mount 55B).

The valve flap 55 and the inlet port 47b may be substantially circular, with the valve flap 55 having a smaller diameter than the diameter of the inlet port 47b.

Gap 407 allows for the two mould tool halves M1, M2 to completely form the cavity in which the valve flap 55 is moulded, ensuring that any flash from the moulding process does not stick the valve flap 55 to the inlet connector 7 and become stuck in a closed position. The valve flap 55 is also unable to get stuck in an inverted position in this embodiment as the valve flap 55 is smaller than the inlet port 47b.

The valve flap 55 may be substantially planar: the upper and lower rims 55E, 55F may be omitted.

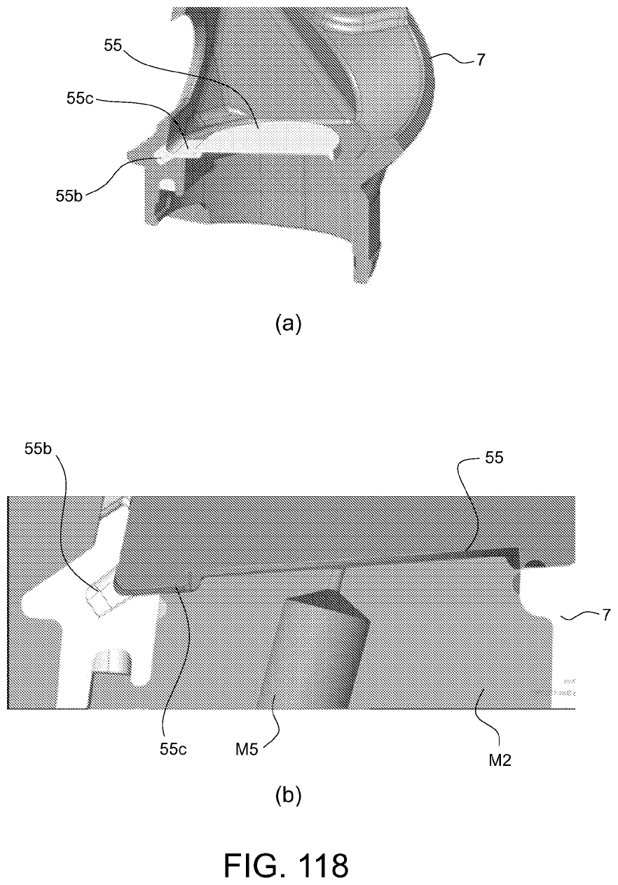

With reference to FIG. 118, in this embodiment the valve flap 55 is the same size as the valve opening 47b, such that the valve flap 55 does not overlap with the valve seat 255 (other than at the valve mount 55B).

The periphery of the valve flap 55 is not spaced from the valve opening 47b, such that there is no gap between the two components.

The valve flap 55 and the inlet port 47b may be substantially circular, with the valve flap 55 having substantially the same diameter as the inlet port 47b.

This configuration minimises leak when the AA valve 53 is closed, and means that the valve flap 55 cannot get stuck in an inverted position (by being pushed downwards towards the conduit 9).

The valve flap 55 may comprise a lower rim 55F.

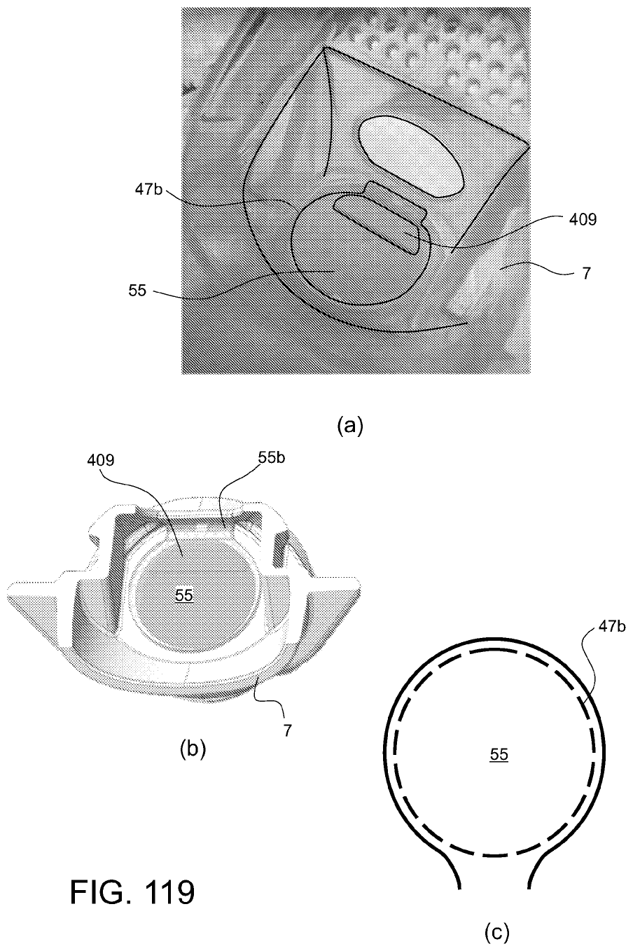

With reference to FIG. 119, in this embodiment the valve flap 55 is bigger than the valve opening 47b, such that the periphery of the valve flap 55 overlaps with the valve seat 255 around the inlet port 47b (including at the valve mount 55B).

With reference to FIG. 119c), the periphery of the valve flap 55 extends onto the valve seat 255, around the periphery of the valve opening 47b, such that there is no gap between the two components. The periphery of the valve flap 55 therefore overlaps the valve seat 255 around the entire periphery of the valve flap 55.

The valve flap 55 and the inlet port 47b may be substantially circular, with the valve flap 55 having a larger diameter than the inlet port 47b.

This configuration assists in preventing the valve flap 55 from inverting under normal CPAP and breathing pressures, without an anti-inversion feature (such as lugs 401 or ribs 403 as described above).

In this embodiment, the valve flap 55 may not have upper or lower rims 55E, 55F.

With reference to FIG. 119 a) and b), valve flap 55 comprises a reduced thickness region 409 near the valve mount 55B, the reduced thickness region 409 being semi-circular, and adjacent the valve mount 55B in this example. The reduced thickness region 409 reduces the weight of the valve flap 55, to facilitate the valve flap 55 moving as required at the desired pressure differentials in the inlet connector 7. The reduced thickness region 409 can provide more flexibility close to the valve mount 55B, to enable the valve flap 55 to pivot between the required positions.

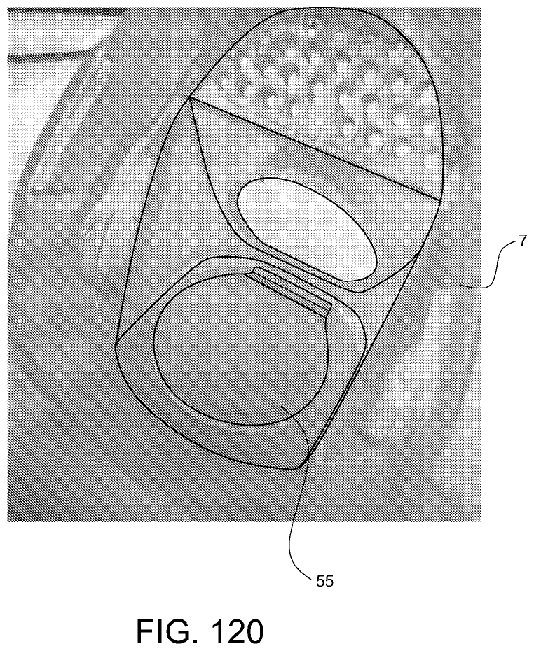

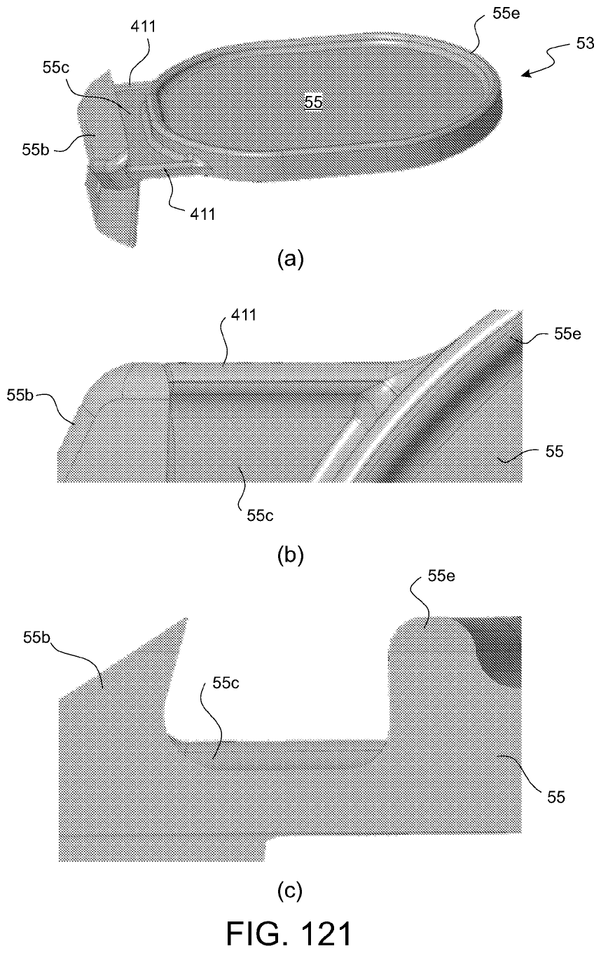

With reference to FIG. 120, an AA valve 53 is provided with features similar to those of the AA valve 53 of FIG. 119. In this embodiment, the reduced thickness region 409 is omitted. The valve flap 55 comprises a lower peripheral rim 55F.

Referring to FIG. 121, an AA valve 53 comprises similar features to those described with reference to FIG. 100 wherein AA valve 53 comprises a valve flap 55 connected to the valve mount 55B by a thinned region 55C.

In this embodiment, thinned region 55C is modified to include a reinforcing wall 411 along each side margin, each reinforcing wall 411 extending along a respective side margin of the thinned region 55C between the valve flap 55 and the valve mount 55B.

The reinforcing walls 411 can reduce or prevent tearing of the thinned region 55C, whilst the relatively thin cross section of the remainder of the thinned region 55C still retains sufficient flexibility between the valve flap 55 and the valve mount 55B to allow the valve flap 55 to pivot between the required positions.

The thickness of the thinned region 55C, in cross section, may be between 25 and 75% of the height of the reinforcing walls 411, and preferably between 35 and 55%. The reinforcing walls 411, in one example, may be about 0.5 mm high, and the remainder of the thinned region 55C about 0.2 mm thick.

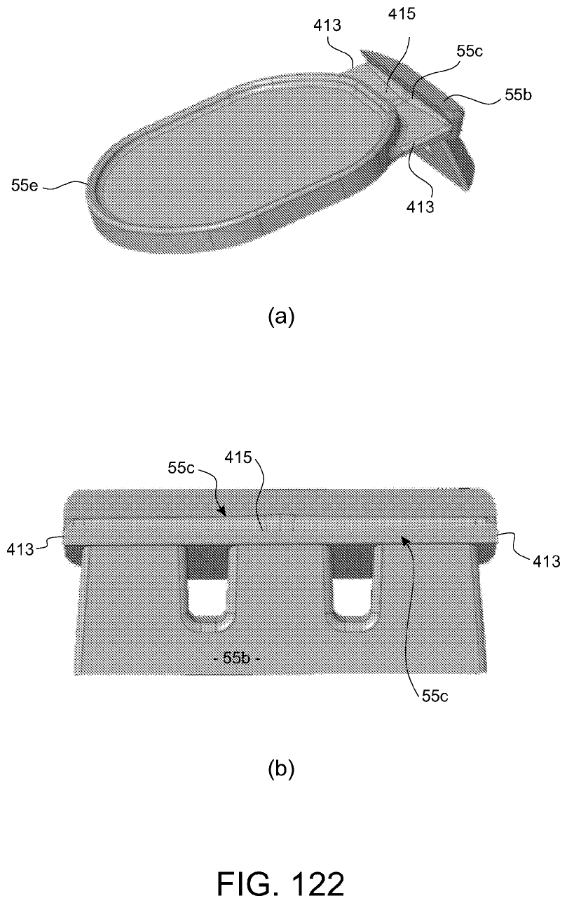

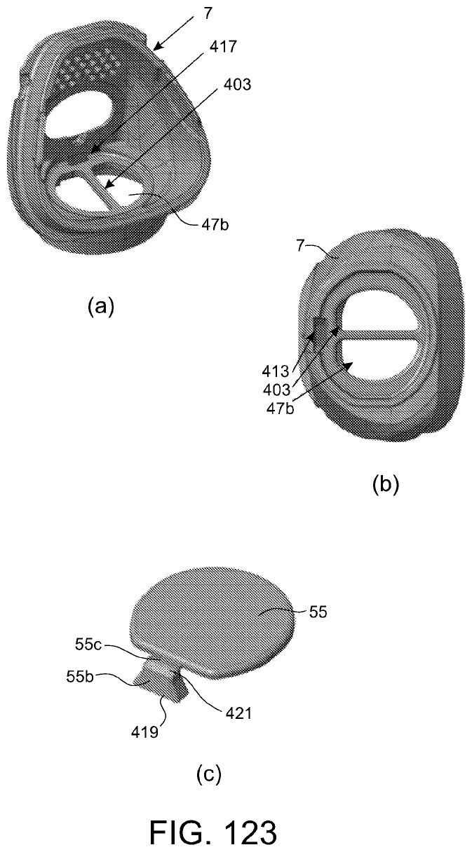

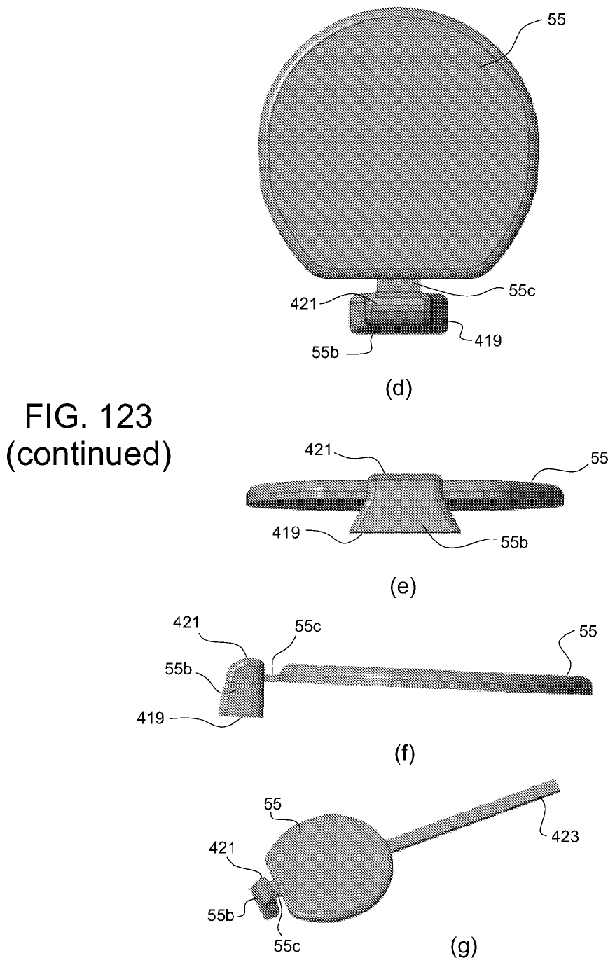

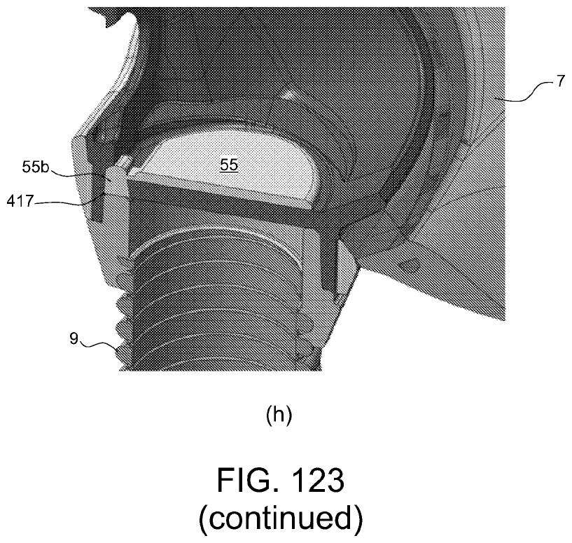

Referring to FIG. 122, an AA valve 53 comprises similar features to those described with reference to FIG. 100.

AA valve 53 comprises a thinned region 55C having a non-constant thickness across its width, that is, between the opposed side margins 413 of the thinned region 55C in a direction parallel with the pivot axis of the valve flap 55. The thinned region 55C is thicker proximal each side margin 413, and thinner at a central region 415 distal the side margins 413.

Referring to FIG. 122, the thinned region 55C, when viewed along the valve flap 55 in a direction perpendicular to the pivot axis of the valve flap 55, tapers from the side margins 413 towards the central region 415, such that the thinned region 55C is thicker at the side margins 413 and thinner between the side margins 413.

The thicker material helps prevent tears, while the thinner central region 415 allows sufficient flexibility for the valve flap 55 to open and close.

The following embodiments provide AA valves 53 that are formed separately, prior to mounting in the inlet connector 7, without requiring the AA valve 53 to be overmoulded inside the inlet connector 7.

Referring to FIG. 123, the AA valve 53 in this embodiment, during assembly, is pulled through a slot 417 in the inlet connector 7, to mount the AA valve 53 in the inlet connector 7.

Referring to FIG. 123 a), b), the slot 417 is tapered so as to become narrower in a direction along the axis of the inlet port 47b. The slot 417 becomes narrower along an insertion direction of the AA valve 53.

The inlet connector 7 comprises an anti-inversion feature comprising a single rib 403 that extends across the centre of the inlet opening 47b from front to rear.

The valve mount 55B is generally tapered as can be seen in FIG. 123 c) to f), such that the base 419 of the valve mount 55B is larger than the top 421 of the valve mount 55B. One or more of the sides and/or front and/or rear faces of the valve mount 55B may be inclined to provide the tapered shape.

Referring to FIG. 123g), the AA valve 53 is moulded with an elongate pull tab 423 which extends from the valve flap 55, distal from the valve mount 55B.

To mount the AA valve 53 on the inlet connector 7, the pull tab 423 is inserted through the slot 417 so that the end of the pull tab 423 projects through the slot 417 into the inlet connector 7. The end of the pull tab 423 can then be pulled, so that the remainder of the AA valve 53 is pulled through the slot 417 into the inlet connector 7.

Continued pulling on the pull tab 423 pulls the valve mount 55B into the slot 417. The tapered exterior shape of the valve mount 55B mates with the tapered interior shape of the slot 417 such that the valve mount 55B engages with the slot 417 to prevent any further movement of the AA valve 53 through the slot 417. The valve flap 55 is inside the inlet connector 7 and can rest on the valve seat 255 and rib 403. The pull tab 423 can then be trimmed off.

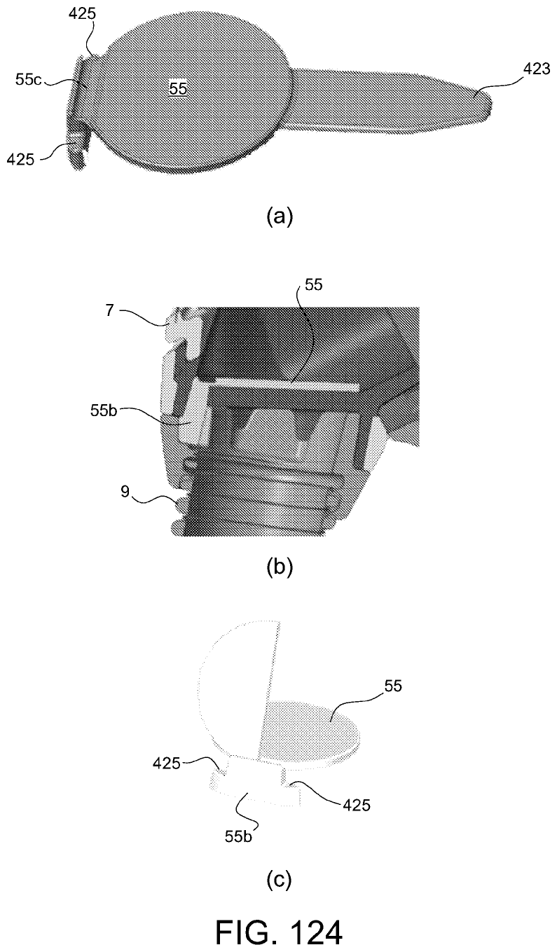

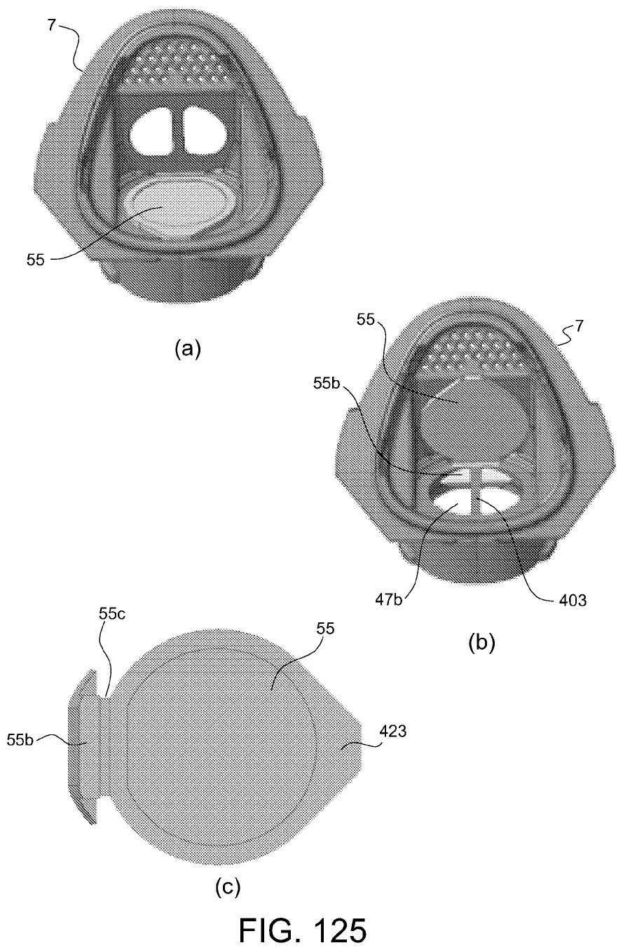

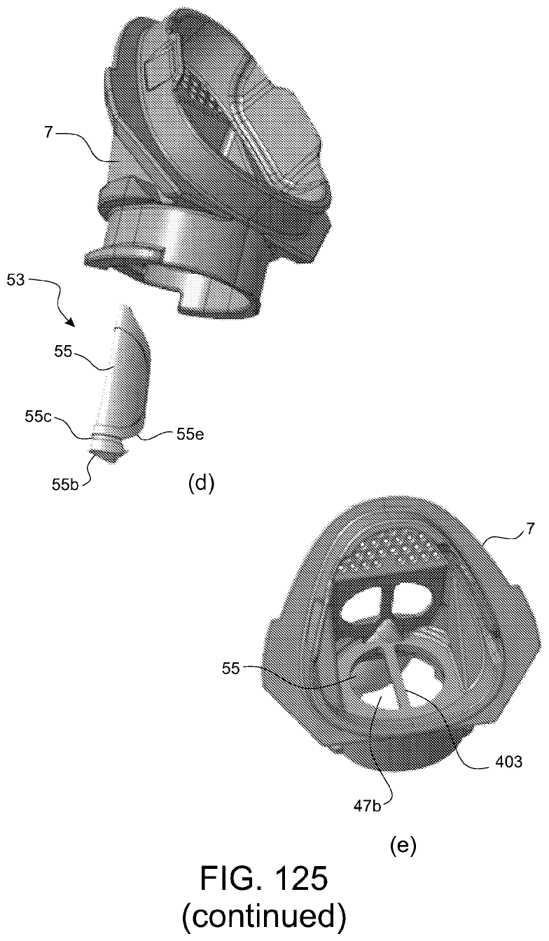

With reference to FIG. 124, a similar embodiment to that of FIG. 123 is shown, wherein the AA valve 53 comprises a wider valve mount 55B, and a wider pull tab 423, when the AA valve 53 is viewed from above.

In this embodiment, the valve mount 55B comprise a base 419 that is wider than the remainder of the valve mount 55B to comprise steps 425. The base 419 of the valve mount 55B is wider than the narrowest part of the slot 417 in the inlet connector 7, such that the steps 425 abut against corresponding surfaces inside the slot 417, to limit movement of the AA valve 53 through the slot 417 during assembly. The wider pull tab 423 may be more resistant to tearing during assembly.

Referring to FIG. 125, in this embodiment, the AA valve 53 comprises a short pull tab 423 formed by an extended tip of the valve flap 55.

With reference to FIG. 125 d) and e), the AA valve 53 is assembled onto the inlet connector 7 by folding the AA valve 53, and inserting the folded AA valve 53 through the slot 417 in the inlet connector 7. Once the pull tab 423 is exposed through the slot 417, the pull tab 423 can be gripped and pulled, to pull the AA valve 53 through the slot 417 until the valve mount 55B is fully received in and retained by the slot 417. The remainder of the valve flap 53 unfolds when inside the inlet connector 7 such that the valve flap 55 rests on the valve seat 255 and rib 403, closing the inlet port 47b. In this embodiment the pull tab 423 does not need to be trimmed off after assembly.

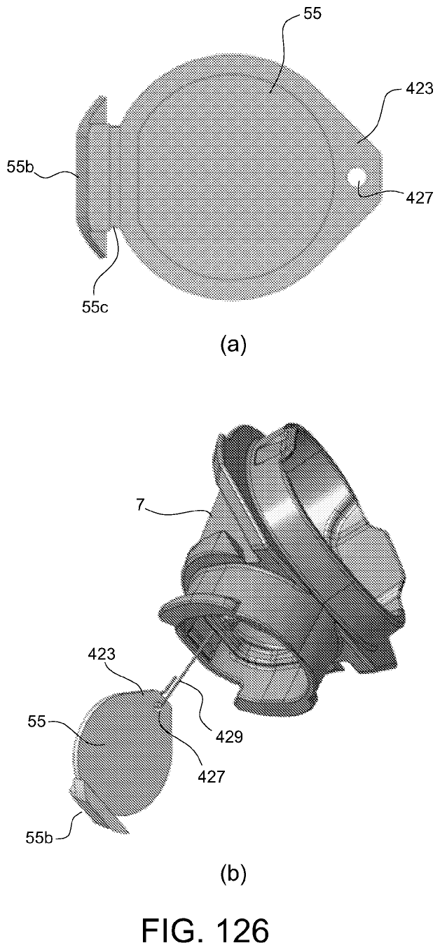

Referring to FIG. 126, this embodiment is similar to that of FIG. 125 except that the pull tab 423 comprises an aperture 427. During assembly a mounting hook tool 429 is inserted into the aperture 427, the hook tool 429 being used to pull the AA valve 53 through the slot 417 into the inlet connector 7. Once the AA valve 53 is fully received in the inlet connector 7, the hook tool 429 can be removed from the aperture 427.

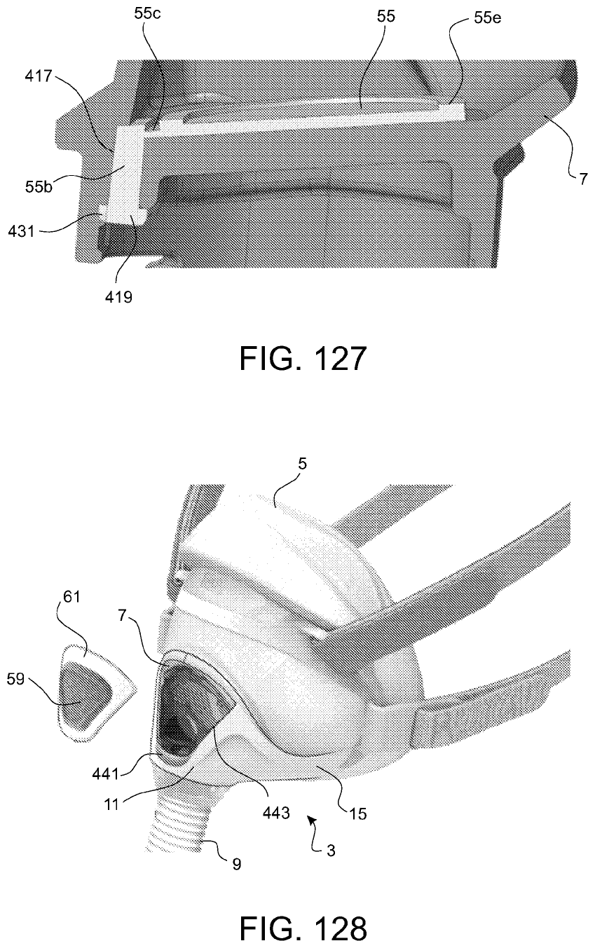

Referring to FIG. 127, the AA valve 53 in this embodiment, comprises a valve mount 55B which is barbed 431 at the base 419 of the valve mount 55B, distal from the pivot of the AA valve 53. In this embodiment, the valve mount 55B is inserted into the slot 417 from inside the inlet connector 7, that is, in a generally downward direction away from the outlet boss 45, towards the inlet conduit 9. The valve mount 55B is pushed into the slot 417, with the barb 431 deforming and compressing during movement through the slot 417, and then expanding once the mount 55B is fully inserted into the slot 417. The barb 431 extends outside the slot 417 and engages with the inlet connector 7 around the slot 417, to help prevent the valve mount 55B from being removed from the slot 417.

An upper and/or lower rim 55E/55F can be optionally provided on any of the valve flap 55 embodiments described above.

Referring to FIG. 128, the diffuser frame 61 in this example comprises connection features, to connect the diffuser frame 61 to the frame 3, that are located centrally at the top and bottom of the diffuser frame 61, that is, in a position that corresponds to the likely location of a force applied by the user to release the diffuser frame 61 from the frame 3. These connection features may be similar to those described above with reference to FIG. 106.

In this embodiment, a scalloped gripping region 441 is provided on the central portion 11 of the frame 3, adjacent the lower margin of the diffuser frame 61. The gripping region 441 is recessed to allow a user's finger to be inserted into the region 441 and partially behind the lower margin of the diffuser frame 61, to lever the lower margin of the diffuser frame 61 away from frame 3, to remove the diffuser frame 61 from the frame 3.

The side and upper margins of the diffuser frame 61 are shaped and sized to fit into a correspondingly shaped diffuser pocket 443 formed in the central portion 11 of the frame 3.

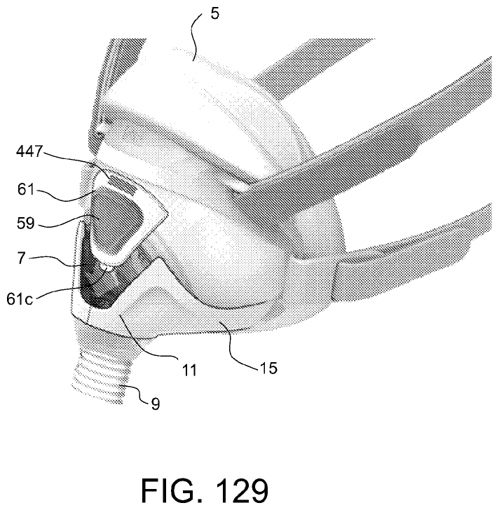

Referring to FIG. 129, the diffuser frame 61 comprises connection features similar to those described above with reference to FIG. 106. These connection features include a lower, central mounting tongue 61C that is received in a lower portion of the frame 3.

Diffuser frame 61 in this embodiment is configured to be slidingly mounted on the central region of frame 3 by sliding the diffuser frame 61 downwardly on the frame 3 so that the tongue 61C is received in the central portion 11 of the frame 3.

The side margins 61G of the diffuser frame 61 may be configured to engage with corresponding side margins 445 of the diffuser pocket 443 into which the diffuser frame 61 is received. For example, the diffuser frame side margins 61G may comprise one or more tabs or fingers (not shown) that slidingly engage the rear surface of the side margins 445 of the diffuser pocket 443.

An upper portion of the diffuser frame 61 is provided with a gripping portion 447 to assist in allowing the user to move the diffuser frame 61 relative to frame 3.

The gripping portion 447 may comprise one or more horizontal ridges, or other protrusions, that protrude from the front surface of the diffuser frame 61.

As discussed above, it may be desirable for the frame clip 14 and/or inlet connector 7 to comprise anti-rotation features to resist rotation of the frame 3 relative to the inlet connector 7 and/or cushion 5.

It may be desirable that the frame 3 is not unintentionally disconnected when the frame 3 is rotated up or down, for example due to tensile forces from the headgear, or movement of the mask assembly 1 by the user.

The following embodiments comprise features on the frame clip 14, inlet connector 7 and/or conduit connector cuff 9A that prevent or reduce rotation of the frame and/or unintentional disconnection.

As described above with reference to FIG. 71, the frame 3 and the inlet connector 7 may be provided with engagement features that engage with one another when the frame 3 is mounted on the inlet connector 7. When engaged, the engagement features help retain the inlet connector 7 on the frame 3, and help resist relative rotation between the inlet connector 7 and the frame 3. In the embodiment of FIG. 71, the frame 3 comprises a female engagement feature 183 that receives a male engagement feature in the form of a lug 185 on the inlet connector 7. In that embodiment the engagement features are positioned laterally of the central portion 11 of the frame 3.

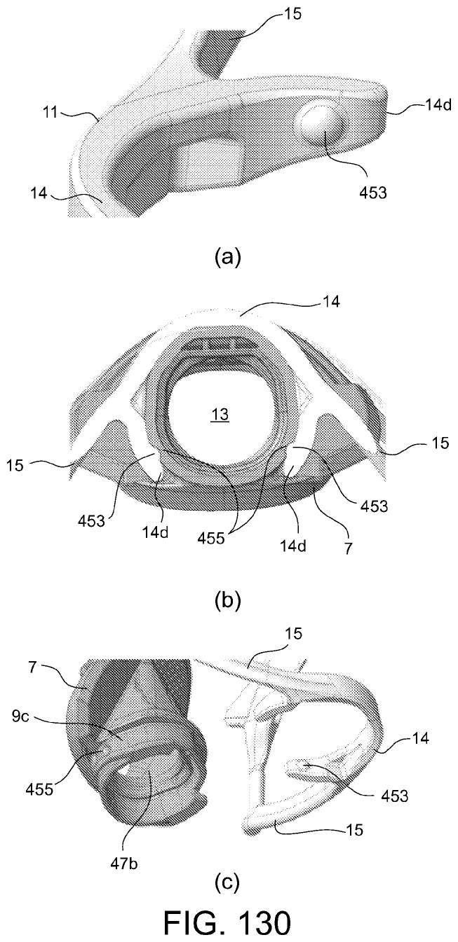

With reference to FIG. 130, the frame 3 in this embodiment comprises male engagement features 453, and the inlet connector 7 comprises female engagement features 455.

The male engagement features 453 each comprise a part spherical bump, one bump 453 being provided on the inside of each finger 14d of the frame clip 14.

The female engagement features 455 each comprise a part spherical dimple sized and shaped to correspond to and to receive a respective bump 453. The indents 455 are provided towards the rear of the recess 9c on the inlet connector 7 that receives the mask frame clip 13, distal from the forwardmost portion of the inlet connector 7.

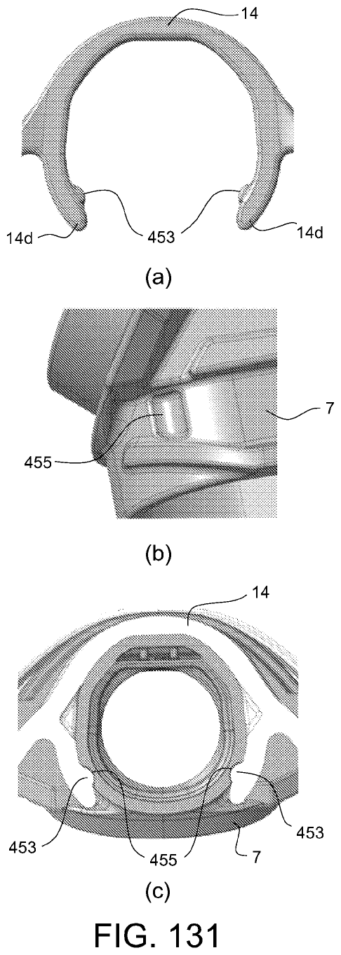

With reference to FIG. 131, a similar embodiment is shown, but in which the bumps 453 and indents 455 are part cylindrical.

With reference to FIG. 110, above, we described a frame clip 14 similar to that of FIG. 80 wherein the underside of the frame clip 14 is provided with a pair of opposed cut-outs 14m, at the region of the frame clip 14 where the fingers 14d extend from the forward part of the frame clip 14.

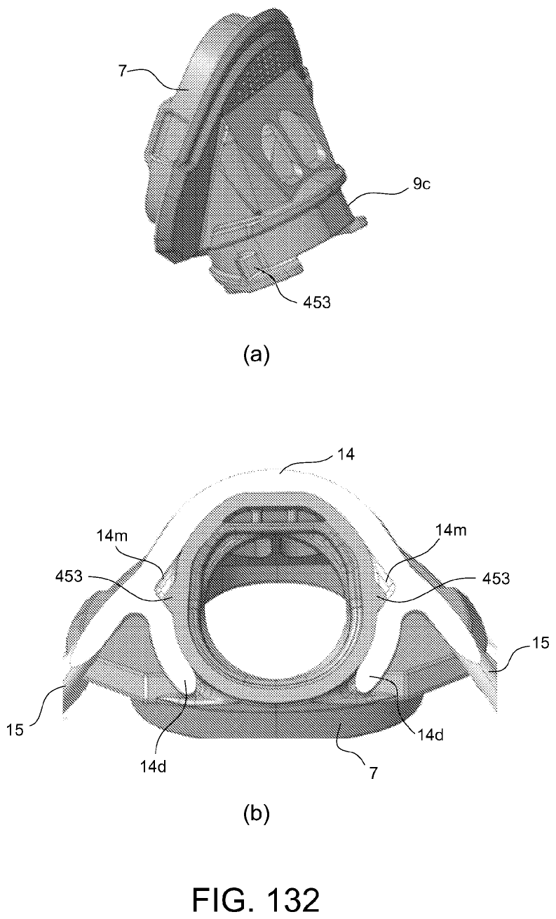

With reference to FIG. 132, in this embodiment, the recess 9c of the inlet connector 7 comprises a pair of protruding bumps 453, on opposed sides of the recess 9c, that are each received in the respective cut-outs 14m of the frame clip 14.

Engagement of the bumps 453 with the cut-outs 14m helps prevent relative rotation between the frame clip 14 and inlet connector 7.

In this embodiment, each bump 453 comprises forward and rearward surfaces, only the rearward surfaces engaging the cut-outs 14m. In this embodiment the shape of the cut-outs 14m, when viewed in a direction aligned with the axis of the inlet port 47b partially corresponds to the shape of the bumps 453.

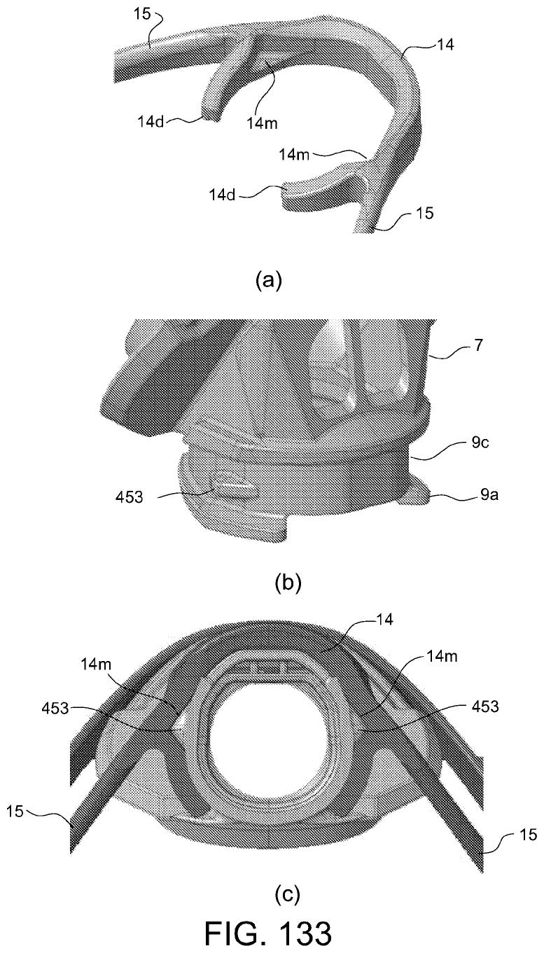

With reference to FIG. 133, an inlet connector 7 and frame clip 14 are provided which are similar to those of FIG. 132 above. However, the forward and rear surfaces of the bumps 453 both engage a respective cut-out 14m, when the frame clip 14 is connected to the inlet connector 7. In this embodiment the shape of the cut-outs 14m, when viewed in a direction aligned with the axis of the inlet port 47b corresponds to the shape of the bumps 453.

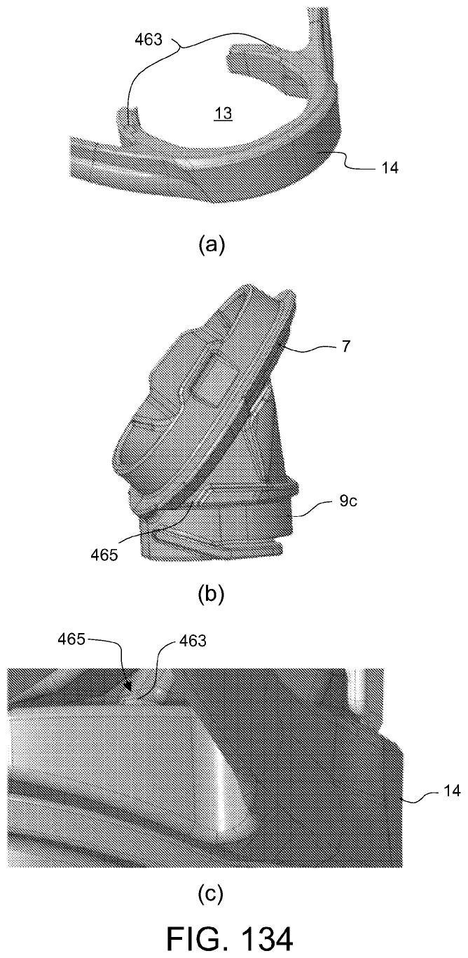

With reference to FIG. 134, male and female engagement features are again provided. However, in this embodiment, the frame clip 14 comprises male engagement features comprising bumps 463 on an upper surface of the frame clip 14. The bumps 463 extend in a direction aligned with the axis of the opening 13. In this example a pair of bumps 463 are provided, at opposite sides of the frame clip 14. Corresponding cut-outs 465 are provided on the inlet connector 7, on a downwardly directed surfaces of the recess 9c of the inlet connector 7. The cut-outs 465 extend in a direction aligned with the axis of the inlet port 47b. Engagement of the bumps 463 with the cut-outs 465 increases the force required to disconnect the frame 3 from the inlet connector 7.

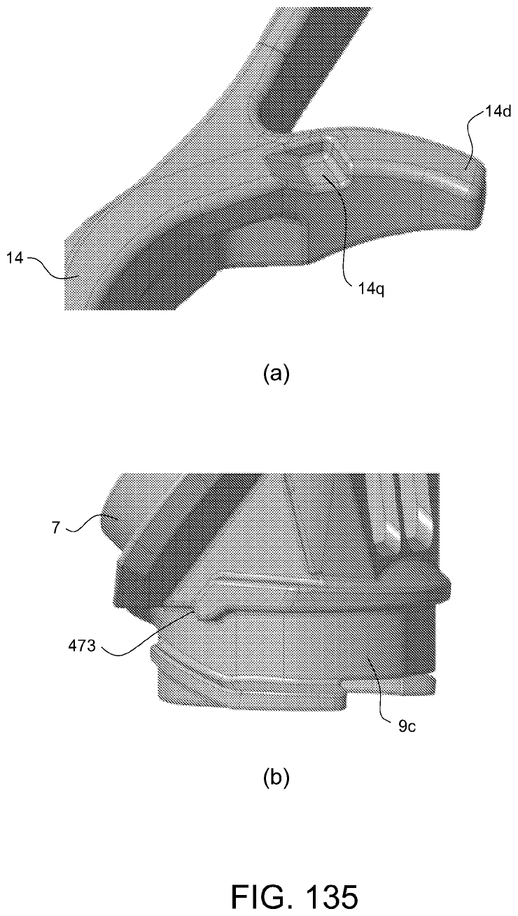

With reference to FIG. 135, a similar embodiment is shown to that of FIG. 134 except that in this embodiment an upper surface of the recess 9c of the inlet connector 7 comprises downwardly directed bumps 473, and the upper surface of the frame clip 14 comprises cut-outs 14q.

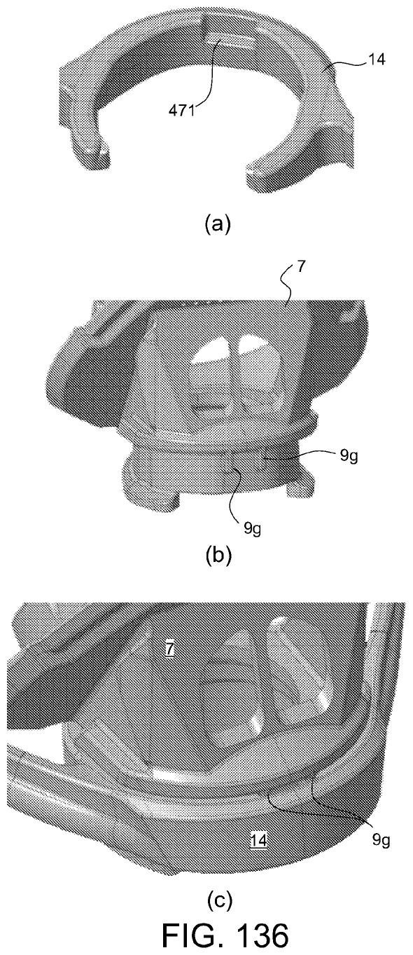

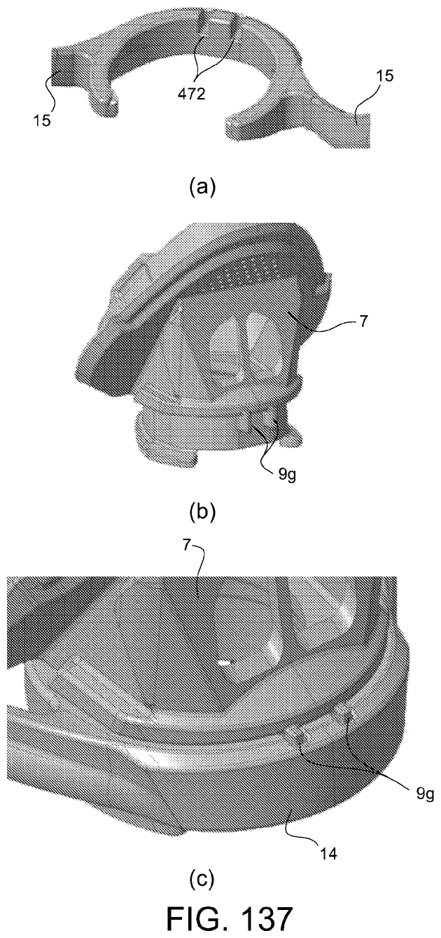

With reference to FIG. 136, a frame clip 14 is provided comprising an oblong indentation 471 at a central forward portion of the frame clip 14, on a rear surface, so as to be exposed to the opening 13. A forward portion of the recess 9c of the inlet connector 7 comprises a pair of vertical ribs 9g that project out from the recess 9c, the ribs 9g extending in a direction parallel with the axis of the inlet port 47b. When the frame clip 14 is connected to the inlet connector 7, the ribs 9g are received in the indentation 471 and engage with the sides of the indentation 471 to resist rotation of the frame clip 14 relative to the inlet connector 7.

With reference to FIG. 137 a similar embodiment is shown to that of FIG. 136. The single oblong indentation 471 instead comprises a pair of smaller oblong indentations 472, each slightly larger than a rib 9g such that when the frame clip 14 is connected to the inlet connector 7, a rib 9g is received in a respective indentation 472.

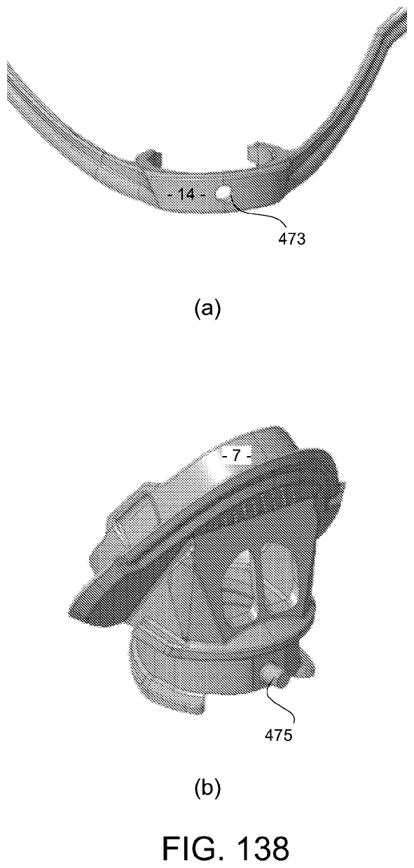

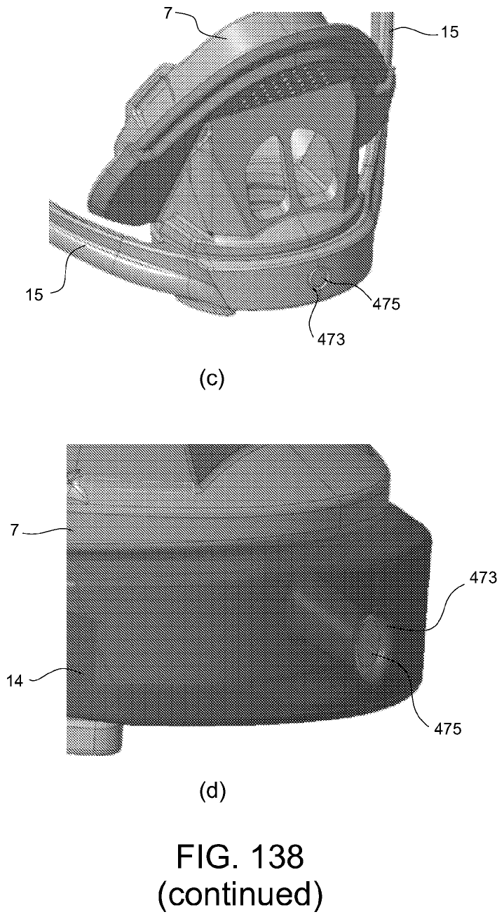

With reference to FIG. 138a) to c), a central forward part of the frame clip 14 comprises an aperture 473 configured to receive a forwardly projecting central post 475 of the recess 9c of the inlet connector 7. When the post 475 is received in the aperture 473, the two features engage and resist relative side to side rotation between the frame clip 14 and the inlet connector 7 about the axis of the inlet port 47b. The aperture 473 and post 475 also resist relative front to back rotation between the frame clip 14 and the inlet connector 7 about a transverse axis that is perpendicular to the axis of the inlet port 47b.

With reference to FIG. 138d) the post 475 and the axis of the aperture 473 are inclined, and are downwardly inclined from back to front of the mask assembly in this example.

With reference to FIG. 77 above, a lower part of the inlet connector 7 comprises a skirt 7e that extends downwardly over the front and sides of the conduit connector cuff 9a. The skirt 7e comprises a recess 7f configured to received and engage with the frame clip 14 of the mask frame 3, and a guide flange 7g that projects outwardly from the skirt 7e below the recess 7f.

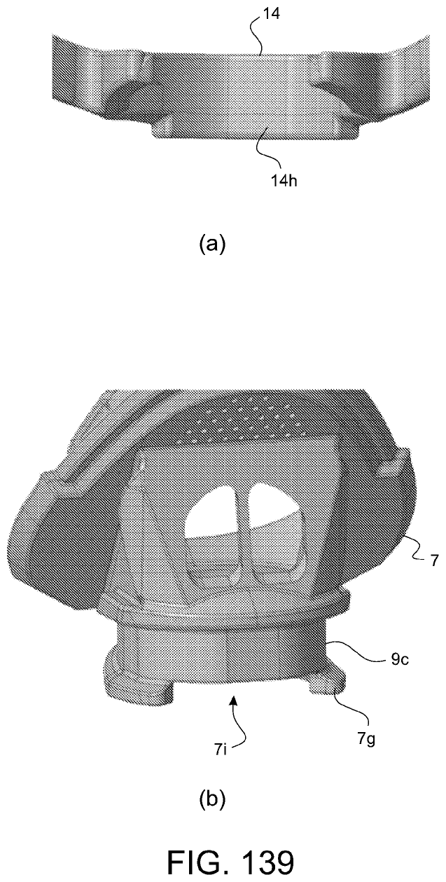

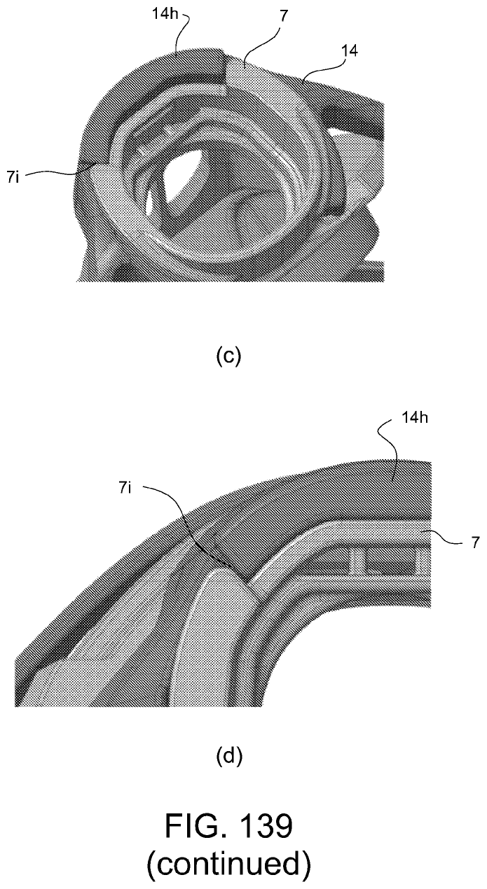

With reference to FIG. 139b), an inlet connector similar to that of FIG. 77 is provided in which a central, forward portion of the guide flange 7g is omitted to define a flange cut-out 7i. With reference to FIG. 139a), a central, lower portion of the frame clip 14 comprises a downwardly directed elongate tab 14h that projects away from the frame clip 14 in a direction along the axis of the opening 13.

With reference to FIG. 139c) and d), the tab 14h is received in the flange cut-out 7i, when the frame clip 14 is mounted on the inlet connector 7. The tab 14h engages the cut-out 7i to resist relative rotation.

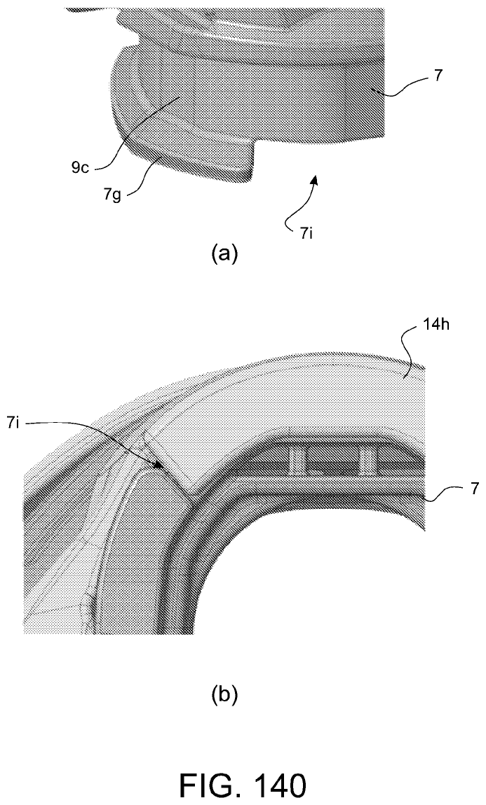

With reference to FIG. 140, a similar embodiment is shown, in which the guide flange 7g extends further radially outwardly from the inlet connector 7, to provide a greater contact area between the flange 7g and the tab 14h.

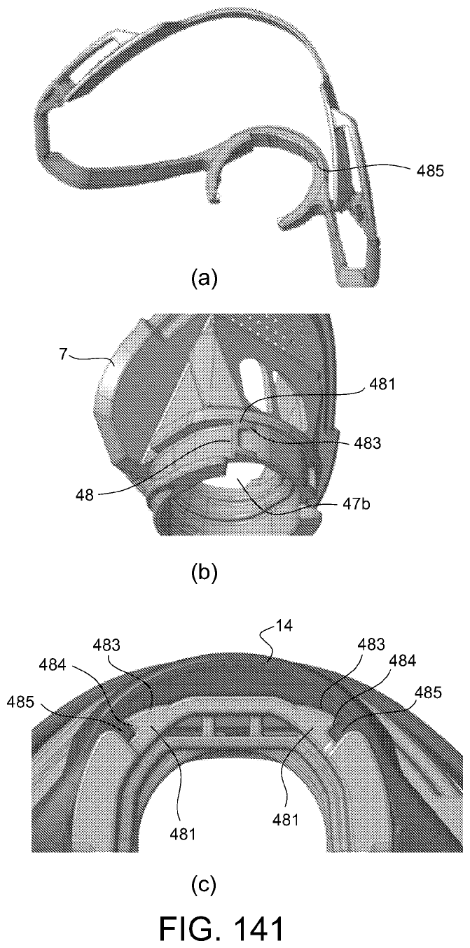

With reference to FIG. 141, a forward portion of the recess 9c of inlet connector 7 comprises a pair of outwardly extending lugs 481, each lug 481 comprises a curved forward guide surface 483, and a straight, rearward engagement surface 483. A forward, inner surface of frame clip 14 comprises a pair of radially inwardly directed projections 485. As the frame clip 14 is pushed onto the inlet connector 7, the projections 485 slide over the guide surfaces 483, and the frame clip 14 deforms outwardly. Further movement of the frame clip 14 onto the inlet connector 7 moves the projections 485 to a position adjacent a respective engagement surface 483, the frame clip 14 reverting to its original shape. The projections 485 then engage a respective engagement surface 483 to resist relative rotation of the frame clip 14 relative to the inlet connector 7.

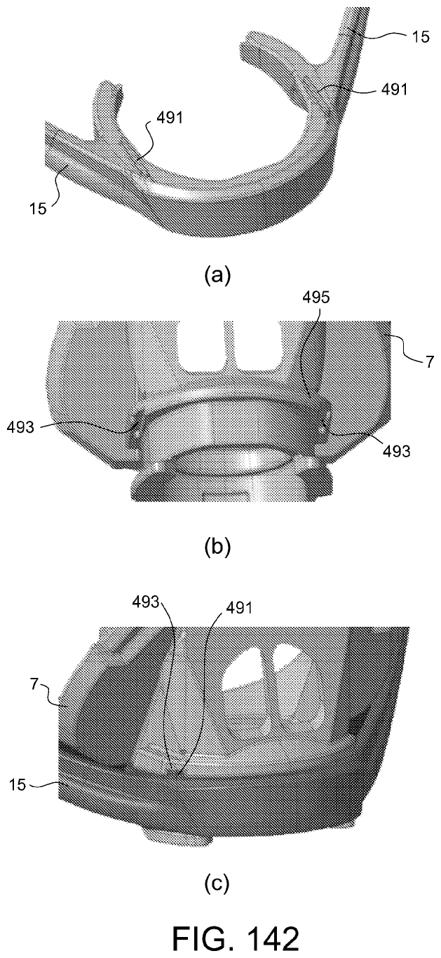

With reference to FIG. 142, the upper surfaces of the sides for the frame clip 14 are each provided with respective guide rails 491 that are received in corresponding elongate slots 493 provided along an upper flange 495 of the inlet connector 7, the upper flange 495 defining the top of the recess 9c. Engagement of the guide rails 491 in the slots 493 assists in connecting the frame clip 14 to the inlet connector 7, and resists relative rotation between the frame clip 14 and the inlet connector 7.

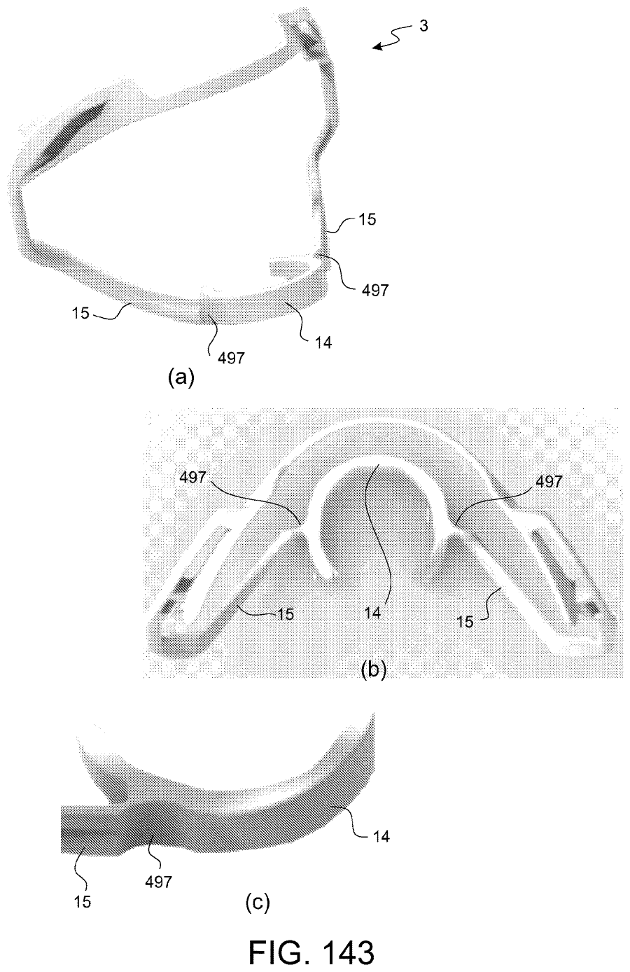

With reference to FIG. 143, the frame clip 14 comprises a plurality of thinned portions 497 configured to provide increased flexibility of the frame clip 14 at those portions 497.

In this example, a thinned portion 497 is provided at each intersection of the frame clip 14 with the lateral arms 15, to increase flexibility of the lateral arms 15 relative to the frame clip 14. This enables the lateral arms 15 to flex without forces from the flexing of the lateral arms 15 also moving the frame clip 14 and accidentally disconnecting the frame clip 14 from the inlet connector 7. This can be beneficial for example when the headgear is being disconnected from the frame 3.

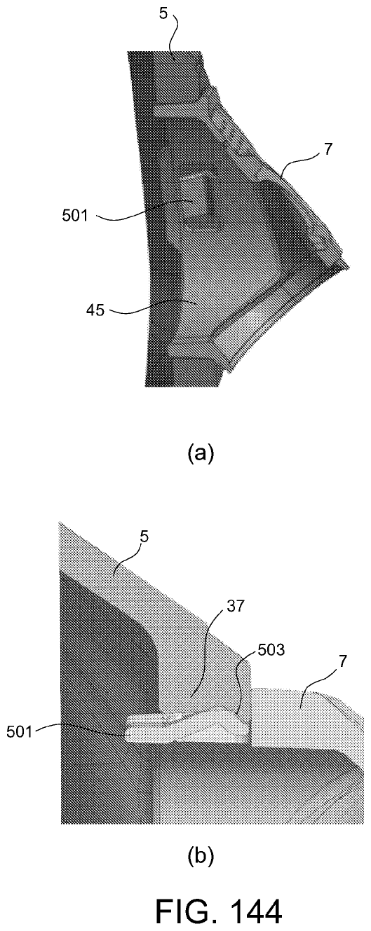

With reference to FIG. 144, the outlet boss 45 of inlet connector 7 comprises a pair of movably mounted cushion retainers, comprising flexible fingers 501, a finger 501 being provided on each side of the outlet boss 45. Each finger 501 is configured to engage with a ridge 503 provided by, or adjacent, the rigid ring 37 of cushion 5. A separate ridge 503 may be provided for each finger 501, or a continuous ridge 501 may extend around the entire periphery of the opening of the cushion 5. The flexibility of the fingers 501 allow connection and disconnection of the inlet connector 7 and cushion 5.

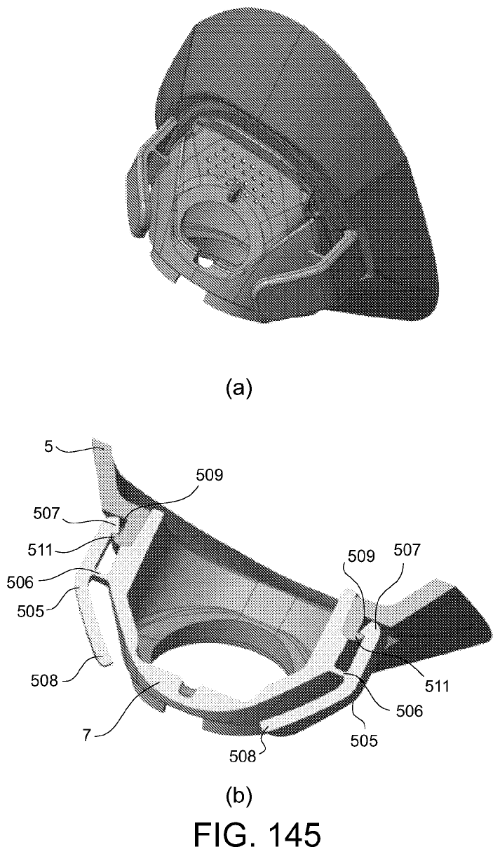

With reference to FIG. 145, the inlet connector 7 comprises a pair of movably mounted cushion retainers 505, comprising flexible fingers one at each side of the inlet connector 7.

Each cushion retainer 505 is elongate. A proximal end of each cushion retainer 505 comprises a hook 507 configured to engage with the cushion 5 to connect the inlet connector 7 to the cushion 5. Each cushion retainer 505 is mounted on a respective mount 506 that extends outwardly from the sides of the inlet connector 7 so that the cushion retainers 505 are spaced from the sides of the inlet connector 7. A distal end 508 of each cushion retainer 505 extends forwardly beyond the mounts 506.

The cushion 5 may be provided with a pair of recesses 509, each recess comprising an engagement formation 511. The hooks 507 of the cushion retainers 505 engage with a respective engagement formation 511 to connect the cushion 5 to the inlet connector 7. As the inlet connector 7 is pushed onto the cushion 5, the distal ends of the cushion retainers 505 move outwardly and then snap back so that the hooks 507 engage with engagement formations 511. When so engaged the cushion 5 is retained on the inlet connector 7.

To disconnect the cushion 5 from the inlet connector 7, a forward portion of each cushion retainer 505 is pressed, pivoting each cushion retainer 505 about a respective mount 506. The forward end 508 of each cushion retainer 505 therefore moves inwardly towards the inlet connector 7, and the distal end and hook 507 of each cushion retainer 505 moves outwardly away from the inlet connector 7, disengaging each hook 507 from the respective engagement formation 511 on the cushion 5.

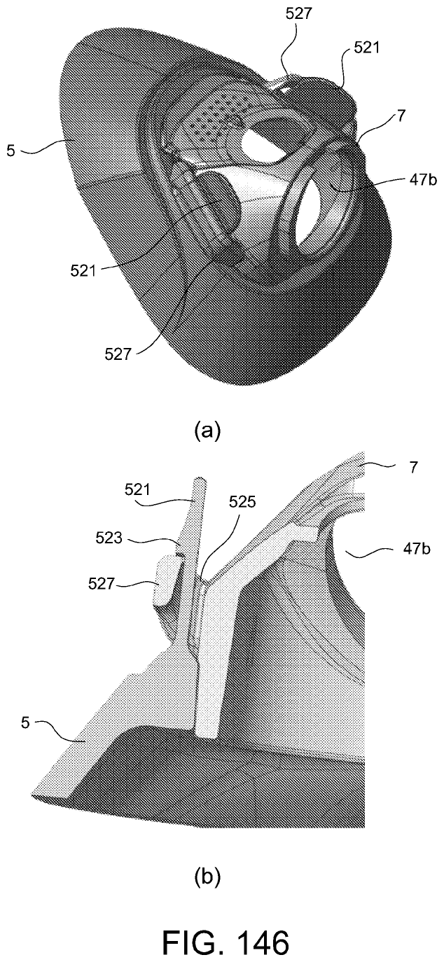

With reference to FIG. 146, cushion 5 comprises a pair of forwardly directed tabs 521, one at each side of the cushion opening. Each tab 521 comprises a hook 523. The inlet connector 7 comprises a pair of slots 525, formed by arcuate bars 527 at each side of the inlet connector 7. Each tab 521 is received in a respective slot 525, with the hooks 523 engaging respective bars 527 to connect the cushion 5 to the inlet connector 7. The distal end of each tab 521 extends beyond the respective slot 521 and can be moved by the user to disengage the hooks 523 from the bars 527, to disconnect the cushion 5 from the inlet connector 7.

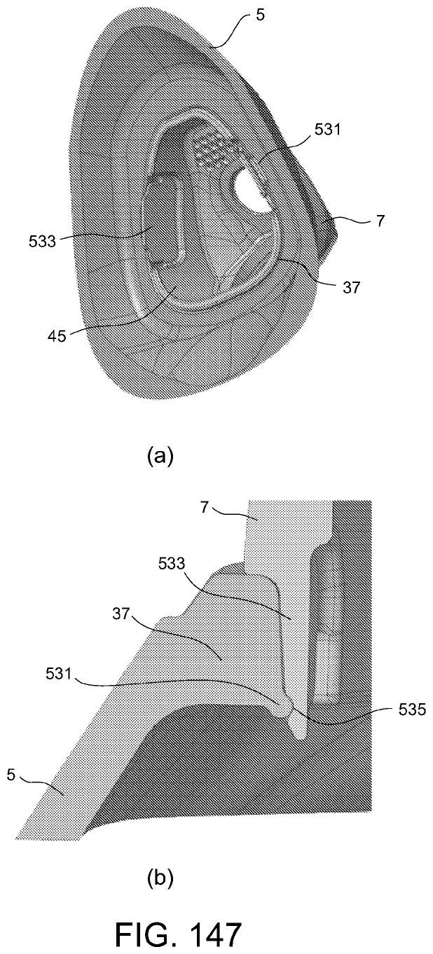

With reference to FIG. 147, the rigid ring 37 of cushion 5 comprises a pair of laterally spaced apart ridges 531, one on each side of the rigid ring 37. The outlet boss 45 of the inlet connector 7 comprises a pair of laterally spaced apart thinned regions 533, each region 533 comprising a recess 535 configured to receive a respective ridge 531. The relatively thin material of the thinned regions 533 allows the thinned regions 533 to be relatively flexible such that the thinned regions move as the ridges 531 are received in, and removed from, the recesses 535 to connect and disconnect the cushion 5 and the inlet connector 7.

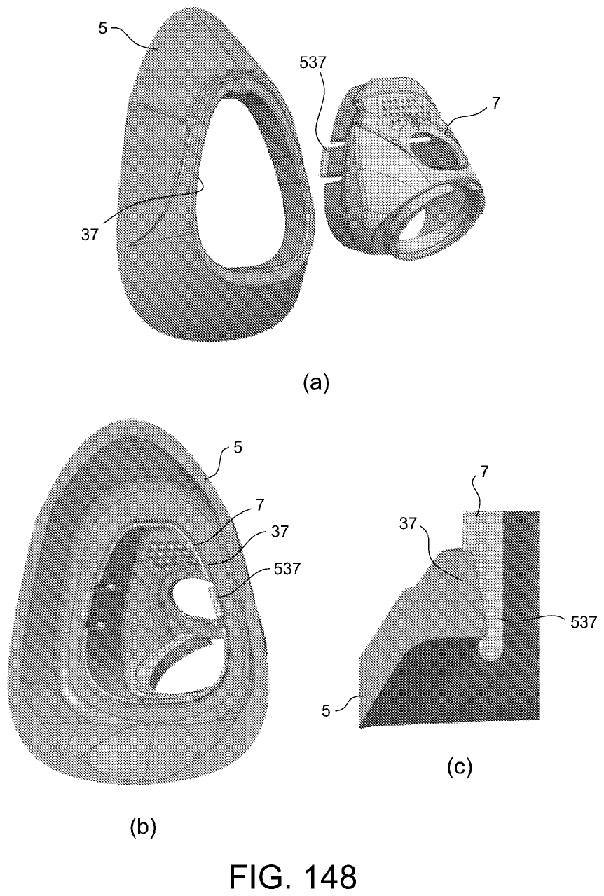

With reference to FIG. 148, a similar embodiment is shown to that of FIG. 147, except that the thinned regions 533 are replaced with fingers 537, each finger 537 comprising a ridge 539 that engages with the rigid ring 37 of the cushion 5 to connect the cushion 5 to the inlet connector 7.

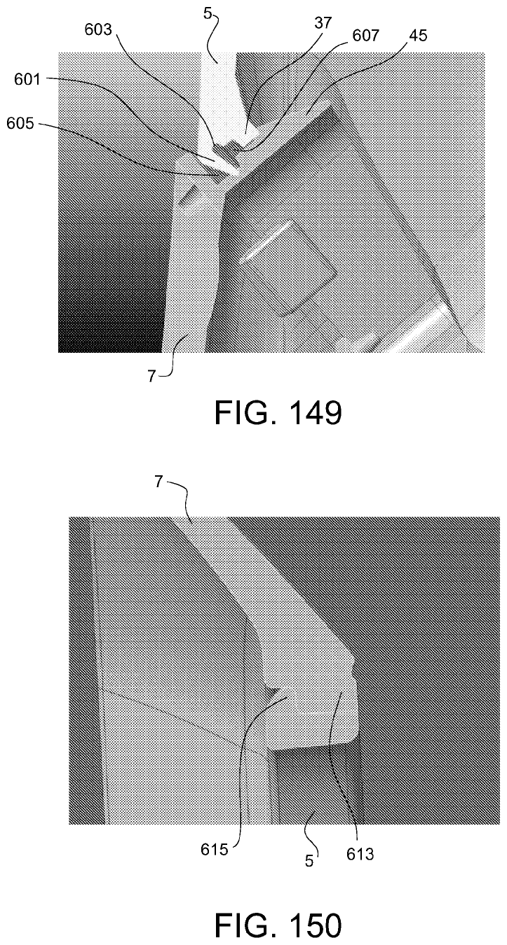

With reference to FIG. 149, cushion 5 comprises rigid ring 37, and an additional annular blade 601 that extends around the periphery of, but is spaced from, the rigid ring 37. An annular recess 603 is provided between the blade 601 and the rigid ring 37. The outlet boss 45 of the inlet connector 7 comprises first and second peripheral grooves 605, 607. During assembly, the blade 601 folds into the annular recess 603, as the cushion 5 flexes over the outlet boss 45. Once the outlet boss 45 is fully seated on the cushion 5, the blade 601 partially unfolds into the first groove 605. The rigid ring is received in the second groove 607.

The blade 601, when received in the first groove 605 and viewed in cross section, is inclined rearwardly, that is, the blade 601 is inclined away from the front of the inlet connector 7 towards the cushion 5, so that the distal end of the blade 601 is directed towards the cushion 5. This first condition of the blade 601 can be seen in FIG. 149. To disconnect the cushion 5 from the inlet connector 7, the inlet connector 7 and the cushion 5 are pulled away from the other in the direction of the axis of the outlet boss 45. As the inlet connector 7 and cushion 5 disconnect, the blade 601 pivots towards a position in which the blade 601 is inclined forwardly, that is, towards the front of the inlet connector 7 and away from the cushion 5, with the distal end of the blade 601 directed away from the cushion 5. As the blade 601 inverts by rotating and/or deforming from being inclined rearwardly to being inclined forwardly, force is required to be applied by the user. This requirement for force to be applied means that the blade 601 assists in maintaining the connection between the inlet connector 7 and the cushion 5, and that more force is required to disconnect these components than if the blade 601 was not present.

Referring now to FIG. 150, in this embodiment cushion 5 does not comprise a rigid ring 37. The part of the cushion 5 that surrounds the inlet aperture 26 is therefore relatively soft. Omitting the rigid ring 37 reduces component count, and can simplify the cushion 5 moulding process. A softer cushion connection may require lower forces during assembly and disassembly, making it easier for users.

The outlet boss 45 comprises a peripheral, outwardly extending ridge 613 over which a lip 615 of the inlet aperture 26 passes during assembly, the ridge 613 engaging the rear of the lip 615 of the inlet aperture 26 once the cushion 5 is fully mounted on the outlet boss 45. As the cushion 5, and lip 615 are deformable, a relatively large ridge 613 can be used, reducing the risk of unintentional disassembly of the cushion 5 and inlet connector 7.

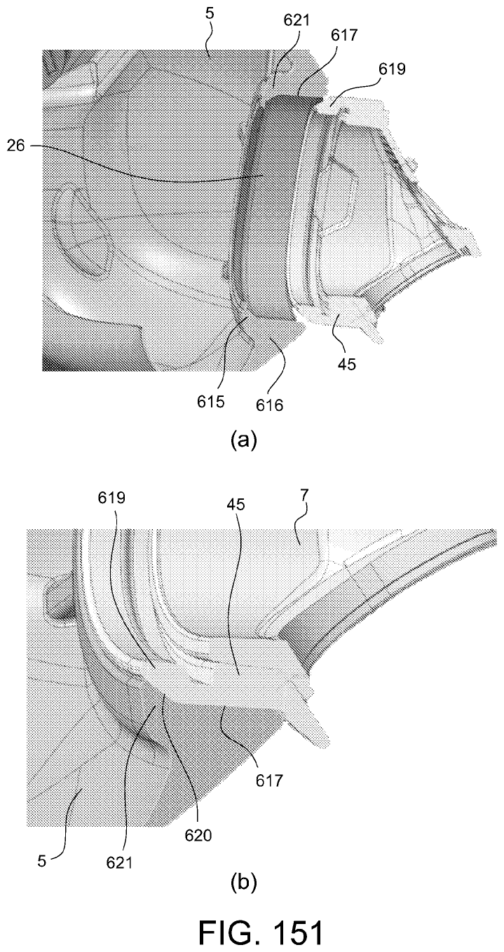

Referring now to FIG. 151, the inlet aperture 26 of the cushion 5 is surrounded by a relatively thick region of material 616, that extends relatively far into the cushion 5 from the inlet aperture 26, and therefore comprises a relatively large sealing surface 617, for engagement and sealing with the outlet boss 45 of the inlet connector 7.

The distal end of the outlet boss 45 is chamfered 619, chamfer 619 sealing against a corresponding inclined surface 620 of sealing lip 615 of cushion 5. This configuration allows for a relatively easy assembly mechanism where the cushion 5 is pushed directly onto the inlet connector outlet boss 45 without needing a large amount of force. A large lead in section at the inlet aperture 26 enables easy assembly.

The thicker cushion region at the inlet aperture 26 adds some strength to the connection and allows for a good seal between the cushion 5 and the inlet connector 7.

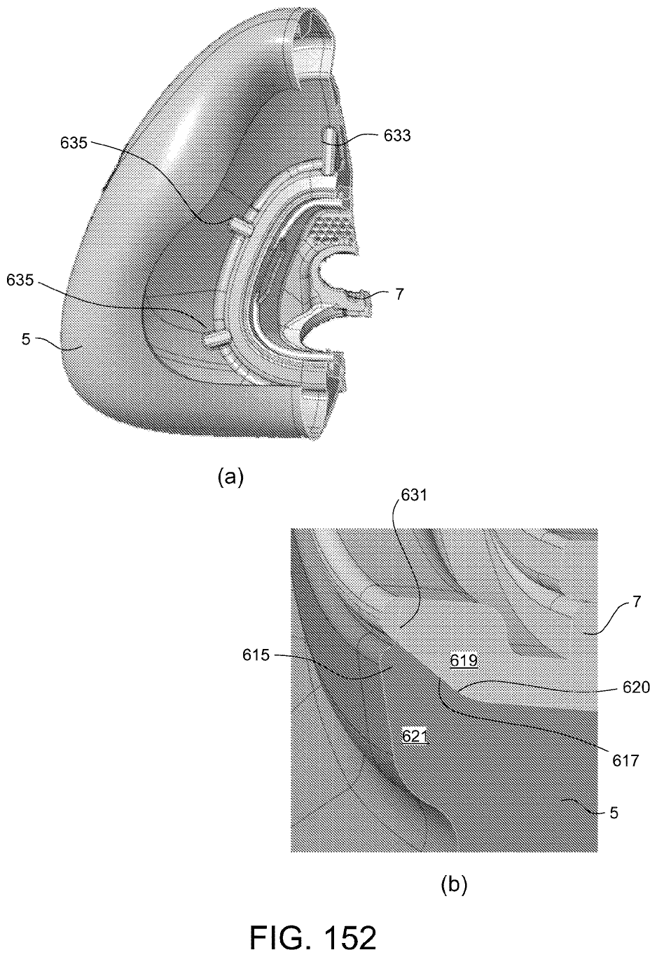

With reference to FIG. 152, cushion 5 is the same as that of FIG. 151, but the cushion 5 further comprises a blade seal 631 that extends outwardly from the lip 615 into the inlet aperture 26, and which improves the seal between the cushion 5 and the inlet connector 7.

With further reference to FIG. 152, the cushion 5 may comprise a plurality of internal ribs 633, spaced around the inlet aperture 26, and configured to reduce deformation of the cushion 5 at the inlet aperture 26, providing more strength to the connection between the cushion 5 and the inlet connector 7.

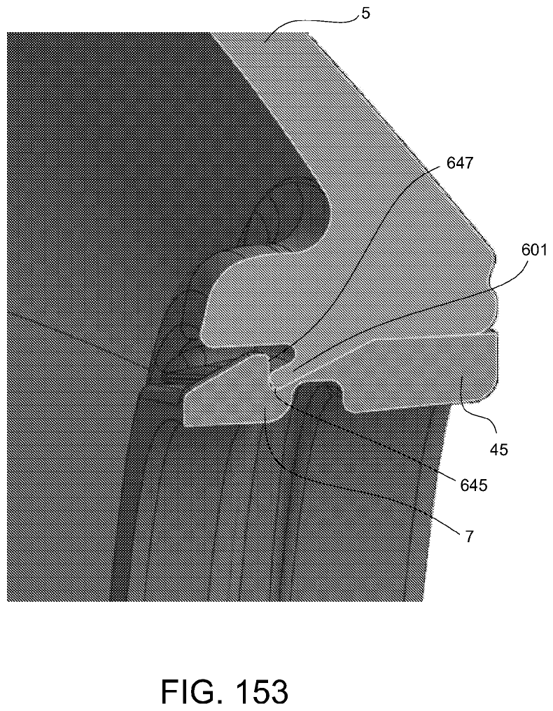

With reference to FIG. 153, a cushion 5 comprises similar features to the cushion 5 of FIG. 149, and comprises an annular blade 601 that extends around the periphery of the inlet aperture 26. The blade 601 is inclined rearwardly into the cushion 5, and consequently deforms relatively easily as the inlet aperture 26 is slid onto the outlet boss 45 of the inlet connector 7, with the blade 601 ultimately being received in a recess 645 on the outside of the outlet boss 45. Recess 645 terminates in a flat surface 647 against which the distal tip of the blade 601 abuts, when the cushion 5 is fully mounted on the outlet connector 7. This abutment of the tip of the blade 601 with the flat surface 647, and the generally rearwardly inclined configuration of the blade 601, means that a larger force is required to disconnect the cushion 5 from the inlet connector 7 than to connect these components. The inclined blade 601 can therefore resist unintentional disconnection of the cushion 5 and the inlet connector 7.

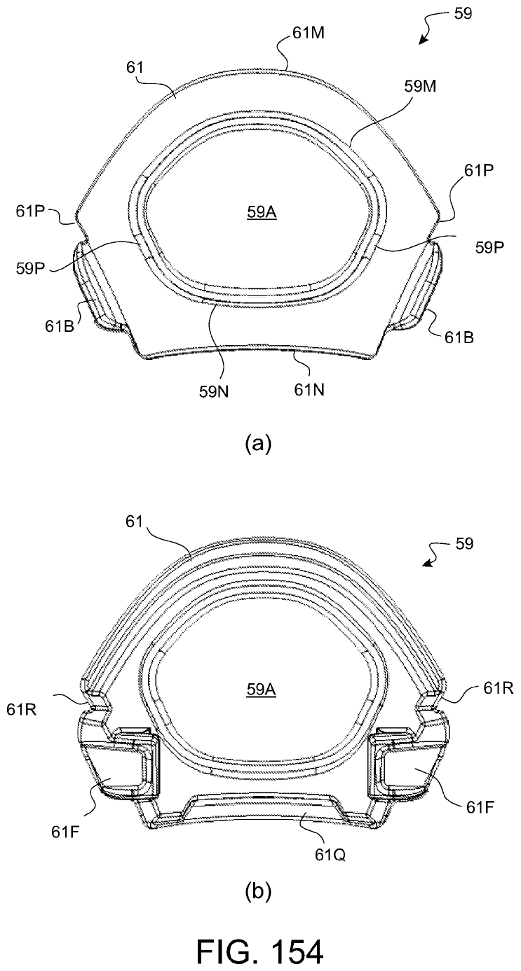

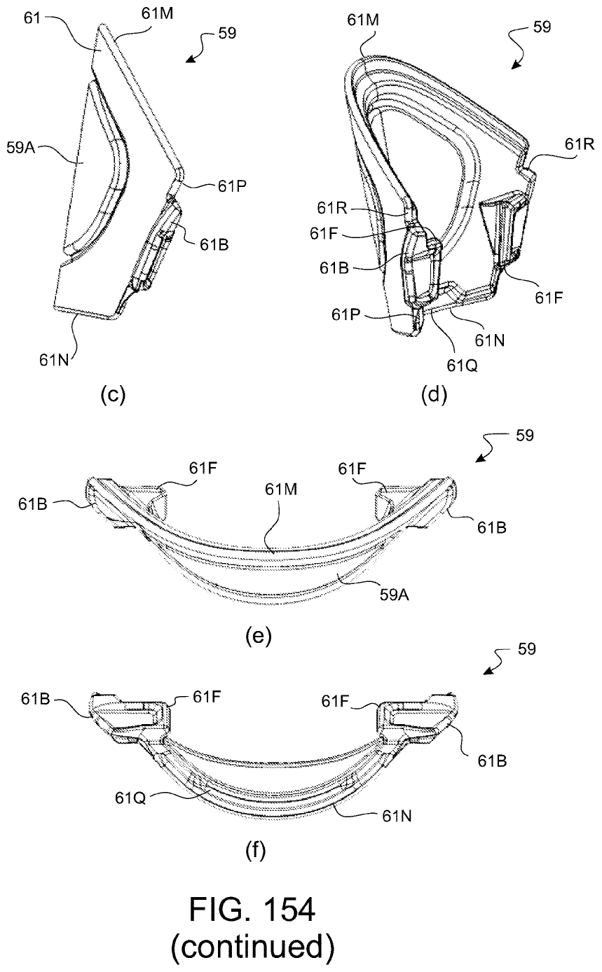

With reference to FIG. 154, a diffuser 59 comprises a diffuser frame 61 on which a pad or mat of diffuser material 59A is provided. Diffuser frame 61 comprises similar features to the diffuser frame 61 of FIG. 111, and comprises a pair of laterally opposed finger tabs 61B. Adjacent each finger tab 61B, is a respective clip 61F that releasably engages respective protrusions 7P on the sides of the front of the inlet connector 7. The diffuser 59, comprising the diffuser material 59A and diffuser frame 61, is mounted on the inlet connector 7. With reference to FIG. 154, it is the diffuser frame 61 that is mounted, removably, on the inlet connector 7.

The diffuser frame 61, when viewed from the front, comprises an upper portion defined by an arcuate upper margin 61M, and a lower portion defined by an arcuate lower margin 61N and substantially straight side margins 61P. A finger tab 61B extends from each side margin 61P.

The side margins 61P are inclined outwardly away from the lower margin 61N, the lower margin 61N being shorter than the upper margin 61M such that that the diffuser frame 61 flares outwardly away from the lower margin 61N towards the upper margin 61M. The upper margin 61N curves downwardly from an apex of the diffuser frame 61, to meet the upper ends of the side margins 61P. The lower margin 61N has a relatively shallow curvature, compared to the curvature of the upper margin 61M.

The diffuser frame 61 is non-planar, and in this example is curved across its width, that is, when viewed from above or below. The upper and lower margins 61M, 61N are therefore arcuate when viewed from the front, and also arcuate when viewed from above or below.

The pad or mat of diffuser material 59A of diffuser 59 has an outer shape that substantially matches the shape of the outer periphery of the diffuser frame 61. Diffuser material 59A thus comprises arcuate upper and lower margins 59M, 59N, joined by inclined substantially straight side margins 59P. The diffuser frame 61 extends around the periphery of the diffuser material 59A. The diffuser frame 61 surrounds the periphery of the diffuser material 59A. The diffuser material 59A is held by the diffuser frame 61, for example via adhesive or overmoulding.

As can be seen with reference to FIG. 154b) and d), the rear of the diffuser frame 61 comprises an elongate recessed portion 61Q that extends partially along the lower margin 61N of the diffuser frame 61. The recessed portion 61Q forms a channel that at least partially defines an opening that forms a vent as will be described in more detail below.

The diffuser 59, and the inlet connector 7 may be provided with respective alignment/locating features configured to engage when the diffuser 59 is mounted on the inlet connector 7, to help correctly locate and align the diffuser 59 on the inlet connector 7.

As can also be seen with reference to FIG. 154b) and d) the rear of the diffuser frame 61 comprises alignment/locating features comprising a pair of laterally spaced apart locating notches 61R. Each locating notch 61R is formed at the intersection between an end of upper margin 61M and a side margin 61P. When the diffuser frame 61 is viewed from the side each locating notch 61R forms a right angled locating face that extends through approximately 90°. Each locating notch 61R locates the diffuser frame 61 against the inlet connector 7 as will be described further below.

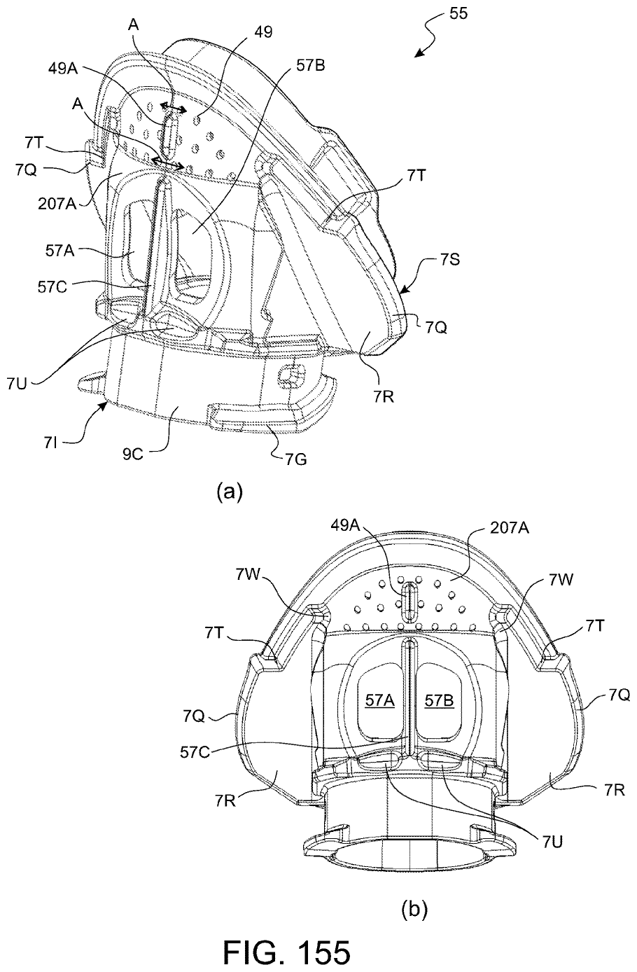

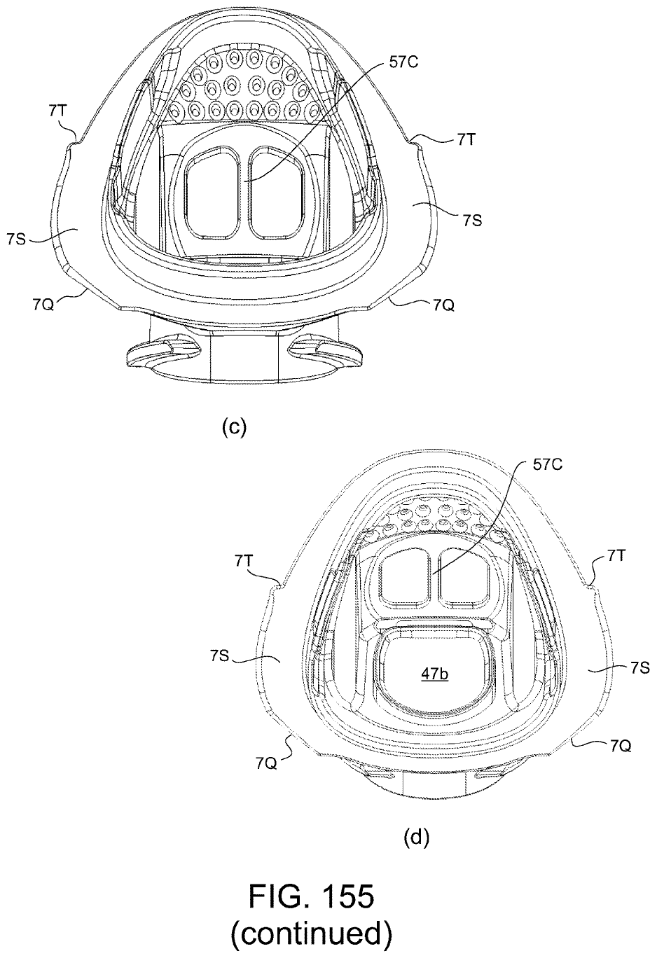

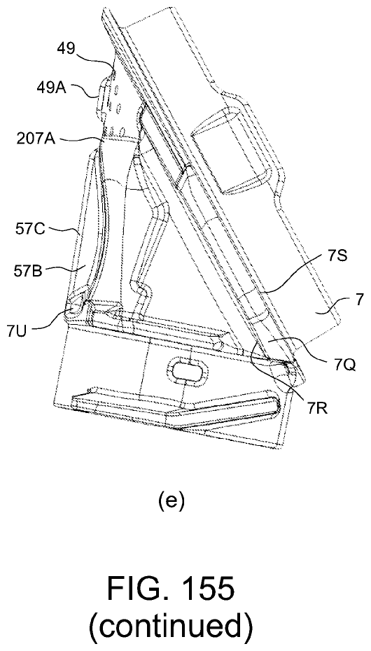

With reference to FIG. 155, the inclined forward wall 207A of the inlet connector 5 is arcuate so as to curve across its width, when viewed from above or below. The curvature of the inclined forward wall 207A substantially matches the curvature of the diffuser frame 61 described above with reference to FIG. 154.

The curvature of the inclined forward wall 207A provides a curved surface on which the bias vent holes 49 are provided. This allows for a constant, or at least more consistent, distance to be provided between the bias vent holes 49 and the diffuser 59, in particular the rear surface of the diffuser 59. This constant distance assists in improving the performance of the diffuser 59.

The curved surfaces also reduces the occurrence of sharp internal angles, which can assist in reducing noise caused by bias flow through the bias vent holes 49.

The bias vent holes 49 and valve opening 57 are formed on a central body of the inlet connector 7.

With reference to FIGS. 97, 102, 112, 114-117, 125, 132-144, and 155, the inlet valve opening 57, that is closed and opened by the anti-asphyxia valve 53, may comprise sub-openings 57A, 57B, formed by a rib 57C that extends substantially vertically from top to bottom of the valve opening 57, dividing the inlet valve opening 57 into the sub-openings 57A, 57B.

Rib 57C projects forwardly of the inclined forward wall 207A of the inlet connector 7, when the inlet connector 7 is viewed from the side.

With reference to FIG. 155, the rib 57C terminates adjacent the top of the valve opening 57.

A vent hole rib 49A is provided, above the rib 57C and valve opening 57. Vent hole rib 49A extends substantially vertically along part of the array of bias vent holes 59. The vent hole rib 49A does not extend fully across the array of bias vent holes 49, so as to assist in maintain fluid communication, indicated by arrows A, laterally across the array of bias vent holes 49. In other words, the vent hole rib 49A is configured such that the bias vent holes 49 to one side of the vent hole rib 49A can be fluidly exposed to the bias vent holes 49 on the other side of the vent hole rib 49A.

The vent hole rib 49A may be provided without rib 57C, that is, rib 57C may be omitted.

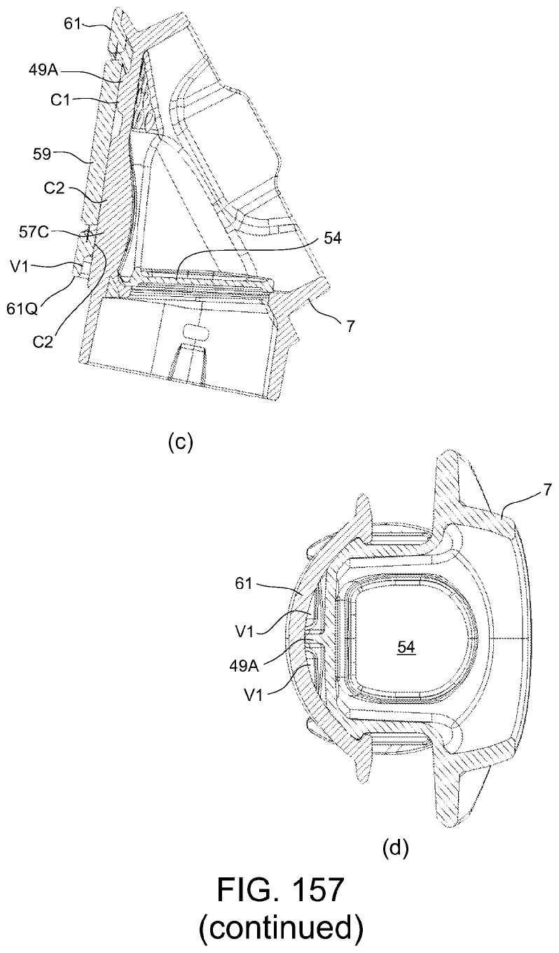

Vent hole rib 49A spaces diffuser material 59A from the bias vent holes 49, and may contact the diffuser material 59A at region C1 in FIG. 157c).

Rib 57C spaces the diffuser material 59A from the AA valve opening 57, and may contact the diffuser material 59A at region C2 in FIG. 157c). Rib 57C also contacts a lower portion of the diffuser frame 61, at region C3 in FIG. 157c).

The ribs 49A, 57C help ensure that the material of diffuser 59 cannot be deformed towards the inlet connector 7 in such a way that the diffuser material becomes damaged. Ribs 49A, 57C can assist in minimising the diffuser 59 from blocking one or more of the bias vent holes 49 and/or the valve opening 57, for example if the diffuser material were to become saturated.

With further reference to FIG. 155, the inlet connector 7 comprises an outwardly extending portion, that extends outwardly from the central body. The outwardly extending portion comprises a radially outwardly extending flange comprising a pair of lateral wings 7Q that extend laterally outwardly from the inclined forward wall 207A, and valve opening 57. Each lateral wing 7Q comprises a substantially planar front face 7R against which the rear of the diffuser frame 61 abuts, when the diffuser frame 61 is mounted on the inlet connector 7. Each lateral wing 7Q comprises a substantially planar rear face 7S against which the rib seal 40 of the cushion 5 seals, when the cushion 5 is mounted on the inlet connector 7.

The outer margin of an upper portion of the inlet connector 7 comprises locating/alignment features comprising a pair of laterally spaced apart locating corners 7T that are configured to engage with the locating notches 61R of the diffuser frame 61, when the diffuser frame 61 is mounted on the inlet connector 7. This engagement assists in orienting the diffuser frame 61 correctly with the inlet connector 7 during assembly, and helps to prevent relative twisting between the diffuser frame 61 and the inlet connector 7. Each locating corner 7T is provided part way up the side margin of the inlet connector 7, on the lateral wings 7Q.

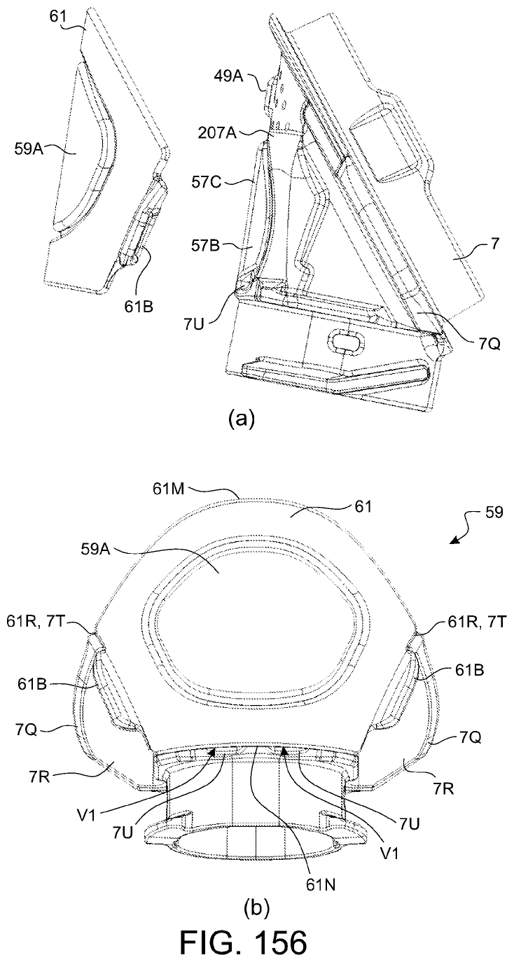

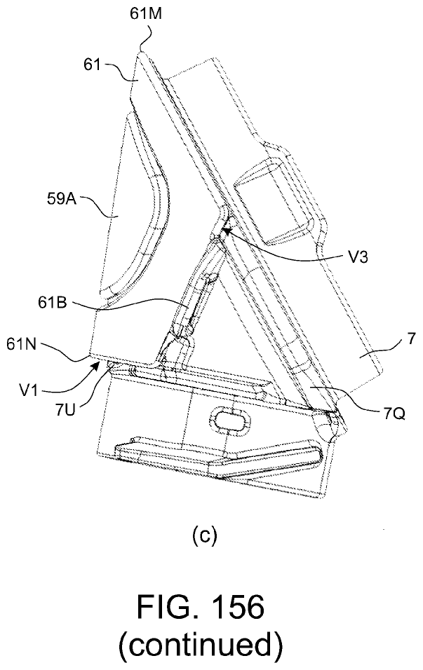

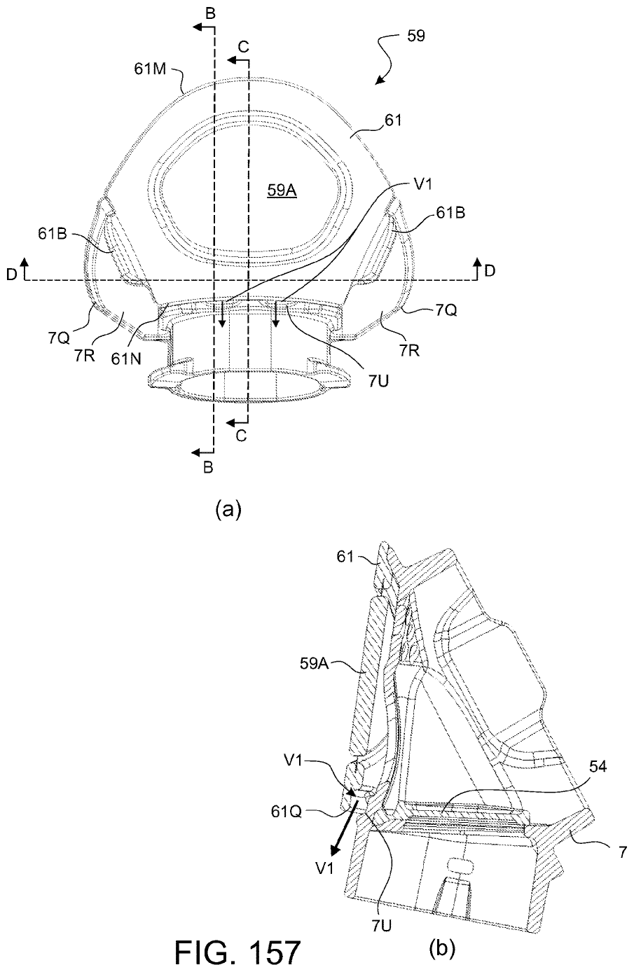

With reference to FIGS. 155 and 156, inlet connector 7 comprises a pair of indents 7U adjacent and just below the rib 57C and valve opening 57. The indents 7U are positioned adjacent the recessed portion 61Q of diffuser frame 61, when the diffuser frame 61 is mounted on the inlet connector 7, and together define a channel that forms vent V1 as can be seen in FIG. 156b).

With reference to FIGS. 155 and 156, the diffuser frame 61 and inlet connector 7 comprise features that allow airflow in and out of the chamber defined by the cushion 5. For example, if the diffuser material of diffuser 59 becomes saturated, one or more alternative airflow pathways may provide the desired venting to clear CO2. One or more additional airflow pathways may also allow the user to breathe when the breathing apparatus is switched off, that is, when the breathing apparatus is not delivering a flow of breathable gases to the mask assembly. One or more additional airflow pathways can provide an airflow pathway with lower resistance to flow, than for example, airflow solely being through the diffuser 59. One or more additional airflow pathways can therefore make it easier for the user to continue breathing, in these circumstances. Any additional moisture may also exit through such airflow pathways.

With reference to FIGS. 155 to 157, the diffuser frame 61 and inlet connector 7 are configured to provide a central, lower vent V1, and a pair of upper, lateral vents V2, V3.

Central, lower vent V1 is defined between the indents 7U of inlet connector 7 and the recessed portion 61Q of diffuser frame 61, as described below. Vent V1 defines a permanent airflow pathway between the external ambient atmosphere, and the internal cavity of the cushion 5, below the valve opening 57, and forward of the anti-asphyxia valve 53.

With reference to for example FIG. 157A, the diffuser frame 61 is dimensioned and shaped to cover the bias vent holes 49, the valve opening 57 and vent V1, when the mask assembly is viewed from the front. Arcuate lower margin 61N of diffuser frame 61 extends across the indents 7U, such that indents 7U are substantially covered when viewed from the front. The diffuser frame 61 and inlet connector 7 together define a space between these components that forms vent V1.

This configuration provides a clean, neat and visually desirable appearance, without vent V1 either extending through the front of the mask assembly, or otherwise being visible from the front. Air can pass through vent V1 into or out of the cushion 5 in a generally vertically downward direction indicated by arrows A in FIG. 157a) and b), generally aligned with the direction of inlet opening axis 13A.

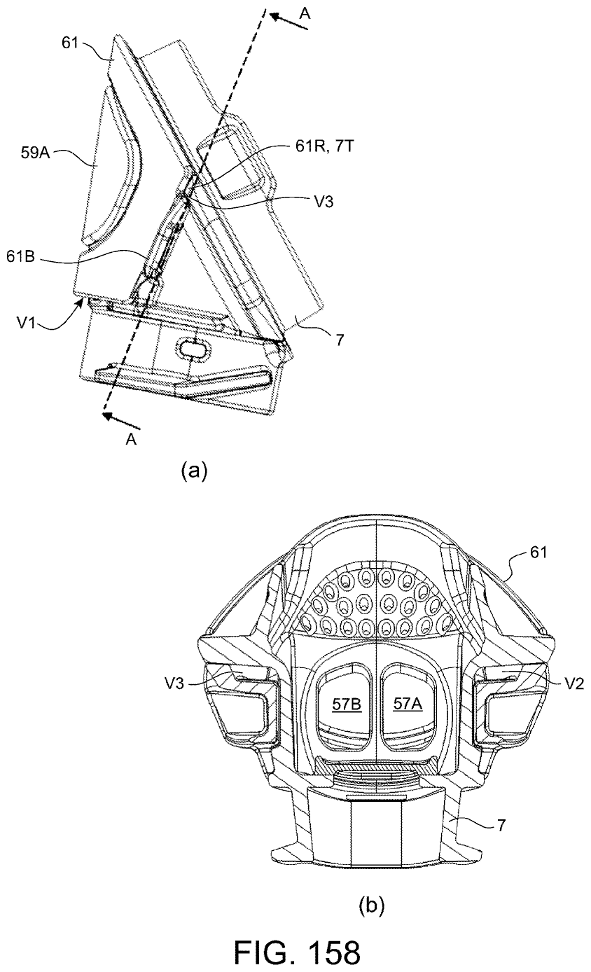

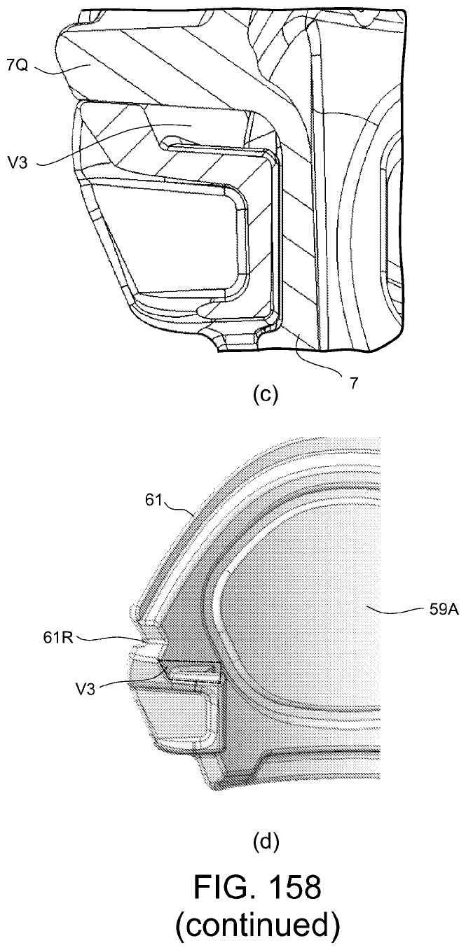

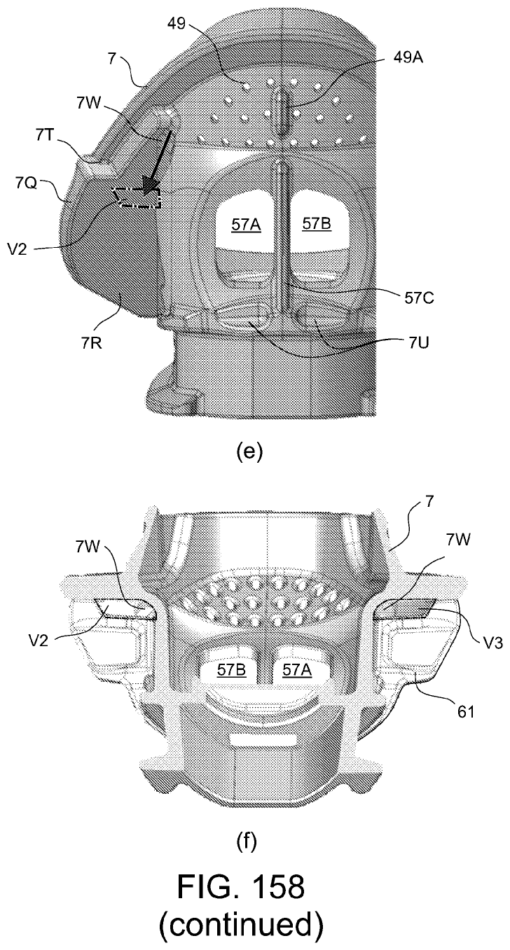

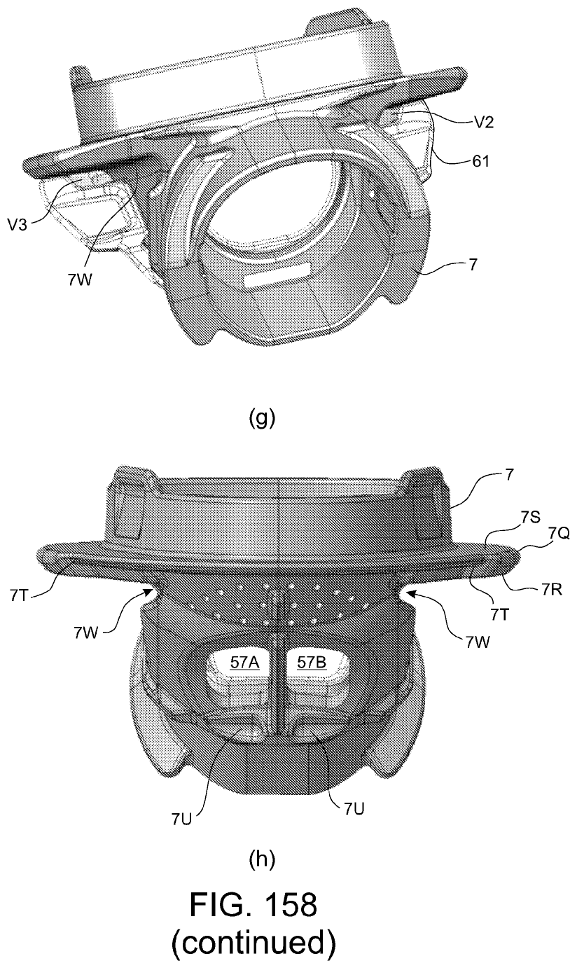

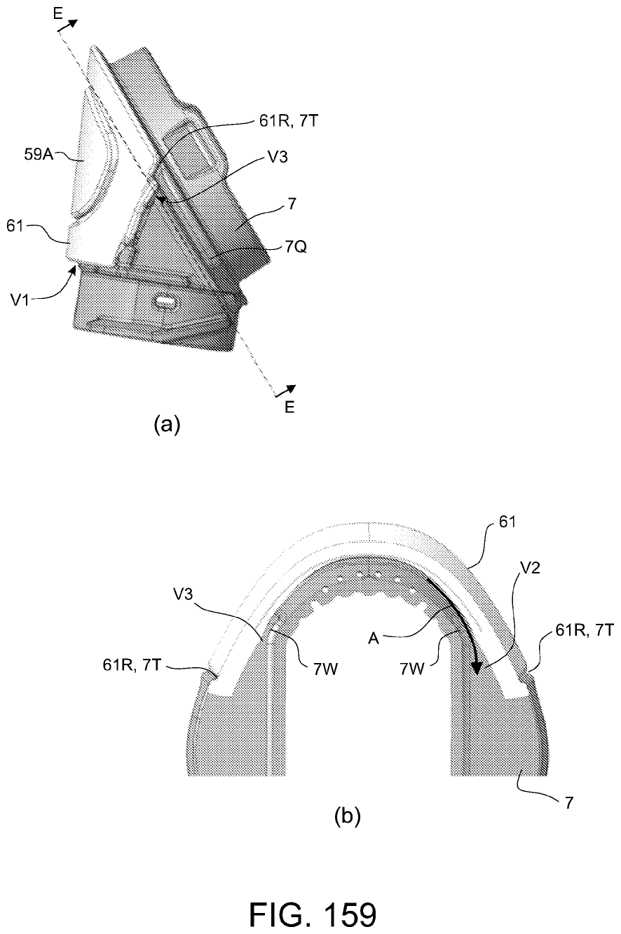

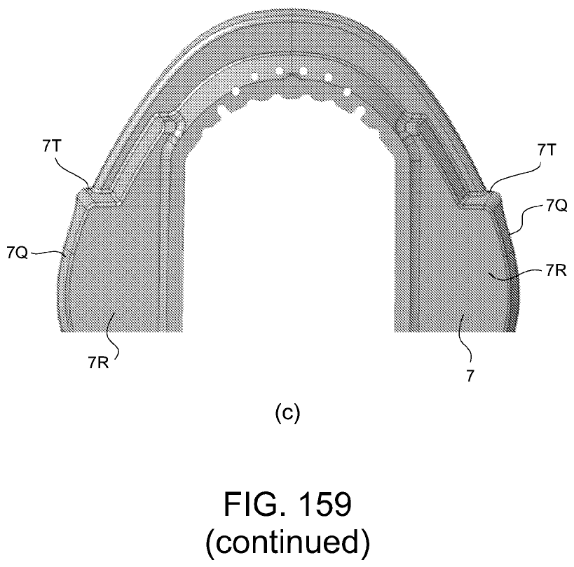

With reference to FIGS. 158 and 159, the upper lateral vents V2, V3 are provided in the region of the locating notches 61R and the locating corners 7T. As can be seen in FIGS. 158e) and 159b), an airflow pathway, indicated by arrow A in FIG. 159b), is provided by an elongate channel 7W defined adjacent these two features and between the wings 7Q and the rear of the diffuser frame 61. Channel 7W extends from an upper part of each lateral wing 7Q, away from the array of bias vent holes 49, and downwardly to the side margin 61P of the diffuser frame. Each vent V2, V3 therefore comprises an airflow path that extends generally downwardly and laterally outwardly, across the front face of the lateral wings 7Q.

Vents V2, V3 provide additional venting from the cushion 5, from a region adjacent the bias flow holes 49.

As with vent V1, the diffuser frame 61 and inlet connector 7 together define a space between these components to form vents V2 and V3. This configuration provides a clean, neat and visually desirable appearance, without vents V2 and V3 either extending through the front of the mask assembly, or otherwise being visible from the front.

Vents V1, V2 and V3 provide the mask assembly with a plurality of separate, additional airflow pathways.

Vents V1, V2 and V3 provide the mask assembly with separate airflow pathways in different locations on the mask assembly.

Vent V1 provides an additional airflow pathway at a lower portion of the mask assembly, and in particular at a lower portion of the inlet connector 7, adjacent the lower margin 61N.

Vents V2, V3, provide additional airflow pathways at an upper portion of the mask assembly, and in particular at an upper portion of the inlet connector 7, adjacent side margins 61P.

Vents V1, V2 and V3 provide the mask assembly with an additional airflow pathway adjacent the valve opening 57 of the anti-asphyxia valve 53, and an additional airflow pathway adjacent the bias flow holes 49.

The vent opening defined by vent V1 is a different configuration from either of the vent openings defined by vents V2, V3. For example, the vent opening defined by vent V1 may be larger than the vent openings defined by vents V2, V3. The length of the airflow pathway through vent V1 is different from that of vents V2, V3. The direction of the airflow pathways of all three vents V1, V2, V3 are different.

When the user is fitting the mask, or for other reasons, the user may wear the mask assembly without therapy being provided. In this situation, the AA valve is closed such that the user must breathe through the vents V1, V2, V3 in the mask assembly. The diffuser material may add restriction to flow such that if all gases were passing through the diffuser material, it may be difficult for a user to breathe.

The lower central vent V1 provides a relatively large venting pathway for the user to breathe through when therapy is not being provided, for example. It also provides a safety vent for CO2 clearance if the diffuser material becomes saturated or otherwise blocked when therapy is being provided.

The upper lateral vents V2, V3 are secondary vents that may provide lower venting flows than the lower central vent V1, such that the majority of any venting not through the diffuser material is through the lower central vent V1.

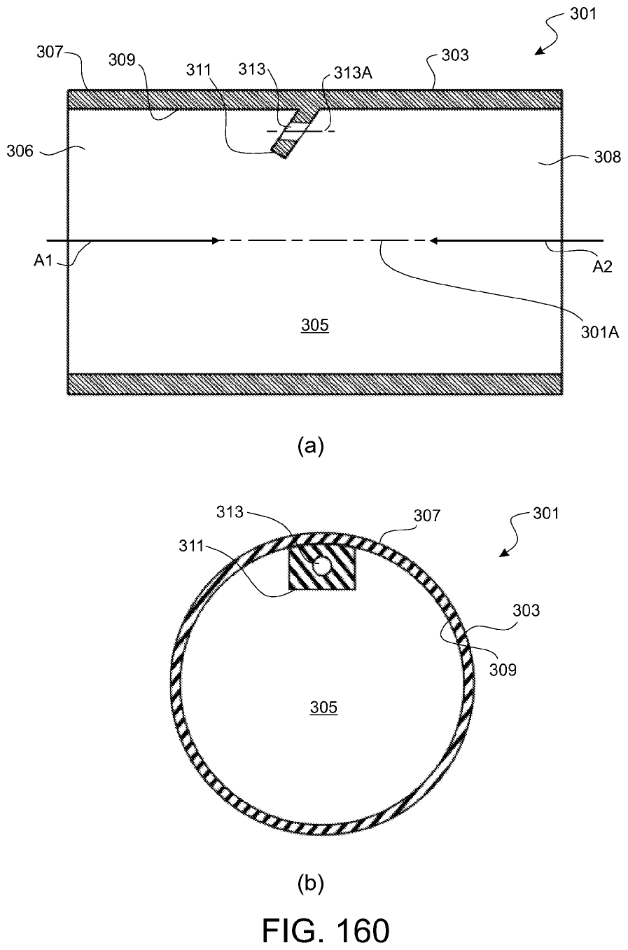

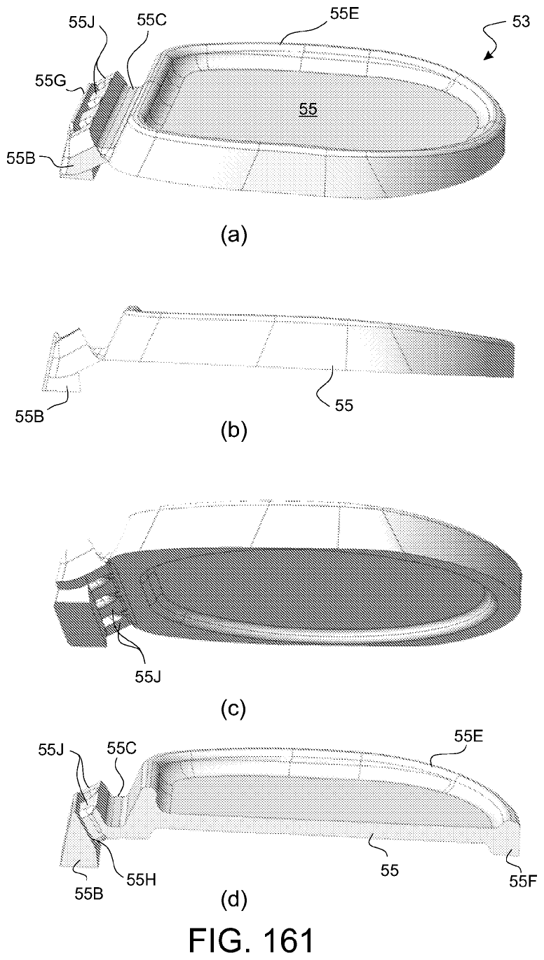

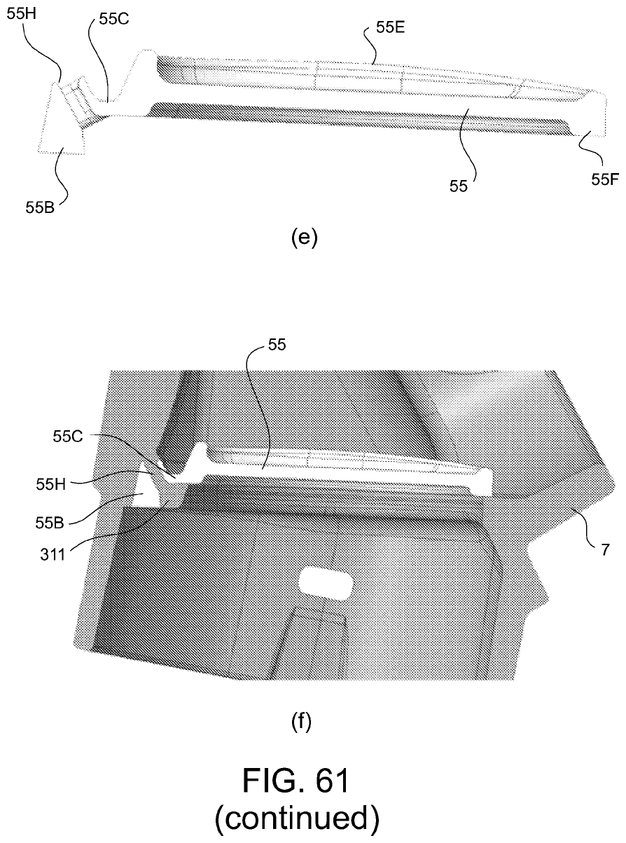

Referring to FIGS. 160 and 161, a respiratory apparatus flow path component is shown schematically for use with an anti-asphyxia valve 53, and is similar to that described above with reference to FIGS. 96-101.

Referring to FIG. 160, a respiratory apparatus flow path component is shown schematically in the form of a conduit 301, comprising an outer wall 303 defining a lumen 305 being a gases flow path through the conduit 301, extending between an inlet 306 and an outlet 308 of the conduit 301. The outer wall 303 comprises an outer surface 307 and an inner surface 309.

An AA valve anchoring formation in the form of anchoring tab 311 is provided inside the gases flow path, extending radially inwardly at least partially across the gases flow path. In this example, the anchoring tab 311 extends from the inner surface 309. Anchoring tab 311 is provided with a through bore 313 which is an open bore, each end of which is exposed to the gases flow path. The through bore 313 extends along the conduit 301 such that a central axis 313A of the bore 313 is aligned with a central axis 301A of the conduit 301. As with the example of FIG. 86, this configuration enables valve mount 55B of AA valve 53 to be received in the through bore 313, using the moulding process described above.

With reference to FIG. 160, the anchoring tab 311 extends from inner surface 309 into the gases flow path. However, the longitudinal axis of anchoring tab 311 is inclined relative to the inner surface 309. The through-bore 313 remains substantially parallel with the central axis 301A of the conduit 301. One or more posts 263 extend across the through-bore 313. Because the anchoring tab 311 is inclined relative the central axis 301A of the conduit 301, the posts 263 are also inclined relative to the central axis 301A.

With reference to FIG. 161, AA valve 53 is similar to that described above with reference to FIGS. 98 and 100. Valve mount 55B is provided with an upstanding flange 55G that projects upwardly from the rear and top of the valve mount 55B, and extends substantially across the width of the valve mount 55B. The flange 55G increases the strength of the valve mount 55B by providing a larger surface area that is in contact with the inlet connector 7, and provides greater resistance against the valve flap 55 being pulled upwardly into the inlet connector 7.

When viewed from the side, valve mount 55B is substantially triangular. The thinned region 55C joins the valve flap 55 to the valve mount 55B via a plurality of struts 55J that extend between the valve flap 55 and the inclined surface 55H. A plurality of gaps are defined, one gap between each pair of struts 55J.

When the AA valve 54 is moulded in the inlet connector 7, as described above with reference to FIGS. 97-100, the overmould material can shrink. This could cause the positioning of the AA valve 54 to be inaccurate. For example, the AA valve flap 55 may lift up due to the shrinkage, and be stuck in an open position. The changes described above in relation to FIGS. 160 and 161 to the anchoring tab 311, valve mount 55B and thinned region 55C can assist in alleviating or avoiding issues caused by the overmould material shrinking during cooling. The inclined posts 263 are configured such that then the valve mount 55B shrinks around the posts 263, the valve flap 55 is not levered upwardly. Further, the triangular shaped valve mount 55B, and the thinned region 55C being inclined upwardly away from the inclined surface 55H of the valve mount 55B, can help in balancing the effects of shrinkage, and reducing adverse effects on valve flap position.

With reference to FIG. 102, we describe above that the front wall 207A of the inlet connector 207, and in particular the inner surface of that front wall 207A that forms the valve sealing surface with the upper rim 55E of the valve flap 55A, comprises at least a non-planar portion 207B having a shaped, non-planar profile when viewed in cross-section, such that the shape of the non-planar portion 207B in cross section to some extent mimics the shape that the sealing surface of the valve flap 55 tends to adopt in use, when respiratory gases are flowing through the conduit 9 into the inlet connector 7.

With reference to FIG. 161, AA valve 54 comprises a valve flap 55 that is configured to better seal against the inner surface of the front wall 207A of inlet connector 7. In particular the upper rim 55E is shaped to better match the shape of the inner surface of the front wall 207A surrounding the AA valve opening 57 to ensure a good seal when the AA valve 54 is open.

The upper rim 55E of the valve flap 55 has also been altered to match the surface of the inlet connector 7. The upper rim 55E is thicker adjacent and towards the valve mount 55B, and thinner towards the flap tip, distal from the valve mount 55B. The upper rim 55E is continuous and extends around the entire periphery of the valve flap 55.

Valve flap 55 comprises a continuous lower rim 55F, that comprises a substantially constant thickness along its length. The lower rim 55F is thinner than the thickness of the upper rim 55E adjacent the valve mount 55B.

With reference to FIGS. 104, 105, 108, and 162, cushion 5 comprises side walls 31 that each comprise a scalloped portion 31A.

The scalloped portions 31A are each provided on a lower portion of a respective cushion side wall 31A, and extend downwardly from about halfway up the side wall 31A below a scallop line 31B. The scalloped portions 31A extend along the cushion 5, between the proximal and distal portions 27, 29 of the cushion 5.

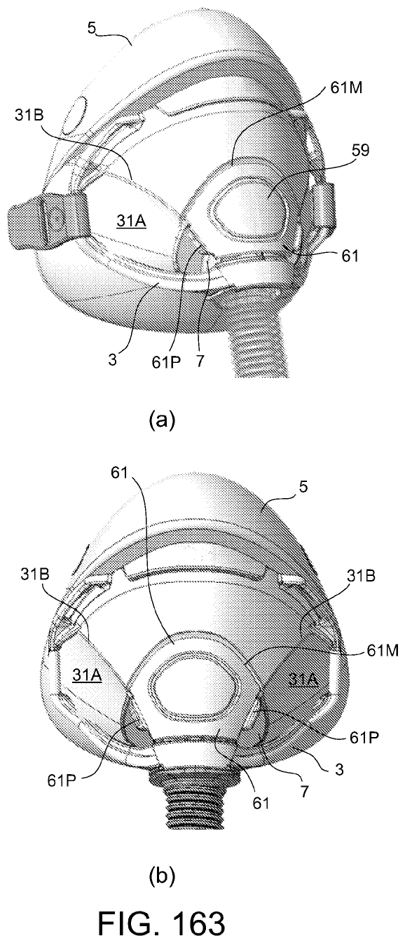

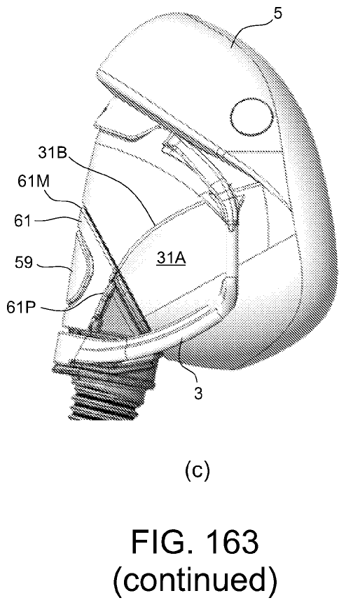

With reference to FIG. 163, the distal end of each scallop line 31B is positioned adjacent a respective intersection between the upper margin 61M and side margin 61P of the diffuser frame 61. Each scallop line 31B smoothly curves to the cushion proximal portion 27.

The scallop lines 31B are therefore contiguous with the side margins 61P of the diffuser frame 61, so as to each define a continuous and visually appealing contour line extending from the lower margin 61N of the diffuser frame 61, along the side margins 61P, and along the sides of the cushion 5 to the proximal portion 27 of the cushion 5.

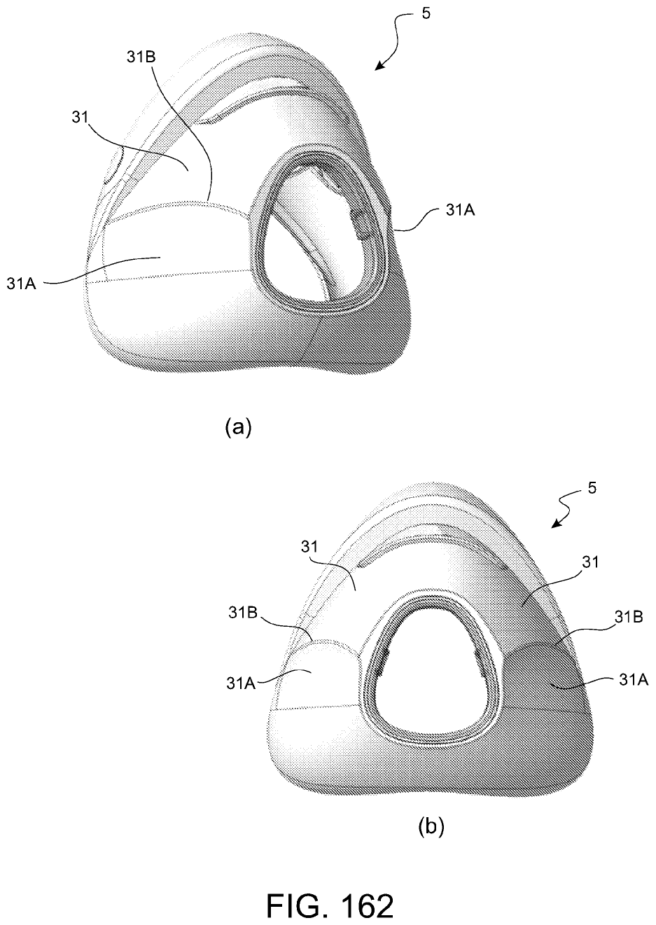

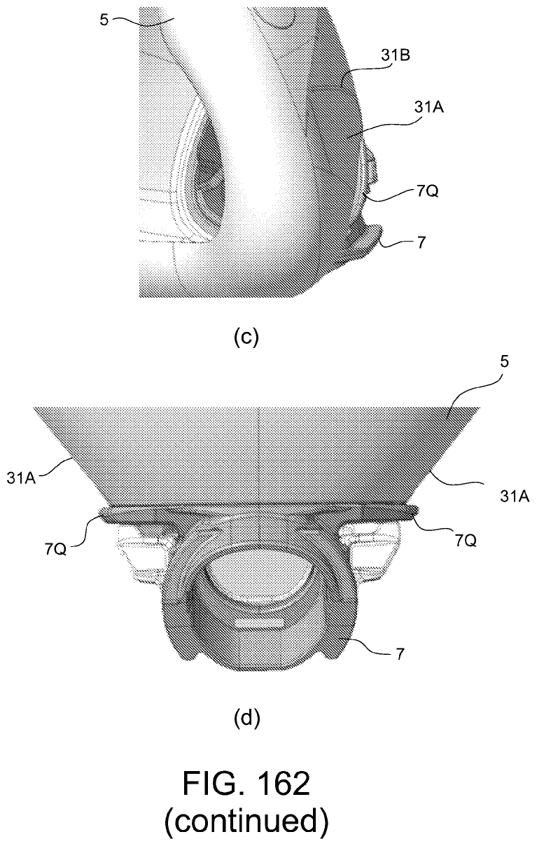

With reference to FIG. 162c) and d), these scalloped portions 31A recess the cushion 5 behind the substantially planar rear face 7S of the lateral wings 7Q of the inlet connector 7. This allows for easier attachment of the lower headgear strap clips, as there is less interference between the cushion 5 and the clips as they are being attached or detached to the frame 3.

Further, the scalloped portions 31A expose the rear face 7S of the lateral wings 7Q on the inlet connector 7, such that the lateral wings 7Q providing finger grips for a user when removing the inlet connector 7 from the cushion 5.

With reference to FIG. 163, a mask assembly comprises a frame 3, and the inlet connector 7, diffuser 59 and diffuser frame 61, and cushion 5 described above with reference to FIGS. 154 to 162.

The mask assembly 1 can be provided in a range of sizes, for example small, medium and large.

Certain terminology may be used in the description for the purpose of reference only, and thus are not intended to be limiting. For example, terms such as “above” and “below” refer to directions in the drawings to which reference is made.

Terms such as “front,” “back,” “left,” “right,” “rear,” and “side” describe the orientation and/or location of portions of the components or elements within a consistent but arbitrary frame of reference which is made clear by reference to the text and the associated drawings describing the components or elements under discussion. For example, as the context may dictate, the terms “front” and/or forward can be used relative to components described herein positioned relatively or entirely distal to the user's face when the mask assembly as described herein is worn by the user. As the context may dictate, the terms “rear” and/or “back” can be used relative to components described herein positioned relatively or entirely proximal to the user's face and/or components that are forward or at the front of the mask assembly when the mask assembly as described herein is worn by the user. Moreover, terms such as “first,” “second,” “third,” and so on may be used to describe separate components. Such terminology may include the words specifically mentioned above, derivatives thereof, and words of similar import.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”. Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

The term “plurality” refers to two or more of an item. Recitations of quantities, dimensions, sizes, formulations, parameters, shapes and other characteristics should be construed as if the term “about” or “approximately” precedes the quantity, dimension, size, formulation, parameter, shape or other characteristic. The terms “about” or “approximately” mean that quantities, dimensions, sizes, formulations, parameters, shapes and other characteristics need not be exact, but may be approximated and/or larger or smaller, as desired, reflecting acceptable tolerances, conversion factors, rounding off, measurement error and the like and other factors known to those of skill in the art. Recitations of quantities, dimensions, sizes, formulations, parameters, shapes and other characteristics should also be construed as if the term “substantially” precedes the quantity, dimension, size, formulation, parameter, shape or other characteristic. The terms “approximately,” “about,” and “substantially” as used herein represent an amount close to the stated amount that still performs a desired function or achieves a desired result. For example, in some embodiments, as the context may dictate, the terms “approximately”, “about”, and “substantially” may refer to an amount that is within less than or equal to 10% of the stated amount. The term “generally” as used herein represents a value, amount, or characteristic that predominantly includes, or tends toward, a particular value, amount, or characteristic. For example, as the context may dictate, the term “generally linear” can mean something that departs from exactly parallel by less than or equal to 15°.

Numerical data may be expressed or presented herein in a range format. It is to be understood that such a range format is used merely for convenience and brevity and thus should be interpreted flexibly to include not only the numerical values explicitly recited as the limits of the range, but also interpreted to include all of the individual numerical values or sub-ranges encompassed within that range as if each numerical value and sub-range is explicitly recited. As an illustration, a numerical range of “1 to 5” should be interpreted to include not only the explicitly recited values of about 1 to about 5, but should also be interpreted to also include individual values and sub-ranges within the indicated range. Thus, included in this numerical range are individual values such as 2, 3 and 4 and sub-ranges such as “1 to 3,” “2 to 4” and “3 to 5,” etc. This same principle applies to ranges reciting only one numerical value (e.g., “greater than 1”) and should apply regardless of the breadth of the range or the characteristics being described.

Any dimensions included in this disclosure are example dimensions of one or more components of a mask assembly in accordance with this disclosure, and are in mm.

A plurality of items may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. Furthermore, where the terms “and” and “or” are used in conjunction with a list of items, they are to be interpreted broadly, in that any one or more of the listed items may be used alone or in combination with other listed items. The term “alternatively” refers to selection of one of two or more alternatives, and is not intended to limit the selection to only those listed alternatives or to only one of the listed alternatives at a time, unless the context clearly indicates otherwise.

Reference to any prior art in this specification is not, and should not be taken as, an acknowledgement or any form of suggestion that that prior art forms part of the common general knowledge in the field of endeavour in any country in the world.

The disclosure may also be said broadly to consist in the parts, elements and features referred to or indicated in the specification of the application, individually or collectively, in any or all combinations of two or more of said parts, elements or features.

Where, in the foregoing description reference has been made to integers or components having known equivalents thereof, those integers are herein incorporated as if individually set forth.

It should be noted that various changes and modifications to the presently preferred embodiments described herein will be apparent to those skilled in the art.

Such changes and modifications may be made without departing from the spirit and scope of the disclosure and without diminishing its attendant advantages. For instance, various components may be repositioned as desired. It is therefore intended that such changes and modifications be included within the scope of the disclosure. Moreover, not all of the features, aspects and advantages are necessarily required to practice the present disclosure. Accordingly, the scope of the present disclosure is intended to be defined only by the claims that follow.

1-65. (canceled)

66. A mask assembly for respiratory therapy, comprising:
a mask frame comprising at least one headgear connector;
a cushion; and
an inlet connector configured to receive breathable gases from, and be connected to, a gas delivery conduit, the inlet connector being in fluid communication with the cushion;
wherein the mask frame and/or the inlet connector comprise one or more engagement features configured to resist relative rotation between the mask frame and inlet connector; wherein the one or more engagement features comprises a tab on one of the mask frame and the inlet connector, and a cut-out on the other of the mask frame and the inlet connector, the tab being received in the cut-out.

67. The mask assembly of claim 66, wherein the tab is provided at a bottom of the mask frame.

68. The mask assembly of claim 66, wherein the cut-out is provided at a bottom of the inlet connector.

69. The mask assembly of claim 66, wherein the tab and the cut-out are oblong.

70. The mask assembly of claim 66, wherein the mask frame comprises a central connection portion and wherein the inlet connector is mounted on the central connection portion.

71. The mask assembly of claim 70, wherein the mask frame comprises a frame clip configured to removably mount the mask frame on the inlet connector; wherein preferably the central connection portion comprises the frame clip.

72. The mask assembly of claim 71 wherein the frame clip comprises opposed ends between which is defined a mouth configured to receive the inlet connector to mount the frame clip on the mask assembly.

73. The mask assembly of claim 72 wherein the frame clip comprises a front wall, and side walls extending rearwardly from the front wall, each side wall comprising an opposed end, the opposed ends being spaced apart to define the mouth at a rear of the frame clip.

74. The mask assembly of claim 73 wherein a lower part of the inlet connector comprises:
a skirt that extends downwardly, wherein the skirt comprises a recess configured to received and engage with the frame clip of the mask frame; and
a guide flange that projects outwardly from the skirt below the recess.

75. The mask assembly of claim 74 wherein a central, forward portion of the guide flange is omitted to define the cut-out.

76. The mask assembly of claim 74 wherein the guide flange comprises sides which are inclined upwardly from front to rear, to guide the mask frame upwardly as the mask frame is pushed onto the inlet connector.

77. The mask assembly of claim 66, wherein the cut-out is provided in a lowermost flange of the inlet connector.

78. The mask assembly of claim 66, wherein the inlet connector comprises an inlet port configured to be connected to a source of pressurised gas, the inlet port comprising a central inlet axis.

79. The mask assembly of claim 78 wherein the central inlet axis is substantially vertical when the mask assembly is viewed from a front; and/or extends substantially downwardly when viewed from a side; and/or extends substantially downwardly when viewed from the front.

80. The mask assembly of claim 78 wherein the mask frame is removably mounted on the inlet connector by moving the mask frame onto the inlet connector in a mounting direction, the mounting direction intersecting the central inlet axis.

81. The mask assembly of claim 70 wherein the mask frame comprises lateral portions that extend away from the central connection portion, the lateral portions being relatively flexible.

82. The mask assembly of claim 66, wherein the mask frame comprises:
lateral arms, and an upper portion comprising a connecting frame member which extends over an upper portion of the cushion between the lateral arms.

83. A mask assembly for respiratory therapy, comprising:
a cushion;
an inlet connector configured to receive breathable gases from, and be connected to, a gas delivery conduit, the inlet connector being in fluid communication with the cushion and including an inlet connector portion with an outer curved connector surface; and
a mask frame comprising at least one headgear connector and a frame connector portion having an inner curved connector portion configured to engage with the outer curved connector surface;
wherein the frame connector portion and the inlet connector portion are configured to resist relative rotation between the mask frame and inlet connector.

84. The mask assembly of claim 83, wherein the inner curved connector portion comprises a concave, curved surface and the outer curved connector surface is a convexly curved surface.

85. The mask assembly of claim 83, additionally comprising a tab on one of the inlet connector portion and the frame connector portion, and a cut-out on the other of the inlet connector portion and the frame connector portion, wherein the tab is configured to be positioned within the cut-out when the connector portion is engaged with the frame connector portion.

86. The mask assembly of claim 83, wherein the frame connector portion and the inlet connector portion are configured to resist relative sliding of the inner curved connector portion around the outer curved connector surface.
